(19) United States
(12) Patent Application Publication
(10) Pub. No.: US 2023/0338501 A1
(43) Pub. Date: Oct. 26, 2023

(54) LIVE-ATTENUATED RNA HYBRID VACCINE TECHNOLOGY

(71) Applicant: ACCESS TO ADVANCED HEALTH INSTITUTE, Seattle, WA (US)

(72) Inventors: Neal VAN HOEVEN, Seattle, WA (US); Emily VOIGT, Seattle, WA (US)

(21) Appl. No.: 18/024,703

(22) PCT Filed: Jul. 4, 2021
(86) PCT No.: PCT/US2021/040393

§ 371 Date: Mar. 3, 2023

Related U.S. Application Data
(60) Provisional application No. 63/075,053, filed on Sep. 4, 2020.

(51) Int. Cl. A61K 39/12; C12N 7/00; A61P 31/14; A61K 9/107

(52) U.S. Cl. CPC A61K 39/12; C12N 7/00; A61P 31/14; A61K 9/1075; A61K 2039/53

(57) ABSTRACT

This disclosure provides ribonucleic acid (RNA) polynucleotides encoding replication-competent viral genomes that, when introduced to a subject, induce an active viral replication. The RNA may be provided naked or with an artificial RNA delivery system. The viral genome may be a full-length genome of an attenuated viral strain. For example, the RNA may encode an attenuated Chikungunya or yellow fever virus. The artificial RNA delivery system may be a lipid particle such as a lipid nanoparticle (LNP), a nanostructure lipid carrier (NLC), or a cationic nanoemulsion (CNE). This disclosure also provides methods of inducing an immune response, including protective immunity, by administering to a subject an RNA polynucleotide that encodes a replication-competent viral genome in an amount sufficient to cause viral replication in the subject. The immune response may include inducing the production of neutralizing antibodies at a level comparable to inoculation with a live-attenuated virus.

### CROSS REFERENCE TO RELATED APPLICATIONS

This application claims to priority to U.S. Provisional Application No. 63/075,053 entitled “Live-attenuated RNA Hybrid Vaccine Technology,” filed on Sep. 4, 2020, the disclosure of which is incorporated herein by reference in its entirety.

### STATEMENT REGARDING FEDERALLY SPONSORED RESEARCH OR DEVELOPMENT

This invention was made with government support under award number R43AI127053 from the National Institute of Allergy and Infectious Diseases of the National Institutes of Health. The government has certain rights in the invention.

### SEQUENCE LISTING

The Sequence Listing associated with this application is provided in text format in lieu of a paper copy and is hereby incorporated by reference into the specification. The name of the text file containing the Sequence Listing is 54PCT Sequence_Listing_ST25.txt. The text file is 180 KB, was created on Jul. 3, 2021, and is being submitted electronically concurrent with the filing of the specification.

### FIELD

The present disclosure relates generally to the field vaccines, specifically RNA vaccines.

### BACKGROUND

Nucleic acid-based vaccines represent attractive alternatives to traditional live-attenuated vaccines due to their ability to be rapidly adapted to new targets, and reliably manufactured using pre-developed sequence-independent methods. Recent advances in engineering the structure (Tavernier, G.; Andries, O.; Demeester, J.; Sanders, N. N.; De Smedt, S. C.; Rejman, J., mRNA as gene therapeutic: how to control protein expression. J Control Release 2011, 150 (3), 238-47) and formulation (Midoux, P.; Pichon, C., Lipid-based mRNA vaccine delivery systems. Expert Rev Vaccines 2015, 14 (2), 221-34) of RNA-based vaccines has led to advancement of RNA vaccine platforms targeting emerging infectious diseases. Recently, the SARS-CoV-2 pandemic has driven rapid development of mRNA vaccines against the coronavirus. mRNA vaccines induce immunity by encoding one or several antigenic proteins rather than a full viral genome.

Nucleic acid-based vaccine technology may be able to overcome manufacturing and safety challenges typical of traditional live-attenuated vaccines. Manufacture of many attenuated viral vaccines using traditional culture methods can be difficult with a significant failure rate. (Rodrigues, A. F.; Soares, H. R.; Guerreiro, M. R.; Alves, P. M.; Coroadinha, A. S., Viral vaccines and their manufacturing cell substrates: New trends and designs in modern vaccinology. Biotechnol J 2015, 10 (9), 1329-44; Plotkin, S.; Robinson, J. M.; Cunningham, G.; Iqbal, R.; Larsen, S., The complexity and cost of vaccine manufacturing—An overview. Vaccine 2017, 35 (33), 4064-4071). The level of viral attenuation in vaccine strains is often high, limiting the rapid replication of virus to high titers. The number of biological substrates allowed for viral culture by regulatory agencies is also highly limited. Even should an excellent culture system exist, high viral titers are often only achieved in adherent cell culture, limiting production capabilities (Genzel, Y.; Rodig, J.; Rapp, E.; Reichl, U., Vaccine production: upstream processing with adherent or suspension cell lines. Methods Mol Biol 2014, 1104, 371-93). Resulting vaccine product characteristics are often highly variable based on the biological system and culture conditions used, (Butler, M.; Reichl, U., Animal Cell Expression Systems. Adv Biochem Eng Biotechnol 2017; Ng, S.; Gisonni-Lex, L.; Azizi, A., New approaches for characterization of the genetic stability of vaccine cell lines. Hum Vaccin Immunother 2017, 13 (7), 1669-1672) as are the methods used to analyze the resulting materials. (Plotkin et al. supra; Minor, P. D., Live attenuated vaccines: Historical successes and current challenges. Virology 2015, 479-480, 379-92.) This results in a high regulatory burden, increased vaccine costs, high failure rates of manufacturing lots, and can lead to severe vaccine shortages. (Plotkin et al. supra; Gershman, M. D.; Angelo, K. M.; Ritchey, J.; Greenberg, D. P.; Muhammad, R. D.; Brunette, G., et al; Addressing a Yellow Fever Vaccine Shortage—United States, 2016-2017. MMWR Morb Mortal Wkly Rep 2017, 66 (17), 457-459; Ulmer, J. B.; Valley, U.; Rappuoli, R., Vaccine manufacturing: challenges and solutions. Nat Biotechnol 2006, 24 (11), 1377-83; Vidor, E.; Soubeyrand, B., Manufacturing DTaP-based combination vaccines: industrial challenges around essential public health tools. Expert Rev Vaccines 2016, 15 (12), 1575-1582; Robbins, M. J.; Jacobson, S. H., Analytics for vaccine economics and pricing: insights and observations. Expert Rev Vaccines 2015, 14 (4), 605-16.) For example, a live-attenuated vaccine against yellow fever has long been available but is notoriously difficult to manufacture. Indeed, difficulties in manufacturing have led to massive shortages in worldwide vaccine supplies, (Gershman et al. supra; Barrett, A. D., Yellow Fever in Angola and Beyond—The Problem of Vaccine Supply and Demand. N Engl J Med 2016, 375 (4), 301-3) contributing to the emergence of yellow fever outbreaks throughout Brazil and other endemic countries. (Goldani, L. Z., Yellow fever outbreak in Brazil, 2017. Braz J Infect Dis 2017, 21 (2), 123-124; Paules, C. I.; Fauci, A. S., Yellow Fever—Once Again on the Radar Screen in the Americas. N Engl J Med 2017, 376 (15), 1397-1399; The, L., Yellow fever: a global reckoning. Lancet 2016, 387 (10026), 1348.)

Safety issues are also inherent in the use of biological culture for vaccine manufacture. Contamination of vaccine materials has resulted from biological culture contamination during manufacture. (Pastoret, P. P., Human and animal vaccine contaminations. Biologicals 2010, 38 (3), 332-4; In Immunization Safety Review: SV40 Contamination of Polio Vaccine and Cancer, Stratton, K.; Almario, D. A.; McCormick, M. C., Eds. Washington (DC), 2002). Viral source material must also be consistent and regulated, as passage and expansion of live-attenuated viral strains during manufacturing may lead to genetic drift, which may in turn affect vaccine safety and immunogenicity profiles. (Minor et al. supra; Skowronski, D. M.; Janjua, N. Z.; De Serres, G.; Sabaiduc, S.; Eshaghi, A.; Dickinson, J. A., et al.; Low 2012-13 influenza vaccine effectiveness associated with mutation in the egg-adapted H3N2 vaccine strain not antigenic drift in circulating viruses. PLoS One 2014, 9 (3), e92153; Kew, O., Reaching the last one percent: progress and challenges in global polio eradication. Curr Opin Virol 2012, 2 (2), 188-98.)

CHIKV is an emerging tropical arbovirus transmitted by the mosquito A. aegypti that typically results in fever, rash, and debilitating arthralgia and arthritis that can last months to years after infection (Weaver, S. C.; Lecuit, M., Chikungunya virus and the global spread of a mosquito-borne disease. N Engl J Med 2015, 372 (13), 1231-9; Goupil, B. A.; Mores, C. N., A Review of Chikungunya Virus-induced Arthralgia: Clinical Manifestations, Therapeutics, and Pathogenesis. Open Rheumatol J 2016, 10, 129-140.) No approved vaccine against CHIKV yet exists. Reactogenicity problems plagued the original traditionally-developed live-attenuated CHIKV vaccine (181/25 strain) derived in the 1980s. (Levitt, N. H.; Ramsburg, H. H.; Hasty, S. E.; Repik, P. M.; Cole, F. E., Jr.; Lupton, H. W., Development of an attenuated strain of chikungunya virus for use in vaccine production. Vaccine 1986, 4 (3), 157-62.) Despite efficacy demonstrated in Phase I and II clinical trials, arthralgia was reported in approximately 8% of 181/25 vaccinees, leading to the halt of 181/25-based vaccine development. (Edelman, R.; Tacket, C. O.; Wasserman, S. S.; Bodison, S. A.; Perry, J. G.; Mangiafico, J. A., Phase II safety and immunogenicity study of live chikungunya virus vaccine TSI-GSD-218. Am J Trop Med Hyg 2000, 62 (6), 681-5). CHIKV strain 181/25 was also demonstrated to be transmitted by the natural A. aegypti mosquito vector, leading to further concerns about vaccine containment. (Turell, M. J.; Malinoski, F. J., Limited potential for mosquito transmission of a live, attenuated chikungunya virus vaccine. Am J Trop Med Hyg 1992, 47 (1), 98-103.) Later studies of the 181/25 viral strain indicated that viral attenuation was due to only two point mutations in the CHIKV envelope protein. (Gorchakov, R.; Wang, E.; Leal, G.; Forrester, N. L.; Plante, K.; Rossi, S. L. et al. Attenuation of Chikungunya virus vaccine strain 181/clone 25 is determined by two amino acid substitutions in the E2 envelope glycoprotein. J Virol 2012, 86 (11), 6084-96.) This led to serious concerns about the genetic stability of the 181/25 vaccine virus strain. Indeed, the noted arthralgia in many vaccinees may be attributable to reversion of the 181/25 virus strain to a fully pathogenic phenotype during or post manufacture, as evidence of such reversion has been observed in experimental 181/25 infection of mice followed by viral sequencing. (Gorchakov et al., supra).

While non-replicating inactivated or virus-like particle (VLP)-based CHIKV vaccines have been described that would overcome such safety concerns, (Akahata, W.; Yang, Z. Y.; Andersen, H.; Sun, S.; Holdaway, H. A.; Kong, W. P. et al. A virus-like particle vaccine for epidemic Chikungunya virus protects nonhuman primates against infection. Nat Med 2010, 16 (3), 334-8; Chang, L. J.; Dowd, K. A.; Mendoza, F. H.; Saunders, J. G.; Sitar, S.; Plummer, S. H. et al., Safety and tolerability of chikungunya virus-like particle vaccine in healthy adults: a phase 1 dose-escalation trial. Lancet 2014, 384 (9959), 2046-52; Metz, S. W.; Gardner, J.; Geertsema, C.; Le, T. T.; Goh, L.; Vlak, J. M. et al., Effective chikungunya virus-like particle vaccine produced in insect cells. PLoS Negl Trop Dis 2013, 7 (3), e2124; Saraswat, S.; Athmaram, T. N.; Parida, M.; Agarwal, A.; Saha, A.; Dash, P. K., Expression and Characterization of Yeast Derived Chikungunya Virus Like Particles (CHIK-VLPs) and Its Evaluation as a Potential Vaccine Candidate. PLoS Negl Trop Dis 2016, 10 (7), e0004782.) VLP-based vaccines often require the use of adjuvants and booster doses, (Cimica, V.; Galarza, J. M., Adjuvant formulations for virus-like particle (VLP) based vaccines. Clin Immunol 2017, 183, 99-108) while high manufacturing costs often pose a significant challenge to the clinical practicality of such vaccine strategies. Live-replicating CHIKV strains with additional, more stable attenuating mutations and live-replicating chimeric CHIKV vaccines have been created as potential viral vaccines (Plante, K.; Wang, E.; Partidos, C. D.; Weger, J.; Gorchakov, R.; Tsetsarkin, K. et al., Novel chikungunya vaccine candidate with an IRES-based attenuation and host range alteration mechanism. PLoS Pathog 2011, 7 (7), e1002142; Hallengard, D.; Kakoulidou, M.; Lulla, A.; Kummerer, B. M.; Johansson, D. X.; Mutso, M. et al., Novel attenuated Chikungunya vaccine candidates elicit protective immunity in C57BL/6 mice. J Virol 2014, 88 (5), 2858-66; Rogues, P.; Ljungberg, K.; Kummerer, B. M.; Gosse, L.; Dereuddre-Bosquet, N.; Tchitchek, N. et al., Attenuated and vectored vaccines protect nonhuman primates against Chikungunya virus. JCI Insight 2017, 2 (6), e83527; Erasmus, J. H.; Auguste, A. J.; Kaelber, J. T.; Luo, H.; Rossi, S. L.; Fenton, K. et al., A chikungunya fever vaccine utilizing an insect-specific virus platform. Nat Med 2017, 23 (2), 192-199; Ramsauer, K.; Schwameis, M.; Firbas, C.; Mullner, M.; Putnak, R. J.; Thomas, S. J. et al., Immunogenicity, safety, and tolerability of a recombinant measles-virus-based chikungunya vaccine: a randomised, double-blind, placebo-controlled, active-comparator, first-in-man trial. Lancet Infect Dis 2015, 15 (5), 519-27) and appear to be the most practical candidates for safe and effective single-dose immunization against CHIKV. Manufacture of such live-attenuated CHIKV strains, however, involves all the manufacturing challenges and safety issues mentioned above. Introduction of such live-attenuated RNA vaccine strains using a hybrid live-attenuated RNA vaccine technology could streamline manufacture of such vaccines, as well as reducing potential for culture contamination and genetic drift.

DNA vaccines against CHIKV have previously been created by several scientific teams with a similar goal of harnessing the safety, manufacturability, and reliability of nucleic acid-based vaccines. (Hallengard et al., supra; Rogues et al., surpa; Muthumani, K.; Block, P.; Flingai, S.; Muruganantham, N.; Chaaithanya, I. K.; Tingey, C. et al., Rapid and Long-Term Immunity Elicited by DNA-Encoded Antibody Prophylaxis and DNA Vaccination Against Chikungunya Virus. J Infect Dis 2016, 214 (3), 369-78; Muthumani, K.; Lankaraman, K. M.; Laddy, D. J.; Sundaram, S. G.; Chung, C. W.; Sako, E. et al., Immunogenicity of novel consensus-based DNA vaccines against Chikungunya virus. Vaccine 2008, 26 (40), 5128-34; Mallilankaraman, K.; Shedlock, D. J.; Bao, H.; Kawalekar, O. U.; Fagone, P.; Ramanathan, A. A. et al., A DNA vaccine against chikungunya virus is protective in mice and induces neutralizing antibodies in mice and nonhuman primates. PLoS Negl Trop Dis 2011, 5 (1), e928; Tretyakova, I.; Hearn, J.; Wang, E.; Weaver, S.; Pushko, P., DNA vaccine initiates replication of live attenuated chikungunya virus in vitro and elicits protective immune response in mice. J Infect Dis 2014, 209 (12), 1882-90.) Indeed, several groups have used DNA to launch full-length replication-competent live attenuated CHIKV strains in a similar bid to harness the advantages of nucleic acid vaccine technology in combination with the proven immunogenicity and reliability of live-attenuated vaccines. “Immunization DNA” was used to deliver full-length cDNA of attenuated CHIKV virus genomes to BALB/c mice and resulted in the development of CHIKV-neutralizing antibodies and protection of mice against virulent CHIKV challenge. (Tretyakova, I.; Hearn, J.; Wang, E.; Weaver, S.; Pushko, P., DNA vaccine initiates replication of live attenuated chikungunya virus in vitro and elicits protective immune response in mice. J Infect Dis 2014, 209 (12), 1882-90.) Similarly, another group has administered DNA encoding genomes of the live-attenuated CHIKV strains CHIKV 181/25-Δ5nsP3 and CHIKV 181/25-A6K by electroporation of C57BL/6 mice, resulting in antibody responses and protection against viremia and joint swelling. (Hallengard, et al., supra.) However, all of these DNA-based vaccine platforms require electroporation of vaccine-injected mouse muscle to enable DNA entry into target cells.

Nucleic acid-based vaccines such as mRNA vaccines and DNA vaccines address some of the problems with live-attenuated vaccines. However, each comes with its own challenges and limitations. It would be beneficial to harness the strengths of both vaccine types, combining the ease, reliability, and safety inherent in nucleic acid vaccine manufacture with the proven immunogenicity of live-attenuated viral vaccines. The present disclosure fulfills these needs and offers other related advantages.

### BRIEF SUMMARY

In one aspect, this disclosure provides a composition for causing viral infection in a subject. The composition may be a vaccine. The composition includes a ribonucleic acid (RNA) polynucleotide encoding a replication-competent viral genome and an artificial RNA delivery system. The RNA is present in an amount sufficient to cause to viral replication in the subject. The RNA may encode the genome of an attenuated virus and it may be a full-length genome. The viral genome may be a genome of an alphavirus, a flavivirus, a coronavirus, or other type of positive stranded virus. The RNA delivery system may be any system effective for delivering RNA to a cell. Examples of RNA delivery systems include lipid nanoparticles (LNP), nanostructured lipid carriers (NLCs), cationic nanoemulsions (CNE), and amphiphilic diblock oligomers containing a sequence of lipid monomers and a sequence of cationic monomers.

In one aspect, this disclosure provides a method of inducing an immune response in a subject by administering to an RNA polynucleotide encoding a replication-competent viral genome in an amount sufficient to cause viral replication in the subject. The immune response may be an immune response that provides protective immunity against the virus encoded by the RNA polynucleotide. For example, the immune response may induce the production of neutralizing antibodies. The amount of RNA polynucleotide administered to the subject may be sufficient to cause viral replication in the subject. In some implementations, the RNA polynucleotide is administered with an artificial RNA delivery system such as a lipid particle (e.g., LNP, NLC, or CNE). The immune response may be induced by a single dose of vaccine. Additionally, administration may be performed without electroporation or use of a biolistic particle delivery system.

This disclosure also provides a hybrid live-attenuated RNA vaccine, in which full-length replication-competent attenuated viral genomes are delivered in vitro to the site of vaccination. An RNA vaccine delivery vehicle is used in some implementations. This vaccine technology is broadly applicable to positive stranded viruses. This vaccine is an easily manufactured product with no need for biological culture, resulting in a reliable and stable genetic profile ensuring consistent safety and reactogenicity. This technology allows ready manufacturing in a cell-free environment, regardless of viral attenuation level, and promises to avoid many safety and manufacturing challenges of traditional live-attenuated vaccines.

In one illustrative implementation, this technology is demonstrated through development and testing of live-attenuated RNA hybrid vaccines against chikungunya virus (CHIKV) and yellow fever virus (YF), comprised of an in vitro-transcribed highly-attenuated viral genome delivered by a highly stable nanostructured lipid carrier (NLC) formulation as an intramuscular or subcutaneous injection. A single-dose immunization of immunocompetent C57BL/6 mice with either the chikungunya virus or yellow fever virus live-attenuated RNA hybrid vaccine results in induction of high CHIKV- or YFV-neutralizing antibody titers, and demonstrated protection against mortality and footpad swelling after lethal CHIKV challenge in the CHIKV vaccine case.

Such hybrid live-attenuated nucleic acid vaccines may be reliably and rapidly manufactured in a cell-free, sequence-independent process that overcomes many of the ongoing production and safety challenges inherent in the manufacture of live-attenuated viral vaccines. As a sequence-independent process, this hybrid live-attenuated/RNA vaccine technology allows for the use of highly-attenuated virus strains in vaccines, thereby increasing both the genetic attenuation stability and safety profile of the vaccine.

It is to be understood that one, some, or all of the properties of the various implementations described herein may be combined to form other implementations of the present invention. These and other aspects of the present invention will become evident upon reference to the following detailed description and attached drawings.

### BRIEF DESCRIPTION OF THE DRAWINGS

FIG. 1 shows schematics of RNA constructs used as CHIK vaccine candidates.

FIG. 2 is agarose gels showing free RNA from each NLC-formulated RNA vaccine candidate (none), extracted RNA from each NLC-formulated RNA vaccine candidate, and extracted RNA from vaccine candidates after challenge with RNase A.

FIG. 3 shows virus-like particles (VLPs) collected by ultracentrifugation of transfected cell supernates 72 hours post-transfection, resuspension of VLP pellets in PBS, BCA assay for total protein quantification, and western blot with equal protein loading across samples, alongside purified Chikungunya E1 protein (˜50 kDa).

FIG. 4 shows growth curves of infectious attenuated viral strains rescued from RNA-transfected Vero cells. Rescued virus strains were passaged twice and level of attenuation relative to CHIKV-LR was measured by growth curves (MOI=0.01) on Vero cells. Supernate virus content was measured by qRT-PCR of viral genomes (FIG. 4A) or plaque assay (FIG. 4B). Datapoints represent mean values from biological triplicate samples±SEM.

FIG. 5 shows CHIKV-neutralizing antibody titers in wild-type C57BL/6 mice (n=20/group) 28 days post-vaccination with 1 μg (whole-genome) or 5 (mRNA) RNA vaccine candidates complexed with NLC or CHIKV 181/25 virus as a positive control (one-way ANOVA (6 DoF, F=58.5) followed by Dunnett's multiple comparisons test. Datapoints represent arithmetic means±SEM. *<0.05, **<0.005, ***<0.0005 by Dunnett's multiple comparisons test.

FIG. 6 shows survival rates of vaccinated wild-type C57BL/6 mice challenged lethally with 10³ pfu/mouse of CHIKV-LR after IP injection of 2 mg Marl IFNAR-blocking antibody (n=10/group). Mouse survival was monitored daily.

FIG. 7 shows serum virus titers in transiently immunocompromised wild-type C57BL/6 mice challenged lethally with 10³ pfu/mouse of CHIKV-LR (FIG. 7A) and in mice challenged non-lethally with 10⁵ pfu/mouse of CHIKV-LR (FIG. 7B) (n=10/group)(one-way ANOVA, 6 DoF, F=14.1 and 7.1, respectively). Serum samples were taken from a subset of mice (n=5) 2 days post-challenge for measurement of viremia. *<0.05, **<0.005, ***<0.0005 by Dunnett's multiple comparisons test.

FIG. 8 shows footpad breadth of vaccinated wild-type C57BL/6 mice challenged non-lethally with 10⁵ pfu/mouse of CHIKV-LR (n=10/group) to monitor CHIKV-induced arthralgia. Footpad breadth was measured daily. Datapoints represent arithmetic means±SEM.

FIG. 9 shows CHIKV-neutralizing antibody titers 28 days post-vaccination in wild-type C57BL/6 mice (n=10/group) immunized i.m. with 0.1 μg, 1 μg, or 10 μg of whole-genome RNA vaccine candidates CHIKV 181/25 or CHIKV 181/25Δ5nsP3. Vaccination with 10⁴ pfu/mouse of each attenuated virus served as positive vaccination control groups. Two-tailed homoscedastic t-tests were used on log-normalized PRNT data to compare neutralizing Ab titers induced by the 10 μg RNA vaccine dose with Ab titers induced by the respective live-attenuated viral vaccine control (DoF=9).

FIG. 10 shows post-lethal challenge viremia measured by plaque assay in wild-type C57BL/6 mice (n=10/group) immunized i.m. with 0.1 μg, 1 μg, or 10 μg of whole-genome RNA vaccine candidates CHIKV 181/25 or CHIKV 181/25Δ5nsP3. Vaccination with 10⁴ pfu/mouse of each attenuated virus served as positive vaccination control groups.

FIG. 11 shows survival rates of wild-type C57BL/6 mice challenged lethally with 10³ pfu/mouse of CHIKV-LR after IP injection of 2 mg Marl IFNAR-blocking antibody (n=10/group). Mouse survival was monitored daily. The mice were vaccinated with the indicated doses of CHIKV 181/25 (FIG. 11A) or CHIKV 181/25-Δ5nsP3 (FIG. 11B) RNA based vaccines. Vaccination with 10⁴ pfu/mouse of each attenuated virus served as positive vaccination control groups (“virus”).

FIG. 12 shows mouse footpad swelling as indicated by footpad width×breadth measurements after immunocompromisation and lethal challenge (n=10/group) as shown in FIG. 11. The mice were vaccinated with the indicated doses of CHIKV 181/25 (FIG. 12A) or CHIKV 181/25-Δ5nsP3 (FIG. 12B) RNA based vaccines. Vaccination with 10⁴ pfu/mouse of each attenuated virus served as positive vaccination control groups (“virus”). Datapoints represent arithmetic means±SEM.

FIG. 13 shows schematics of an RNA construct used as a yellow fever vaccine candidate.

FIG. 14 shows in vivo immune response in wild-type C57BL/6 mice (n=4/group) 28 days post-vaccination with 1 μg or 10 μg of YFV RNA vaccine candidates complexed with NLC. SEAP rvRNA complexed with NLC is used as the negative “mock-vaccinated” control. FIG. 14A shows yellow fever neutralizing antibody titers. Accepted correlate of protection is a PRNT titer of 1:10. FIG. 14B shows yellow fever E protein-specific IgG antibody titers detected by ELISA. Data is shown as geometric mean+/−geometric standard deviation.

### DETAILED DESCRIPTION

This disclosure presents a proof-of-principle that RNA polynucleotides encoding genomes of positive stranded viruses can be used to create infections in subjects without inoculation of live-attenuated virus, a method referred to herein as “live-attenuated RNA hybrid vaccines.” Although broadly applicable to any positive stranded virus, examples provided show that this technology can produce protective immune responses against chikungunya and yellow fever.

This disclosure demonstrates that an effective CHIKV vaccine can be created by delivering replication-competent attenuated CHIKV genomes to the site of vaccination using RNA vaccine technology. This vaccine technology allowed for the production of replication-competent virus-like particles in vitro capable of presenting CHIKV epitopes to appropriate immune cells in vivo. In vivo studies demonstrate the ability of this CHIKV hybrid live-attenuated RNA vaccine to induce significant CHIKV-neutralizing antibody titers in immunocompetent mice after a single immunization in a dose-dependent manner. A transiently-immunocompromised murine lethal challenge model demonstrates vaccine-induced protection against CHIKV-mediated morbidity and mortality. The vaccine demonstrated the ability to protect even transiently-immunocompromised mice from death, viremia, and footpad swelling after lethal challenge with virulent CHIKV-LR.

This disclosure also establishes a model for CHIKV lethal challenge in interferon-competent mice. By intraperitoneal injection (IP) injection of IFNAR blocking antibodies prior to CHIKV-LR virus challenge, wild-type C57BL/6 mice are sufficiently immunocompromised to achieve reliable lethality in unprotected mice. Use of immunocompetent mice with intact innate immune signaling systems is important for live, replicating vaccine efficacy testing to prevent overestimation of vaccine immunogenicity. This model accordingly allows for the progression of normal immune responses to vaccination, while also providing a challenge model for proof of vaccine efficacy beyond footpad swelling measures alone. Indeed, in such transiently immunocompromised mice, footpad swelling in this model appears to be a more sensitive measure of vaccine protection from CHIKV than is footpad swelling in wild-type C57BL/6 mice, a common model of CHIKV protection.

Manufacturing for both the RNA and artificial RNA delivery systems of these live-attenuated RNA hybrid vaccines is done in cell-free environments, avoiding the potential for biological materials to become contaminated and affect vaccine quality, as has been a rare but serious issue in the manufacture of live-attenuated vaccines. An additional safety benefit is conferred by the nature of the RNA vaccine material, which does not need to be passaged and expanded as do live-attenuated virus strains. As a result of direct translation from a DNA backbone by the relatively low-error polymerase, such as T7 RNA polymerase, the vaccine RNA has a consistent and easily characterized sequence, unlike the genetically diverse pseudospecies typically found in live-attenuated vaccines against RNA viruses.

Even live-attenuated Chikungunya vaccine strains engineered to have a particularly high-fidelity polymerase (fidelity variants), which demonstrated efficacy in mice (Weiss, C. M.; Liu, H.; Riemersma, K. K.; Ball, E. E.; Coffey, L. L., Engineering a fidelity-variant live-attenuated vaccine for chikungunya virus. NPJ Vaccines 2020, 5, 97), showed stable or even increased accumulation of mutations after passaging in cell culture (Weiss et al., supra; Riemersma, K. K.; Steiner, C.; Singapuri, A.; Coffey, L. L., Chikungunya Virus Fidelity Variants Exhibit Differential Attenuation and Population Diversity in Cell Culture and Adult Mice. J Virol 2019, 93 (3)), resulting in safety concerns. Increased genetic diversity of live-attenuated Chikungunya vaccines have also been suggested as potentially impairing the development of neutralizing antibodies (Riemersma et al., supra).

Others have demonstrated that DNA-launched 181/25-derived Chikungunya vaccine virus genomes have a higher level of genetic uniformity than even a minimally-passaged 181/25 viral strain, with significantly lower frequency of single-nucleotide polymorphisms, including at the two mutation sites in the 181/25 virus that are responsible for attenuation (Hidajat, R.; Nickols, B.; Forrester, N.; Tretyakova, I.; Weaver, S.; Pushko, P., Next generation sequencing of DNA-launched Chikungunya vaccine virus. Virology 2016, 490, 83-90). A similarly high level of uniformity and reduced genetic diversity is also expected with the hybrid live-attenuated RNA vaccine technology of this disclosure. Any polymerase-introduced mutations to the original genome will be randomly assorted across the genome rather than due to selective pressure. Thus, use of in vitro transcription direct from a plasmid can result in better genetic stability and safety profiles for RNA-delivered genomes, free of genetic drift.

One advantage of nucleic acid vaccines is their reliable, sequence-independent manufacturability. Such manufacturing requires little to no specialized equipment not already found in standard GMP facilities. DNA plasmid manufacture is established GMP technology; in vitro RNA transcription and NLC formulation manufacture are GMP-friendly and easily adapted to new vaccine sequences.

This method of vaccine development may be applied to other positive-stranded RNA viruses besides chikungunya and yellow fever, allowing for reliable manufacture of live-attenuated RNA hybrid vaccines of even highly-attenuated virus strains. Positive-stranded RNA viruses comprise a broad class of viruses, causing numerous important human pathogens such as SARS, hepatitis C, Coxsackie virus, West Nile, and polio, among many others. This method of vaccine development allows for straightforward, sequence-independent, cell-free manufacturing compared to traditional live-attenuated vaccine manufacturing methods. Thus, the techniques of this disclosure may be used to supplement stores of already-existing viral vaccines limited by cell-based manufacturing difficulties, and/or scale-up and commercialize otherwise un-manufacturable highly-attenuated vaccine strains. For example, this hybrid RNA vaccine technology has use in the manufacture and delivery of yellow fever vaccines for which there is an existing attenuated viral strain YF-17D. The vaccine virus RNA may be administered by standard intramuscular (IM) injection, bypassing the current cell-based YF vaccine manufacturing processes and relieving vaccine shortages due to the challenges of manufacturing.

### I. Definitions

The following terms have the following meanings unless otherwise indicated. Any undefined terms have their art recognized meanings.

In the present description, the terms “about,” “around,” “approximately,” and similar referents mean±20% of the indicated range, value, or structure, unless otherwise indicated.

The use of the alternative (e.g., “or”) should be understood to mean either one, both, or any combination thereof of the alternatives.

As used herein, the terms “include,” “have” and “comprise” are used synonymously, which terms and variants thereof are intended to be construed as nonlimiting.

As used herein and in the appended claims, the singular forms “a,” “an,” and “the” include plural reference unless the context clearly indicates otherwise.

By “disease” is meant any condition or disorder that damages or interferes with the normal function of an organism, cell, tissue, or organ. Examples of diseases include viral infections including but not limited to those caused by positive strand RNA viruses such as chikungunya and yellow fever.

As used herein, the term “vaccine” refers to a formulation which contains an antigen or nucleic acid encoding an antigen, which is in a form that is capable of being administered to a subject and which induces a protective immune response sufficient to induce immunity to prevent and/or ameliorate an infection and/or to reduce at least one symptom of an infection and/or to enhance the efficacy of a subsequent vaccine dose. Typically, the vaccine comprises a conventional saline or buffered aqueous solution medium in which the composition of the present invention is suspended or dissolved. Upon introduction into a subject, the vaccine is able to provoke an immune response including, but not limited to, the production of antibodies and/or cytokines and/or the activation of cytotoxic T cells, antigen presenting cells, helper T cells, dendritic cells and/or other cellular responses.

An “infectious” virus particle is one that can introduce the virus genome into a permissive cell, typically by viral transduction. Upon introduction into the target cell, the genomic nucleic acid serves as a template for RNA transcription (i.e., gene expression). The “infectious” virus-like particle may be “replication-competent” (i.e., results in a productive infection in which new virus particles are produced). In embodiments of the invention, the “infectious” virus-like particle includes a replicon particle that can introduce the genomic nucleic acid (i.e., replicon) into a host cell and is “replication-competent”.

A “highly-attenuated virus” or “highly-attenuated strain” is a virus strain that is unable to replicate or replicates poorly in human cells. In contrast, a viral strain is considered non-highly attenuated if the virus maintains its capacity to replicate productively in mammalian cells.

“Purified” means that the molecule has been increased in purity, such that it exists in a form that is more pure than it exists in its natural environment and/or when initially synthesized and/or amplified under laboratory conditions. Purity is a relative term and does not necessarily mean absolute purity.

A “polynucleotide,” “oligonucleotide,” or “nucleic acid,” as used interchangeably herein, refer to polymers of nucleotides of any length, include DNA and RNA. The nucleotides can be, for example, deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer.

An “individual” or a “subject” is any mammal. Mammals include, but are not limited to humans, primates, farm animals, sport animals, pets (such as cats, dogs, horses), and rodents.

A “replicon” as used herein includes any genetic element, for example, a plasmid, cosmid, bacmid, phage or virus that is capable of replication largely under its own control. A replicon may be either RNA or DNA and may be single or double stranded.

As used herein, the terms “express,” “expresses,” “expressed” or “expression,” and the like, with respect to a nucleic acid sequence (e.g., RNA or DNA) indicates that the nucleic acid sequence is transcribed and, optionally, translated. Thus, a nucleic acid sequence may express a polypeptide of interest or a functional untranslated RNA.

The term “recombinant” as used herein to describe a nucleic acid molecule means a polynucleotide of genomic, cDNA, viral, semisynthetic, or synthetic origin which does not occur in nature or by virtue of its origin or manipulation is associated with or linked to another polynucleotide in an arrangement not found in nature. The term “recombinant” as used with respect to a protein or polypeptide means a polypeptide produced by expression of a recombinant polynucleotide.

Ranges provided herein are understood to be shorthand for all of the values and sub-ranges within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all sub-ranges such as 2-50, 3-50, 5-45, 1-49, 1-48, etc.

The practice of the present disclosure will employ, unless otherwise indicated, conventional techniques of molecular biology, recombinant DNA, biochemistry, and chemistry, which are within the skill of the art. Such techniques are explained fully in the literature. See, e.g., Molecular Cloning A Laboratory Manual, 2nd Ed., Sambrook et al., ed., Cold Spring Harbor Laboratory Press: (1989); DNA Cloning, Volumes I and II (D. N. Glover ed., 1985); Oligonucleotide Synthesis (M. J. Gait ed., 1984); Mullis et al., U.S. Pat. No. 4,683,195; Nucleic Acid Hybridization (B. D. Hames & S. J. Higgins eds. 1984); B. Perbal, A Practical Guide to Molecular Cloning (1984); the treatise, Methods in Enzymology (Academic Press, Inc., N.Y.); and in Ausubel et al., Current Protocols in Molecular Biology, John Wiley and Sons, Baltimore, Maryland (1989).

### II. Positive Stranded RNA Viruses

Positive-strand RNA viruses (+ssRNA viruses) are a group of related viruses that have positive-sense, single-stranded genomes made of ribonucleic acid. The positive-sense genome can act as messenger RNA (mRNA) and can be directly translated into viral proteins by the host cell's ribosomes. Positive-strand RNA viruses encode an RNA-dependent RNA polymerase (RdRp) which is used during replication of the genome to synthesize a negative-sense antigenome that is then used as a template to create a new positive-sense viral genome.

Positive-strand RNA virus genomes usually contain relatively few genes, usually between three and ten, including an RNA-dependent RNA polymerase. Coronaviruses have the largest known RNA genomes, between 27 and 32 kilobases in length, and likely possess replication proofreading mechanisms in the form of an exoribonuclease within nonstructural protein nsp14.

Positive-strand RNA viruses have genetic material that can function both as a genome and as messenger RNA; it can be directly translated into protein in the host cell by host ribosomes. The first proteins to be expressed after infection serve genome replication functions; they recruit the positive-strand viral genome to viral replication complexes formed in association with intracellular membranes. These complexes contain proteins of both viral and host cell origin and may be associated with the membranes of a variety of organelles—often the rough endoplasmic reticulum, but also including membranes derived from mitochondria, vacuoles, the Golgi apparatus, chloroplasts, peroxisomes, plasma membranes, autophagosomal membranes, and novel cytoplasmic compartments.

The replication of the positive-sense RNA genome proceeds through double-stranded RNA intermediates, and the purpose of replication in these membranous invaginations may be the avoidance of cellular response to the presence of dsRNA. In many cases subgenomic RNAs are also created during replication. After infection, the entirety of the host cell's translation machinery may be diverted to the production of viral proteins as a result of the very high affinity for ribosomes by the viral genome's internal ribosome entry site (IRES) elements; in some viruses, such as poliovirus and rhinoviruses, normal protein synthesis is further disrupted by viral proteases degrading components required to initiate translation of cellular mRNA.

All positive-strand RNA virus genomes encode an RNA-dependent RNA polymerase, a viral protein that synthesizes RNA from an RNA template. Host cell proteins recruited by +ssRNA viruses during replication include RNA-binding proteins, chaperone proteins, and membrane remodeling and lipid synthesis proteins, which collectively participate in exploiting the cell's secretory pathway for viral replication.

RNA viruses can be subdivided into groups based on type of RNA that serves as the genome. Positive or plus (+)-strand RNA viruses have genomes that are functional mRNAs. Upon penetration into the host cell, ribosomes assemble on the genome to synthesize viral proteins. Genomes of positive-strand RNA viruses are single-stranded molecules of RNA and may be capped and polyadenylated. During the replication cycle of positive-strand RNA viruses, among the first proteins to be synthesized are those needed to synthesize additional genomes and mRNAs. Thus, the infecting genome has two functions: It is an mRNA and also serves as the template for synthesis of additional viral RNAs. A functional definition of a positive-strand virus is that purified or chemically synthesized genomes are infectious.

A. Attenuated Viruses

The methods of the present invention may also be carried out with the viral genome of an attenuated virus. An “attenuated” or “live-attenuated” virus strain refers to a mutated, modified, variant and/or recombinant virus having reduced or no virulence or pathogenicity or propensity to cause a disease or infection in healthy individuals as normally associated with the wildtype or unmodified, non-mutated virus. In general, an “attenuated” or “live-attenuated” virus has been modified to decrease or eliminate its pathogenicity, while maintaining its viability for replication within a target host and while remaining sufficiently immunogenic to prevent or inhibit wild-type viral infection and/or pathogenicity. The phrases “attenuating mutation” and “attenuating amino acid,” as used herein, mean a nucleotide sequence containing a mutation, or an amino acid encoded by a nucleotide sequence containing a mutation, which mutation results in a decreased probability of causing disease in its host (i.e., reduction in virulence), in accordance with standard terminology in the art. See, e.g., B. Davis et al., Microbiology 132 (3d ed. 1980). The phrase “attenuating mutation” excludes mutations or combinations of mutations that would be lethal to the virus.

Those skilled in the art may identify attenuating mutations other than those specifically disclosed herein using other methods known in the art, e.g., looking at neurovirulence in weanling or adult mice following intracerebral injection. Methods of identifying attenuating mutations in alphaviruses are described by Olmsted et al., (1984) Science 225:424 and Johnston and Smith, (1988) Virology 162:437; the disclosures of which are incorporated herein in their entireties.

To identify other attenuating mutations other than those specifically disclosed herein, amino acid substitutions may be based on any characteristic known in the art, including the relative similarity or differences of the amino acid side-chain substituents, for example, their hydrophobicity, hydrophilicity, charge, size, and the like.

B. Alphaviruses

As used herein “alphavirus” is meant to refer to RNA-containing viruses that belong to the group IV Togaviridae family of viruses. Alphaviruses includes Eastern Equine Encephalitis virus (EEE), Venezuelan Equine Encephalitis virus (VEE), Everglades virus, Mucambo virus, Pixuna virus, Western Encephalitis virus (WEE), Sindbis virus, South African Arbovirus No. 86 (S.A.AR86), Girdwood S.A. virus, Ockelbo virus, Semliki Forest virus, Middelburg virus, Chikungunya virus, O'Nyong-Nyong virus, Ross River virus, Barmah Forest virus, Getah virus, Sagiyama virus, Bebaru virus, Mayaro virus, Una virus, Aura virus, Whataroa virus, Babanki virus, Kyzlagach virus, Highlands J virus, Fort Morgan virus, Ndumu virus, Buggy Creek virus, and any other virus classified by the International Committee on Taxonomy of Viruses (ICTV) as an alphavirus. The alphavirus genus consists of 31 distinct species (along with O'nyong'nyong virus, Ross River virus, Sindbis virus, Semliki Forest virus, VEE and others) that either cause encephalitis, febrile illness with arthralgia, or are not known to cause disease in humans. Members of this genus are primarily vector-borne; nearly all of them are utilizing mosquitoes as their invertebrate vectors (Powers and Brault, 2009).

Like all alphaviruses, CHIKV has a genome consisting of a linear, positive sense, single-stranded RNA molecule of approximately 12 kb in length (Khan et al., 2002). The nonstructural proteins required for viral replication are encoded in the 5′ two thirds of the genome and are regulated from 49S promoter, while the structural genes are collinear with the 3′ one-third and utilize 26S internal promoter. The 5′ end of the genome has a 7-methylguanosine cap while the 3′ end is polyadenylated. There are also 3′ noncoding repeat sequence elements that generate predicted secondary structures (Khan et al., 2002).

C. Flaviviruses

The family Flaviviridae is a group of single, positive-stranded RNA viruses with a genome size from 9-15 kb. They are enveloped viruses of approximately 40-50 nm. Flaviviruses are small, enveloped viruses containing a single, positive-strand, genomic RNA, approximately 10,500 nucleotides in length containing short 5′ and 3′ non-translated regions (NTRs), a single long open reading frame, a 5′ cap, and a nonpolyadenylated 3′ terminus. The complete nucleotide sequence of numerous flaviviral genomes, including all four dengue serotypes, yellow fever virus, Japanese encephalitis virus, West Nile virus and tick-borne encephalitis virus have been reported. All flaviviral proteins are derived from a single long polyprotein through precise processing events mediated by host as well as virally encoded proteases. The ten gene products encoded by the single open reading frame are translated as a polyprotein organized in the order, capsid (C), preMembrane (prM, which is processed to Membrane (M) just prior to virion release from the cell), Envelope (E) and the seven non-structural (NS) proteins: NS1, NS2a, NS2b, NS3, NS4a, NS4b and NS5 (Leyssen, De Clercq et al. 2000; Brinton 2002).

Within the Flaviviridae family is the flavivirus genus which includes the prototype yellow fever virus (YFV), the four serotypes of dengue virus (DEN-1, DEN-2, DEN-3, and DEN-4), Japanese encephalitis virus (JEV), Murray Valley encephalitis virus (MVEV), Kunjin virus (KUN), St. Louis encephalitis virus (SLEV), West Nile virus (WNV), Tick-borne encephalitis virus (TBEV), and about 70 other disease causing viruses. The term “flavivirus” has its conventional meaning in the art, and includes tick-borne encephalitis virus, Central European Encephalitis virus, Far Eastern Encephalitis virus, Kunjin virus, Murray Valley Encephalitis virus, St. Louis Encephalitis virus, Rio Bravo virus, Japanese Encephalitis virus, Tyuleniy virus, Ntaya virus, Uganda virus, Dengue virus, Modoc virus, yellow fever virus, West Nile virus, pestiviruses, bovine viral diarrhea virus (including BVDV-1 and BVDV-2), Border disease virus, hepaciviruses, hepatitis C virus, GB virus-A, GB virus-.beta. and GB virus-C and any other virus classified by the International Committee on Taxonomy of Viruses (ICTV) as a flavivirus.

Yellow fever is caused by yellow fever virus, an enveloped RNA virus 40-50 nm in width. The positive-sense, single-stranded RNA is around 10,862 nucleotides long and has a single open reading frame encoding a polyprotein. Host proteases cut this polyprotein into three structural (C, prM, E) and seven nonstructural proteins (NS1, NS2A, NS2B, NS3, NS4A, NS4B, NS5); the enumeration corresponds to the arrangement of the protein coding genes in the genome.

D. Coronaviruses

“Coronavirus” as used herein refers to a genus in the family Coronaviridae, which family is in turn classified within the order Nidovirales. The coronaviruses are large, enveloped, positive-stranded RNA viruses. They have the largest genomes of all RNA viruses and replicate by a unique mechanism that results in a high frequency of recombination. The coronaviruses include antigenic groups I, II, and III. Coronaviruses (CoVs) constitute a group of phylogenetically diverse enveloped viruses that encode the largest plus strand RNA genomes and replicate efficiently in most mammals. Members of the Coronaviridae include the human coronaviruses that cause 10 to 30% of common colds and other respiratory infections, and murine hepatitis virus. Nonlimiting examples of coronaviruses include the viruses that cause severe acute respiratory syndrome (SARS), Middle East respiratory syndrome (MERS-CoV), and Covid-19 (SARS-CoV-2).

### III. RNA Delivery Systems

RNA therapeutics have been previously formulated using a range of delivery systems, wherein the overarching principle is to use a cationic/ionizable lipid or polymer to electrostatically complex the anionic RNA molecules, reducing the size of the particle and facilitating cellular uptake. There are many types of artificial RNA delivery systems known to those of ordinary skill in the art. One common class of artificial RNA delivery system is the lipid particle which includes nanostructured lipid carriers (NLC), lipid nanoparticles (LNP), and cationic nanoemulsions (CNE). Any of these or other delivery systems capable to delivering ribonucleic acid (RNA) polynucleotide encoding a replication-competent viral genome to the cytosol of a cell may be used.

In an illustrative implementation, the RNA polynucleotide which is negatively charged is complexed with components of an artificial RNA delivery system by association with a cationic surface. The association of the negatively-charged RNA with the NLC surface may be a non-covalent or a reversible covalent interaction. The association of the negatively-charged RNA with the NLC surface may be through electrostatic attraction.

Combination of a ribonucleic acid (RNA) polynucleotide encoding a replication-competent viral genome with as suitable artificial RNA delivery system can provide an infection composition that functions as a “manufactured virus” or “artificial virus platform.” Inoculation of a subject with a manufacture virus as provided in this disclosure in an amount sufficient to cause to viral replication in the subject will cause an active viral infection in the subject.

A. Nanostructured Lipid Carriers

In one implementation, compositions of this disclosure may use nanostructured lipid carriers (NLC) as an artificial RNA delivery system for ribonucleic acid (RNA) polynucleotide encoding a replication-competent viral genome. NLC compositions are made up of NLC particles comprising (a) an oil core comprising a liquid phase lipid and a solid phase lipid, (b) a cationic lipid (c) a hydrophobic surfactant, preferably a sorbitan ester (e.g., sorbitan monoester, diester, or triester), and (d) a surfactant (preferably, a hydrophilic surfactant). NLCs typically comprise an unstructured or amorphous solid lipid matrix made up of a mixture of blended solid and liquid lipids dispersed in an aqueous phase. One or more of the surfactants can be present in the oil phase, the aqueous phase, or at the interface between the oil and aqueous phase. In certain aspects the sorbitan ester and the cationic lipid are present at the interface between the oil and aqueous phase.

NLCs are composed of a blend of solid and liquid lipids. The liquid and solid lipids to be used in the NLCs can be any lipid capable of forming an unstructured or amorphous solid lipid matrix and forming a stable composition. The oil core of the NLC comprises a liquid phase lipid. Preferably, although not necessarily, the liquid phase lipid is a metabolizable, non-toxic oil; more preferably one of about 6 to about 30 carbon atoms including, but not limited to, alkanes, alkenes, alkynes, and their corresponding acids and alcohols, the ethers and esters thereof, and mixtures thereof. The oil may be, for example, any vegetable oil, fish oil, animal oil or synthetically prepared oil that can be administered to a subject. In some aspects, the liquid phase lipid will be non-metabolizable.

Any suitable oils from an animal, fish or vegetable source may be used. Sources for vegetable oils include nuts, seeds and grains, and suitable oils include, for example, peanut oil, soybean oil, coconut oil, and olive oil and the like. Other suitable seed oils include safflower oil, cottonseed oil, sunflower seed oil, sesame seed oil and the like. In the grain group, corn oil, and the oil of other cereal grains such as wheat, oats, rye, rice, teff, triticale and the like may also be used. The technology for obtaining vegetable oils is well developed and well known. The compositions of these and other similar oils may be found in, for example, the Merck Index, and source materials on foods, nutrition, and food technology.

Most fish contain metabolizable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. Naturally occurring or synthetic terpenoids, also referred to as isoprenoids, can be used herein as a liquid phase lipid. Squalene, is a branched, unsaturated terpenoid. A major source of squalene is shark liver oil, although plant oils (primarily vegetable oils), including amaranth seed, rice bran, wheat germ, and olive oils, are also suitable sources. Squalane is the saturated analog to squalene. Oils, including fish oils such as squalene and squalane, are readily available from commercial sources or may be obtained by methods known in the art. Oils to be used herein may also be made using synthetic means, including genetic engineering (e.g., oils made from bioengineered yeast, including squalene.) Synthetic squalene has been successfully produced from bioengineered yeast and exhibits immunomodulating characteristics equal to squalene obtained from sharks. (Mizuki Tateno et al., Synthetic Biology-derived triterpenes as efficacious immunomodulating adjuvants, Sci Rep 10, 17090 (2020).) Squalene has also been synthesized by the controlled oligomerization of isoprene. (Kevin Adlington et al., Molecular Design of Squalene/Squalane Countertypes via the Controlled Oligomerization of Isoprene and Evaluation of Vaccine Adjuvant Applications, Biomacromolecules, 17(1) pages 165-172 (2016).)

The oil core of the NLC comprises a solid phase lipid. A wide variety of solid phase lipids can be used, including for example, glycerolipids. Glycerolipids are a fatty molecules composed of glycerol linked esoterically to a fatty acid. Glycerolipids include triglycerides and diglycerides. Illustrative solid phase lipids include, for example, glyceryl palmitostearate (Precitol ATO® 5), glycerylmonostearate, glyceryl dibehenate (Compritol® 888 ATO), cetyl palmitate (Crodamol™ CP), stearic acid, tripalmitin, or a microcrystalline triglyceride. Illustrative microcrystalline triglycerides include those sold under the trade name Dynasan® (e.g., trimyristin (Dynasan® 114) or tristearin (Dynasan® 118) or tripalmitin (Dynasan® 116)).

The solid phase lipid can be, for example, a microcrystalline triglyceride, for example, one selected from trimyristin (Dynasan® 114) or tristearin (Dynasan® 118). Preferably, the solid phase lipid of the oil core is solid at ambient temperature. When indoors, ambient temperature is typically between 15° C. and 25° C.

The NLCs described herein comprise a cationic lipid. The cationic lipid is useful for interacting with negatively charged bioactive agents on the surface on the NLC. Any cationic lipid capable of interacting with negatively charged bioactive agents that will not disturb the stability of the NLC and can be administered to a subject may be used. Generally, the cationic lipid contains a nitrogen atom that is positively charged under physiological conditions. Suitable cationic lipids include, benzalkonium chloride (BAK), benzethonium chloride, cetrimide (which contains tetradecyltrimethylammonium bromide and possibly small amounts of dodecyltrimethylammonium bromide and hexadecyltrimethyl ammonium bromide), cetylpyridinium chloride (CPC), cetyl trimethylammonium chloride (CTAC), primary amines, secondary amines, tertiary amines, including but not limited to N,N′,N′-polyoxyethylene (10)-N-tallow-1,3-diaminopropane, other quaternary amine salts, including but not limited to dodecyltrimethylammonium bromide, hexadecyltrimethyl-ammonium bromide, mixed alkyl-trimethyl-ammonium bromide, benzyldimethyldodecylammonium chloride, benzyldimethylhexadecyl-ammonium chloride, benzyltrimethylammonium methoxide, cetyldimethylethylammonium bromide, dimethyldioctadecyl ammonium bromide (DDAB), methylbenzethonium chloride, decamethonium chloride, methyl mixed trialkyl ammonium chloride, methyl trioctylammonium chloride, N,N-dimethyl-N-[2 (2-methyl-4-(1,1,3,3tetramethylbutyl)-phenoxyl-ethoxy)ethyl]-benzenemethanaminium chloride (DEBDA), dialkyldimethylammonium salts, [1-(2,3-dioleyloxy)-propyl]-N,N,N,trimethylammonium chloride, 1,2-diacyl-3-(trimethylammonio) propane (acyl group=dimyristoyl, dipalmitoyl, distearoyl, dioleoyl), 1,2-diacyl-3(dimethylammonio)propane (acyl group=dimyristoyl, dipalmitoyl, distearoyl, dioleoyl), 1,2-dioleoyl-3-(4′-trimethyl-ammonio)butanoyl-sn-glycerol, 1,2-dioleoyl 3-succinyl-sn-glycerol choline ester, cholesteryl (4′-trimethylammonio) butanoate), N-alkyl pyridinium salts (e.g. cetylpyridinium bromide and cetylpyridinium chloride), N-alkylpiperidinium salts, dicationic bolaform electrolytes (C12Me6; C12Bu6), dialkylglycetylphosphorylcholine, lysolecithin, L-α dioleoylphosphatidylethanolamine, cholesterol hemisuccinate choline ester, lipopolyamines, including but not limited to dioctadecylamidoglycylspermine (DOGS), dipalmitoyl phosphatidylethanol-amidospermine (DPPES), lipopoly-L (or D)-lysine (LPLL, LPDL), poly (L (or D)-lysine conjugated to N-glutarylphosphatidylethanolamine, didodecyl glutamate ester with pendant amino group (C12GluPhCnN+), ditetradecyl glutamate ester with pendant amino group (C14GluCnN+), cationic derivatives of cholesterol, including but not limited to cholesteryl-3β-oxysuccinamidoethylenetrimethylammonium salt, cholesteryl-3β-oxysuccinamidoethylenedimethylamine, cholesteryl-3β-carboxyamidoethylenetrimethylammonium salt, cholesteryl-3β-carboxyamidoethylenedimethylamine, and 3γ-[N—(N′,N-dimethylaminoetanecarbomoyl]cholesterol) (DC-Cholesterol), 1,2-dioleoyloxy-3-(trimethylammonio)propane (DOTAP), dimethyldioctadecylammonium (DDA), 1,2-Dimyristoyl-3-TrimethylAmmoniumPropane (DMTAP), dipalmitoyl (C16:0)trimethyl ammonium propane (DPTAP), distearoyltrimethylammonium propane (DSTAP), and combination thereof.

Other cationic lipids suitable for use in the invention include, e.g., the cationic lipids described in U.S. Patent Pub. No. 2008/0085870 (published Apr. 10, 2008) and 2008/0057080 (published Mar. 6, 2008).

Other cationic lipids suitable for use in the invention include, e.g., Lipids E0001-E0118 or E0119-E0180 as disclosed in Table 6 (pages 112-139) of WO2011/0776807 (which also discloses methods of making, and method of using these cationic lipids). Additional suitable cationic lipids include N-[1-(2,3-dioleyloxy)propyl]-N,N,N-trimethylammonium chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), 1,2-dilinoleyloxy-3-dimethylaminopropane (DLinDMA).

The NLCs may comprise one or any combination of two or more of the cationic lipids described herein.

In some cases, it may be desirable to use a cationic lipid that is soluble in the oil core. For example, DOTAP DOEPC, DODAC, and DOTMA are soluble in squalene or squalane. In other cases, it may be desirable to use a cationic lipid that is not soluble in the oil core. For example, DDA and DSTAP are not soluble in squalene. It is within the knowledge in the art to determine whether a particular lipid is soluble or insoluble in the oil and choose an appropriate oil and lipid combination accordingly. For example, solubility can be predicted based on the structures of the lipid and oil (e.g., the solubility of a lipid may be determined by the structure of its tail). For example, lipids having one or two unsaturated fatty acid chains (e.g., oleoyl tails), such as DOTAP, DOEPC, DODAC, DOTMA, are soluble in squalene or squalane; whereas lipids having saturated fatty acid chains (e.g., stearoyl tails) are not soluble in squalene. Alternatively, solubility can be determined according to the quantity of the lipid that dissolves in a given quantity of the oil to form a saturated solution).

The NLC may comprise additional lipids (i.e., neutral and anionic lipids) in combination with the cationic lipid so long as the net surface charge of the NLC prior to mixing with the bioactive agent is positive. Methods of measuring surface charge of a NLC are known in the art and include for example, as measured by Dynamic Light Scattering (DLS), Photon Correlation Spectroscopy (PCS), or gel electrophoresis.

A sorbitan ester when added to the NLC can act to enhance the effectiveness of the NLC in delivering the bioactive agent to a cell and/or in eliciting antibodies to an antigen in a subject where the bioactive agent is an antigen or encodes antigen and the composition is administered to a subject. The term “sorbitan ester” as used herein refers to an ester of sorbitan. Sorbitan is as shown in Formula A

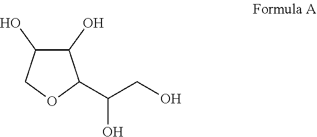 [SMILES: OCC(O)C1OCC(O)C1O]

Suitable sorbitan esters are sorbitan alkyl esters, wherein the alkyl is a C₁-C₃₀ alkyl group, preferably a saturated or unsaturated C₁-C₂₀ alkyl group, more preferably a saturated or unsaturated C₁₀-C₂₀ alkyl group.

Illustrative sorbitan monoesters are commercially available under the tradenames SPAN® or ARLACEL®. An illustrative sorbitan monoester for use herein can be represented as a compound of Formula I or a stereoisomer thereof (including, but not limited to, Formula Ia, Ib, Ic, or Id) wherein R is a saturated or unsaturated C1-C30 alkyl group, preferably a saturated or unsaturated C1-C20 alkyl group, more preferably a saturated or unsaturated C10-C20 alkyl group. In illustrative implementations, the alkyl group is non-cyclic. Illustrative sorbitan monoesters also include positional isomers of Formulas I, Ia, Ib, Ic or Id (e.g., one of the hydroxy functional groups is replaced by an ester functional group (e.g., an alkyl ester wherein the alkyl is a saturated or unsaturated C1-C30 alkyl group, preferably a saturated or unsaturated C1-C20 alkyl group, more preferably a saturated or unsaturated C10-C20 alkyl group and R is OH). The skilled artisan will appreciate that illustrative sorbitan monoesters may be salt forms (e.g., pharmaceutically acceptable salts) of Formulas I, Ia, Ib, Ic, Id and stereoisomers or positional isomers thereof.

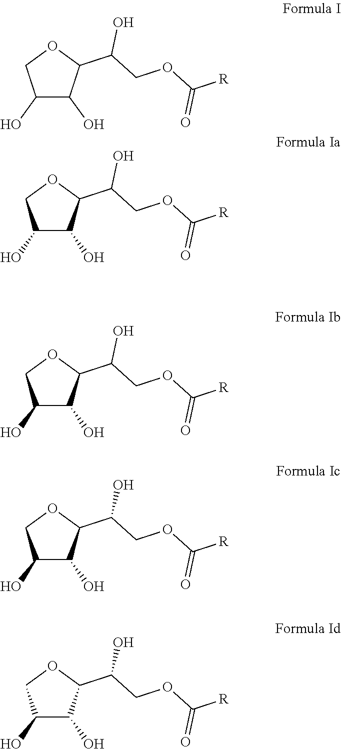

Suitable sorbitan monoesters in this regard are sorbitan monostearate (also knowns as Span® 60 and shown below) and sorbitan monooleate (also known as Span® 80 and shown below), although other sorbitan monoesters can be used (including, but not limited to, sorbitan monolaurate (Span® 20), sorbitan monopalmitate (Span® 40)). Illustrative sorbitan monostearate is represented by Formula II or IIa or a salt form thereof and illustrative sorbitan monooleate is represented by Formula III or Ma or a salt form thereof.

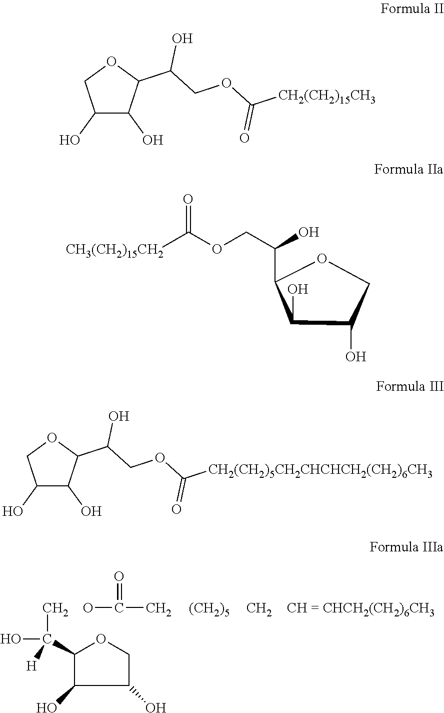

In addition to providing sorbitan monoesters as a component of a NLC, also contemplated is the substitution of the sorbitan monoester for an alternative hydrophobic surfactant, including alternative sorbitan-based non-ionic surfactants. Accordingly, also provided herein are NLC particles comprising an oil core comprising a liquid phase lipid and a solid phase lipid, a cationic lipid, a hydrophobic surfactant (e.g., non-ionic surfactants including sorbitan-based non-ionic surfactants) and a hydrophilic surfactant. Sorbitan-based non-ionic surfactants include sorbitan esters other than sorbitan monoesters, for example sorbitan diesters and sorbitan triesters, such as for example, sorbitan trioleate (SPAN85™) and sorbitan tristearate (SPAN65™). Generally, the non-ionic surfactant (including sorbitan-based non-ionic surfactant) will have a hydrophilic-lipophilic balance (HLB) number between 1.8 to 8.6. All of the implementations provided herein for the NLCs comprising a sorbitan monoester are applicable and contemplated for the NLCs comprising an alternative hydrophobic surfactant in place of the sorbitan monoester, e.g., NLCs comprising a sorbitan diester or triester in place of the sorbitan monoester. The sorbitan diester and triester or other hydrophobic surfactant can be present in the same concentrations as the sorbitan monoester. In some aspects, the acyl chains of the sorbitan diester or triester will be saturated.

Generally, the sorbitan esters (e.g., sorbitan monoesters) have a hydrophile-lipophile balance (HLB) value from 1 to 9. In some implementations, the sorbitan esters (e.g., sorbitan monoesters) have an HLB value from 1 to 5. In some implementations, the hydrophobic surfactant has a HLB value from about 4 to 5.

An illustrative sorbitan diester for use herein can be represented as a compound of Formula IV below or a stereoisomer thereof (e.g., wherein R is a saturated or unsaturated C1-C30 alkyl group, preferably a saturated or unsaturated C1-C20 alkyl group, more preferably a saturated or unsaturated C10-C20 alkyl group and at least one of R1 is H while the other is —C(═O)Y wherein Y is a saturated or unsaturated C1-C30 alkyl group, preferably a saturated or unsaturated C1-C20 alkyl group, more preferably a saturated or unsaturated C10-C20 alkyl group). In illustrative implementations, the alkyl group is non-cyclic. Illustrative sorbitan diesters also include positional isomers of Formulas IV. The skilled artisan will appreciate that illustrative sorbitan diesters may be salt forms (e.g., pharmaceutically acceptable salts) of Formula IV and stereoisomers or positional isomers thereof.

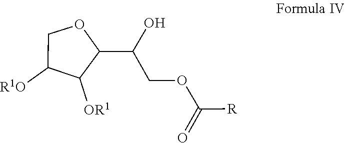

As illustrative sorbitan triester for use herein can be represented as a compound of Formula V below or a stereoisomer thereof (including, but not limited to, Formula Va, Vb, or Vc) wherein R is a saturated or unsaturated C1-C30 alkyl group, preferably a saturated or unsaturated C1-C20 alkyl group, more preferably a saturated or unsaturated C10-C20 alkyl group and R1 is —C(═O)Y wherein Y can be the same or different in each instance and is a saturated or unsaturated C1-C30 alkyl group, preferably a saturated or unsaturated C1-C20 alkyl group, more preferably a saturated or unsaturated C10-C20 alkyl group. In illustrative implementations, the alkyl group is non-cyclic. Illustrative sorbitan triesters also include positional isomers of Formulas V, Va, Vb, or Vc (e.g., the hydroxy functional group is replaced by an ester functional group (e.g., an alkyl ester wherein the alkyl is a saturated or unsaturated C1-C30 alkyl group, preferably a saturated or unsaturated C1-C20 alkyl group, more preferably a saturated or unsaturated C10-C20 alkyl group) and one of the alkyl esters (e.g., a ring alkyl ester or non-ring alkyl ester) is replaced by a hydroxy functional group). The skilled artisan will appreciate that illustrative sorbitan triesters may be salt forms (e.g., pharmaceutically acceptable salts) of Formulas V, Va, Vb, or Vc and stereoisomers or positional isomers thereof.

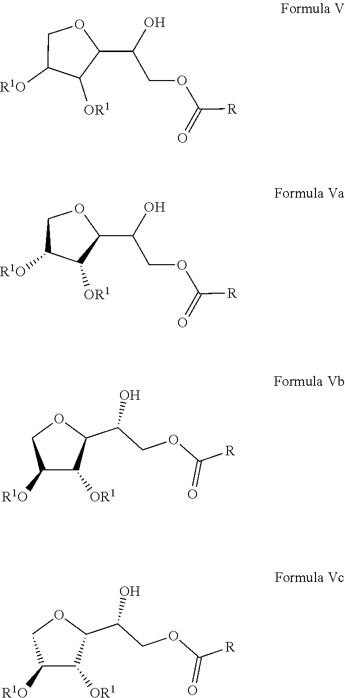

With respect to stereoisomers, the skilled artisan will understand that the sorbitan esters may have chiral centers and may occur, for example, as racemates, racemic mixtures, and as individual enantiomers and diastereomers.

The NLCs described herein comprise a surfactant, in addition to the sorbitan-based non-ionic surfactants (e.g., sorbitan ester). There are a number of surfactants specifically designed for and commonly used in biological applications. Such surfactants are divided into four basic types and can be used in the present invention: anionic, cationic, zwitterionic and nonionic. A particularly useful group of surfactants are the hydrophilic non-ionic surfactants and, in particular, polyoxyethylene sorbitan monoesters and polyoxyethylene sorbitan triesters. These materials are referred to as polysorbates and are commercially available under the mark TWEEN® and are useful for preparing the NLCs. TWEEN® surfactants generally have a HLB value falling between 9.6 to 16.7. TWEEN® surfactants are commercially available. Other non-ionic surfactants which can be used are, for example, polyoxyethylene fatty acid ethers derived from lauryl, acetyl, stearyl and oleyl alcohols, polyoxyethylene fatty acids made by the reaction of ethylene oxide with a long-chain fatty acid, polyoxyethylene, polyol fatty acid esters, polyoxyethylene ether, polyoxypropylene fatty ethers, bee's wax derivatives containing polyoxyethylene, polyoxyethylene lanolin derivative, polyoxyethylene fatty glycerides, glycerol fatty acid esters or other polyoxyethylene fatty acid, alcohol or ether derivatives of long-chain fatty acids of 12-22 carbon atoms.

In some implementations, it is preferable to choose a non-ionic surfactant which has an HLB value in the range of about 7 to 16. This value may be obtained through the use of a single non-ionic surfactant such as a TWEEN® surfactant or may be achieved by the use of a blend of surfactants. In certain implementations, the NLC comprises a single non-ionic surfactant, most particularly a TWEEN® surfactant, as the emulsion stabilizing non-ionic surfactant. In an illustrative implementation, the emulsion comprises TWEEN® 80, otherwise known as polysorbate 80.

Additional components can be included in the NLCs of the present invention including, for examples, components that promote NLC formation, improve the complex formation between the negatively charged molecules and the cationic particles, facilitate appropriate release of the negatively charged molecules (such as an RNA molecule), and/or increase the stability of the negatively charged molecule (e.g., to prevent degradation of an RNA molecule).

The aqueous phase (continuous phase) of the NLCs is typically a buffered salt solution (e.g., saline) or water. The buffered salt solution is typically an aqueous solution that comprises a salt (e.g., NaCl), a buffer (e.g., a citrate buffer), and can further comprise, for example, an osmolality adjusting agent (e.g., a saccharide), a polymer, a surfactant, or a combination thereof. If the emulsions are formulated for parenteral administration, it is preferable to make up final buffered solutions so that the tonicity, i.e., osmolality is essentially the same as normal physiological fluids in order to prevent undesired post-administration consequences, such as post-administration swelling or rapid absorption of the composition. It is also preferable to buffer the aqueous phase in order to maintain a pH compatible with normal physiological conditions. Also, in certain instances, it may be desirable to maintain the pH at a particular level in order to ensure the stability of certain components of the NLC. For example, it may be desirable to prepare a NLC that is isotonic (i.e., the same permeable solute (e.g., salt) concentration as the normal cells of the body and the blood) and isosmotic. To control tonicity, the NLC may comprise a physiological salt, such as a sodium salt. In some aspects, sodium chloride (NaCl), for example, may be used at about 0.9% (w/v) (physiological saline). Other salts that may be present include, for example, potassium chloride, potassium dihydrogen phosphate, disodium phosphate, magnesium chloride, calcium chloride, and the like. Non-ionic tonicifying agents can also be used to control tonicity. Monosaccharides classified as aldoses such as glucose, mannose, arabinose, and ribose, as well as those classified as ketoses such as fructose, sorbose, and xylulose can be used as non-ionic tonicifying agents in the present invention. Disaccharides such a sucrose, maltose, trehalose, and lactose can also be used. In addition, alditols (acyclic polyhydroxy alcohols, also referred to as sugar alcohols) such as glycerol, mannitol, xylitol, and sorbitol are non-ionic tonicifying agents that can be useful in the present invention. Non-ionic tonicity modifying agents can be present, for example, at a concentration of from about 0.1% to about 10% or about 1% to about 10%, depending upon the agent that is used.

The aqueous phase may be buffered. Any physiologically acceptable buffer may be used herein, such as water, citrate buffers, phosphate buffers, acetate buffers, tris buffers, bicarbonate buffers, carbonate buffers, succinate buffer, or the like. The pH of the aqueous component will preferably be between 4.0-8.0 or from about 4.5 to about 6.8. In another illustrative implementation, the aqueous phase is, or the buffer prepared using, RNase-free water or DEPC treated water. In some cases, high salt in the buffer might interfere with complexation of negatively charged molecule to the emulsion particle therefore is avoided. In other cases, certain amount of salt in the buffer may be included.

In an illustrative implementation, the buffer is citrate buffer (e.g., sodium citrate) with a pH between about 5.0 and 8.0. The citrate buffer may have a concentration of between 1-20 mM such as, 5 mM, 10 mM, 15 mM, or 20 mM. In another illustrative implementation, the aqueous phase is, or the buffer is prepared using, RNase-free water or DEPC treated water. In other illustrative implementations, the compositions of the present invention do not comprise a citrate buffer.

The aqueous phase may also comprise additional components such as molecules that change the osmolarity of the aqueous phase or molecules that stabilize the negatively charged molecule after complexation. Preferably, the osmolarity of the aqueous phase is adjusting using a non-ionic tonicifying agent, such as a sugar (e.g., trehalose, sucrose, dextrose, fructose, reduced palatinose, etc.), a sugar alcohol (such as mannitol, sorbitol, xylitol, erythritol, lactitol, maltitol, glycerol, etc.), or combinations thereof. If desired, a nonionic polymer (e.g., a poly(alkyl glycol) such as polyethylene glycol, polypropylene glycol, or polybutylene glycol) or nonionic surfactant can be used.

As provided herein, one method of making the NLCs described herein comprises (a) mixing the solid phase lipid, the liquid phase lipid, the cationic lipid, and the hydrophobic surfactant (e.g., sorbitan ester) to form an oil phase mixture; (b) mixing the hydrophilic surfactant and water to form an aqueous phase; and (c) mixing the oil phase mixture with the aqueous phase mixture to form the NLC. In some implementations, a further step comprises combining the bioactive agent with the NLC such that the bioactive agent associates with the surface of the NLC by non-covalent interactions or by reversible covalent interactions. Such implementations are possible where the bioactive agent is negatively charged, such as an RNA molecule or a DNA molecule. The negative charges on the bioactive agent interact with the cationic lipid in the NLC, thereby associating the negatively charged bioactive agent with the NLC. In other implementations, where the bioactive agent is hydrophobic, it is combined with the components in step (a) to form part of the oil phase mixture. In some implementations, the bioactive agent may be attached to a component of the surface of the NLC via covalent interactions.

Mixing the solid phase lipid, the liquid phase lipid, the cationic lipid, and the hydrophobic surfactant (e.g., sorbitan ester) to form an oil phase mixture may be achieved, for example, by heating and sonication. Mixing the oil phase mixture with the aqueous phase mixture may be achieved, for example, by various emulsification methods, including, without limitation, high shear emulsification and microfluidization.

B. Lipid Nanoparticles

In one implementation, compositions of this disclosure may use lipid nanoparticles (LNP) as an artificial RNA delivery system for ribonucleic acid (RNA) polynucleotide encoding a replication-competent viral genome. LNPs are one example of lipid particles. RNA polynucleotides of this disclosure may be complexed or combined with LNP either on the outside or inside of the particle. LNPs are spherical vesicles made of ionizable lipids, which are positively charged at low pH (enabling RNA complexation) and neutral at physiological pH (reducing potential toxic effects, as compared with positively charged lipids, such as liposomes). Owing to their size and properties, lipid nanoparticles are taken up by cells via endocytosis, and without being bound by theory it is believed that the ionizability of the lipids at low pH enables endosomal escape, which allows release of the cargo into the cytoplasm.

In addition, LNPs usually may contain any or all of a helper lipid to promote cell binding, cholesterol to fill the gaps between the lipids, and a polyethylene glycol (PEG) to reduce opsonization by serum proteins and reticuloendothelial clearance. The relative amounts of ionizable lipid, helper lipid, cholesterol and PEG can affect the efficacy of lipid nanoparticles and may be optimized for a given application and administration route. Moreover, lipid type, size and surface charge impact the behavior of lipid nanoparticles in vivo.

Lipid nanoparticle (LNP) delivery systems are discussed in (L. A. Jackson et al., An mRNA Vaccine against SARS-CoV-2—Preliminary Report. N Engl J Med 383, 1920-1931 (2020); Y. Y. Tam, S. Chen, P. R. Cullis, Advances in Lipid Nanoparticles for siRNA Delivery. Pharmaceutics 5, 498-507 (2013); Y. Zhao and L. Huang, Lipid nanoparticles for gene delivery. Adv Genet 88, 13-36 (2014); A. M. Reichmuth et al., mRNA vaccine delivery using lipid nanoparticles. Therapeutic Delivery 7, 319-334 (2016); K. Bahl et al., Preclinical and Clinical Demonstration of Immunogenicity by mRNA Vaccines against H10N8 and H7N9 Influenza Viruses. Mol Ther 25, 1316-1327 (2017)). LNP formulations may contain cationic and ionizable lipids with RNA associated with either the interior or exterior of the particle. (A. K. Blakney et al., Inside out: optimization of lipid nanoparticle formulations for exterior complexation and in vivo delivery of saRNA. Gene Ther 26, 363-372 (2019)).

C. Cationic Nanoemulsions

In one implementation, compositions of this disclosure may use cationic nanoemulsions (CNE) as an artificial RNA delivery system for ribonucleic acid (RNA) polynucleotide encoding a replication-competent viral genome. CNE is one example of a lipid particle. CNE consists of a dispersion of an oil phase stabilized by an aqueous phase containing the cationic lipid. These nanoemulsions present a droplet size distribution of about 200 nm and are used to formulate RNA vaccines. (L. A. Brito et al., A cationic nanoemulsion for the delivery of next-generation RNA vaccines. Mol Ther 22, 2118-2129 (2014).

D. Charge-Altering Releasable Transporters CART

Charge-altering releasable transporters (CART) are single component amphiphilic diblock oligomers containing a sequence of lipid monomers and a sequence of cationic monomers that provide an alternative delivery vehicle RNA besides lipid particles. In one implementation, compositions of this disclosure may use amphiphilic diblock oligomers containing a sequence of lipid monomers and a sequence of cationic monomers as an artificial RNA delivery system for ribonucleic acid (RNA) polynucleotide encoding a replication-competent viral genome. CARTs electrostatically encapsulate mRNA (or other coformulated nucleotides like CpG) and deliver the genetic cargo into cells. A unique feature of CARTs is their ability to undergo a charge-altering rearrangement to produce neutral diketopiperazine small molecules (DKPs). This transformation facilitates the release of mRNA and eliminates any toxic issues associated with persistent cations. The CART technology is described in Ole A. W. Haabeth et al., An mRNA SARS-CoV-2 vaccine employing Charge Altering Releasable Transporters with a TLR-9 agonist induces neutralizing antibodies and T cell memory, (2021) bioRxiv 2021.04.14.439891.

E. Loading Capacities

The loading capacity of the artificial RNA delivery system can be manipulated by modulating the ratio of components thereby changing the average particle size. Illustrative lipid particle formulations have loading capacity for RNA of at least about 10 μg/ml RNA, at least about 20 μg/ml RNA, at least about 50 μg/ml RNA, at least about 100 μg/ml RNA, at least about 200 μg/ml RNA, at least about 300 μg/ml, or at least about 400 μg/ml RNA. Lipid particle formulations having an average particle size of from 20 nm to about 110 nm, from about 20 nm to about 80 nm, from about 20 nm to about 70 nm, from about 20 nm to about 60 nm typically have increased loading capacity. Persons of ordinary skill in the art will appreciate how to adjust the formulation of the artificial RNA delivery system to achieve a desired loading capacity.

### IV. Methods of Manufacturing

The ribonucleic acid (RNA) polynucleotide encoding a replication-competent viral genome of this disclosure may be produced by transcription from a DNA construct. The DNA construct may be a plasmid such as an expression vector comprising a eukaryotic or viral promotor. Fully-functional, capped RNA can be created from a DNA construct as a template using in vitro transcription and capping reactions.

The present invention includes expression vectors that comprise a cDNA copy of a live-attenuated virus genome of the invention. In particular suitable viruses include any strains which are known and available in the art. Generally, the viral genomes and cDNA clones thereof will comprise the entire viral genome (modified to include the attenuating mutations). In some embodiments, the genomic sequences will have at least 40, 50, 60, 70, 80 or 85%, more particularly at least 86, 87, 88, 89, 90, 91, 92, 93, 94, 95, 96, 97, 98 or 99%, sequence identity to a wildtype genomic sequence of the corresponding virus. Such expression vectors are routinely constructed in the art of molecular biology and may for example involve the use of plasmid DNA and appropriate initiators, promoters, enhancers, and other elements, such as for example polyadenylation signals, which may be necessary, and which are positioned in the correct orientation, in order to allow for RNA transcription and protein expression. Other suitable vectors would be apparent to persons skilled in the art.

In various implementations, the DNA constructs may contain genome derived from ZIKV or YFV and thus may comprise DNA copies of the genomes of attenuated variants of any strain of ZIKV, YFV, or other positive strand virus. For example, the source of the ZIKV DNA copy can be an attenuated variant of any one of the following strains: MR766-NIID, P6-740, ArD71 17, lbH_30656, ArB1362, ARB13565, ARB7701, ARB15076, ArD_41519, ArD128000, ArD158084, ArD157995, FSM, FSS13025, PHL/2012/CPC-0740-Asian, H/PF/2013, PLCal_ZV, Haiti/1225/2014, SV0127_14_Asian, Natal_RGN_Asian, Brazil_ZKV2015_Asian, ZikaSPH2015, BeH815744, BeH819015, BeH819966, BeH823339, BeH828305, SSABR1-Asian, FLR, 103344, 8375, PRVABC59, Z1 106033, MRS_OPY_Martinique, VE_Ganxian_Asian, GD01_Asian, GDZ16001, ZJ03, Rio-111 or Rio-S1 ZIKV strains. In one implementation, the source of the yellow fever DNA may be YF17D.

The cDNA copy of a live-attenuated virus genome for use in the invention in a vector is operably linked to control sequence(s) which can provide for transcription of the RNA virus and expression of the viral genomic RNA. The term “operably linked” refers to a juxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence.

Promoters and other expression regulation signals may be selected to be compatible with the cell system for which expression is designed. Examples of promoters which are suitable for use with the DNA sequences of the present invention include, but are not limited to T3 promoters, T7 promoters, cytomegalovirus (CMV) promoters, and SP6 promoters.

In some embodiments, the DNA copy of the live-attenuated plus-sense single stranded RNA virus is contained in a plasmid, which optionally comprises a promoter, a ribosome-translated sequence and/or a polyadenylation (pA) signal sequence.

As a further aspect, the invention provides nucleic acids encoding the viral genomes of the invention. For example, the present invention provides DNA sequences (e.g., cDNA sequences) and vectors encoding infectious modified alphavirus genomic RNA transcripts (e.g., VEE genomic transcripts) as described herein. The present invention further provides vectors and constructs comprising a DNA sequence encoding a genomic RNA of a positive strand virus operably associated with a promoter that drives transcription of the DNA sequence. The DNA sequence may be embedded within any suitable vector known in the art, including but not limited to, plasmids, naked DNA vectors, yeast artificial chromosomes (yacs), bacterial artificial chromosomes (bacs), phage, viral vectors, and the like.

The DNA plasmids may include a subgenomic promoter that directs expression of a heterologous sequence. If desired, the heterologous sequence (e.g., the RNA viral genome) may be fused in frame to other coding regions, with or without a ribosomal skipping peptide sequence in the self-amplifying RNA and/or may be under the control of an internal ribosome entry site (IRES).

Further provided are cells containing the DNA sequences, genomic RNA transcripts, and alphavirus vectors of the invention. Exemplary cells include, but are not limited to, fibroblast cells, Vero cells, Baby Hamster Kidney (BHK) cells, Chinese Hamster Ovary (CHO) cells, macrophages, dendritic cells, and the like.

Genomic RNA transcripts may be synthesized from the DNA template by any method known in the art. Preferably, the RNA is synthesized from the DNA sequence in vitro using purified RNA polymerase in the presence of ribonucleotide triphosphates and cap analogs in accordance with conventional techniques.

### VI. Compositions and Dosing

Provided herein are formulations, compositions, and pharmaceutical compositions comprising the RNA polynucleotides described herein. The compositions can optionally further comprise a pharmaceutically acceptable carrier, excipient, or diluent. Formulation of pharmaceutical compositions is well known in the pharmaceutical arts (see, e.g., Remington's Pharmaceutical Sciences, (15th Edition, Mack Publishing Company, Easton, Pa. A. R. Gennaro edit. (1985).

“Pharmaceutically acceptable carriers” for therapeutic use are well known in the pharmaceutical arts. Id. For example, sterile saline and phosphate-buffered saline at physiological pH may be used. Preservatives, stabilizers, and even dyes may be provided in the pharmaceutical composition. For example, sodium benzoate, sorbic acid and esters of p-hydroxybenzoic acid may be added as preservatives. Id. at 1449. In addition, antioxidants and suspending agents may be used. Id. By “pharmaceutically acceptable” it is meant a material that is not biologically or otherwise undesirable, i.e., the material can be administered to a subject without causing any undesirable biological effects such as toxicity. The formulations of the invention can optionally comprise additional medicinal agents, pharmaceutical agents, carriers, buffers, adjuvants, dispersing agents, diluents, and the like.

The compositions described herein can be administered to a subject for any vaccination, therapeutic or diagnostic purposes. The composition may be administered to a subject in an amount sufficient to cause to viral replication in the subject.

In some implementations provided herein, the pharmaceutical compositions provided herein capable of being filtered through a 0.45 micron filter. In some implementations, the pharmaceutical composition is capable of being filtered through a 0.22 micron filter. In some implementations, the pharmaceutical composition is capable of being filtered through a 0.20 micron filter.

In implementation, the compositions include “naked RNA” which is an RNA polynucleotide without an artificial RNA delivery system. In one implementation, the present invention is drawn to a pharmaceutical composition comprising ribonucleic acid (RNA) polynucleotide encoding a replication-competent viral genome and an associated artificial RNA delivery system. Such a composition may be administered to a subject in order to stimulate an immune response, e.g., an antigen-specific immune response. In some implementations, the pharmaceutical composition is specifically a vaccine composition that comprises the compositions described herein in combination with a pharmaceutically acceptable carrier, excipient or diluent. Illustrative carriers are usually nontoxic to recipients at the dosages and concentrations employed.

In some aspects, the pharmaceutical compositions provided herein are administered to a subject to generate a response in the subject, for example, for generating an immune response in the subject. Typically, a therapeutically effective amount is administered to the subject.

The term “effective amount” or “therapeutically effective amount” in the context of vaccines is the amount of vaccine composition, antigen, or antigen encoding nucleic acid that when administer to a subject induces a protective immune response. A protective immune response includes protection against symptoms or decrease in severity of symptoms as well as prevention of infection. An effective amount of the RNA polynucleotide is administered in an “effective regime.” The term “effective regime” refers to a combination of amount of the composition being administered and dosage frequency adequate to accomplish the desired effect. A single dose may be sufficient for the vaccine compositions of this disclosure to induce an immune response such as generating protective immunity. Thus, in such implementations multiple doses are not required to generate protective immunity.

Actual dosage levels may be varied so as to obtain an amount that is effective to achieve a desired response for a particular patient, composition, and mode of administration, without being toxic to the patient. The selected dosage level will depend upon a variety of pharmacokinetic factors in combination with the particular compositions employed, the age, sex, weight, condition, general health, and prior medical history of the subject being treated, and like factors well-known in the medical arts.

Suitable dosages of the RNA polynucleotide will vary depending upon the condition, age and species of the subject, the nature of the virus, the presence of any adjuvants, the level of immunogenicity and enhancement desired, and like factors, and can be readily determined by those skilled in the art. Single or multiple (i.e., booster) dosages of viral adjuvant and/or immunogen can be administered. In an implementation, a single dose may induce an immune response such as protective immunity. In an implementation, two or more doses may be necessary to induce protective immunity.

In illustrative vaccine-based implementations provided herein, about 1 μg-100 μg of the antigen or 0.1 μg-10 mg of the nucleic acid encoding the antigen will be administered per dose. Illustrative formulations of the present permit a dose of from about 0.1 μg, about 1 μg, about 5 μg, or about 10 ug, or about 100 μg to about 500 μg of replicon RNA. Illustrative formulations of the present permit a human dose of about 1 μg to about 800 μg RNA.

It will be evident to those skilled in the art that the number and frequency of administrations will be dependent upon the response of the subject. Illustrative formulations allow for therapeutic efficacy after as little as one immunization.

The pharmaceutical compositions may be implemented as a vaccine. Typically vaccines are prepared in an injectable form, either as a liquid solution or as a suspension. Solid forms suitable for injection may also be prepared as emulsions, or with the polypeptides encapsulated in liposomes. Vaccine antigens are usually combined with a pharmaceutically acceptable carrier, which includes any carrier that does not induce the production of antibodies harmful to the subject receiving the carrier. Suitable carriers typically comprise large macromolecules that are slowly metabolized, such as proteins, polysaccharides, polylactic acids, polyglycolic acids, polymeric amino acids, amino acid copolymers, lipid aggregates, and inactive virus particles. Such carriers are well known to those skilled in the art. These carriers may also function as adjuvants.

The pharmaceutical compositions may be in any form which allows for the composition to be administered to a patient. For example, the composition may be in the form of a solid, liquid, or gas (aerosol). Typical routes of administration include, without limitation, oral, topical, parenteral, sublingual, buccal, rectal, vaginal, intravenous, intradermal, transdermal, intranasal, intramucosal, pulmonary or subcutaneous. The term parenteral as used herein includes iontophoretic, sonophoretic, thermal, transdermal administration and also subcutaneous injections, intravenous, intramuscular, intrasternal, intracavernous, intrathecal, intrameatal, intraurethral injection or infusion techniques. In some implementations, a composition as described herein (including vaccine and pharmaceutical compositions) is administered intradermally by a technique selected from iontophoresis, microcavitation, sonophoresis, jet injection, or microneedles. In one implementation, a composition as described herein is administered intradermally using the microneedle device manufactured by NanoPass Technologies Ltd., Nes Ziona, Israel, e.g., MicronJet600 (see, e.g., U.S. Pat. Nos. 6,533,949 and 7,998,119 and Yotam, et al., Human vaccines & immunotherapeutics 11(4): 991-997 (2015).

In certain implementations, the compositions of the present disclosure may be delivered by intranasal sprays, inhalation, and/or other aerosol delivery vehicles. Methods for delivering genes, polynucleotides, and peptide compositions directly to the lungs via nasal aerosol sprays has been described e.g., in Southam et al., Distribution of intranasal instillations in mice: effects of volume, time, body position, and anesthesia, Am J Physiol Lung Cell Mol Physiol, Volume 282, 2002, pages L833-L839, U.S. Pat. Nos. 5,756,353 and 5,804,212. Likewise, the delivery of drugs using intranasal microparticle resins (Takenaga et al., Microparticle resins as a potential nasal drug delivery system for insulin, Journal of Controlled Release, Volume 52, Issues 1-2, 1998, Pages 81-87,) and lysophosphatidyl-glycerol compounds (U.S. Pat. No. 5,725,871) are also well-known in the pharmaceutical arts. Likewise, transmucosal drug delivery in the form of a polytetrafluoroetheylene support matrix is described in U.S. Pat. No. 5,780,045.

The pharmaceutical composition can be formulated so as to allow the RNA polynucleotides contained therein to enter the cytoplasm of a cell upon administration of the composition to a subject. Compositions that will be administered to a subject take the form of one or more dosage units, where for example, a vial or ampule may contain a single dosage unit, and a container of one or more compounds of the invention in aerosol form may hold a plurality of dosage units.

For oral administration, an excipient and/or binder may be present. Examples are sucrose, kaolin, glycerin, starch dextrins, sodium alginate, carboxymethylcellulose and ethyl cellulose. Coloring and/or flavoring agents may be present. A coating shell may be employed.

The composition may be in the form of a liquid, e.g., an elixir, syrup, solution, emulsion or suspension. The liquid may be for oral administration or for delivery by injection, as two examples. When intended for oral administration, compositions can contain one or more of a sweetening agent, preservatives, dye/colorant and flavor enhancer. In a composition intended to be administered by injection by needle and syringe or needle free jet injection, one or more of a surfactant, preservative, wetting agent, dispersing agent, suspending agent, buffer, stabilizer and isotonic agent may be included.

A liquid pharmaceutical composition as used herein, whether in the form of a solution, suspension or other like form, may include one or more of the following carriers or excipients: sterile diluents such as water for injection, saline solution, preferably physiological saline, Ringer's solution, isotonic sodium chloride, fixed oils such as squalene, squalane, mineral oil, a mannide monooleate, cholesterol, and/or synthetic mono or digylcerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose.

In another implementation, a composition of the present disclosure is formulated in a manner which can be aerosolized.

It may also be desirable to include other components in a pharmaceutical composition, such as delivery vehicles including but not limited to aluminum salts, water-in-oil emulsions, biodegradable oil vehicles, oil-in-water emulsions, biodegradable microcapsules, and liposomes. Examples of additional immunostimulatory substances (co-adjuvants) for use in such vehicles are also described above and may include N-acetylmuramyl-L-alanine-D-isoglutamine (MDP), glucan, IL-12, GM-CSF, gamma interferon and IL-12.

In some implementations, the compositions of the present invention comprise a buffering agent. Buffering agents useful as excipients in the present invention include Tris acetate, Tris base, Tris-HCl, ammonium phosphate, citric acid, sodium citrate, potassium citrate, tartic acid, sodium phosphate, zinc chloride, arginine, and histidine. Concentration of the buffering agents may range between 1-20 mM such as, for example 5 mM, 10 mM, or 20 mM. In some implementations buffering agents include pH adjusting agents such as hydrochloric acid, sodium hydroxide, and meglumine.

While any suitable carrier known to those of ordinary skill in the art may be employed in the pharmaceutical compositions of the present disclosure, the type of carrier will vary depending on the mode of administration and whether a sustained release is desired. For parenteral administration, such as subcutaneous injection, the carrier can comprise water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactic galactide) may also be employed as carriers for the pharmaceutical compositions of this invention. Suitable biodegradable microspheres are disclosed, for example, in U.S. Pat. Nos. 4,897,268 and 5,075,109. In this regard, it is preferable that the microsphere be larger than approximately 25 microns.

Pharmaceutical compositions may also contain diluents such as buffers, antioxidants such as ascorbic acid, polypeptides, proteins, amino acids, carbohydrates including glucose, sucrose or dextrins, chelating agents such as EDTA, glutathione and other stabilizers and excipients. Neutral buffered saline or saline mixed with nonspecific serum albumiskilln are illustrative appropriate diluents. For example, a product may be formulated as a lyophilizate using appropriate excipient solutions (e.g., sucrose) as diluents.

The pharmaceutical composition may be intended for topical administration, in which case the carrier may suitably comprise a solution, emulsion, ointment or gel base. The base, for example, may comprise one or more of the following: petrolatum, lanolin, polyethylene glycols, beeswax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the antigen (e.g., GLA-antigen vaccine composition) or GLA (e.g., immunological adjuvant composition; GLA is available from Avanti Polar Lipids, Inc., Alabaster, AL; e.g., product number 699800) of from about 0.1 to about 10% w/v (weight per unit volume).

The composition may be intended for rectal administration, in the form, e.g., of a suppository which can melt in the rectum and release the drug. The composition for rectal administration may contain an oleaginous base as a suitable nonirritating excipient. Such bases include, without limitation, lanolin, cocoa butter and polyethylene glycol. In the methods of the invention, the pharmaceutical compositions/adjuvants may be administered through use of insert(s), bead(s), timed-release formulation(s), patch(es) or fast-release formulation(s).

Optionally, to control tonicity, the NLC may comprise a physiological salt, such as a sodium salt. Sodium chloride (NaCl), for example, may be used at about 0.9% (w/v) (physiological saline). Other salts that may be present include potassium chloride, potassium dihydrogen phosphate, disodium phosphate, magnesium chloride, calcium chloride, etc. Non-ionic tonicifying agents can also be used to control tonicity. Monosaccharides classified as aldoses such as glucose, mannose, arabinose, and ribose, as well as those classified as ketoses such as fructose, sorbose, and xylulose can be used as non-ionic tonicifying agents in the presently disclosed compositions. Disaccharides such a sucrose, maltose, trehalose, and lactose can also be used. In addition, alditols (acyclic polyhydroxy alcohols, also referred to as sugar alcohols) such as glycerol, mannitol, xylitol, and sorbitol are non-ionic tonicifying agents useful in the presently disclosed compositions. Non-ionic tonicity modifying agents can be present at a concentration of from about 0.1% to about 10% or about 1% to about 10%, depending upon the agent that is used. If pharmaceutical compositions are formulated for parenteral administration, it is preferable to make the osmolarity of the pharmaceutical composition the same as normal physiological fluids, preventing post-administration consequences, such as post-administration swelling or rapid absorption of the composition.

Optionally, pharmaceutical compositions may be formulated with cryoprotectants comprising, Avicel PH102 (microcrystalline cellulose), Avicel RC591 (mixture of microcrystalline cellulose and sodium carboxymethyl cellulose), Mircrocelac® (mixture of lactose and Avicel), or a combination thereof. Optionally, pharmaceutical compositions may be formulated with a preservative agent such as, for example, Hydrolite 5.

### VII. Methods of Using the Compositions of the Present Disclosure

A. Vaccine

This disclosure provides vaccines against positive stranded RNA viruses. In an implementation, this disclosure provides a Chikungunya virus (CHIKV) vaccine that includes a ribonucleic acid (RNA) polynucleotide encoding an attenuated, replication-competent CHIKV genome. The CHIKV genome may be a genome of any strain of the chikungunya virus such as CHIKV 181/25, CHIKV-Δ5nsp3, or CHIKV-Δ5nsp3. In a further implementation, this disclosure provides a yellow fever (YF) vaccine that includes a ribonucleic acid (RNA) polynucleotide encoding an attenuated, replication-competent yellow fever genome. The YF genome may be a genome of any strain of the yellow fever virus such as YF17D.

The present disclosure thus provides compositions for altering (i.e., increasing or decreasing in a statistically significant manner, for example, relative to an appropriate control as will be familiar to persons skilled in the art) immune responses in a host capable of mounting an immune response. As will be known to persons having ordinary skill in the art, an immune response may be any active alteration of the immune status of a host, which may include any alteration in the structure or function of one or more tissues, organs, cells or molecules that participate in maintenance and/or regulation of host immune status. Typically, immune responses may be detected by any of a variety of well-known parameters, including but not limited to in vivo or in vitro determination of: soluble immunoglobulins or antibodies; soluble mediators such as cytokines, lymphokines, chemokines, hormones, growth factors and the like as well as other soluble small peptide, carbohydrate, nucleotide and/or lipid mediators; cellular activation state changes as determined by altered functional or structural properties of cells of the immune system, for example cell proliferation, altered motility, induction of specialized activities such as specific gene expression or cytolytic behavior; cellular differentiation by cells of the immune system, including altered surface antigen expression profiles or the onset of apoptosis (programmed cell death); or any other criterion by which the presence of an immune response may be detected.

Determination of the induction of an immune response by the compositions of the present disclosure may be established by any of a number of well-known immunological assays with which those having ordinary skill in the art will be readily familiar. Such assays include, but need not be limited to, to in vivo or in vitro determination of: soluble antibodies; soluble mediators such as cytokines, lymphokines, chemokines, hormones, growth factors and the like as well as other soluble small peptide, carbohydrate, nucleotide and/or lipid mediators; cellular activation state changes as determined by altered functional or structural properties of cells of the immune system, for example cell proliferation, altered motility, induction of specialized activities such as specific gene expression or cytolytic behavior; cellular differentiation by cells of the immune system, including altered surface antigen expression profiles or the onset of apoptosis (programmed cell death). Procedures for performing these and similar assays are widely known and may be found, for example in Lefkovits (Immunology Methods Manual: The Comprehensive Sourcebook of Techniques, 1998; see also Current Protocols in Immunology; see also, e.g., Weir, Handbook of Experimental Immunology, 1986 Blackwell Scientific, Boston, MA; Mishell and Shigii (eds.) Selected Methods in Cellular Immunology, 1979 Freeman Publishing, San Francisco, CA; Green and Reed, 1998 Science 281:1309 and references cited therein.).

Detection of the proliferation of antigen-reactive T cells may be accomplished by a variety of known techniques. For example, T cell proliferation can be detected by measuring the rate of DNA synthesis, and antigen specificity can be determined by controlling the stimuli (such as, for example, a specific desired antigenor a control antigen-pulsed antigen presenting cells) to which candidate antigen-reactive T cells are exposed. T cells which have been stimulated to proliferate exhibit an increased rate of DNA synthesis. A typical way to measure the rate of DNA synthesis is, for example, by pulse-labeling cultures of T cells with tritiated thymidine, a nucleoside precursor which is incorporated into newly synthesized DNA. The amount of tritiated thymidine incorporated can be determined using a liquid scintillation spectrophotometer. Other ways to detect T cell proliferation include measuring increases in interleukin-2 (IL-2) production, Ca2+ flux, or dye uptake, such as 3-(4,5-dimethylthiazol-2-yl)-2,5-diphenyl-tetrazolium. Alternatively, synthesis of lymphokines (such as interferon-gamma) can be measured or the relative number of T cells that can respond to a particular antigen may be quantified.

Detection of antigen-specific antibody production may be achieved, for example, by assaying a sample (e.g., an immunoglobulin containing sample such as serum, plasma or blood) from a host treated with a vaccine according to the present disclosure using in vitro methodologies such as radioimmunoassay (RIA), enzyme linked immunosorbent assays (ELISA), equilibrium dialysis or solid phase immunoblotting including Western blotting. In implementations ELISA assays may further include antigen-capture immobilization of the target antigen with a solid phase monoclonal antibody specific for the antigen, for example, to enhance the sensitivity of the assay. Elaboration of soluble mediators (e.g., cytokines, chemokines, lymphokines, prostaglandins, etc.) may also be readily determined by enzyme-linked immunosorbent assay (ELISA), for example, using methods, apparatus and reagents that are readily available from commercial sources (e.g., Sigma, St. Louis, MO; see also R & D Systems 2006 Catalog, R & D Systems, Minneapolis, MN).

Any number of other immunological parameters may be monitored using routine assays that are well known in the art. These may include, for example, antibody dependent cell-mediated cytotoxicity (ADCC) assays, secondary in vitro antibody responses, flow immunocytofluorimetric analysis of various peripheral blood or lymphoid mononuclear cell subpopulations using well established marker antigen systems, immunohistochemistry or other relevant assays. These and other assays may be found, for example, in Rose et al. (Eds.), Manual of Clinical Laboratory Immunology, 5th Ed., 1997 American Society of Microbiology, Washington, DC.

Accordingly, it is contemplated that the compositions provided herein will be capable of eliciting or enhancing in a host at least one immune response that is selected from a Th1-type T lymphocyte response, a TH2-type T lymphocyte response, a cytotoxic T lymphocyte (CTL) response, an antibody response, a cytokine response, a lymphokine response, a chemokine response, and an inflammatory response. In certain implementations the immune response may comprise at least one of production of one or a plurality of cytokines wherein the cytokine is selected from interferon-gamma (IFN-γ), tumor necrosis factor-alpha (TNF-α), production of one or a plurality of interleukins wherein the interleukin is selected from IL-1, IL-2, IL-3, IL-4, IL-6, IL-8, IL-10, IL-12, IL-13, IL-16, IL-18 and IL-23, production one or a plurality of chemokines wherein the chemokine is selected from MIP-1α, MIP-1β, RANTES, CCL2, CCL4, CCL5, CXCL1, and CXCLS, and a lymphocyte response that is selected from a memory T cell response, a memory B cell response, an effector T cell response, a cytotoxic T cell response and an effector B cell response.

In one embodiment, an immune response protects the subject from a CHIKV infection, or inflammatory consequences thereof (e.g., arthritis). The administration of this immunological composition may be used either therapeutically in subjects already experiencing a CHIKV infection or may be used prophylactically to prevent a CHIKV infection.

In one embodiment, an immune response protects the subject from a yellow fever infection, or symptoms thereof. The administration of this immunological composition may be used either therapeutically in subjects already experiencing a yellow fever infection or may be used prophylactically to prevent a yellow fever infection.

B. Methods of Administration

Methods of administering the composition include, without limitation, oral, topical, parenteral, sublingual, buccal, rectal, vaginal, intravenous, intradermal, transdermal, intranasal, intramucosal, or subcutaneous. In implementations, administration of the composition is intramuscular, parenteral, or intradermal. In such implementations, the subject is a mammal (e.g., an animal including farm animals (cows, pigs, goats, horses, etc.), pets (cats, dogs, etc.), and rodents (rats, mice, etc.), or a human). In one implementation, the subject is a human. In another implementation, the subject is a non-human mammal. In another implementation, the non-human mammal is a dog, cow, or horse.

The vaccines and compositions of this disclosure may be delivered to the cytosol of a cell of a subject. In some implementations, the vaccines and compositions of this disclosure are delivered to the cytosol without delivery to the nucleus. The vaccines and compositions of this disclosure may be administered without electroporation. The vaccines and compositions of this disclosure may be administered without use of a biolistic particle delivery system. Examples of biolistic particle delivery systems include devices such as a “gene gun,” air pistol or a HELIOS™ gene gun (Bio-Rad Laboratories, Hercules, CA).

In an implementation the mode of delivery is intradermal. The intradermal delivery can be conducted by the use of microneedles, with height of less than 1 mm or 1000 micron; and more preferably with height of 500-750 micron. A microneedle injection device preferably has multiple needles, typically 3 microneedles.

In some implementations, multiple modes of delivery may be used to obtain greater immune response. For example, the composition can be administered 1, 2, 3, 4, 5, 6, or more times. In some implementation, the one or more administrations may occur as part of a so-called “prime-boost” protocol. In some implementations the “prime-boost” approach comprises administration in in several stages that present the same antigen through different vectors or multiple doses. In some implementations, administration may occur more than twice, e.g., three times, four times, etc., so that the first priming administration is followed by more than one boosting administration. When multiple vectors or doses are administered, they can be separated from one another by, for example, one week, two weeks, three weeks, one month, six weeks, two months, three months, six months, one year, or longer.

### VIII. Methods of Generating an Immune Response

This disclosure provides a method of producing an immune response against an immunogen in a subject, the method comprising administering to the subject ribonucleic acid (RNA) polynucleotide encoding a replication-competent viral genome in an amount sufficient to cause to viral replication in the subject. In some implementations the RNA polynucleotide is complexed with or contained within an artificial RNA delivery system. In some implementations, methods of boosting or enhancing an immune response are provided. Optionally, an immunogenically effective amount is sufficient to produce a protective immune response. The degree of protection conferred need not be complete or permanent. A “protective” immune response or “protective” immunity as used herein indicates that the immune response confers some benefit to the subject in that it prevents or reduces the incidence and/or severity of disease.

Immune response may be generated by causing a viral infection that includes actively replicating virus particles. Thus, in some implementations the compositions and vaccines at this disclosure may be used in a method of causing a viral infection in a cell.

CHIKV-reactive antibodies are generally considered to be appropriate correlates of protection for CHIKV and YFV vaccines (Milligan, G. N.; Schnierle, B. S.; McAuley, A. J.; Beasley, D. W. C., Defining a correlate of protection for chikungunya virus vaccines. Vaccine 2019, 37 (50), 7427-7436; Justin G. Julander, Dennis W. Trent, Thomas P. Monath, Immune correlates of protection against yellow fever determined by passive immunization and challenge in the hamster model, Vaccine, 29 (35), 2011, 6008-6016; Reinhardt, B., Jaspert, R., Niedrig, M., Kostner, C. and L'age-Stehr, J. (1998), Development of viremia and humoral and cellular parameters of immune activation after vaccination with yellow fever virus strain 17D: A model of human flavivirus infection. J. Med. Virol., 56: 159-167). Thus, a protective immune response to CHIKV or YFV may be detected by antibody titers as well as by survival studies.

In some implementations, the composition induces an immune response (e.g., neutralizing antibody titers) in the subject at a level that is at least 80% of the immune response induced in the subject by a traditional live-attenuated vaccine. The level of immune response may be 80%, 85%, 90%, 95%, 99%, 100%, or even higher than the immune response induced the corresponding vaccine comprising a live-attenuated virus. Immune response may be, for example, innate, cellular or antibody responses. Neutralizing antibody titers may be determined by any assay known to one of skill in the art, including, without limitation, a plaque reduction neutralization titer analysis (Ratnam, S et al. J. Clin. Microbiol (2011), 33 (4): 811-815; Timiryazova, T et al. Am J Trop Med Hyg (2013), 88(5): 962-970).

Typical routes of administration of the therapeutically effective amount of the composition include, without limitation, oral, topical, parenteral, sublingual, buccal, rectal, vaginal, intravenous, intradermal, transdermal, intranasal, intramucosal, or subcutaneous. In some illustrative implementations, administration of the composition is intramuscular, ocular, parenteral, or pulmonary.

In illustrative implementations, the compositions disclosed herein are vaccine compositions and are used as vaccines. The compositions described herein can be used for generating an immune response in the subject (including a non-specific response and an antigen-specific response). In some implementations, the immune response comprises a systemic immune response. In some implementations, the immune response comprises a mucosal immune response. Generation of an immune response includes stimulating an immune response, boosting an immune response, or enhancing an immune response.

The compositions described herein may be used to enhance protective immunity against a positive strand virus. Such viruses and viral antigens include, for example, coronaviruses (such as SARS, MERS, and SARS-CoV-2), flaviviruses (e.g., dengue virus, Japanese encephalitis virus, yellow fever virus, Zika virus, Poswassan virus, tick-borne encephalitis virus), and alphaviruses.

Methods for determining whether a composition of the present inventions is capable of effectively delivering the bioactive agent and/or having the desired effect in a subject are known in the art and not described herein in detail. In one aspect, immune responses against an antigen can be determined by monitoring the level antigen-specific antibody before and after administration (e.g., systemic IgM, IgG (IgG1, IgG2a, et al.) or IgA) in blood samples or from mucosal sites. Cellular immune responses also can be monitored after administration by assessing T and B cell function after antigen stimulation.

Another way of assessing the immunogenicity of the compositions or vaccines disclosed herein where the nucleic acid molecule (e.g., the RNA) encodes a protein antigen is to express the recombinant protein antigen for screening patient sera or mucosal secretions by immunoblot and/or microarrays. A positive reaction between the protein and the patient sample indicates that the patient has mounted an immune response to the protein in question. This method may also be used to identify immunodominant antigens and/or epitopes within protein antigens.

The efficacy of the compositions can also be determined in vivo by challenging appropriate animal models of the pathogen of interest infection.

In the implementations provided herein, the subject is a mammal (e.g., an animal including farm animals (cows, pigs, goats, horses, etc.), pets (cats, dogs, etc.), and rodents (rats, mice, etc.), or a human). In one implementation, the subject is a human. In another implementation, the subject is a non-human mammal. In another implementation, the non-human mammal is a dog, cow, or horse.

### IX. Kits and Articles of Manufacture

Also contemplated in certain implementations are kits comprising the ribonucleic acid (RNA) polynucleotide encoding a replication-competent viral genome and optionally an artificial RNA delivery system, which may be provided in one or more containers. In one implementation, all components of the compositions are present together in a single container. In other implementations, components of the compositions may be in two or more containers.

In some implementations, one vial of the kit comprises ribonucleic acid (RNA) polynucleotide encoding a replication-competent viral genome as provided herein, and a second vial of the kit contains an artificial RNA delivery system. In some implementations, the kit comprises a third vial containing an optional component.

The kits of the invention may further comprise instructions for use as herein described or instructions for mixing the materials contained in the vials. In some implementations, the material a vial is dry or lyophilized. In some implementations, the material in a vial is liquid.

A container according to such kit implementations may be any suitable container, vessel, vial, ampule, tube, cup, box, bottle, flask, jar, dish, well of a single-well or multi-well apparatus, reservoir, tank, or the like, or other device in which the herein disclosed compositions may be placed, stored and/or transported, and accessed to remove the contents. Typically, such a container may be made of a material that is compatible with the intended use and from which recovery of the contained contents can be readily achieved. Nonlimiting examples of such containers include glass and/or plastic sealed or re-sealable tubes and ampules, including those having a rubber septum or other sealing means that is compatible with withdrawal of the contents using a needle and syringe. Such containers may, for instance, by made of glass or a chemically compatible plastic or resin, which may be made of, or may be coated with, a material that permits efficient recovery of material from the container and/or protects the material from, e.g., degradative conditions such as ultraviolet light or temperature extremes, or from the introduction of unwanted contaminants including microbial contaminants. The containers are preferably sterile or sterilizable, and made of materials that will be compatible with any carrier, excipient, solvent, vehicle or the like, such as may be used to suspend or dissolve the herein described vaccine compositions and/or immunological adjuvant compositions and/or antigens and/or recombinant expression constructs, etc.

### X. Illustrative Implementations

Implementation 1. A composition for causing viral infection in a subject, the composition comprising: a. a ribonucleic acid (RNA) polynucleotide encoding a replication-competent viral genome; and b. an artificial RNA delivery system, wherein the RNA is present in an amount sufficient to cause to viral replication in the subject.
Implementation 2. The composition of implementation 1, wherein the RNA is transcribed from a DNA plasmid.
Implementation 3. The composition of any of implementations 1-2, wherein the viral genome is a genome of an attenuated virus.
Implementation 4. The composition of implementation 3, wherein the viral genome is a full-length genome.
Implementation 5. The composition of any of implementations 1-4, wherein the RNA is single-stranded.
Implementation 6. The composition of any of implementations 1-5, wherein the RNA is present in an amount sufficient to induce neutralizing antibodies in the subject.
Implementation 7. The composition of implementation 6, wherein a titer of neutralizing antibodies is the same as induced by live viral vaccination.
Implementation 8. The composition of implementation 6, wherein a titer of the neutralizing antibodies exceeds a titer that is a correlate of protection.
Implementation 9. The composition of any of implementations 1-8, wherein the composition does not include an additional adjuvant.
Implementation 10. The composition of any of implementations 1-9, wherein the viral genome is a genome of a positive strand virus.
Implementation 11. The composition of implementation 10, wherein the positive strand virus is an Alphavirus.
Implementation 12. The composition of implementation 11, wherein the alphavirus is Chikungunya (CHIKV).
Implementation 13. The composition of implementation 12, wherein the CHIKV is CHIKV 181/25.
Implementation 14. The composition of implementation 12, wherein the CHIKV is CHIKV-Δ5nsp3.
Implementation 15. The composition of implementation 12, wherein the CHIKV is CHIKV-Δ6K.
Implementation 16. The composition of implementation 10, wherein the positive strand virus is a flavivirus.
Implementation 17. The composition of implementation 16, wherein the flavivirus is yellow fever virus, Zika virus, Japanese encephalitis virus, West Nile virus, hepatitis C virus, tick-borne encephalitis, Powassan virus, or dengue virus.
Implementation 18. The composition of implementation 17, wherein the positive strand virus is yellow fever.
Implementation 19. The composition of implementation 18, wherein the yellow fever is YF17D.
Implementation 20. The composition of implementation 10, wherein the positive strand virus is a coronavirus.
Implementation 21. The composition of implementation 20, wherein the coronavirus is MERS, SARS, or SARS-CoV-2.
Implementation 22. A Chikungunya virus (CHIKV) vaccine, comprising: a. a ribonucleic acid (RNA) polynucleotide encoding an attenuated, replication-competent CHIKV genome; and b. an artificial RNA delivery system, wherein the RNA is present in an amount sufficient to cause to viral replication in the subject.
Implementation 23. The vaccine of implementation 22, wherein the CHIKV genome is CHIKV 181/25.
Implementation 24. The vaccine of implementation 22, wherein the CHIKV genome is CHIKV-Δ5nsp3.
Implementation 25. The vaccine of implementation 22, wherein the CHIKV genome is CHIKV-Δ6K.
Implementation 26. A yellow fever virus vaccine, comprising: a. a ribonucleic acid (RNA) polynucleotide encoding an attenuated, replication-competent yellow fever genome; and b. an artificial RNA delivery system, wherein the RNA is present in an amount sufficient to cause to viral replication in the subject.
Implementation 27. The vaccine of implementation 26, wherein the yellow fever genome is YF17D.
Implementation 28. The vaccine of any of implementations 22-27, wherein the RNA is transcribed from a DNA plasmid.
Implementation 29. The vaccine of any of implementations 22-29, wherein the viral genome is a full-length genome.
Implementation 30. The vaccine of any of implementations 22-29, wherein the RNA is single-stranded.
Implementation 31. The vaccine of any of implementations 22-30, wherein the RNA is present in an amount sufficient to induce neutralizing antibodies in a subject.
Implementation 32. The vaccine of implementation 31, wherein a titer of neutralizing antibodies is the same as induced by live viral vaccination.
Implementation 33. The vaccine of implementation 31, wherein a titer of neutralizing antibodies exceeds a titer that is a correlate of protection.
Implementation 34. The vaccine of any of implementations 22-33, wherein the composition does not include an additional adjuvant.
Implementation 35. The composition or vaccine of any of implementations 1-34, wherein the artificial RNA delivery system comprises a lipid particle.
Implementation 36. The composition or vaccine of implementation 35, wherein the lipid particle is a lipid nanoparticle (LNP).
Implementation 37. The composition or vaccine of implementation 35, wherein the lipid particle is a nanostructured lipid carrier (NLC).
Implementation 38. The composition or vaccine of implementation 37, wherein the NLC comprises a liquid oil, a solid lipid, a hydrophobic sorbitan ester, a hydrophilic ethoxylated sorbitan ester, and a cationic lipid.
Implementation 39. The composition or vaccine of implementation 38, wherein liquid oil is squalene or synthetic squalene, solid lipid is Glyceryl trimyristate, the hydrophobic sorbitan ester is sorbitan monostearate, the hydrophilic ethoxylated sorbitan ester is polysorbate 80, and the cationic lipid is DOTAP (N-[1-[2,3-Dioleoyloxy)propyl]-N,N,N-trimethylammonium chloride).
Implementation 40. The composition or vaccine of implementation 35, wherein the lipid particle is a cationic nanoemulsion (CNE).
Implementation 41. The composition or vaccine of any of implementations 1-35, wherein the artificial RNA delivery system comprises amphiphilic diblock oligomers containing a sequence of lipid monomers and a sequence of cationic monomers.
Implementation 42. A pharmaceutical composition comprising the composition or vaccine of any of implementations 1-41, and at least one pharmaceutically acceptable carrier, excipient, and/or adjuvant.
Implementation 43. A method of inducing an immune response in a subject comprising, administering to the subject ribonucleic acid (RNA) polynucleotide encoding a replication-competent viral genome in an amount sufficient to cause to viral replication in the subject.
Implementation 44. A method of causing a viral infection in a cell, comprising contacting the cell with ribonucleic acid (RNA) polynucleotide encoding a replication-competent viral genome complexed with or contained within an artificial RNA delivery system.
Implementation 45. The method of any of implementations 43-44, wherein the RNA is transcribed from a DNA plasmid.
Implementation 46. The composition of any of implementations 43-45, wherein the viral genome is a genome of an attenuated virus.
Implementation 47. The method of implementation 46, wherein the viral genome is a full-length genome.
Implementation 48. The method of any of implementations 43-47, wherein the RNA is single-stranded.
Implementation 49. The method of any of implementations 43-44, wherein the viral genome is a genome of a positive strand virus.
Implementation 50. The method of implementation 45, wherein the positive strand virus is an Alphavirus.
Implementation 51. The method of implementation 50, wherein the alphavirus is Chikungunya (CHIKV).
Implementation 52. The method of implementation 51, wherein the CHIKV is CHIKV 181/25.
Implementation 53. The method of implementation 51, wherein the CHIKV is CHIKV-Δ5nsp3.
Implementation 54. The method of implementation 51, wherein the CHIKV is CHIKV-Δ6K.
Implementation 55. The method of implementation 45, wherein the positive strand virus is a flavivirus.
Implementation 56. The method of implementation 55, wherein the flavivirus is yellow fever virus, ZIKA virus, Japanese encephalitis virus, West Nile virus, hepatitis C virus, tick-borne encephalitis, or dengue virus.
Implementation 57. The method of implementation 45, wherein the positive strand virus is yellow fever.
Implementation 58. The method of implementation 57, wherein the yellow fever is YF17D.
Implementation 59. The method of implementation 45, wherein the positive strand virus is a coronavirus.
Implementation 60. The method of implementation 59, wherein the coronavirus is MERS, SARS, or SARS-CoV-2.
Implementation 61. A method of inducing protective immunity in a subject against Chikungunya virus (CHIKV) comprising, administering to the subject a ribonucleic acid (RNA) polynucleotide encoding an attenuated, replication-competent CHIKV genome in an amount sufficient to cause to viral replication in the subject.
Implementation 62. The method of implementation 61, wherein the CHIKV genome is CHIKV 181/25.
Implementation 63. The method of implementation 61, wherein the CHIKV genome is CHIKV-Δ5nsp3.
Implementation 64. The method of implementation 61, wherein the CHIKV genome is CHIKV-Δ6K.
Implementation 65. A method of inducing protective immunity in a subject against yellow fever comprising, administering to the subject a ribonucleic acid (RNA) polynucleotide encoding an attenuated, replication-competent yellow fever genome in an amount sufficient to cause to viral replication in the subject.
Implementation 66. The method of implementation 65, wherein the yellow fever genome is YF17D.
Implementation 67. The method of any of implementations 43-66, wherein the RNA administered to the subject is complexed with or contained within an artificial RNA delivery system.
Implementation 68. The method of implementation 67, wherein the artificial RNA delivery system comprises a lipid particle.
Implementation 69. The method of implementation 68, wherein the lipid particle is a lipid nanoparticle (LNP).
Implementation 70. The method of implementation 68, wherein the lipid particle is a nanostructure lipid carrier (NLC).
Implementation 71. The method of implementation 70, wherein the NLC comprises a liquid oil, a solid lipid, a hydrophobic sorbitan ester, a hydrophilic ethoxylated sorbitan ester, and a cationic lipid.
Implementation 72. The method of implementation 71, wherein liquid oil is squalene or synthetic squalene, solid lipid is Glyceryl trimyristate, the hydrophobic sorbitan ester is sorbitan monostearate, the hydrophilic ethoxylated sorbitan ester is polysorbate 80, and the cationic lipid is DOTAP (N-[1-[2,3-Dioleoyloxy)propyl]-N,N,N-trimethylammonium chloride).
Implementation 73. The method of implementation 68, wherein the lipid particle is a cationic nanoemulsion (CNE).
Implementation 74. The method of implementation 67, wherein the artificial RNA delivery system comprises amphiphilic diblock oligomers containing a sequence of lipid monomers and a sequence of cationic monomers.
Implementation 75. The method of any one of implementations 43-74, wherein the immune response is induced after a single dose.
Implementation 76. The method of any one of implementations 43-75, wherein the administering does not include electroporation.
Implementation 77. The method of any one of implementations 43-76, wherein the administering does not include a biolistic particle delivery system.
Implementation 78. The method of any one of implementations 43-77, wherein the immune response comprises neutralizing antibodies.
Implementation 79. The method of implementation 78, wherein a titer of the neutralizing antibodies is the same as induced by live viral vaccination.
Implementation 80. The method of implementation 78, wherein a titer of the neutralizing antibodies exceeds a titer that is a correlate of protection.
Implementation 81. The method of any one of implementations 43-80, wherein the administering is intramuscular.
Implementation 82. The method of any one of implementations 43-80, wherein the administering is subcutaneous.
Implementation 83. The method of any one of implementations 43-80, wherein the administering is intranasal.
Implementation 84. The method of any one of implementations 43-83, wherein the amount is 1 μg.
Implementation 85. The method of any one of implementations 43-83, wherein the amount is 10 μg.
Implementation 86. The method of any one of implementations 43-83, wherein the amount is 100 μg.

### EXAMPLES

The following Examples are offered by way of illustration and not by way of limitation.

### Example 1: RNA Successfully Complexes with NLC and is Protected from RNase Challenge

Viral Plasmids and Cloning

To test the use of whole-genome CHIKV RNA as safe and effective vaccines, we created DNA constructs containing the entire genome of four live-attenuated CHIKV variants. Construct CHIKV 181/25 contains the full-length 181/25 CHIKV strain sequence. (SEQ ID NO. 1) Three further constructs added additional previously-described attenuating mutations to the 181/25 sequence in order to achieve genetically stable attenuation and effectively compare whole-genome RNA vaccines to current live-attenuated vaccine candidates, as follows: Construct CHIKV 181/25-Δ5nsP3 contains the 181/25 CHIKV strain sequence with a deletion in the P1234 polyprotein of the nsP3 replicase gene, encoding for residues 1656 to 1717 (SEQ ID NO. 2) (Hallengard, D.; Kakoulidou, M.; Lulla, A.; Kummerer, B. M.; Johansson, D. X.; Mutso, M. et al., Novel attenuated Chikungunya vaccine candidates elicit protective immunity in C57BL/6 mice. J Virol 2014, 88 (5), 2858-66; Rogues, P.; Ljungberg, K.; Kummerer, B. M.; Gosse, L.; Dereuddre-Bosquet, N.; Tchitchek, N. et al., Attenuated and vectored vaccines protect nonhuman primates against Chikungunya virus. JCI Insight 2017, 2 (6), e83527). Construct CHIKV Δ6K contains the 181/25 CHIKV sequence with a deletion in the 46K genomic region, (Hallengard supra) representing amino acid residues 749 to 809 (SEQ ID NO. 3). Construct CHIKV 181/25-ECMV IRES substitutes an ECMV IRES for the native CHIKV subgenomic promoter (SEQ ID NO. 4), a method previously successfully used to attenuate the virulent La Reunion strain of CHIKV (CHIKV-LR) (Plante, K.; Wang, E.; Partidos, C. D.; Weger, J.; Gorchakov, R.; Tsetsarkin, K. et al., Novel chikungunya vaccine candidate with an IRES-based attenuation and host range alteration mechanism. PLoS Pathog 2011, 7 (7), e1002142; Roy, C. J.; Adams, A. P.; Wang, E.; Plante, K.; Gorchakov, R.; Seymour, R. L. et al., Chikungunya vaccine candidate is highly attenuated and protects nonhuman primates against telemetrically monitored disease following a single dose. J Infect Dis 2014, 209 (12), 1891-9; Plante, K. S.; Rossi, S. L.; Bergren, N. A.; Seymour, R. L.; Weaver, S. C., Extended Preclinical Safety, Efficacy and Stability Testing of a Live-attenuated Chikungunya Vaccine Candidate. PLoS Negl Trop Dis 2015, 9 (9), e0004007).

For comparison with other RNA vaccine technology, we also created construct CHIKV 181/25-CE mRNA, an mRNA-based CHIKV vaccine candidate that expresses the 181/25 strain structural proteins C, E1, and E2 but contains no full-length genomic RNA (SEQ ID NO. 5).

A plasmid containing the full-length CHIKV 181/25 genomic sequence under control of an SP6 promoter were modified with standard cloning techniques to replace the SP6 promoter with a T7 promoter to create the plasmid CHIKV-181/25. Plasmids CHIKV 181/25-Δ5nsP3, CHIKV-Δ6K and CHIKV 181/25-ECMV IRES, each containing CHIKV 181/25 genomes with additional published attenuating deletions, (Plante, K.; Wang, E.; Partidos, C. D.; Weger, J.; Gorchakov, R.; Tsetsarkin, K. et al., Novel chikungunya vaccine candidate with an IRES-based attenuation and host range alteration mechanism. PLoS Pathog 2011, 7 (7), e1002142; Hallengard supra; Rogues, P.; Ljungberg, K.; Kummerer, B. M.; Gosse, L.; Dereuddre-Bosquet, N.; Tchitchek, N. et al., Attenuated and vectored vaccines protect nonhuman primates against Chikungunya virus. JCI Insight 2017, 2 (6), e83527) (Roy, C. J.; Adams, A. P.; Wang, E.; Plante, K.; Gorchakov, R.; Seymour, R. L. et al., Chikungunya vaccine candidate is highly attenuated and protects nonhuman primates against telemetrically monitored disease following a single dose. J Infect Dis 2014, 209 (12), 1891-9; Plante, K. S.; Rossi, S. L.; Bergren, N. A.; Seymour, R. L.; Weaver, S. C., Extended Preclinical Safety, Efficacy and Stability Testing of a Live-attenuated Chikungunya Vaccine Candidate. PLoS Negl Trop Dis 2015, 9 (9), e0004007) were created from the CHIKV-181/25 plasmid by standard cloning methods.

Briefly, gene fragments containing the desired gene edits were synthesized (Integrated DNA Technologies), and cloned into digested, purified CHIKV-181/25 plasmid backbones using InFusion enzyme mix (Clontech) between PpuMI and SfiI (CHIKV 181/25-ECMV IRES), XhoI and SgrAI (CHIKV Δ6K), or PasI and Bpu10I (CHIKV 181/25-Δ5nsP3) restriction enzyme sites. All plasmid sequences were confirmed by Sanger sequencing. Plasmid sequences have been uploaded to GenBank as follows:

| Construct | Accession # | SEQ ID NO: |
|---|---|---|
| CHIKV 181/25-CE mRNA | 2452168 | 6 |
| CHIKV 181/25 | 2453076 | 7 |
| CHIKV 181/25-Δ5nsP3 | 2453451 | 8 |
| CHIKV Δ6K | 2453457 | 9 |
| CHIKV 181/25-ECMV IRES | 2453462 | 10 |

RNA Production

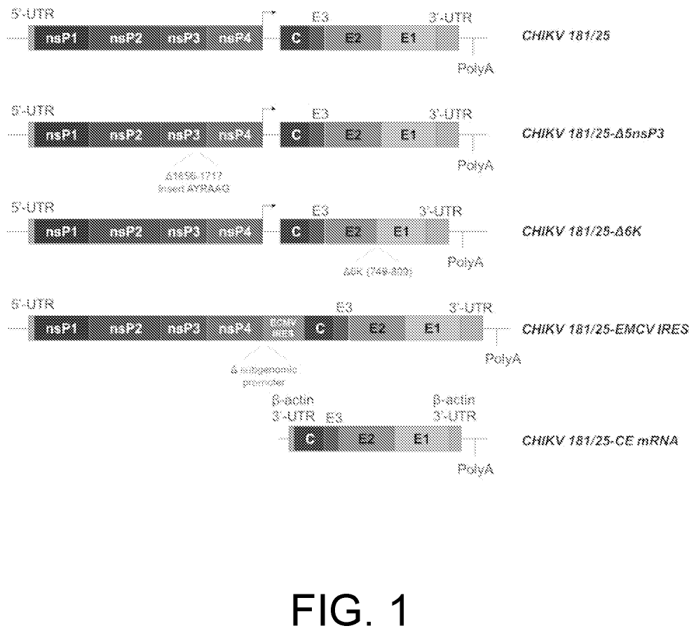

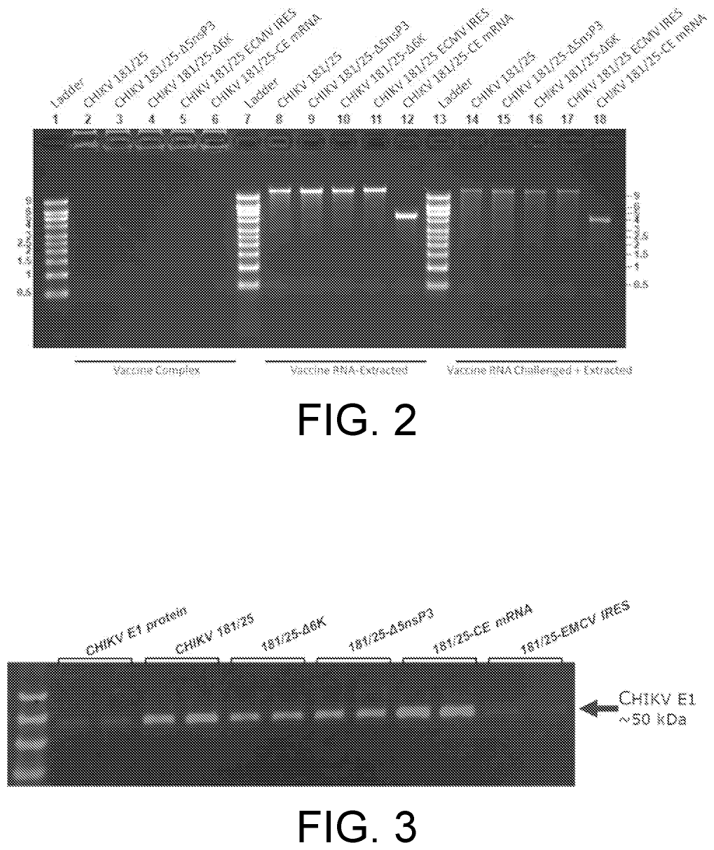

We created fully-functional, capped RNA using each of the DNA constructs as templates using in vitro transcription and capping reactions. Schematics of the RNA constructs are shown in FIG. 1. Viral genome-containing plasmids were amplified in Top10 cells (Invitrogen) and isolated using Qiagen Maxiprep kits. Purified plasmids were then linearized with NotI restriction digestion, and phenol-chloroform purified. RNA was transcribed in vitro with a standard protocol using T7 polymerase, RNase inhibitor, and pyrophosphatase enzymes (Aldevron), followed by a DNase incubation (DNase I, Aldevron) and LiCl precipitation. Cap0 structures were added to the RNA by a reaction with vaccinia capping enzyme, GTP, and S-adenosyl methionine (New England Biolabs). Capped RNA was then precipitated using LiCl and resuspended in nuclease-free water prior to quantification by UV absorbance (NanoDrop 1000) and analysis by agarose gel electrophoresis using Ambion NorthernMax′ reagents (Invitrogen). All transcribed and capped vaccine RNA was stored at −80° C. until use.

RNA Vaccine Formulation and Testing

We then formulated RNA vaccines with each RNA by complexing with a nanostructured lipid carrier (NLC) for effective delivery into target cells, as described previously (Erasmus, J. H.; Khandhar, A. P.; Guderian, J.; Granger, B.; Archer, J.; Archer, M. et al., A Nanostructured Lipid Carrier for Delivery of a Replicating Viral RNA Provides Single, Low-Dose Protection against Zika. Mol Ther 2018, 26 (10), 2507-2522; Erasmus, J. H.; Archer, J.; Fuerte-Stone, J.; Khandhar, A. P.; Voigt, E.; Granger, B. et al., Intramuscular Delivery of Replicon RNA Encoding ZIKV-117 Human Monoclonal Antibody Protects against Zika Virus Infection. Mol Ther Methods Clin Dev 2020, 18, 402-414.). RNA was complexed with a stable nanostructured lipid carrier (NLC) colloidal delivery formulation whose structure and manufacture has previously been described (Erasmus et al. 2018, supra). Briefly, a blend of liquid oil (squalene) and solid lipid (Dynasan 114) form a semi-crystalline nanostructure core, stabilized in an aqueous buffer by a hydrophobic sorbitan ester (Span 60), a hydrophilic ethoxylated sorbitan ester (Tween 80), and the cationic lipid DOTAP (N-[1-[2,3-Dioleoyloxy)propyl]-N,N,N-trimethylammonium chloride) which together allow for long-term colloidal stability.

The formulation was prepared as previously described (Erasmus et al. 2018, supra). Briefly, the oil phase was first prepared by mixing a liquid phase lipid squalene (Sigma), a solid phase lipid trimyristin (IOI Oleochemical), a positively charged lipid DOTAP (Corden), and a hydrophobic surfactant sorbitan monostearate (Sigma) in a blend vessel, which was placed in a sonicating water bath (70±5° C.) to facilitate solubilization. Separate preparation of the aqueous phase involved dilution of the hydrophilic surfactant polysorbate 80 (Fisher Scientific), in an aqueous buffer of 10 mM sodium citrate, followed by stirring for complete dissolution. The aqueous composition was also heated (70±5° C.) in a bath sonicator before blending with the oil phase.

After all components were dissolved, a high-speed laboratory emulsifier (Silverson Machines) was used to combine the oil and aqueous phases by blending at 7,000 RPM for a period of ten minutes to one hour to produce a crude mixture containing micron-sized oil droplets. The positioning of the Silverson mixing probe was adjusted as necessary for uniform dispersal of oil and complete emulsification. Further particle size reduction was achieved by high-shear homogenization in a M-110P microfluidizer (Microfluidics, Corp.). Each emulsion was processed for approximately 11 passes on the microfluidizer at 30,000 psi. The final pH was between 6.5-6.8. The resulting NLC particle suspension was terminally filtered with a 0.22 μm polyethersulfone filter (e.g., syringe filter) in order to collect the final NLC formulation. The final NLC formulation was stored at 2-8° C. until use.

RNA Vaccine Complexing

Vaccine RNA was complexed with NLC formulation at a NLC nitrogen:RNA phosphate ratio of 15. RNA, which is negatively charged, complexes electrostatically to the outside surface of the NLC. Briefly, RNA was diluted in nuclease-free water to 2× the desired final vaccine RNA concentration, and dilution of NLC was done in an aqueous sucrose citrate solution to a final concentration of 20% sucrose, 10 mM citrate. The diluted RNA and diluted NLC solutions where then combined at a 1:1 ratio and quickly mixed by pipet, to form a final 1×RNA concentration complexed with NLC in a 10% sucrose 5 mM citrate isotonic aqueous solution. The resulting vaccine solution was allowed to incubate on ice for 30 minutes to form stable nanoparticles.

To verify full and equal loading of RNA onto the nanoparticles, as well as nanoparticle-mediated protection of the RNA from degradation by RNases, we ran a sample of each complexed vaccine, RNA extracted from each vaccine, and RNA extracted from each vaccine after challenge with RNase on an agarose gel (FIG. 3). We saw complete RNA complexing for each RNA vaccine candidate, as indicated by no free RNA present in the vaccine solution. RNA extracted from each vaccine candidate was of the appropriate sizes and showed excellent integrity and equal loading across vaccine candidates. The vaccine nanoparticles also allowed for retention of significant amounts of full-length RNA after challenge with ample RNAse to fully degrade non-protected RNA, with protection of vaccine RNA from the action of RNases equal across vaccine candidates.

For characterization of nanoparticle-loaded RNA, vaccine samples were diluted to a final RNA concentration of 40 ng/μL in nuclease-free water. For verification of full RNA loading on the nanoparticles, vaccine samples containing 200 ng of RNA were mixed 1:1 by volume with Glyoxal load dye (Invitrogen), loaded directly on a denatured 1% agarose gel and run at 120 V for 45 minutes in Northern Max Gly running buffer (Invitrogen). Millennium RNA marker (ThermoFisher) was included on each gel with markers at 0.5, 1, 1.5, 2, 2.5, 3, 4, 5, 6, and 9 kilobases. Gels were imaged using ethidium bromide protocol on a ChemiDoc MP imaging system (BioRad). Lack of RNA bands being successfully electrophoresed indicates full complexing of RNA to the nanoparticles.

For verification of nanoparticle-loaded RNA integrity, RNA was extracted from the vaccine complexes by addition of 25:24:1 phenol:chloroform:isoamyl alcohol (Invitrogen) 1:1 by volume, vortexing, and centrifuging at 17,000 g for 15 minutes. The supernatant for each sample was then mixed 1:1 by volume with Glyoxal load dye and incubated at 50° C. for 20 minutes prior to being loaded onto a 1% agarose gel and run as described above.

For verification of equal protection of different vaccine RNAs from RNases by the complexes, the diluted vaccine complexes were incubated with RNase A (Thermo Scientific) for 30 minutes at room temperature at amounts ample to completely degrade un-complexed RNA (ratios of 1:40 RNase:RNA). This treatment was followed by treatment with recombinant Proteinase K (Thermo Scientific) at a ratio of 1:100 RNase A:Proteinase K for 10 minutes at 55° C. RNA was then extracted from the challenged samples and run on a 1% agarose gel as described above.

### Example 2: Vaccine Candidates Create VLPs In Vitro

We tested the ability of the whole-genome CHIK RNAs to successfully transfect cells when complexed with NLC and induce cellular production of VLPs. HEK cells (293T, ATCC CRL-3216) and African green monkey cells (Vero, ATCC CCL-81) were obtained from the American Type Culture collection and passaged in antibiotic-free DMEM medium with GlutaMAX (Invitrogen) supplemented with 10% fetal bovine serum. All cell lines were maintained in a humidified incubator at 37° C. in a 5% CO₂ atmosphere, and prescreened for mycoplasma contamination. Chikungunya virus strains 181/25 and CHIKV-LR (OPY1, passaged 5× in Vero cells) were obtained from the World Reference Center for Emerging Viruses and Arboviruses at the University of Texas Medical Branch in Galveston, TX, and propagated on Vero cells (MOI=0.02).

For demonstration of VLP creation by the whole-genome CHIK RNAs in vitro, HEK293T cells were plated in 12-well plates at a density of 5×10⁵ cells/well 24 hours prior to transfection. Shortly before transfection, media was removed from cells and replaced with 450 μl of serum-free Optimem medium (Invitrogen). 500 ng of NLC-complexed RNA was added into each well in a 50 μl volume, and cells were incubated at 37° C. and 5% CO₂ for four hours to allow for transfection. After the 4-hour incubation, transfection solutions were removed and replaced with 2 mL of DMEM supplemented with 2% FBS. Transfected cell supernatants were collected 72 hours post-transfection, concentrated by centrifugation through 30,000 MWCO Amicon Ultra-15 centrifugal filter tubes (Millipore) at 2000×g for 10-15 minutes, and finally ultracentrifuged through a 20% sucrose in PBS cushion (100,000×g, 10° C., 2 hr) in order to pellet cellular-produced VLPs. Pelleted VLPs were resuspended in 100 μL of PBS.

Western blots were conducted on the resulting isolated VLPs to confirm successful expression of CHIKV proteins from in vitro transcribed RNAs from each construct (FIG. 3). VLP solutions were reduced with NuPage 10× Reducing Agent and NuPage LDS Sample buffer (Invitrogen) and denatured by incubation at 95° C. for 10 minutes before loading on duplicate Novex Wedgewell 4-20% gradient precast PAGE gels and being run at 120 V in NuPAGE MES SDS running buffer for one hour. The gels were then transferred to PVDF membranes using the Invitrogen iBlot semi-dry transfer system with a six-minute transfer step. The membranes were then blocked overnight in a PBS solution with 5% nonfat dry milk. The blots were then rinsed and incubated for two hours in a 1:5000 dilution of anti-CHIK envelope protein (E1) antibody in 5% nonfat dry milk. After 3× rinsing in PBST, the membranes were incubated in a 1:200 dilution of goat anti-mouse HRP-conjugated secondary antibody for one hour. After 4× rinsing in PBST, the membranes were developed using West Pico Plus reagents (ThermoFisher Scientific) and signal was detected on a BioRad GelDoc XR+ system. All four full-genome CHIKV RNAs and the CHIKV structural protein mRNA successfully produced VLPs (FIG. 3).

Infectious Virus Rescue and Verification of Viral Attenuation

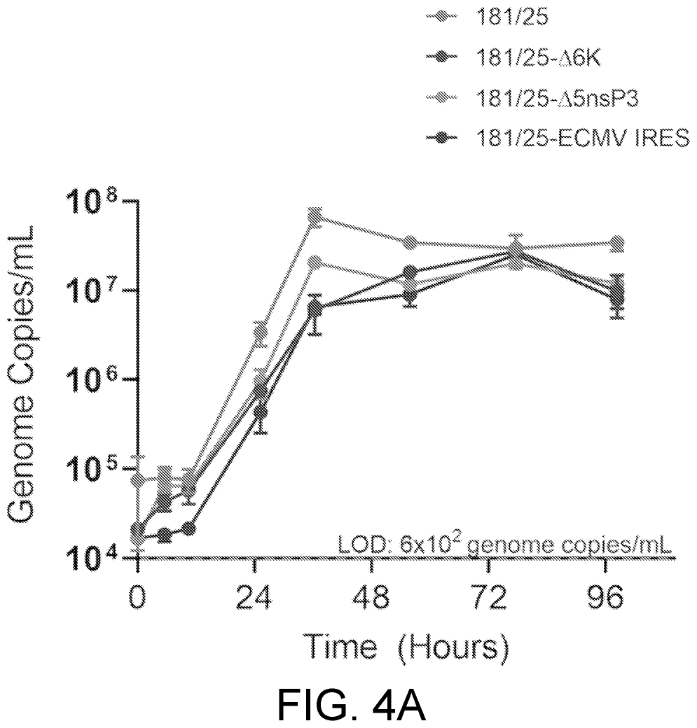

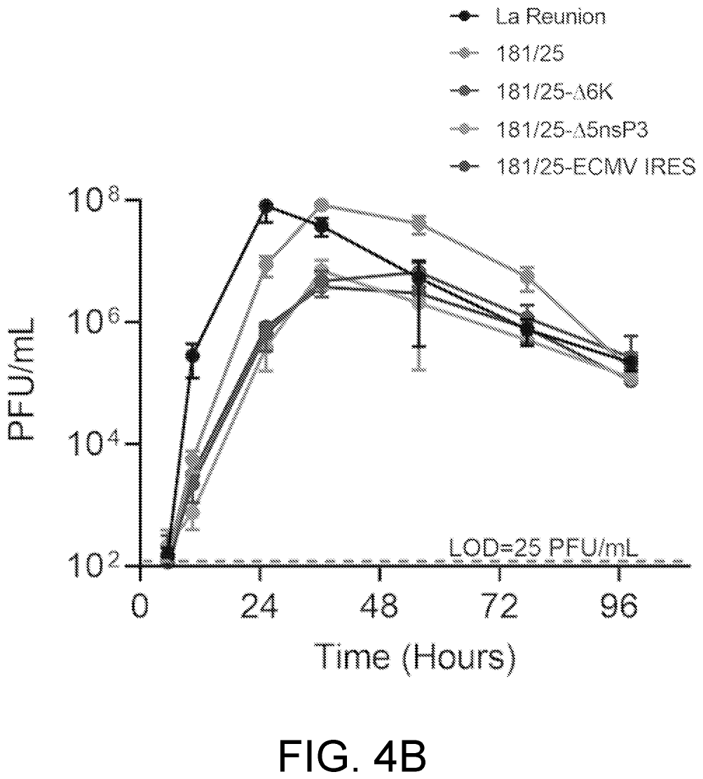

Infectious CHIKV vaccine virus strains were rescued from full-genome RNAs by 2× passage of VLP-containing supernates from RNA vaccine-transfected HEK293T, harvested as described above, in Vero cells. CHIKV variant viability and attenuation relative to wild-type CHIKV was measured by infection of Vero cells followed by timecourse measurements of supernate viral titers by qPCR (viral genomes FIG. 4A) and plaque assay (infectious particles FIG. 4B). Briefly, infection of Vero cells was conducted by removing growth medium from 90% confluent monolayers of Vero cells in 12-well tissue culture plates (approximately 1×10⁶ cells/well), and adding 100 μl/well of virus solution diluted to achieve an MOI of 0.01. After 1 hour of adsorption at 37° C. and 5% CO₂ with gentle rocking every 20 minutes, the inoculum was removed. One ml of DMEM supplemented with 1% FBS was then added. Supernates were harvested from independent biological triplicate wells at the times indicated post-infection, and frozen in aliquots for later plaque and qPCR assays. Similarly, a growth curve for virulent CHIKV-LR (“La Reunion”) was conducted under BSL3 conditions for comparison.

Viral Genome Quantification by Quantitative Reverse-Transcription PCR (FIG. 4A)

Frozen viral timecourse supernate samples were thawed and viral genomic RNA was extracted from samples using QIAamp Viral RNA Mini kits (Qiagen). Carrier RNA (Qiagen) was added to each sample to normalize the extraction/reverse transcription process between samples. Total RNA concentration was normalized between samples to obtain 750 ng total RNA per random hexamer reverse transcription reaction, conducted using the QuantiTect Reverse Transcription Kit (Qiagen). Quantitative PCR was then conducted on 1 μl of the resulting cDNA, using the qPCR primers described in Lanciotti, R. S.; Kosoy, O. L.; Laven, J. J.; Panella, A. J.; Velez, J. O.; Lambert, A. J. et al., Chikungunya virus in US travelers returning from India, 2006. Emerg Infect Dis 2007, 13 (5), 764-7 that detect a region of the CHIKV NSP4 gene conserved between all virus strains used in this work.

Forward:
(SEQ ID NO: 11)
5′-TCACTCCCTGTTGGACTTGATAGA
Reverse:
(SEQ ID NO: 12)
5′-TTGACGAACAGAGTTAGGAACATACC

A standard curve was formed by serial dilution of NotI-linearized CHIKV 181/25 genomic plasmid of known concentration spanning the entire dynamic range of sample concentrations. This standard curve (genomic plasmid copy number vs. CT) was fit with a semi-log line (R²=0.993) and used to interpolate absolute CHIKV genome copy numbers in the infection samples. Quantitative PCR was performed with technical duplicates of the biological triplicates collected at each time point for each viral variant.

Plaque Assays (FIG. 4B)

For quantification of infectious virus particles in infection supernates, samples were serially diluted in 1:10 dilutions of DMEM supplemented with 1% FBS and 2 mM Glutamax. Vero cells were plated 18 hours prior to assay at a concentration of 5×10⁵ cells/well in 6-well tissue culture plates and allowed to form monolayers. Cell monolayers were infected with 200 μl of virus dilution and incubated for one hour with gentle rocking every 20 minutes. The virus-containing sample was then removed, and cell monolayers were overlaid with 2 ml of DMEM supplemented with 1% FBS, 2 mM Glutamax, and 0.6% melted agar. The plates were cooled until agar solidified, and incubated at 37° C., 5% CO₂ for approximately 48 hours, until plaques appeared. Agar layers were then removed; cells were fixed for 20 minutes with a formalin solution, and cell layers were stained with 0.1% crystal violet in 20% ethanol to visualize plaques. The rescued CHIKV 181/25 virus grew to a higher titer (6.6×10⁷ genome copies/mL by qPCR and 8.5×10⁷ pfu/mL by plaque assay) than the more-attenuated CHIKV 181/25-Δ5nsP3, CHIKV 181/25-46K, and CHIKV 181/25-ECMV IRES rescued viral strains (titers of 2.1×10⁷, 2.7×10⁷, and 2.5×10⁷ genome copies/mL by qPCR and 7.0×10⁶, 6.7×10⁶, and 3.8×10⁶ pfu/mL by plaque assay, respectively; p<0.05 for all). CHIKV-LR replicated to similar titers as CHIKV 181/25 (8.2×10⁷ versus 8.5×10⁷ pfu/mL, p=0.93) but reached full titer approximately 12 hours sooner. As expected, CHIKV 181/25-CE mRNA VLPs did not allow for rescue of infectious virus.

### Example 3: Whole-Genome RNA Vaccines are Immunogenic in Immunocompetent Mice and Protect Against Virulent CHIKV Challenge

In Vivo Studies

We tested these RNA vaccine candidates for immunogenicity by injecting groups of immunocompetent C57BL/6 mice with 1 μg (full-length genome RNA and mRNA) or 5 μg (mRNA) of the individual RNA constructs formulated with NLC by i.m. injection of 50 μl of vaccine formulation in each rear quadriceps muscle for a total of 100 μl vaccine per mouse.

While type I interferon (IFN) receptor−/− mice are often used for studies of CHIKV pathogenesis, (Plante, K. S.; Rossi, S. L.; Bergren, N. A.; Seymour, R. L.; Weaver, S. C., Extended Preclinical Safety, Efficacy and Stability Testing of a Live-attenuated Chikungunya Vaccine Candidate. PLoS Negl Trop Dis 2015, 9 (9), e0004007; Haese, N. N.; Broeckel, R. M.; Hawman, D. W.; Heise, M. T.; Morrison, T. E.; Streblow, D. N., Animal Models of Chikungunya Virus Infection and Disease. J Infect Dis 2016, 214 (suppl 5), S482-S487; Chan, Y. H.; Lum, F. M.; Ng, L. F. P., Limitations of Current in Vivo Mouse Models for the Study of Chikungunya Virus Pathogenesis. Med Sci (Basel) 2015, 3 (3), 64-77) interferon-competent mice are necessary for studies involving replicating viral vaccines to accurately reflect typical viral replication and immune responses; (Haese et al. supra) thus wild-type C57BL/6 mice were used throughout this work.

Female 6-8 week old immunocompetent C57BL/6 mice were used for all vaccine immunogenicity studies (The Jackson Laboratory). All-female mice were used in order to maximize statistical power to detect immunogenicity differences between vaccine variants. Mice were non-specifically and blindly distributed into their respective groups. No exclusion criteria were established prior to beginning the studies.

All animal work was carried out in the IDRI Vivarium under ABSL1, ABSL2, or ABSL3 conditions as appropriate under the oversight of the IDRI Institutional Animal Care and Use Committee (IACUC). All challenged mice were monitored daily for weight loss and signs of disease. Mice that lost over 20% of their pre-challenge weight, or demonstrated lack of mobility, lethargy, or a hunched back that did not resolve were humanely euthanized by CO₂ inhalation. All remaining mice were euthanized at the end of the scheduled study period. All animals were cared for in accordance with the guidelines of the Committee on Care and Use of Laboratory Animals (Institute of Laboratory Animal Resources, National Research Council).

Mice were inoculated with full-genome RNA vaccines at doses of 1 μg of RNA complexed with NLC, by i.m. injection of 50 μl of vaccine formulation in each rear quadriceps muscle for a total of 100 μl vaccine per mouse. Mice were inoculated with mRNA vaccine at doses of 1 or 5 μg of RNA complexed with NLC by i.m. injection with the same volume and injection strategy as the full-genome RNA vaccines. Positive vaccination control mice were inoculated by s.c. footpad injection of 20 μl containing 10⁴ pfu of CHIKV-181/25 virus. The mock group was injected with a saline solution.

PRNT Assays

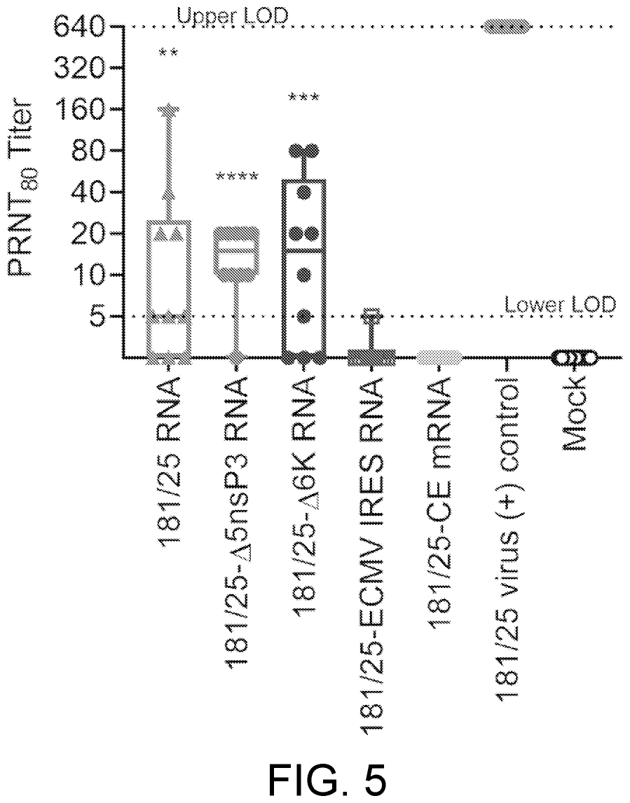

Mouse serum samples were tested for the presence of CHIKV-neutralizing antibody titers by plaque reduction neutralization tests (PRNT₈₀). Plaque-reduction neutralization titers were measured in serum samples taken 21 days post-inoculation and compared to control mice vaccinated by footpad injection of 10⁴ pfu of CHIKV-181/25 virus (FIG. 5). The CHIKV-181/25, CHIKV-Δ5nsp3, and CHIKV-Δ6K full-genome RNA vaccines induced significant serum neutralizing antibody titers in vaccinated mice relative to mock-immunized control mouse sera (adjusted p-value<0.005 for each), though these were low relative to PRNT titers resulting from mouse immunization with CHIKV 181/25 virus (adjusted p-value<0.0001 for all). PRNT₈₀ titers were calculated as the mouse serum dilution that resulted in neutralization of >80% of the number of CHIKV-181/25 plaques found in control (non-immunized mouse serum) samples.

Blood samples were taken from all vaccinated and control mice three days post-vaccination to check for post-vaccination viremia by plaque assay. Briefly, samples were serially diluted in 1:10 dilutions of DMEM supplemented with 1% FBS and 2 mM Glutamax. Vero cells were plated 18 hours prior to assay at a concentration of 5×10⁵ cells/well in 6-well tissue culture plates and allowed to form monolayers. Cell monolayers were infected with 200 μl of virus dilution and incubated for one hour with gentle rocking every 20 minutes. The virus-containing sample was then removed, and cell monolayers were overlaid with 2 ml of DMEM supplemented with 1% FBS, 2 mM Glutamax, and 0.6% melted agar. The plates were cooled until agar solidified, and incubated at 37° C., 5% CO₂ for approximately 48 hours, until plaques appeared. Agar layers were then removed; cells were fixed for 20 minutes with a formalin solution, and cell layers were stained with 0.1% crystal violet in 20% ethanol to visualize plaques. We detected low levels of post-vaccination viremia in five of the ten 181/25 virally-immunized mice, but no viremia in any of the RNA-vaccinated mice. CHIKV 181/25-CE mRNA vaccination did not result in neutralizing antibody titers at either 1 or 5 μg doses.

Post-Vaccination Challenge

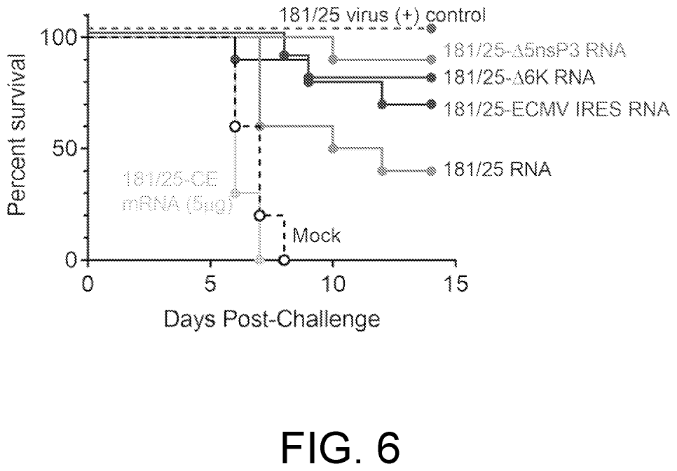

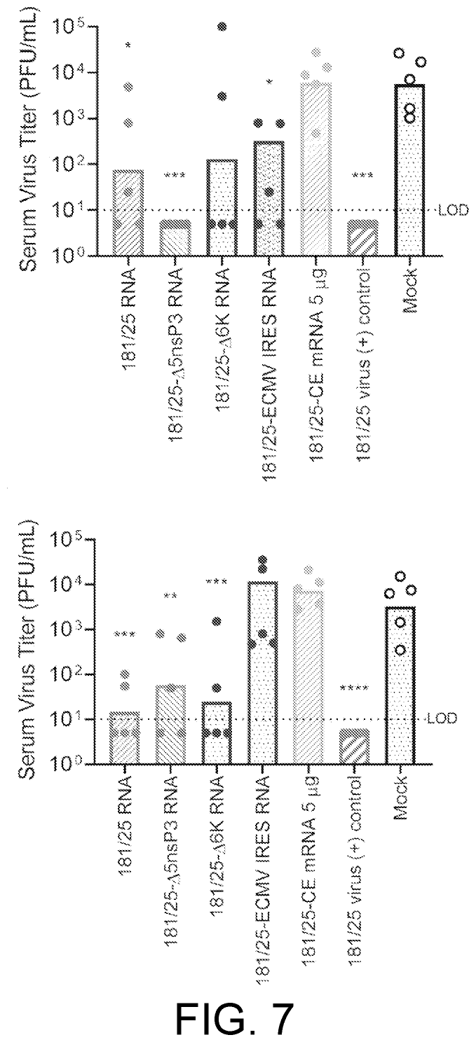

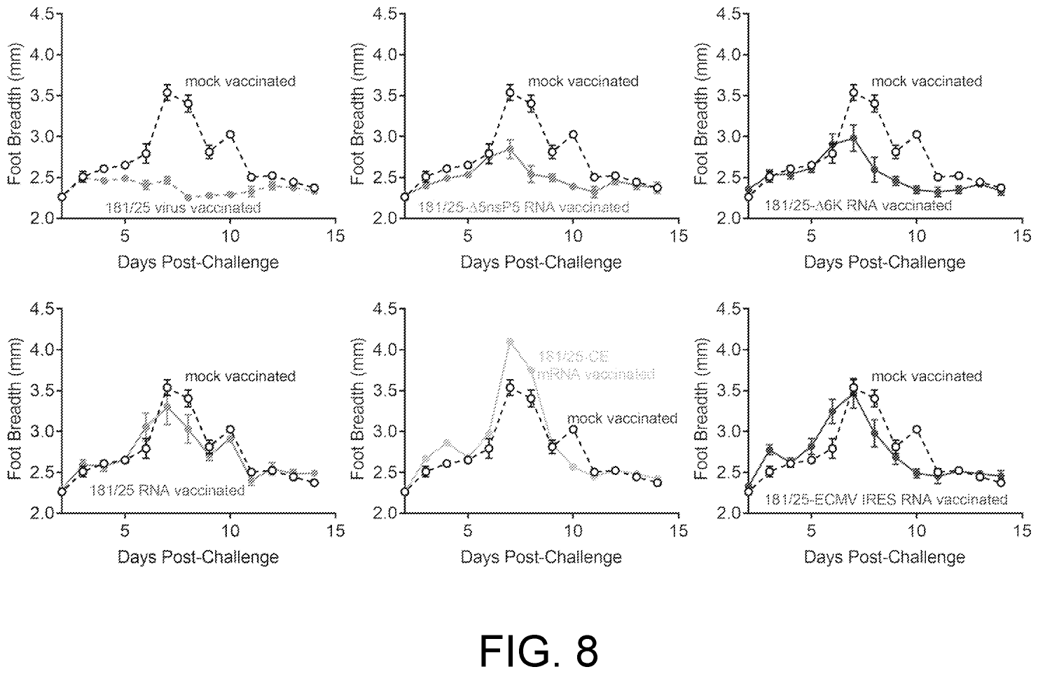

Twenty-eight days post-vaccination, the vaccinated mice from each group were then each split into two groups and challenged. A lethal challenge group was used to determine vaccine-induced protection from death, and a nonlethal challenge group was used to examine vaccine-induced protection from footpad swelling, under immunocompetent conditions, as is standard in the field of CHIK vaccine studies. Survival data are shown in FIG. 6. Viremia data from both lethal and non-lethal challenged mice is shown in FIG. 7 panels A and B, respectively. Footpad swelling data is shown in FIG. 8.

While CHIKV-181/25 is nonlethal in immunocompetent C57BL/6 mice, (Haese et al. supra) CHIKV is known to be type I interferon sensitive (Reynaud, J. M.; Kim, D. Y.; Atasheva, S.; Rasalouskaya, A.; White, J. P.; Diamond, M. S. et al., IFIT1 Differentially Interferes with Translation and Replication of Alphavirus Genomes and Promotes Induction of Type I Interferon. PLoS Pathog 2015, 11 (4), e1004863; Couderc, T.; Chretien, F.; Schilte, C.; Disson, O.; Brigitte, M.; Guivel-Benhassine, F. et al., A mouse model for Chikungunya: young age and inefficient type-I interferon signaling are risk factors for severe disease. PLoS Pathog 2008, 4 (2), e29) and temporary inhibition of type I IFN signaling is necessary and sufficient to obtain lethal challenge conditions with CHIKV-LR in otherwise immunocompetent C57BL/6 mice.

To create a CHIKV lethal challenge model, we transiently immunocompromised the mice in the lethal challenge group by i.p. injection of 2 mg of InVivoMAb anti-mouse IFNAR-1 blocking antibody (clone MAR1-5A3, BioXCell) 18 hours prior to challenge with 10³ pfu/mouse of virulent CHIKV-LR (from WRCEVA at UTMB, TVP20521) via footpad injection. For lethal challenge, each mouse was injected i.p. with 2 mg of InVivoMAb anti-mouse IFNAR-1 blocking antibody (clone MAR1-5A3, BioXCell) in 300 μl volume 18 hours prior to s.c. footpad injection of 80 μl containing 10³ pfu/mouse of CHIKV-LR (40 μl/rear footpad). Lethally-challenged mice were monitored daily for weight loss and signs of disease.

For lethal challenge, 28 days post-vaccination mice were challenged with virulent CHIKV-LR (from WRCEVA at UTMB, TVP20521). Each mouse was injected i.p. with 2 mg of InVivoMAb anti-mouse IFNAR-1 blocking antibody (clone MAR1-5A3, BioXCell) in 300 μl volume 18 hours prior to s.c. footpad injection of 80 μl containing 10³ pfu/mouse of CHIKV-LR (40 μl/rear footpad). Lethally-challenged mice were monitored daily for weight loss and signs of disease.

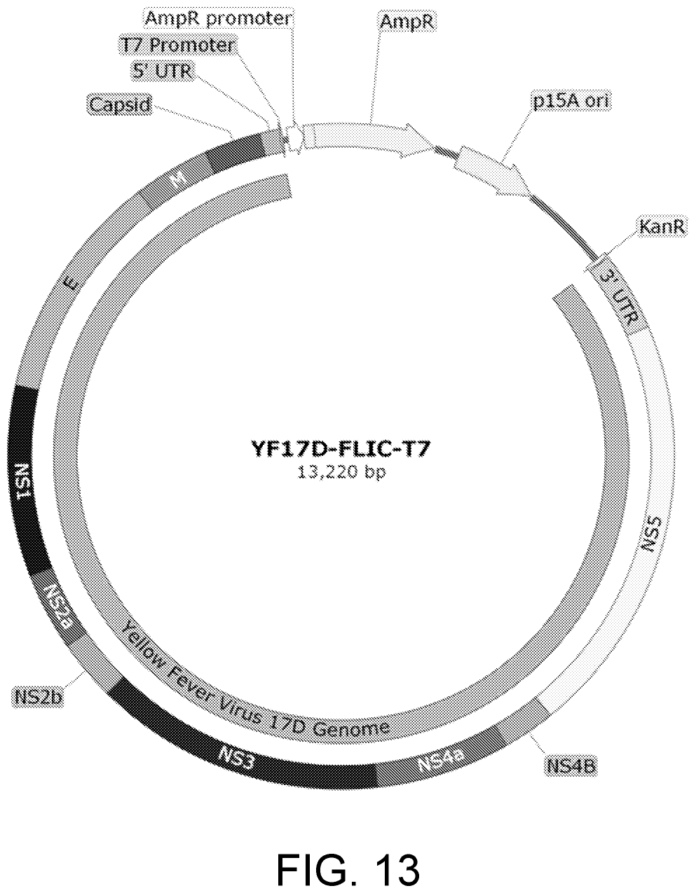

For non-lethal challenge, each mouse was injected with 10⁵ pfu of CHIKV-LR s.c. into the footpad, and mice were monitored daily for signs of disease, weight loss and footpad swelling by measurement of footpad breadth (FIG. 8, initial vaccine immunogenicity screen) or footpad width×breadth (FIG. 13, detailed lead candidate dosing and efficacy study). Blood samples were taken from all challenged mice three days post-challenge by the retro-orbital route to check for post-challenge viremia.

Non-lethal challenge mice for examination of CHIKV-induced footpad swelling did not receive IFNAR-blocking antibody and were challenged 28 days post-vaccination by s.c. footpad injection of 10⁵ pfu of CHIKV-LR per mouse. Serum samples were taken 2 days after challenge from a subset of the challenged mice (n=5) to measure viremia in lethally challenged mice (FIG. 7A) and non-lethally challenged mice (FIG. 7B). Footpad breadth was measured daily for each non-lethally challenged mouse for 14 days (FIG. 8). All challenged mice were weighed daily.

Mice were monitored daily for signs of disease, weight loss and footpad swelling by measurement of footpad breadth (FIG. 8, initial vaccine immunogenicity screen) or footpad width×breadth (FIG. 13, detailed lead candidate dosing and efficacy study).

Mice vaccinated with CHIKV 181/25 virus showed 100% survival, total suppression of viremia after lethal challenge in the transiently-immunocompromised mice, and total suppression of CHIKV-induced footpad swelling in the immunocompetent mice untreated with Marl IFNAR-blocking antibody. CHIKV 181/25-CE mRNA inoculation did not result in neutralizing antibody titers at either 1 μg (data not shown) or 5 μg doses, and the 5 μg dose failed to provide any protection against viremia, death, or footpad swelling relative to unvaccinated mice. CHIKV 181/25-CE mRNA was thus removed from further candidacy. Each whole-genome, live-attenuated RNA vaccine candidate induced partial protection from post-challenge mortality, viremia, and footpad swelling.

The wide range of neutralizing antibody titers induced by any one CHIKV whole-genome RNA vaccine candidate suggested that vaccine dosing was not optimal, leading to launch of the RNA virus in some but not all mice. The full-genome CHIKV 181/25 and CHIKV 181/25Δ5nsP3 RNA vaccine candidates were chosen—based on their induction of neutralizing antibody titers and ability to protect mice against viremia, death, and footpad swelling—for further dosing and immunogenicity studies.

### Example 4: Whole-Genome RNA Vaccine Immunogenicity and Efficacy is Dose-Dependent and Rivals that of Live Virus Vaccine

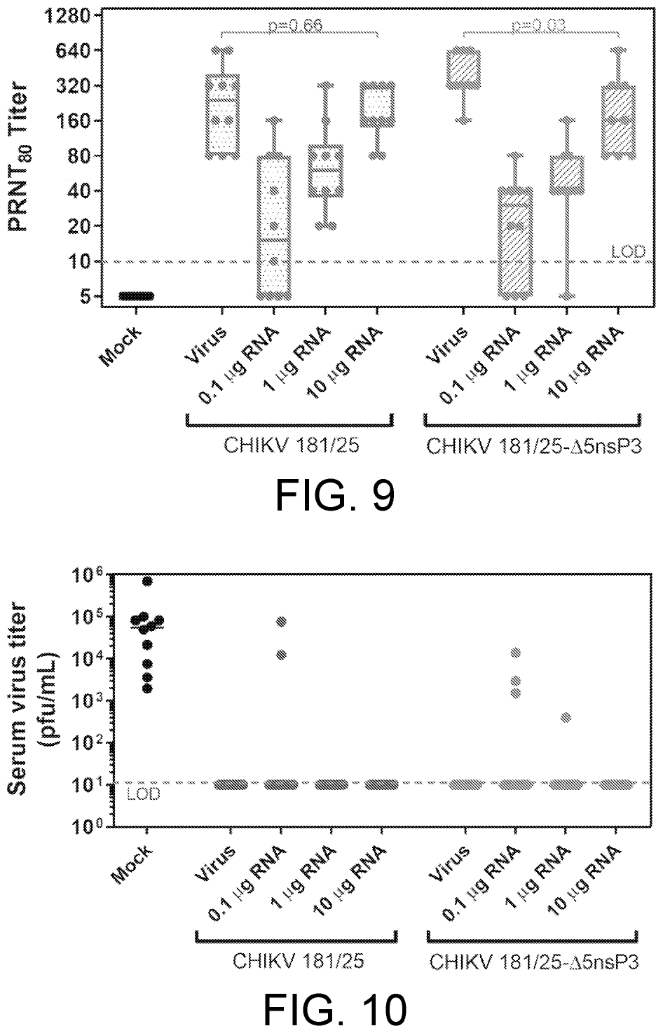

To confirm immunogenicity of and determine suitable dosing, we immunized mice with 0.1 μg, 1 μg, or 10 μg of each of the two lead RNA vaccines CHIKV 181/25 and CHIKV 181/25-Δ5nsP3. Vaccination with 10⁴ pfu/mouse of each attenuated virus or plain PBS served as positive and negative vaccination control groups. Serum antibody titers 28 days post-vaccination were measured by PRNT (FIG. 9). The PRNT assay was performed as described above. Both the CHIKV 181/25 and CHIKV 181/25-Δ5nsP3 RNA-based vaccines induced significant neutralizing antibody serum titers 28 days post-vaccination. Clear dose-dependences was observed for both whole-genome RNA vaccines. Indeed, the highest dose of each whole genome RNA vaccine (10 μg/mouse) resulted in induction of serum antibody titers not significantly different than antibody titers induced by live viral vaccination (p>0.05).

Blood samples were taken from all challenged mice three days post-challenge by the retro-orbital route to check for post-challenge viremia by plaque assay (FIG. 10). Briefly, samples were serially diluted in 1:10 dilutions of DMEM supplemented with 1% FBS and 2 mM Glutamax. Vero cells were plated 18 hours prior to assay at a concentration of 5×10⁵ cells/well in 6-well tissue culture plates and allowed to form monolayers. Cell monolayers were infected with 200 μl of virus dilution and incubated for one hour with gentle rocking every 20 minutes. The virus-containing sample was then removed, and cell monolayers were overlaid with 2 ml of DMEM supplemented with 1% FBS, 2 mM Glutamax, and 0.6% melted agar. The plates were cooled until agar solidified, and incubated at 37° C., 5% CO₂ for approximately 48 hours, until plaques appeared. Agar layers were then removed; cells were fixed for 20 minutes with a formalin solution, and cell layers were stained with 0.1% crystal violet in 20% ethanol to visualize plaques.

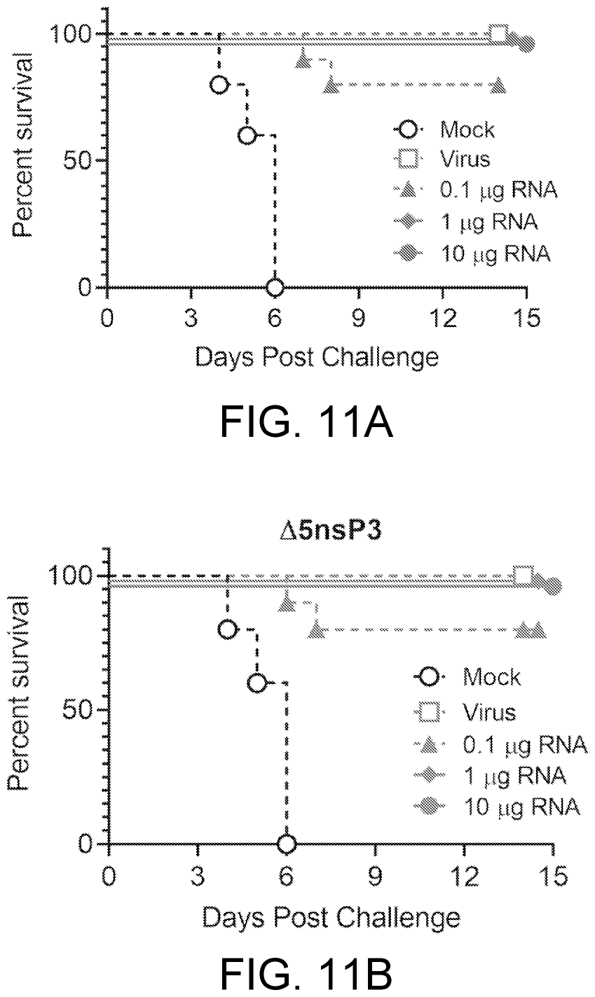

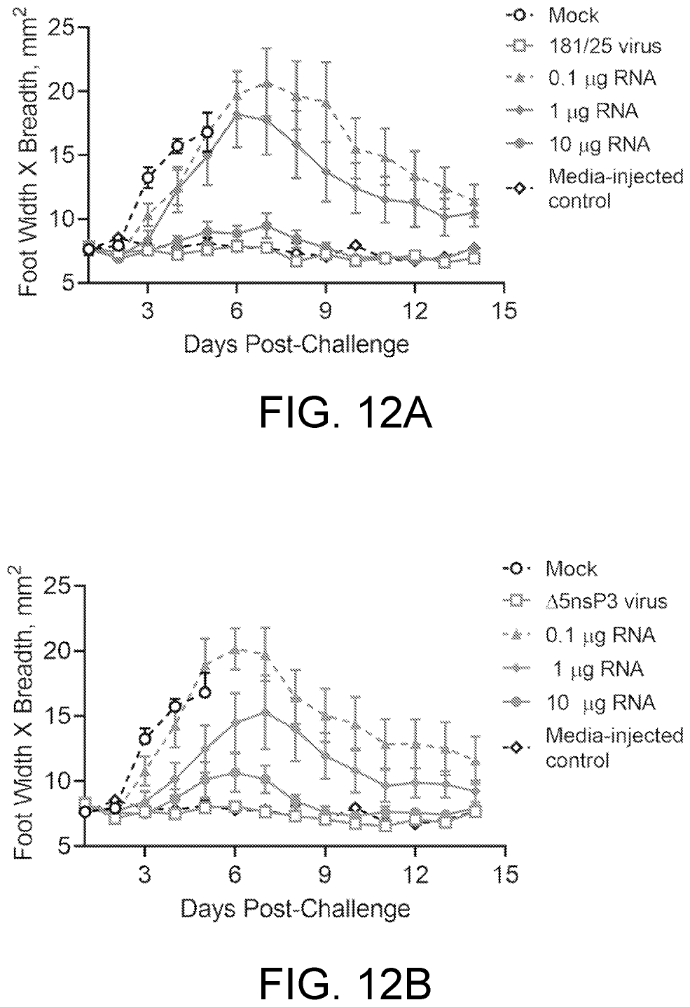

Each animal group was then injected with 2 mg of murine IFNAR-blocking antibody, and lethally challenged with 10³ pfu of virulent CHIKV-LR 18 hours later. Survival data is displayed in FIG. 11. Footpad area measurements (width×breadth) from the lethally-challenged mice were also taken for CHIKV 181/25-vaccinated (FIG. 12A) and CHIKV 181/25-Δ5nsP3-vaccinated (FIG. 12B) mice. All mice were weighed daily.

Mock-vaccinated mice uniformly died by Day 6 post-challenge as shown in FIG. 11A and FIG. 11B. Both whole-genome RNA vaccines protected 100% of mice against death at doses of 10 μg and 1 μg/mouse, and partially protected mice at the lowest RNA vaccine dose (0.1 μg).

Monitoring of CHIKV-induced footpad swelling in this transiently-immunocompromised challenge model was highly informative; significant footpad swelling occurred in a dose-dependent manner inversely proportional to vaccine dose (FIG. 12A and FIG. 12B). Interestingly, even mice completely protected from mortality by the mid-range 1 μg dose of either full-genome RNA vaccine showed significant footpad swelling, indicating incomplete protection against morbidity. Little to no footpad swelling was seen in virally-vaccinated mice, or in mice vaccinated with the highest dose (10 μg RNA) of each RNA vaccine, indicating a high level of protection even upon vaccination with a highly-attenuated CHIKV genomic strain.

Statistical Analyses

Statistical analysis was performed using GraphPad Prism software. Data distribution and variance were evaluated for normality prior to analyses. All data collected by qPCR, PRNT, or plaque assay was log-normalized prior to analysis. Two-tailed T tests or one-parameter ANOVA analyses followed by Dunnett's multiple comparisons test (α=0.05) were conducted to determine statistical differences in antibody titers and post-challenge viremia measures between study groups. Test residuals were checked to confirm data normality.

### Example 5: Whole-Genome Yellow Fever Vaccine is Immunogenic in Immunocompetent Mice

We created DNA constructs for full-genome RNA production by adapting a plasmid containing the full-length infectious live-attenuated yellow fever 17D virus genomic sequence for RNA production by T7 polymerase-mediated in vitro transcription by replacing the existing promoter with a T7 promoter sequence suitable for RNA in vitro transcription (FIG. 13). The resulting plasmid sequence is provided as SEQ ID NO: 13. We then created fully-functional, capped RNA using the DNA construct as a template as described above. The yellow fever genome RNA was complexed with NLC as described above at a N:P ratio of 1:15.

Immunocompetent 6-8 week old C57BL/6 mice (n=4/group) were inoculated s.c. with the full-genome RNA vaccine at doses of 1 or 10 μg of RNA complexed with NLC or with SEAP saRNA/NLC injected intramuscularly as a negative control. Subcutaneous injections were achieved by injection of 30 μl of vaccine in each rear mouse footpad, for a total 60 μl vaccine volume/mouse.

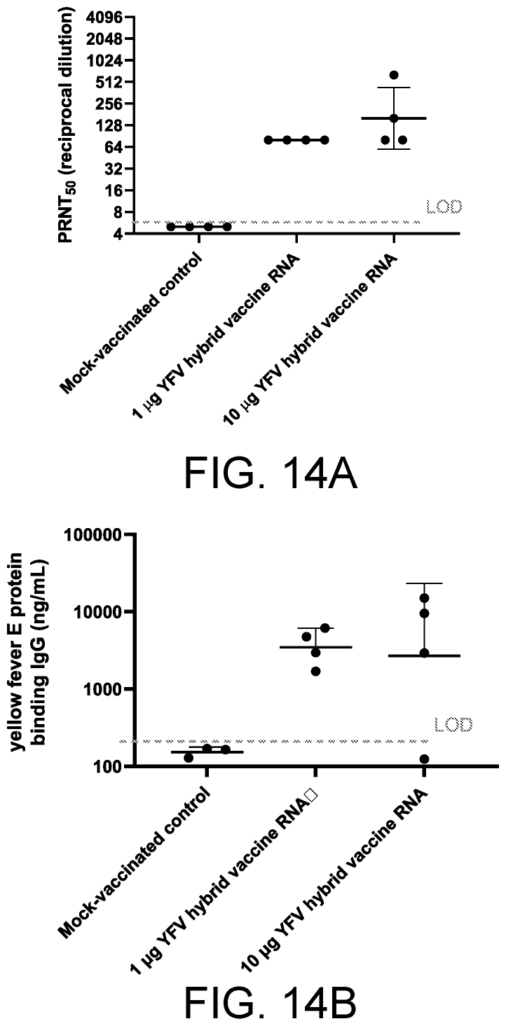

Plaque-reduction neutralization titers (PRNT₅₀) to YFV were measured in serum samples taken 28 days post-inoculation (FIG. 14A). The plaque reduction neutralization tests were performed as described above, using YF-17D as the virus to be neutralized rather than CHIK, and incubating for 5 rather than 2 days for full plaque formation. PRNT₅₀ titers were calculated as the mouse serum dilution that resulted in neutralization of >50% of the number of YF-17D plaques found in control (non-immunized mouse serum) samples.

Vaccination with both the 1 μg and 10 μg of the YFV hybrid vaccine produced neutralizing antibody titers well above the accepted correlate of protection for yellow fever (PRNT titer of 1:10), indicating that inoculation with the RNA vaccine provides protective immunity against yellow fever.

Serum samples collected 28 days following inoculation were also used to detect yellow fever-specific antibodies (FIG. 14B). Yellow fever E specific IgG in the serum was determined by ELISA using recombinant yellow fever E protein-coated microtiter plates for yellow fever E protein-binding antibody capture, dilutions of 4G2 monoclonal flavivirus IgG antibody as an assay standard, and an alkaline phosphatase-conjugated secondary anti-mouse total IgG antibody for detection.

### SEQUENCES

Sequences discussed in this disclosure are included below.

full-length 181/25 CHIKV virus genome
SEQ ID NO: 1
acccaucauggauucuguguacguggacauagacgcugacagcgccuuu
uugaaggcccugcaacgugcguaccccauguuugagguggaaccuaggc
aggucacaucgaaugaccaugcuaaugcuagagcguucucgcaucuagc
cauaaaacuaauagagcaggaaauugaucccgacucaaccauccuggau
auagguagugcgccagcaaggaggaugaugucggacaggaaguaccacu
gcguuugcccgaugcgcagcgcagaagaucccgagagacucgcuaauua
ugcgagaaagcucgcaucugccgcaggaaaaguccuggacagaaacauu
ucuggaaagaucggggacuuacaagcggugauggccgugccagacacgg
agacgccaacauuuugcuuacacacagaugucucauguagacagagagc
agacgucgcgauauaccaagacgucuaugcuguacacgcacccacgucg
cuauaccaccaggcgauuaaaggaguccgaguggcguacuggguagggu
ucgacacaaccccguucauguacaacgcuauggcgggugccuaccccuc
auacucgacaaauugggcggaugagcagguacugaaggouaagaacaua
ggauuauguucaacagaccugacggaagguagacgaggcaaauugucua
ucaugagagggaaaaagcuaaaaccgugcgaccgugugcuguucucagu
agggucaacgcuuuacccggaaagccgcacgcuacucgugugagggcua
cgucguuaagagaauaacgaugagcccaggccuuuauggaaaaaccaua
ggguaugcgguaacccaccacgcagacggauucuugaugugcaagacua
ccgacacgguugacggcgaaagagugucauucucggugugcacguacgu
gccggcgaccauuugugaucaaaugaccggcauccuugcuacagaaguc
acgccggaggaugcacagaagcuguugguggggcugaaccagaggauag
ugguuaacggcagaacgcaacggaacacgaacaccaugaagaacuaccu
acuucccguggucgcccaggccuucaguaagugggcaaaggagugccgg
aaggacauggaagaugagaagcuucugggggucagagaaagaacacuaa
ccugcugcugucuaugggcauuuaagaagcagaaaacacacacggucua
caagaggccugauacccagucaauccagaagguucaggccgaauuugac
agcuuuguaguaccgggccuguggucguccggguugucaaucccguuga
ggacuagaaucaagugguuguuacgcaaggugccgaaaacagaccugau
cccauacagcgggaaugcccaagaagcccaggaugcagaaaaagaagca
gaggaagaacgagaagcagaacugacucaugaggcucuaccaccccuac
aggcagcacaggaagauguccaggucgaaaucgacguggaacagcuuga
ggauagagcuggugcuggaauaauagagacuccgagaggcgcuaucaaa
guuacugcccaacuaacagaccacgucgugggggaguaccugguacuuu
ccccgcagaccguacuacgcagccagaagcucagccugauccacgcuuu
agcggagcaagugaagacguguacgcacagcggacgagcagggagguau
gcggucgaagcguacgauggccgaguccuagugcccucaggcuaugcaa
uuucgccugaagacuuccagagucuaagcgaaagcgcaacgauggugua
caacgaaagagaguucguaaacagaaaguuacaccacauugcgaugcac
ggaccagcccugaacacugacgaagagucguaugagcuggugagggcag
agaggacagaacacgaguacgucuacgacguggaccagagaagaugcug
uaagaaggaagaagcugcaggacugguacuggugggcgacuugacuaau
ccgcccuaccacgaauucgcauacgaagggcuaaaaauucgccccgccu
gcccauacaaaauugcagucauaggagucuucgggguaccaggaucugg
caagucagccauuaucaagaaccuaguuaccaggcaagaccuggugacu
agcggaaagaaagaaaacugccaagaaaucagcaccgacgugaugagac
agagaggucuagagauaucugcacguacgguagauucgcugcucuugaa
uggaugcaacagaccagucgacguguuguacguagacgaggcguuugcg
ugccacucuggaacguuacuugcuuugaucgccuuggugagaccaagac
agaaaguuguacuuuguggugacccgaagcagugoogcuucuucaauau
gaugcagaugaaagucaacuacaaucauaacaucugcacccaaguguac
cacaaaaguaucuccaggogguguacacugccugugacugccauugugu
caucguugcauuacgaaggcaaaaugcgcacuacgaaugaguacaacau
gccgauuguaguggacacuacaggcucaacgaaaccugacccuggagac
cucguguuaacgugcuucagaggguggguuaaacaacugcaaauugacu
aucguggacacgaggucaugacagcagccgcaucccaaggguuaacuag
aaaaggaguuuacgcaguuaggcaaaaaguuaacgaaaacccacucuau
gcaucaacaucagagcacgucaacguacuccuaacgcguacggaaggua
aacugguauggaagacacucucuggugacccguggauaaagacgcugca
gaacccaccgaaaggaaacuucaaagcaacuauuaaggagugggaggug
gagcacgcaucgauaauggcgggcaucugcagucaccaagugaccuuug
acacauuccaaaacaaagccaacguuugcugggcuaagagcuugguccc
uauccucgaaacagcggggauaaaacuaaaugauaggcaguggucccag
auaauucaagccuucaaagaagacaaagcauacucacccgaaguagccc
ugaaugaaauaugcacgcgcauguaugggguggaucuagacagugggcu
auucucuaaaccguugguaucuguguauuacgcggauaaccauugggau
aauaggccgggaggaaagauguucggauucaacccugaggcagcgucca
uucuagaaagaaaguacccauuuacaaaaggaaaguggaacaucaacaa
gcagaucugcgugacuaccaggaggauagaagacuucaacccuaccacc
aacauuauaccggucaacaggagacuaccacacucauuaguggccgaac
accgcccaguaaaaggggaaagaauggaauggcugguuaacaagauaaa
cggacaccacguacuccugguuagcggcuauaaccuugcacugccuacu
aagagagucaccuggguagcgccacuagguguccgcggagcggacuaua
cauacaaccuagagougggucuaccagcaacgcuugguagguaugaccu
aguggucauaaacauccacacaccuuuucgcauacaccauuaccaacag
ugcguagaucacgcaaugaaacugcaaaugcuagggggugacucacuga
gacugcucaaaccggguggcucucuauugaucagagcauacgguuacgc
agauagaaccagugaacgagucaucugcguacugggacgcaaguuuagg
cagaaggaauuuuacaacgcaugucaugaacaaucaacugaaugcagcc
uuuguaggacaggccacccgagcaggaugugcaccaucguaccggguaa
aacgcauggacaucgcgaagaacgaugaagagugcgugguuaacgccgc
caaccccaguaggaaccgcaaaaacaguuaugugcgguacguauccagu
aauccacgccguaggaccaaacuucucaaauuauucggagucugaaggg
gaccgggaauuggcggcugccuaucgagaagucgcaaaggaaguaacua
gacugggaguacucuuuacagccauggacucgacggaugcagacguggu
caucuacugccgagacaaggaaugggagaagaaaauaucugaggccaua
cagaugcggacccaaguggagcugcuggaugagcacaucuccauagacu
gcgaugucauucgcgugcacccugacaguagcuuggcaggcagaaaagg
auacagcaccacggaaggcgcacuguauucauaucuagaagggacacgu
uuucaccagacggcaguggauauggcagagauauacacuauguggccaa
agcaaacagaggccaaugagcaagucugccuauaugcccugggggaaag
uauugaaucaaucaggcagaaaugcccgguggaugaugcagacgcauca
ucucgguaauguuauucgaucacaaugugccaucgcgcguaaguccaag
ggaauacagaucuucccaggagucuguacaggaagugaguacgacaacg
ucauugacgcauagccaguuugaucuaagcgccgauggcgagacacugc
cugucccgucagaccuggaugcugacgccccagcccuagaaccggcccu
agacgacggggcgguacauacauuaccaaccauaaucggaaaccuugcg
gccgugucugacuggguaaugagcaccguaccugucgcgccgccuagaa
gaaggagagggagaaaccugacugugacaugugacgagagagaagggaa
uauaacacccauggcuagcguccgauucuuuagagcagagcuguguccg
gccguacaagaaacagcggagacgcgugacacagcuauuucccuucagg
caccgccaaguaccaccauggaacugagccauccaccgaucuccuucgg
agcaccaagcgagacguuccccaucacauuuggggacuucgacgaagga
gaaauccaaagcuugucuucugagcuacuaacuuucggagacuuccuac
ccggugaaguggaugaucugacagauagcgacugguccacgugcccaga
cacggacgacgaguuaugacuagacagggcagguggguauauauucucg
ucggacacugguccaggccauuuacaacagaagucgguacgccagucag
ugcugccgguaaacacccuggaggaaguccacgaggagaaguguuaccc
accuaagcuggaugaauuaaaggagcaacuacuacuuaagaaacuccag
gagagugcguccauggccaauagaagcagguaucagucacgcaaagugg
aaaauaugaaagcaacaaucauccagagacuaaagagaggcuguaaacu
guauuuaauggcagagaccccgaaagucccgacuuaucggaccauauac
ccggcgccuguguacucgccuccgaucaauguccgauuguccaaccccg
aguccgcaguggcagcauguaaugaguucuuagcuagaaacuacccaac
uguuucaucauaccaaaucaccgacgaguaugaugcauaucuagacaug
guggacgggucggagaguugcuuggaccgagcgacauucaauccgucaa
aacuuaggagcuacccgaaacaacaugcuuaucacgcgccuucuaucag
aagcgcuguaccuuccccauuccagaacacacuacagaauguacuggca
gcagccacgaaaaggaacugcaacgucacacagaugagggaaauuugca
gccagcccuaucaggauaacaacugagaaucuaacaaccuaugucacua
aacuaaaggggccaaaagcagcagcgcuguuugcaaaaacccauaaucu
gcugccacugcaggauguaccaauggauagguucacaguagauaugaaa
agggaugugaagguaacuccugguacaaagcauacagaggaaagaccua
aggugcagguuauacaggcggcugaacccuuggcaacagcguaccuaug
uggaauucacagagaacugguuaggagauugaacgccguccuccuaccc
aaugugcauacacuauuugacaugucugccgaggacuucgaugccauua
uagccgcacacuucaagccaggagacgcuguuuuacaucgcuagccggg
uguuggaagaucgucugacaaaauccgcaugcgcggccuucaucggcga
cgacaacauaauacauggugucgucuccgaugaauugauggcagccaga
ugcgcuacuuggaugaacauggaagugaagaucauagaugcacgggcgc
uggcugaugaaguaaucagauggcaacgaacagggcuaauagaugagcu
ggagaaagcgguguacucuagguacgaagugcaggguauaucaguugcg
guaauguccauggccaccuuugcaagcuccagauccaacuucgagaagc
ucagaggacccgucauaacuuuguacggcgguccuaaauagguacgcac
uacagcuaccuauuuugcagaagccgacagcagguaccuaaauaccaau
cagccauaauggaguuuaucccaacccaaacuuucuacaauaggaggua
ccagccucgaccuuggacuccgcgcccuacuauccaaguuaucagaccc
agaccgcguccgcaaaggaaagccgggcaacuugcccagcugaucucag
caguuaauaaacugacaaugcgcgcgguaccucaacagaagccgcgcaa
gaaucggaagaauaagaagcaaaagcaaaagcagcaggcgccacgaaac
aacaugaaucaaaagaagcagcccccuaaaaagaaaccggcucaaaaga
aaaagaagccgggccguagagagagaaugugcaugaaaaucgaaaauga
uugcaucuucgaagucaagcaugaagguaagguaacagguuacgcgugc
uugguaggggacaaaguaaugaagccagcacacguaaaggggaccaucg
auaaugcggaccuggccaaauuggccuucaagcggucaucuaaguacga
ccuugaaugcgcgcagauacccgugcacaugaaguccgacgcuucgaag
uucacccaugagaaaccggagggguacuacaacuggcaccacggagcag
uacaguacucaggaggccgguucaccaucccuacaggugcgggcaaacc
aggggacagcgguagaccgaucuucgacaacaaggggccaucgucacga
aaaucaccccugagggggccgaagaguggagucuugccauuccaguuau
gugccugcuggcaaauaccacguuccccugcucccagcccccuugcaca
cccugcugcuacgaaaaagagccggagaaaacccugcgcaugcuagaag
acaacgucaugagccccggguacuaucagcugcuacaagcauccuuaac
auguucuccccgccgccagcgacgcaguaunaaggacaacuucaauguc
uauaaagccauaagaccguaccuagcucacugucccgacuguggagaag
ggcacucgugccauagucccguagcgcuagaacgcaucagaaacgaagc
gacagacgggacgcugaaaauccagguuuccuugcaaaucggaauaaag
acggaugauagccaugauuggaccaagcugcguuacauggacaaucaua
ugccagcagacgcagagagggccaggcuauuuguaagaacgucagcacc
gugcacgauuacuggaacaaugggacacuucauccuggcccgauguccg
aaaggagaaacucugacggugggauucacugacgguaggaagaucaguc
acucauguacgcacccauuucaccacgacccuccugugauaggccggga
aaaauuucauucccgaccgcagcacgguagagaacuaccuugcagcacg
uacgcgcagagcaccgcugcaacugccgaggagauagagguacauaugc
ccccagacaccccagaucgcacauugaugucacaacaguccgguaaugu
aaagaucacagucaauagucagacggugcgguacaaguguaauugcggu
gacucaaaugaaggacuaaccacuacagacaaagugauuaauaacugca
agguugaucaaugccaugccgcggucaccaaucacaaaaaauggcagua
uaauuccccucuggucccgcguaaugcugaacucggggaccgaaaagga
aaaguucacauuccguuuccucuggcaaaugugacaugcagggugccua
aggcaaggaaccccaccgugacguacggaaaaaaccaagucauggguga
cgcauaagaaggagaucagguuaaccgugccgacugaagggcucgaggu
cacguggggcaacaacgagccggcuguacccuacuaugacugugguagu
ugugucaguggccucguucguacuccugucgaugguggguguggcagug
gggaugugcaugugugcacgacgcagaugcauuacaccguacgaacuga
caccaggagcuaccgucccuuuccugcuuagccuaauaugcugcauuag
aacagcuaaagcggccacauaccaagaggcugcgguauaccuguggaac
gagcagaacagugaucccgaacacggugggaguaccguauaagacucua
gucaacagaccgggcuacagccccaugguacuggccgucuccguacgug
aaaugcugcgguacagcagagugcaaggacaagagccuaccugauuaca
gcuguaaggucuucaccggcgucuacccauucauguggggggcgccuac
ugcuucugcgacacugaaaauacgcaauugagcgaagcacauguggaga
aguccgaaucaugcaaaacagaauuugcaucagcauauagggcucauac
cgcauccgcaucagcuaagucuacaacauggacuacccgcccuucggcg
caggaagaccaggacaauuuggcgacauccaaagucgcacgccugagag
cgaagacgucuaugcuaacacacaacugguacugcagagaccguccgcg
gguacggugcacgugccguacucucaggcaccaucuggcuucaaguauu
ggcuaaaagaacgaggggcgucgcugcagcacacagcaccauuuggcug
ucaaauagcaacaaacccgguaagagcgaugaacugcgccguagggaac
augccuaucuccaucgacauaccggacgcggccuucacuagggucgucg
acgcgccaucuuuaacggacaugucgugugagguaccagccugcaccca
cuccucagacuuugggggcguagccaucauuaaauaugcagccagcaag
aaaggcaagugugcggugcauucgaugacuaacgccgucacuauucggg
aagcugaaauagaaguagaagggaacucucaguugcaaaucucuuuuuc
gacggcccuagccagcgccgaauuccgcguacaagucuguucuacacaa
guacacugugcagccgagugccauccaccgaaagaccauauagucaauu
acccggcgucacacaccacccucgggguccaagacauuuccguuacggc
gaugucaugggugcagaagaucacgggaggugugggacugguugucgcu
guugcagcacugauccuaaucguggugcuaugcgugucguuuagcaggc
acuaacuugacaacuagguacgaagguauauguguccccuaagagacac
accacauauagcuaagaaucaauagauaaguauagaucaaagggcugaa
caaccccugaauaguaacaaaauauaaaaaucaacaaaaaucauaaaau
agaaaaccagaaacagaaguagguaagaagguauauguguccccuaaga
gacacaccauauauagcuaagaaucaauagauaaguauagaucaaaggg
cugaauaaccccugaauaauaacaaaauauaaaaaucaauaaaaaucau
aaaauagaaaaccauaaacagaaguaguucaaagggcuauaaaaccccu
gaauaguaacaaaacauaaaacuaauaaaaaucaaaugaauaccauaau
uggcaaucggaagagauguagguacuuaagcuuccuaaaagcagccgaa
cucgcuuugagauguaggcguagcacaccgaacucuuccauaauucucc
gaacccacagggacguaggagauguucaaaguggcuauaaaacccugaa
caguaauaaaacauaaaauuaauaaggaucaaaugaguaccauaauugg
caaacggaagagauguagguacuuaagcuuccuaaaagcagccgaacuc
acuuugagauguaggcauagcauaccgaacucuuccacaauucuccgua
cccauagggacguagga
full-length 181/25 CHIKV virus genome with
a deletion in the P1234 polyprotein of the
nsP3 replicase gene, encoding for residues
1656 to 1717
SEQ ID NO: 2
acccaucauggauucuguguacguggacauagacgcugacagcgccuuu
uugaaggcccugcaacgugcguaccccauguuugagguggaaccuaggc
aggucacaucgaaugaccaugcuaaugcuagagcguucucgcaucuagc
cauaaaacuaauagagcaggaaauugaucccgacucaaccauccuggau
auagguagugcgccagcaaggaggaugaugucggacaggaaguaccacu
gcguuugcccgaugcgcagcgcagaagaucccgagagacucgcuaauua
ugcgagaaagcucgcaucugccgcaggaaaaguccuggacagaaacauu
ucuggaaagaucggggacuuacaagcggugauggccgugccagacacgg
agacgccaacauuuugcuuacacacagaugucucauguagacagagagc
agacgucgcgauauaccaagacgucuaugcuguacacgcacccacgucg
cuauaccaccaggcgauuaaaggaguccgaguggcguacuggguagggu
ucgacacaaccccguucauguacaacgcuauggcgggugccuaccccuc
auacucgacaaauugggcggaugagcagguacugaaggcuaagaacaua
ggauuauguucaacagaccugacggaagguagacgaggcaaauugucua
ucaugagagggaaaaagcuaaaaccgugcgaccgugugcuguucucagu
agggucaacgcuuuacccggaaagccgcacgcuacucgugugagggcua
cgucguuaagagaauaacgaugagcccaggccuuuauggaaaaaccaua
ggguaugcgguaacccaccacgcagacggauucuugaugugcaagacua
ccgacacgguugacggcgaaagagugucauucucggugugcacguacgu
gccggcgaccauuugugaucaaaugaccggcauccuugcuacagaaguc
acgccggaggaugcacagaagcuguugguggggcugaaccagaggauag
ugguuaacggcagaacgcaacggaacacgaacaccaugaagaacuaccg
gccugauacccagucaauccagaagguucaggccgaauuugacagcuuu
guaguaccgggccuguggucguccggguugucaaucccguugaggacua
gaaucaagugguuguuacgcaaggugccgaaaacagaccugaucccaua
cagcgggaaugcccaagaagcccaggaugcagaaaaagaagcagaggaa
gaacgagaagcagaacugacucaugaggcucuaccaccccuacaggcag
cacaggaagauguccaggucgaaaucgacguggaacagcuugaggauag
agcuggugcuggaauaauagagacuccgagaggcgcuaucaaaguuacu
gcccaacuaacagaccacgucgugggggaguaccugguacuuuccccgc
agaccguacuacgcagccagaagcucagccugauccacgcuuuagcgga
gcaagugaagacguguacgcacagcggacgagcagggagguaugcgguc
gaagcguacgauggccgaguccuagugcccucaggcuaugcaauuucgc
cugaagacuuccagagucuaagcgaaagcgcaacgaugguguacaacga
aagagaguucguaaacagaaaguuacaccacauugcgaugcacggacca
gcccugaacacugacgaagagucguaugagcuggugagggcagagagga
cagaacacgaguacgucuacgacguggaccagagaagaugcuguaagaa
ggaagaagcugcaggacugguacuggugggcgacuugacuaauccgccc
uaccacgaauucgcauacgaagggcuaaaaauucgccccgccugcccau
acaaaauugcagucauaggagucuucgggguaccaggaucuggcaaguc
agccauuaucaagaaccuaguuaccaggcaagaccuggugacuagcgga
aagaaagaaaacugccaagaaaucagcaccgacgugaugagacagagag
gucuagagauaucugcacguacgguagauucgcugcucuugaauggaug
caacagaccagucgacguguuguacguagacgaggcguuugcgugcagu
gccgcuucuucaauaugaugcagaugaaagucaacuacaaucauaacau
cugcacccaaguguaccacaaaaguaucuccaggcgguguacacugccu
gugacugccauugugucaucguugcauuacgaaggcaaaaugcgcacua
cgaaugaguacaacaugccgauuguaguggacacuacaggcucaacgaa
accugacccuggagaccucguguuaacgugcuucagaggguggguuaaa
caacugcaaauugacuaucguggacacgaggucaugacagcagccgcau
cccaaggguuaacuagaaaaggaguuuacgcaguuaggcaaaaaguuaa
cgaaaacccacucuaugcaucaacaucagagcacgucaacguacuccua
acgcguacggaagguaaacugguauggaagacacucucuggugacccgu
ggauaaagacgcugcagaacccaccgaaaggaaacuucaaagcaacuau
uaaggagugggagguggagcacgcaucgauaauggcgggcaucugcagu
caccaagugaccuuugacacauuccaaaacaaagccaacguuugcuggg
cuaagagcuuggucccuauccucgaaacagcggggauaaaacuaaauga
uaggcaguggucccagauaauucaagccuucaaagaagacaaagcauac
ucacccgaaguagcccugaaugaaauaugcacgcgcauguauggggugg
aucuagacagugggcuauucucuaaaccguugguaucuguguauuacgc
ggauaaccauugggauaauaggccgggaggaaagauguucggauucaac
ccugaggcagcguccauucuagaaagaaaguacccauuuacaaaaggaa
aguggaacaucaacaagcagaucugcgugacuaccaggaggauagaaga
cuucaacccuaccaccaacauuauaccggucaacaggagacuaccacac
ucauuaguggccgaacaccgcccaguaaaaggggaaagaauggaauggc
ugguuaacaagauaaacggacaccacguacuccugguuagcggcuauaa
ccuugcacugccuacuaagagagucaccuggguagcgccacuagguguc
cgcggagcggacuauacauacaaccuaguaccaacagugcguagaucac
gcaaugaaacugcaaaugcuagggggugacucacugagacugcucaaac
cggguggcucucuauugaucagagcauacgguuacgcagauagaaccag
ugaacgagucaucugcguacugggacgcaaguuuaggcagaaggaauuu
uacaacgcaugucaugaacaaucaacugaaugcagccuuuguaggacag
gccacccgagcaggaugugcaccaucguaccggguaaaacgcauggaca
ucgcgaagaacgaugaagagugcgugguuaacgccgccaacccucgcgg
guuaccaggugacgguguuugcaaggcaguauauaaaaaguggccggag
uccuuuaaaaacagugcaacaccaguaggaaccgcaaaaacaguuaugu
gcgguacguauccaguaauccacgccguaggaccaaacuucucaaauua
uucggagucugaaggggaccgggaauuggcggcugccuaucgagaaguc
gcaaaggaaguaacuagacugggaguaaauagcguagcuauaccucucc
ucuccacagguguauacucaggagggaaagacaggcuaacccagucacu
gaaccaccucuuuacagccauggacucgacggaugcagacguggucauc
uacugccgagacaaggaaugggagaagaaaauaucugaggccauacaga
ugcggacccaaguggagcugcuggaugagcacaucuccauagacugcga
ugucauucgcgugcacccugacaguagcuuggcaggcagaaaaggauac
agcaccacggaaggcgcacuguauucauaucuagaagggacacguuuuc
accagacggcaguggauauggcagagauauacacuauguggccaaagca
aacagaggccaaugagcaagucugccuauaugcccugggggaaaguauu
gaaucaaucaggcagaaaugcccgguggaugaugcagacgcaucaucuc
gguaauguuauucgaucacaaugugccaucgcgcguaaguccaagggcg
uaucgugcagccgcaggcggaaaccuugcggccgugucugacuggguaa
ugagcaccguaccugucgcgccgccuagaagaaggagagggagaaaccu
gacugugacaugugacgagagagaagggaauauaacacccauggcuagc
guccgauucuuuagagcagagcuguguccggccguacaagaaacagcgg
agacgcgugacacagcuauuucccuucaggcaccgccaaguaccaccau
ggaacugagccauccaccgaucuccuucggagcaccaagcgagacguuc
cccaucacauuuggggacuucgacgaaggagaaaucgaaagacgugccc
agacacggacgacgaguuaugacuagacagggcagguggguauauauuc
ucgucggacacugguccaggccauuuacaacagaagucgguacgccagu
cagugcugccgguaaacacccuggaggaaguccacgaggagaaguguua
cccaccuaagcuggaugaauuaaaggagcaacuacuacuuaagaaacuc
caggagagugcguccauggccaauagaagcagguaucagucacgcaaag
uggaaaauaugaaagcaacaaucauccagagacuaaagagaggcuguaa
acuguauuuaauggcagagaccccgaaagucccgacuuaucggaccaua
uacccggcgccuguguacucgccuccgaucaauguccgauuguccaacc
ccgaguccgcaguggcagcauguaaugaguucuuagcuagaaacuaccc
aacuguuucaucauaccaaaucaccgacgaguaugaugcauaucuagac
augguggacgggucggagaguugcuuggaccgagcgacauucaauccgu
caaaacuuaggagcuacccgaaacaacaugcuuaucacgcgccuucuau
cagaagcgcuguaccuuccccauuccagaacacacuacagaauguacug
gcagcagccacgaaaaggaacugcaacgucacacagaugagggaauuac
gcagccagcccuaucaggauaacaacugagaaucuaacaaccuauguca
cuaaacuaaaggggccaaaagcagcagcgcuguuugcaaaaacccauaa
ucugcugccacugcaggauguaccaauggauagguucacaguagauaug
aaaagggaugugaagguaacuccugguacaaagcauacagaggaaagac
cuaaggugcagguuauacaggcggcugaacccuuggcaacagcguaccu
auguggaauucacagagaacugguuaggagauugaacgccguccuccua
cccaaugugcauacacuauuugacaugucugccgaggacuucgaugcca
uuauagccgcacacuucaagccaggagacgcuguuuuagaaacggacau
agccuccuuugauaagagccaagaugauucacuugcgcuuaccgccuua
augcuguuagaagauuugggaguggaucacucccuguuggacuugauag
aggcugcuuucggagagauuuccagcugucaucugccgacagguacgcg
cuagccggguguuggaagaucgucugacaaaauccgcaugcgcggccuu
caucggcgacgacaacauaauacauggugucgucuccgaugaauugaug
gcagccagaugcgcuacuuggaugaacauggaagugaagaucauagaug
caguugcgcuggcugaugaaguaaucagauggcaacgaacagggcuaau
agaugagcuggagaaagcgguguacucuagguacgaagugcaggguaua
ucaguugcgguaauguccauggccaccuuugcaagcuccagauccaacu
ucgagaagcucagaggacccgucauaacuuuguacggcgguccuaaaua
gguacgcacuacagcuaccuauuuugcagaagccgacagcagcuaccus
aauaccaaucagccausauggaguuuaucccaacccaaacuuucuacaa
uaggagguaccagccucgaccuuggacuccgcgcccuacuauccaaguu
aucagacccagaccgcguccgcaaaggaaagccgggcaacuugcccagc
ugaucucagcaguuaauaaacugacaaugcgcgcgguaccucaacagaa
gccgcgcaagaaucggaagaauaagaagcaaaagcaaaagcagcaggcg
ccacgaaacaacaugaaucaaaagaagcagcccccuaaaaagaaaccgg
cucaaaagaaaaagaagcccggccguagagagagaaugugcaugaaaau
cgaaaaugauugcaucuucgaagucaagcaugaagguaagguaacaggu
uacgcgugcuugguaggggacaaaguaaugaagccagcacacguaaagg
ggaccaucgauaaugcggaccuggccaaauuggccuucaagcggucauc
uaaguacgaccuugaaugcgcgcagauacccgugcacaugaaguccgac
gcuucgaaguucacccaugagaaaccggagggguacuacaacuggcacc
acggagcaguacaguacucaggaggccgguucaccaucccuacaggugg
ggcaaaccaggggacagcgguagaccgaucuucgacaacaaggggcgcg
ugguggccauaguuuuaggaggagcuaaugaaggagcccguacagcccu
cucgguggugaccuggaacaaagacaucgucacgaaaaucaccccugag
ggggccgaagaguggagucuugccauuccaguuaugugccugcuggcaa
auaccacguuccccugcucccagcccccuugcacacccugcugcuacga
aaaagagccggagaaaacccugcgcaugcuagaagacaacgucaugagc
cccggguacuaucagcugcuacaagcauccuuaacauguucuccccgcc
gccagcgacgcaguauuaaggacaacuucaaugucuauaaagccauaag
accguaccuagcucacugucccgacuguggagaagggcacucgugccau
agucccguagcgcuagaacgcaucagaaacgaagcgacagacgggacgc
ugaaaauccagguuuccuugcaaaucggaauaaagacggaugauagcca
ugauuggaccaagcugcguuacauggacaaucauaugccagcagacgca
gagagggccaggcuauuuguaagaacgucagcaccgugcacgauuacug
gaacaaugggacacuucauccuggcccgauguccgaaaggagaaacucu
gacggugggauucacugacgguaggaagaucagucacucauguacgcac
ccauuucaccacgacccuccugugauaggccgggaaaaauuucauuccc
gaccgcagcacgguagagaacuaccuugcagcacguacgcgcagagcac
cgcugcaacugccgaggagauagagguacauaugcccccagacacccca
gaucgcacauugaugucacaacaguccgguaauguaaagaucacaguca
auagucagacggugcgguacaaguguaauugcggugacucaaaugaagg
acuaaccacuacagacaaagugauuaauaacugcaagguugaucaaugc
caugccgcggucaccaaucacaaaaaauggcaguauaauuccccucugg
ucccgcguaaugcugaacucggggaccgaaaaggaaaaguucacauucc
guuuccucuggcaaaugugacaugcagggugccuaaggcaaggaacccc
accgugacguacggaaaaaaccaagucaucaugcugcuguauccugacc
acccaacgcuccuguccuaccggaauaugggagaagaaccaaacuauca
agaagagugggugacgcauaagaaggagaucagguuaaccgugccgacu
gaagggcucgaggucacguggggcaacaacgagccguauguacccuacu
augacugugguaguugugucaguggccucguucguacuccugucgaugg
uggguguggcaguggggaugugcaugugugcacgacgcagaugcauuac
accguacgaacugacaccaggagcuaccgucccuuuccugcuuagccua
auaugcugcauuagaacagcuaaagcggccacauaccaagaggcugcgg
uauaccuguggaacgagcagcacagugaucccgaacacggugggaguac
cguauaagacucuagucaacagaccgggcuacagccccaugguacugga
gauggagcuucugucagucacuuuggagccaacgcuaucgcuugauuac
aucacgugcgaguauaaaaccgucaucccgucuccguacgugaaaugcu
gcgguacagcagagugcaaggacaagagccuaccugauuacagcuguaa
ggucuucaccggcgucuacccauucauguggggcggcgccuacugcuuc
ugcgacacugaaaauacgcaauugagcgaagcacauguggagaaguccg
aaucaugcaaaacagaauuugcaucagcauauagggcucauaccgcauc
cgcaucagcuaagcucuaaauucauuguggggccaaugucuucagccug
gacaccuuuugacaauaaaaucgugguguacaaaggcgacgucuacaac
auggacuacccgcccuucggcgcaggaagaccaggacaauuuggcgaca
uccaaagucgcacgccugagagcgaagacgucuaugcuaacacacaacu
gguacugcagagaccguccgcggguacggugcacgugccguacucucag
gcaccaucuggcuucaaguauuggcuaaaagaacgaggggcgucgcugc
agcacacagcaccauuuggcugucaaauagcaacaaacccgguaagagc
gaugaacugcgccguagggaacaugccuaucuccaucgacauaccggac
gcggccuucacuagggucgucgacgcgccaucuuuaacggacaugucgu
gugagguaccagccugcacccacuccucagacuuugggggcguagccau
cauuaaauaugcagccagcaagaaaggcaagugugcggugcauucgaug
acuaacgccgucacuauucgggaagcugaaauagaaguagaagggaacu
cucaguugcaaaucucuuuuucgacggcccuagccagcgccgaauuccg
cguacaagucuguucuacacaaguacacugugcagccgagugccaucca
ccgaaagaccauauagucaauuacccggcgucacacaccacccucgggg
uccaagacauuuccguuacggcgaugucaugggugcagaagaucacggg
aggugacaacuagguacgaagguauauguguccccuaagagacacacca
cauauagcuaagaaucaauagauaaguauagaucaaagggcugaacaac
cccugaauaguaacaaaauauaaaaaucaacaaaaaucauaaaauagaa
aaccagaaacagaaguagguaagaagguauauguguccccuaagagaca
caccauauauagcuaagaaucaauagauaaguauagaucaaagggcuga
auaaccccugaauaauaacaaaauauaaaaaucaauaaaaaucauaaaa
uagaaaaccauaaacagaaguaguucaaagggcuauaaaaccccugaau
aguaacaaaacauaaaacuaauaaaaaucaaaugaauaccauaauuggc
aauccgaagagauguagguacuuaagcuuccuaaaagcagccgaacucg
cuuugagauguaggcguagcacaccgaacucuuccauaauucuccgaac
ccacagggacguaggagauguucaaaguggcuauaaaacccugaacagu
aauaaaacauaaaauuaauaaggaucaaaugaguaccauaauuggcaaa
cggaagagauguagguacuuaagcuuccuaaaagcagccgaacucacuu
ugagauguaggcauagcauaccgaacucuuccacaauucuccguaccca
uagggacguaggagaug
full-length 181/25 CHIKV virus genome with a
deletion in the A6K genomic region, representing
amino acid residues 749 to 809
SEQ ID NO: 3
auggcugcgugagacacacguagccuaccaguuucuuacugcucuac
ucugcaaagcaagagauuaaua
acccaucauggauucuguguacguggacauagacgcugacagcgccuuu
uugaaggcccugcaacgugcguaccccauguuugagguggaaccuaggc
aggucacaucgaaugaccaugcuaaugcuagagcguucucgcaucuagc
cauaaaacuaauagagcaggaaauugaucccgacucaaccauccuggau
auagguagugcgccagcaaggaggaugaugucggacaggaaguaccacu
gcguuugcccgaugcgcagcgcagaagaucccgagagacucgcuaauua
ugcgagaaagcucgcaucugccgcaggaaaaguccuggacagaaacauu
ucuggaaagaucggggacuuacaagcggugauggccgugccagacaccg
agacgccaacauuuugcuuacacacagaugucucauguagacagagagc
agacgucgcgauauaccaagacgucuaugcuguacacgcacccacgucg
cuauaccaccaggcgauuaaaggaguccgaguggcguacuggguagggu
ucgacacaaccccguucauguacaacgcuauggcgggugccuaccccuc
auacucgacaaauugggcggaugagcagguacugaaggcuaagaacaua
ggauuauguucaacagaccugacggaagguagacgaggcaaauugucua
ucaugagagggaaaaagcuaaaaccgugcgaccgugugcuguucucagu
agggucaacgcuuuacccggaaagccgcacgcuacuuaagagcuggcac
cuaccaucgguguuccaucuaaagggcaagcuuagcuucacaugccgcu
gugacacagugguuucgugugagggcuacgucguuaagagaauaacgau
gagcccaggccuuuauggaaaaaccauaggguaugcgguaacccaccac
gcagacggauucuugaugugcaagacuaccgacacgguugacggcgaaa
gagugucauucucggugugcacquacgugccggcgaccauuugugauca
aaugaccggcauccuugcuacagaagucacgccggaggaugcacagaag
cuguugguggggcugaaccagaggauagugguuaacggcagaacgcaac
ggaacacgaacaccaugaagaacuaccuacuucccguggucgcccaggc
cuucaguaagugggcaaaggagugccggaaggacauggaagaugagaag
cuucugggggucagagaaagaacacuaaccugcugcugucuaugggcau
uuaagaagcagaaaacacacacggucuacaagaggccugauacccaguc
aauccagaagguucaggccgaauuugacagcuuuguaguaccgggccug
uggucguccggguugucaaucccguugaggacuagaaucaagugguugu
uacgcaaggugccgaaaacagaccugaucccauacagcgggaaugccca
agaagcccaggaugcagaaaaagaagcagaggaagaacgagaagcagaa
cugacucaugaggcucuaccaccccuacaggcagcacaggaagaugucc
aggucgaaaucgacguggaacagcuugaggauagagcuggugcuggaau
aauagagacuccgagaggcgcuaucaaaguuacugcccaacuaacagac
cacgucgugggggaguaccugguacuuuccccgcagaccguacuacgca
gccagaagcucagccugauccacgcuuuagcggagcaagugaagacgug
uacgcacagcggacgagcagggagguaugcggucgaagcguacgauggc
cgaguccuagugcccucaggcuaugcaauuucgccugaagacuuccaga
gucuaagcgaaagcgcaacgaugguguacaacgaaagagaguucguaaa
cagaaaguuacaccacauugcgaugcacggaccagcccugaacacugac
gaagagucguaugagcuggugagggcagagaggacagaacacgaguacg
ucuacgacguggaccagagaagaugcuguaagaaggaagaagcugcagg
acugguacuggugggcgacuugacuaauccgcccuaccacgaauucgca
uacgaagggcuaaaaauucgccccgccugcccauacaaaauugcaguca
uaggagucuucgggguaccaggaucuggcaagucagccauuaucaagaa
ccuaguuaccaggcaagaccuggugacuagcggaaagaaagaaaacugc
caagaaaucagcaccgacgugaugagacagagaggucuagagauaucug
cacguacgguagauucgcugcucuugaauggaugcaacagaccagucga
cguguuguacguagacgaggcguuugcgugcagugccgcuucuucaaua
ugaugcagaugaaagucaacuacaaucauaacaucugcacccaagugua
ccacaaaaguagaguacaacaugccgauuguaguggacacuacaggcuc
aacgaaaccugacccuggagaccucguguuaacgugcuucagagggugg
guuaaacaacugcaaauugacuaucguggacacgaggucaugacagcag
ccgcaucccaaggguuaacuagaaaaggaguuuacgcaguuaggcaaaa
aguuaacgaaaacccacucuaugcaucaacaucagagcacgucaacgua
cuccuaacgcguacggaagguaaacugguauggaagacacucucuggug
acccguggauaaagacgcugcagaacccaccgaaaggaaacuucaaagc
aacuauuaaggagugggagguggagcacgcaucgauaauggcgggcauc
ugcagucaccaagugaccuuugacacauuccaaaacaaagccaacguuu
gcugggcuaagagcuuggucccuauccucgaaacagcggggauaaaacu
aaaugauaggcaguggucccagauaauucaagccuucaaagaagacaaa
gcauacucacccgaaguagcccugaaugaaauaugcacgcgcauguaug
ggguggaucuagacagugggcuauucucuaaaccguugguaucugugua
uuacgcggauaaccauugggauaauaggccgggaggaaagauguucgga
uucaacccugaggcagcguccauucuagaaagaaaguacccauuuacaa
aaggaaaguggaacaucaacaagcagaucugcgugacuaccaggaggau
agaagacuucaacccuaccaccaacauuauaccggucaacaggagacua
ccacacucauuaguggccgaacaccgcccaguaaaaggggaaagaaugg
aauggcugguuaacaagauaaacggacaccacguacuccugguuagcgg
cuauaaccuugcacugccuacuaagagagucaccuggguagcgccacua
gguguccgcggagcggacuauacauacaaccuaguaccaacagugcgua
gaucacgcaaugaaacugcaaaugcuagggggugacucacugagacugc
ucaaaccggguggcucucuauugaucagagcauacgguuacgcagauag
aaccagugaacgagucaucugcguacugggacgcaaguuuaggcagaag
gaauuuuacaacgcaugucaugaacaaucaacugaaugcagccuuugua
ggacaggccacccgagcaggaugugcaccaucguaccggguaaaacgca
uggacaucgcgaagaacgaugaagagugcgugguuaacgccgccaaccc
caguaggaaccgcaaaaacaguuaugugcgguacguauccaguaaucca
cgccguaggaccaaacuucucaaauuauucggagucugaaggggaccgg
gaauuggcggcugccuaucgagaagucgcaaaggaaguaacuagacugg
gaguacucuuuacagccauggacucgacggaugcagacguggucaucua
cugccgagacaaggaaugggagaagaaaauaucugaggccauacagaug
cggacccaaguggagcugcuggaugagcacaucuccauagacugcgaug
ucauucgcgugcacccugacaguagcuuggcaggcagaaaaggauacag
caccacggaaggcgcacuguauucauaucuagaagggacacguuuucac
cagacggcaguggauauggcagagauauacacuauguggccaaagcaaa
cagaggccaaugagcaagucugccuauaugcccugggggaaaguauuga
aucaaucaggcagaaaugcccgguggaugaugcagacgcaucaucucgg
uaauguuauucgaucacaaugugccaucgcgcguaaguccaagggaaua
cagaucuucccaggagucuguacaggaagugaguacgacaacgucauug
acgcauagccaguuugaucuaagcgccgauggcgagacacugccugucc
cgucagaccuggaugcugacgccccagcccuagaaccggcccuagacga
cggggcgguacauacauuaccaaccauaaucggaaaccuugcggccgug
ucugacuggguaaugagcaccguaccugucgcgccgccuagaagaagga
gagggagaaaccugacugugacaugugacgagagagaagggaauauaac
acccauggcuagcguccgauucuuuagagcagagcuguguccggccgua
caagaaacagcggagacgcgugacacagcuauuucccuucaggcaccgc
caaguaccaccauggaacugagccauccaccgaucuccuucggagcacc
aagcgagacguuccccaucacauuuggggacuucgacgaaggagaaauu
gguccacgugcccagacacggacgacgaguuaugacuagacagggcagg
uggguauauauucucgucggacacugguccaggccauuuacaacagaag
ucgguacgccagucagugcugccgguaaacacccuggaggaaguccacg
aggagaaguguuacccaccuaagcuggaugaauuaaaggagcaacuacu
acuuaagaaacuccaggagagugcguccauggccaauagaagcagguau
cagucacgcaaaguggaaaauaugaaagcaacaaucauccagagacuaa
agagaggcuguaaacuguauuuaauggcagagaccccgaaagucccgac
uuaucggaccauauacccggcgccuguguacucgccuccgaucaauguc
cgauuguccaaccccgaguccgcaguggcagcauguaaugaguucuuag
cuagaaacuacccaacuguuucaucauaccaaaucaccgacgaguauga
ugcauaucuagacaugguggacgggucggagaguugcuuggaccgagcg
acauucaauccgucaaaacuuaggagcuacccgaaacaacaugcuuauc
acgcgccuucuaucagaagcgcuguaccuuccccauuccagaacacacu
acagaauguacuggcagcagccacgaaaaggaacugcaacgucacacag
augagggaaauuugcagccagcccuaucaggauaacaacugagaaucua
acaaccuaugucacuaaacuaaaggggccaaaagcagcagcccuguuug
caaaaacccauaaucugcugccacugcaggauguaccaauggauagguu
cacaguagauaugaaaagggaugugaagguaacuccugguacaaagcau
acagaggaaagaccuaaggugcagguuauacaggcggcugaacccuugg
caacagcguaccuauguggaauucacagagaacugguuaggagauugaa
cgccguccuccuacccaaugugcauacacuauuugacaugucugccgag
gacuucgaugccauuauagccgcacacuucaagccaggagacgcuguuu
uagcgaguggaucacucccuguuggacuugauagaggcugcuuucggag
agauuuccagcugucaucugccgacaggucaucgcuagccggguguugg
aagaucgucugacaaaauccgcaugcgcggccuucaucggcgacgacaa
cauaauacauggugucgucuccgaugaauugauggcagccagaugcgcu
acuuggaugaacauggaagugaagaucauagaugcacgggcgcuggcug
augaaguaaucagauggcaacgaacagggcuaauagaugagcuggagaa
agcgguguacucuagguacgaagugcaggguauaucaguugcgguaaug
uccauggccaccuuugcaagcuccagauccaacuucgagaagcucagag
gacccgucauaacuuuguacggcgguccuaaauagguacgcacuacagc
uaccuauuuugcagaagccgacagcagguaccuaaauaccaaucagcca
uaauggaguuuaucccaacccaaacuuucuacaauaggagguaccagcc
ucgaccuuggacuccgcgcccuacuauccaaguuaucagacccagaccg
cguccgcaaaggaaagccgggcaacuugcccagcugaucucagcaguua
auaaacugacaaugcgcgcgguaccucaacagaagccgcgcaagaaucg
gaagaauaagaagcaaaagcaaaagcagcaggcgccacgaaacaacaug
aaucaaaagaagcagcccccuaaaaagaaaccggcucaaaagaaaaaga
agccgggccguagagagagaaugugcaugaaaaucgaaaaugauugcau
cuucgaagucaagcaugaagguaagguaacagguuacgcgugcuuggua
ggggacaaaguaaugaagccagcacacguaaaggggaccaucgauaaug
cggaccuggccaaauuggccuucaagcggucaucuaaguacgaccuuga
augcgcgcagauacccgugcacaugaaguccgacgcuucgaaguucacc
caugagaaaccggagggguacuacaacuggcaccacggagcaguacagu
acucaggaggccgguucaccaucccuacaggugcgggcaaaccagggga
cagcgguagaccgaucuucgacaacaaggggcgcgugguggccauaguu
uuaggaggagcuaaugaaggagcccguacagcccucucgguggugaccu
ggaacaaagacaucgucacgaaaaucaccccugagggggccgaagagug
gagucuugccauuccaguuaugugccugcuggcaaauaccacguucccc
ugcucccagcccccuugcacacccugcugcuacgaaaaagagccggaga
aaacccugcgcaugcuagaagacaacgucaugagccccggguacuauca
gcugcuacaagcauccuuaacauguucuccccgccgccagcgacgcagu
auuaaggacaacuucaaugucuauaaagccauaagaccguaccuagcuc
acugucccgacuguggagaagggcacucgugccauagucccguagcgcu
agaacgcaucagaaacgaagcgacagacgggacgcugaaaauccagguu
uccuugcaaaucggaauaaagacggaugauagccaugauuggaccaagc
ugcguuacauggacaaucauaugccagcagacgcagagagggccaggcu
auuuguaagaacgucagcaccgugcacgauuacuggaacaaugggacac
uucauccuggcccgauguccgaaaggagaaacucugacggugggauuca
cugacgguaggaagaucagucacucauguacgcacccauuucaccacga
cccuccugugauaggccgggaaaaauuucauucccgaccgcagcacggu
agagaacuaccuugcagcacguacgcgcagagcaccgcugcaacugccg
aggagauagagguacauaugcccccagacaccccagaucgcacauugau
gucacaacaguccgguaauguaaagaucacagucaauagucagacggug
cgguacaaguguaauugcggugacucaaaugaaggacuaaccacuacag
acaaagugauuaauaacugcaagguugaucaaugccaugccgcggucac
caaucacaaaaaauggcaguauaauuccccucuggucccgcguaaugcu
gaacucggggaccgaaaaggaaaaguucacauuccguuuccucuggcaa
augugacaugcagggugccuaaggcaaggaaccccaccgugacguacgg
aaaaaaccaagucaucaugcugcuguauccugaccacccaacgcuccug
uccuaccggaauaugggagaagaaccaaacuaucaagaagaguggguga
cgcauaagaaggagaucagguuaaccgugccgacugaagggcucgaggu
cacguggggcaacaacgagccggcuguacccuacuaugacugugguagu
ugugucaguggccucguucguacuccugucgaugguggguguggcagug
gggaugugcaugugugcacgacgcagaugcauuacaccguacgaacuga
caccaggagcuaccgucccuuuccugcuuagccuaauaugcugcauuag
aacagcuaaagcggcguacgaacacguaacagugaucccgaacacggug
ggaguaccguauaagacucuagucaacagaccgggcuacagccccaugg
uacuggagauggagcuucugucagucacuuuggagccaacgcuaucgcu
ugauuacaucacgugcgaguauaaaaccgucaucccgucuccguacgug
aaaugcugcgguacagcagagugcaaggacaagagccuaccugauuaca
gcuguaaggucuucaccggcgucuacccauucauguggggcggcgccua
cugcuucugcgacacugaaaauacgcaauugagcgaagcacauguggag
aaguccgaaucaugcaaaacagaauuugcaucagcauauagggcucaua
ccgcauccgcaucaguaagcuccgcguccuuuaccaaggaaauaauguu
acuguaucugcuuaugcaaacggcgaucaugccgucacaguuaaggacg
cuaaauucauuguggggccaaugucuucagccuggacaccuuuugacaa
uaaaaucgugguguacaaaggcgacgucuacaacauggacuacccgccc
uucggcgcaggaagaccaggacaauuuggcgacauccaaagucgcacgc
cugagagcgaagacgucuaugcuaacacacaacugguacugcagagacc
guccgcggguacggugcacgugccguacucucaggcaccaucuggcuuc
aaguauuggcuaaaagaacgaggggcgucgcugcagcacacagcaccau
uuggcugucaaauagcaacaaacccgguaagagcgaugaacugcgccgu
agggaacaugccuaucuccaucgacauaccggacgcggccuucacuagg
gucgucgacgcgccaucuuuaacggacaugucgugugagguaccagccu
gcacccacuccucagacuuugggggcguagccaucauuaaauaugcagc
cagcaagaaaggcaagugugcggugcauucgaugacuaacgccgucacu
auucgggaagcugaaauagaaguagaaaguacacugugcagccgagugc
cauccaccgaaagaccauauagucaauuacccggcgucacacaccaccc
ucgggguccaagacauuuccguuacggcgaugucaugggugcagaagau
cacgggaggugugggacugguugucgcuguugcuguccccuaagagaca
caccacauauagcuaagaaucaauagauaaguauagaucaaagggcuga
acaaccccugaauaguaacaaaauauaaaaaucaacaaaaaucauaaaa
uagaaaaccagaaacagaaguagguaagaagguauauguguccccuaag
agacacaccauauauagcuaagaaucaauagauaaguauagaucaaagg
gcugaauaaccccugaauaauaacaaaauauaaaaaucaauaaaaauca
uaaaauagaaaaccauaaacagaaguaguucaaagggcuauaaaacccc
ugaauaguaacaaaacauaaaacuaauaaaaaucaaaugaauaccauaa
uuggcaaucggaagagauguagguacuuaagcuuccuaaaagcagccga
acucgcuuugagauguaggcguagcacaccgaacucuuccauaauucuc
cgaacccacagggacguaggagauguucaaaguggcuauaaaacccuga
acaguaauaaaacauaaaauuaauaaggaucaaaugaguaccauaauug
gcaaacggaagagauguagguacuuaagcuuccuaaaagcagccgaacu
cacuuugagauguaggca
full-length 181/25 CHIKV virus genome with ECMV
IRES substituted for the native CHIKV subgenomic
promoter
SEQ ID NO: 4
acccaucauggauucuguguacguggacauagacgcugacagcgccuuu
uugaaggcccugcaacgugcguaccccauguuugagguggaaccuaggc
aggucacaucgaaugaccaugcuaaugcuagagcguucucgcaucuagc
cauaaaacuaauagagcaggaaauugaucccgacucaaccauccuggau
auagguagugcgccagcaaggaggaugaugucggacaggaaguaccacu
gcguuugcccgaugcgcagcgcagaagaucccgagagacucgcuaauua
ugcgagaaagcucgcaucugccgcaggaaaaguccuggacagaaacauu
ucuggaaagaucggggacuuacaagcggugauggccgugccagacacgg
agacgccaacauuuugcuuacacacagaugucucauguagacagagagc
agacgucgcgauauaccaagacgucuaugcuguacacgcacccacgucg
cuauaccaccaggcgauuaaaggaguccgaguggcguacuggguagggu
ucgacacaaccccguucauguacaacgcuauggcgggugccuaccccuc
auacucgacaaauugggcggaugagcagguacugaaggcuaagaacaua
ggauuauguucaacagaccugacggaagguagacgaggcaaauugucua
ucaugagagggaaaaagcuaaaaccgugcgaccgugugcuguucucagu
agggucaacgcuuuacccggaaagccgcacgcuacuuaagagcuggcac
cuaccaucgguguuccaucuaaagggcaagcuuagcuucacaugccgcu
gugacacagugguuucgugugagggcuacgucguuaagagaauaacgau
gagcccaggccuuuauggaaaaaccauaggguaugcgguaacccaccac
gcagacggauucuugaugugcaagacuaccgacacgguugacggcgaaa
gagugucauucucggugugcacguacgugccggcgaccauuugugauca
aaugaccggcauccuugcuacagaagucacgccggaggaugcacagaag
cuguugguggggcugaaccagaggauagugguuaacggcagaacgcaac
ggaacacgaacaccaugaagaacuaccuacuucccguggucgcccaggc
cuucaguaagugggcaaaggagugccggaaggacauggaagaugagaag
cuucuggccugauacccagucaauccagaagguucaggccgaauuugac
agcuuuguaguaccgggccuguggucguccggguugucaaucccguuga
ggacuagaaucaagugguuguuacgcaaggugccgaaaacagaccugau
cccauacagcgggaaugcccaagaagcccaggaugcagaaaaagaagca
gaggaagaacgagaagcagaacugacucaugaggcucuaccaccccuac
aggcagcacaggaagauguccaggucgaaaucgacguggaacagcuuga
ggauagagcuggugcuggaauaauagagacuccgagaggcgcuaucaaa
guuacugcccaacuaacagaccacgucgugggggaguaccugguacuuu
ccccgcagaccguacuacgcagccagaagcucagccugauccacgcuuu
agcggagcaagugaagacguguacgcacagcggacgagcagggagguau
gcggucgaagcguacgauggccgaguccuagugcccucaggcuaugcaa
uuucgccugaagacuuccagagucuaagcgaaagcgcaacgauggugua
caacgaaagagaguucguaaacagaaaguuacaccacauugcgaugcac
ggaccagcccugaacacugacgaagagucguaugagcuggugagggcag
agaggacagaacacgaguacgucuacgacguggaccagagaagaugcug
uaagaaggaagaagcugcaggacugguacuggugggcgacuugacuaau
ccgcccuaccacgaauucgcauacgaagggcuaaaaauucgccccgccu
gcccauacaaaauugcagucauaggagucuucgggguaccaggaucugg
caagucagccauuaucaagaaccuaguuaccaggcaagaccuggugacu
agcggaaagaaagaaaacugccaagaaaucagcaccgacgugaugagac
agagaggucuagagauaucugcacguacgguagauucgcugcucuugaa
uggaugcaacagaccagucgacguguuguacguagacgaggcguuugcg
ugcagugccgcuucuucaauaugaugcagaugaaagucaacuacaauca
uaacaucugcacccaaguguaccacaaaaguagaguacaacaugccgau
uguaguggacacuacaggcucaacgaaaccugacccuggagaccucgug
uuaacgugcuucagaggguggguuaaacaacugcaaauugacuaucgug
gacacgaggucaugacagcagccgcaucccaaggguuaacuagaaaagg
aguuuacgcaguuaggcaaaaaguuaacgaaaacccacucuaugcauca
acaucagagcacgucaacguacuccuaacgcguacggaagguaaacugg
uauggaagacacucucuggugacccguggauaaagacgcugcagaaccc
accgaaaggaaacuucaaagcaacuauuaaggagugggagguggagcac
gcaucgauaauggcgggcaucugcagucaccaagugaccuuugacacau
uccaaaacaaagccaacguuugcugggcuaagagcuuggucccuauccu
cgaaacagcggggauaaaacuaaaugauaggcaguggucccagauaauu
caagccuucaaagaagacaaagcauacucacccgaaguagcccugaaug
aaauaugcacgcgcauguaugggguggaucuagacagugggcuauucuc
uaaaccguugguaucuguguauuacgcggauaaccauugggauaauagg
ccgggaggaaagauguucggauucaacccugaggcagcguccauucuag
aaagaaaguacccauuuacaaaaggaaaguggaacaucaacaagcagau
cugcgugacuaccaggaggauagaagacuucaacccuaccaccaacauu
auaccggucaacaggagacuaccacacucauuaguggccgaacaccgcc
caguaaaaggggaaagaauggaauggcugguuaacaagauaaacggaca
ccacguacuccugguuagcggcuauaaccuugcacugccuacuaagaga
gucaccuggguagcgccacuagguguccgcggagcggacuauacauaca
accuaguaccaacagugcguagaucacgcaaugaaacugcaaaugcuag
ggggugacucacugagacugcucaaaccggguggcucucuauugaucag
agcauacgguuacgcagauagaaccagugaacgagucaucugcguacug
ggacgcaaguuuaggcagaaggaauuuuacaacgcaugucaugaacaau
caacugaaugcagccuuuguaggacaggccacccgagcaggaugugcac
caucguaccggguaaaacgcauggacaucgcgaagaacgaugaagagug
cgugguuaacgccgccaacccucccgggunaccaggugacgguguuugc
aaggcaguauauaaaaaguggccggaguccuuuaaaaacagugcaacac
caguaggaaccgcaaaaacaguuaugugcgguacguauccaguaaucca
cgccguaggaccaaacuucucaaauuauucggagucugaaggggaccgg
gaauuggcggcugccuaucgagaagucgcaaaggaaguaacuagacugg
gaguaaauagguagcuauaccucuccucuccacagguguauacucagga
gggaaagacaggcuaacccagucacugaaccaccucuuuacagccaugg
acucgacggaugcagacguggucaucuacugccgagacaaggaauggga
gaagaaaauaucugaggccauacagaugcggacccaaguggagcugcug
gaugagcacaucuccauagacugcgaugucauucgcgugcacccugaca
guagcuuggcaggcagaaaaggauacagcaccacggaaggcgcacugua
uucauaucuagaagggacacguuuucaccagacggcaguggauauggca
gagauauacacuauguggccaaagcaaacagaggccaaugagcaagucc
ccgaaaacugucccgugucuuugccgguaugccaugacuccugaacgcg
ucacccgacuucgcaugaaccaugucgguaauguuauucgaucacaaug
ugccaucgcgcguaaguccaagggaauacagaucuucccaggagucugu
acaggaagugaguacgacaacgucauugacgcauagccaguuugaucua
agcgccgauggcgagacacugccugucccgucagaccuggaugcugacg
ccccagcccuagaaccggcccuagacgacggggcgguacauacauuacc
aaccauaaucggaaaccuugcggccgugucugacuggguaaugagcacc
guaccugucgcgccgccuagaagaaggagagggagaaaccugacuguga
caugugacgagagagaagggaauauaacacccauggcuagcguccgauu
cuuuagagcagagcuguguccggccguacaagaaacagcggagacgcgu
gacacagcuauuucccuucaggcaccgccaaguaccaccauggaacuga
gccauccaccgaucuccuucggagcaccaagcgagacguuccccaucac
auuuggggacuucgacgaaggagaaauugguccacgugcccagacacgg
acgacgaguuaugacuagacagggcagguggguauauauucucgucgga
cacugguccaggccauuuacaacagaagucgguacgccagucagugcug
ccgguaaacacccuggaggaaguccacgaggagaaguguuacccaccua
agcuggaugaauuaaaggagcaacuacuacuuaagaaacuccaggagag
ugcguccauggccaauagaagcagguaucagucacgcaaaguggaaaau
augaaagcaacaaucauccagagacuaaagagaggcuguaaacuguauu
uaauggcagagaccccgaaagucccgacuuaucggaccauauacccggc
gccuguguacucgccuccgaucaauguccgauuguccaaccccgagucc
gcaguggcagcauguaaugaguucuuagcuagaaacuacccaacuguuu
caucauaccaaaucaccgacgaguaugaugcauaucuagacauggugga
cgggucggagaguugcuuggaccgagcgacauucaauccgucaaaacuu
aggagcuacccgaaacaacaugcuuaucacgcgccuucuaucagaagcg
cuguaccuuccccauuccagaacacacuacagaauguacuggcagcagc
cacgaaaaggaacugcaacgucacacagaugagggaaauuugcagccag
cccuaucaggauaacaacugagaaucuaacaaccuaugucacuaaacua
aaggggccaaaagcagcagcgcuguuugcaaaaacccauaaucugcugc
cacugcaggauguaccaauggauagguucacaguagauaugaaaaggga
ugugaagguaacuccugguacaaagcauacagaggaaagaccuaaggug
cagguuauacaggcggcugaacccuuggcaacagcguaccuauguggaa
uucacagagaacugguuaggagauugaacgccguccuccuacccaaugu
gcauacacuauuugacaugucugccgaggacuucgaugccauuauagcc
gcacacuucaagccaggagacgcuguuuuacaucgcuagccggguguug
gaagaucgucugacaaaauccgcaugcgcggccuucaucggcgacgaca
acauaauacauggugucgucuccgaugaauugauggcagccagaugcgc
uacuuggaugaacauggaagugaagaucauagaugcacgggcgcuggcu
gaugaaguaaucagauggcaacgaacagggcuaauagaugagcuggaga
aagcgguguacucuagguacgaagugcaggguauaucaguugcgguaau
guccauggccaccuuugcaagcuccagauccaacuucgagaagcucaga
ggacccgucauaacgcuuuauggaggaccgaaguaauaaccccucuccc
uccccccccccuaacguuacugugugagggcccggaaaccuggcccugu
cuucuugacgagcauuccuaggggucuuuccccucucgccaaaggaaug
caaggucuguugaaugucgugaaggaagcaguuccucuggaagcuucuu
gaagacaaacaacgucuguagcgacccuuugcaggcagcggaacccccc
accuggcgacaggugccucugcggccaaaagccacguguauaagauaca
ccugcaaagrcggcacaaccccagugccacguugugaguuggauaguug
uggaaagagucaaauggcucuccucaagcguauucaacaaggggcugaa
ggaugcccagaagguaccccauuguaugggaucugaucuggggccucgg
uacacaugcuuuacauguguuuagucgagguuaaaaaaacgucuaggcc
ccccgaaccacggggacgugguuuuccuuugaaaaacacgaugauaaua
uggccacaaccauggaguuuaucccaacccaaacuuucuacaauaggag
guaccagccucgaccuuggacuccgcgcccuacuauccaaguuaucaga
cccagaccgcguccgcaaaggaaagccgggcaacuugcccagcugaucu
cagcaguuaauaaacugacaaugcgcgcgguaccucaacagaagccgcg
caagaaucggaagaauaagaagcaaaagcaaaagcagcaggcgccacga
aacaacaugaaucaaaagaagcagcccccuaaaaagaaaccggcucaaa
agaaaaagaagccgggccguagagagagaaugugcaugaaaaucgaaaa
ugauugcaucuucgaagucaagcaugaagguaagguaacagguuacgcg
ugcuugguaggggacaaaguaaugaagccagcacacguaaaggggacca
ucgauaaugcggaccuggccaaauuggccuucaagcggucaucuaagua
cgaccuugaaugcgcgcagauacccgugcacaugaaguccgacgcuucg
aaguucacccaugagaaaccggagggguacuacaacuggcaccacggag
caguacaguacucaggaggccgguucaccaucccuacaggugcgggcaa
accaggggacagcgguagaccgaucuucgacaacaaggggcgcguggug
gccauaguuuuaggaggagcuaaugaaggagcccguacagcccucucgg
uggugaccuggaacaaagacaucgucacgaaaaucaccccugagggggc
cgaagaguggagucuugccauuccaguuaugugccugcuggcaaauacc
acguuccccugcucccagcccccuugcacacccugcugcuacgaaaaag
agccggagaaaacccugcgcaugcuagaagacaacgucaugagccccgg
guacuaucagcugcuacaagcauccuuaacauguucuccccgccgccag
cgacgcaguauuaaggacaacuucaaugucuauaaagccauaagaccgu
accuagcucacugucccgacuguggagaagggcacucgugccauagucc
cguagcgcuagaacgcaucagaaacgaagcgacagacgggacgcugaaa
auccagguuuccuugcaaaucggaauaaagacggaugauagccaugauu
ggaccaagcugcguuacauggacaaucauaugccagcagacgcagagag
ggccaggcuauuuguaagaacgucagcaccgugcacgauuacuggaaca
augggacacuucauccuggcccgauguccgaaaggagaaacucugacgg
ugggauucacugacgguaggaagaucagucacucauguacgcacccauu
ucaccacgacccuccugugauaggccgggaaaaauuucauucccgaccg
cagcacgguagagaacuaccuugcagcacguacgcgcagagcaccgcug
caacugccgaggagauagagguacauaugcccccagacaccccagaucg
cacauugaugucacaacaguccgguaauguaaagaucacagucaauagu
cagacggugcgguacaaguguaauugcggugacucaaaugaaggacuaa
ccacuacagacaaagugauuaauaacugcaagguugaucaaugccaugc
cgcggucaccaaucacaaaaaauggcaguauaauuccccucuggucccg
cguaaugcugaacucggggaccgaaaaggaaaaguucacauuccguuuc
cucuggcaaaugugacaugcagggugccuaaggcaaggaaccccaccgu
gacguacggaaaaaaccaagucaucaugcugcuguauccugaccaccca
acgcuccuguccuaccggaauaugggagaagaaccaaacuaucaagaag
agugggugacgcauaagaaggagaucagguuaaccgugccgacugaagg
gcucgaggucacguggggcaacaacgagccguacaaguauuacuaugac
ugugguaguugugucaguggccucguucguacuccugucgauggugggu
guggcaguggggaugugcaugugugcacgacgcagaugcauuacaccgu
acgaacugacaccaggagcuaccgucccuuuccugcuuagccuaauaug
cugcauuagaacagcuaaagcggccacauaccaagaggcugcgguauac
cuguggaacgagcagcagccuuuguuuuggcugcaagcccuuauuccgc
uggcagcccugauuguccuaugcaacugucugagacucuuaccaugcuu
uuguaaaacguugacuuuuuuagccguaaugagcgucggugcccacacu
gugagcgcguacgaacacguaacagugaucccgaacacggugggaguac
cguauaagacucuagucaacagaccgggcuacagccccaugguacugga
gauggagcuucupucagucacuuuggagccaacgcuaucgcuugauuac
aucacgugcgaguauaaaaccgucaucccgucuccguacgugaaaugcu
gcgguacagcagagugcaaggacaagagccuaccugauuacagcuguaa
ggucuucaccggcgucuacccauucauguggggcggcgccuacugcuuc
ugcgacacugaaaauacgcaauugagcgaagcacauguggagaaguccg
aaucaugcaaaacagaauuugcaucagcauauagggcucauaccgcauc
cgcaucagcuaagcuccgcguccuuuguggggccaaugucuucagccug
gacaccuuuugacaauaaaaucgugguguacaaaggcgacgucuacaac
auggacuacccgcccuucggcgcaggaagaccaggacaauuuggcgaca
uccaaagucgcacgccugagagcgaagacgucuaugcuaacacacaacu
gguacugcagagaccguccgcggguacggugcacgugccguacucucag
gcaccaucuggcuucaaguauuggcuaaaagaacgaggggcgucgcugc
agcacacagcaccauuuggcugucaaauagcaacaaacccgguaagagc
gaugaacugcgccguagggaacaugccuaucuccaucgacauaccggac
gcggccuucacuagggucgucgacgcgccaucuuuaacggacaugucgu
gugagguaccagccugcacccacuccucagacuuugggggcguagccau
cauuaaauaugcagccagcaagaaaggcaagugugcggugcauucgaug
acuaacgccgucacuauucgggaagcugaaauagaaguagaagggaacu
cucaguugcaaaucucuuuuucgacggcccuagccagcgccgaauuccg
cguacaagucuguucuacacaaguacacugugcagccgagugccaucca
ccgaaagaccauauagucaauuacccggcgucacacaccacccucgggg
uccaagacauuuccguuacggcgaugucaugggugcagaagaucacggg
aggugugggacgcuacgaagguauauguguccccuaagagacacaccac
auauagcuaagaaucaauagauaaguauagaucaaagggcugaacaacc
ccugaauaguaacaaaauauaaaaaucaacaaaaaucauaaaauagaaa
accagaaacagaaguagguaagaagguauauguguccccuaagagacac
accauauauagcuaagaaucaauagauaaguauagaucaaagggcugaa
uaaccccugaauaauaacaaaauauaaaaaucaauaaaaaucauaaaau
agaaaaccauaaacagaaguaguucaaagggcuauaaaaccccugaaua
guaacaaaacauaaaacuaauaaaaaucaaaugaauaccauaauuggca
aucggaagagauguagguacuuaagcuuccuaaaagcagccgaacucgc
uuugagauguaggcguagcacaccgaacucuuccauaauucuccgaacc
cacagggacguaggagauguucaaaguggcuauaaaacccugaacagua
auaaaacauaaaauuaauaaggaucaaaugaguaccauaauuggcaaac
ggaagagauguagguacuuaagcuuccuaaaagcagccgaacucacuuu
gagauguaggcauagcauaccgaacucuuccacaauucuccguacccau
agggacguaggagauguuauuuuguuuuuaauauuu
mRNA encoding structural proteins C, E1,
and E2 from the 181/25
CHIKV strain sequence
SEQ ID NO: 5
auggaguuuaucccaacccaaacuuucuacaauaggagguaccagccuc
gaccuuggacuccgcgcccuacuauccaagucaucagacccagaccgc
guccgcaaaggaaggccgggcaacuugcccagcugaucucagcaguuaa
uaaacugacaaugcgcgugguaccucaacagaagccgcgcaagaaucgg
aagaauaagaagcaaaagcaaaagcagcaggcgccacgaaacaauacga
aucaaaagaagcagccccccaaaaagaaaccgguucaaaagaaaaagaa
gccgggccgcagagagagaaugugcaugaaaaucgaaaaugauugcauc
uucgaagucaagcaugaagguaagguaacagguuacgcgugcuugguag
gggacaaaguaaugaagccagcacacguaaaggggaccaucgauaacgc
ggaccuggccaaauuggccuucaagcggucaucuaaguacgaccuugaa
ugcgcgcagauacccgugcacaugaaguccgacgcuucgaaguucaccc
augagaaaccggagggguacuacaacuggcaccacggagcaguacagua
cucaggaggccgauucaccaucccuacaggugcgggcaaaccaggggau
agugguagaccgaucuucgacaacaaggggcgcgugguggccauaguuu
uaggaggagcuaaugaaggagcccguacagcccucucgguggugaccug
gaacaaagacaucgucacgaaaaucaccccugagggggccgaagagugg
agucuggccauuccaguuaugugccugcuggcaaauaccacguuccccu
gcucccggcccccuugcacacccugcugcuacgaaaaagagccggagaa
aaccuugcgcaugcuugaagacaaugucaugagccccggguacuaucag
cugcuacaagcauccuuaacauguucuccccgacgccagcggcgcagua
uuaaggaccacuucaaugucuauaaagccacaagaccguaccuagcuca
cugucccgacuguggagaagggcacucgugccauagucccguagcgcua
gaacgcaucagaaacgaagcgacagacgggacguugaaaauccagguuu
ccuugcaaaucggaauaaagacggaugauagccaugauuggaccaagcu
gcguuauauggacaaucacaugccagcagacgcagagcgggccgggcua
uuuguaagaacgucagcaccgugcacgauuacuggaacaaugggacacu
ucauucuggcccgauguccgaaaggagaaacucugacgggggguucacu
gacgguaggaagaucagucacucauguacgcacccauuucaccaugacc
cuccugugauaggccgggaaaaauuccauucccgaccgcagcacgguag
ggaacuaccuugcagcacguacgcgcagagcaccgcugcaacugccgag
gagauagagguacacaugcccccagacaccccagaucgcacauuaaugu
cacaacaguccggcaauguaaagaucacagucaauagucagacggugcg
guacaagugcaauuguggugacucaagugaaggauuaaccacuacagau
aaagugauuaauaacugcaaggucgaucaaugccaugccgcggucacca
aucacaaaaaauggcaguauaauuccccucuggucccgcguaaugcuga
auucggggaccggaaaggaaaaguucacauuccauuuccucuggcaaau
gugacaugcagggugccuaaagcaagaaaccccaccgugacguacggaa
aaaaccaagucaucauguugcuguauccugaccacccaacgcuccuguc
cuacaggaauaugggagaagaaccaaacuaucaagaagagugggugacg
cauaagaaggagaucagguuaaccgugccgacugaggggcucgagguca
cgugggguaacaaugagccguacaaguauuggccgcagugacuuuuuua
gccguacugagcgucggugcccacacugugagcgcguacgaacacguaa
cagugaucccgaacacggugggaguaccguauaagacucuagucaacag
accgggcuacagccccaugguauuggagauggagcuucugucugucacc
uuggaaccaacgcuaucgcuugauuacaucacgugcgaguauaaaaccg
uuaucccgucuccguacgugaaaugcugcgguacagcagaguguaagga
caagagccuaccugauuacagcuguaaggucuucaccggcgucuaccca
uucauguggggggcgccuacugcuucugcgacaccgaaaauacgcaauu
gagcgaagcacauguggagaaguccgaaucaugcaaaacagaauuugca
ucagcauacagggcucauaccgcauccgcaucagcuaagcuccgcgucc
uuuaccaaggaaauaauaucacuguggcugcuuaugcaaacggcgacca
ugccgucacaguuaaggacgcuaaauucauaguggggccaaugucuuca
gccuggacaccuuucgacaauaaaaucgugguguacaaaggcgacgucu
acaacauggacuacccgcccuucggcgcaggaagaccaggacaauuugg
cgacauccaaagucgcacgccugagagcgaagacgucuaugcuaauaca
caacugguacugcagagaccguccgcggguacggugcacgugccguacu
cucaggcaccaucuggcuucaaguauuggcuaaaagaacgaggggcguc
gcugcagcacacagcaccauuuggcugucaaauagcaacaaacccggua
agagcgaugaacugcgccguagggaacaugccuaucuccaucgacauac
cggacgcggccuuuaccagggucgucgacgcgccaucuuuaacggacau
gucgugugagguaucagccugcacccauuccucagacuuugggggcgua
gccaucauuaaauaugcagccaguaagaaaggcaagugugcagugcacu
cgaugacuaacgccgucacuauucgggaagcugaaauguucuacacaag
uacacugugcagccgagugccauccaccgaaagaccauauagucaauua
cccggcgucacacaccacccucgggguccaagacauuuccgcuacggcg
augucaugggugcagaagaucacgggaggugugggacugguugu
CHIKV 181/25-CE mRNA plasmid
SEQ ID NO: 6
tcgcgcgtttcggtgatgacggtgaaaacctctgacacatgcagctccc
ggagacggtcacagcttgtctgtaagcggatgccgggagcagacaagc
ccgtcagggcgcgtcagcgggtgttggcgggtgtcggggctggcttaac
tatgcggcatcagagcagattgtactgagagtgcaccatatgcggtgtg
aaataccgcacagatgcgtaaggagaaaataccgcatcaggcgccattc
gccattcaggctgcgcaactgttgggaagggcgatcggtgcgggcctct
tcgctattacgccagctggcgaaagggggatgtgctgcaaggcgattaa
gttgggtaacgccagggttttcccagtcacgacgttgtaaaacgacggc
cagtgaattcggtaatacgactcactataggcgcgtccgccccgcgagc
acagagcctcgcctttgccgatccgccgcccgtccacacccgccgccag
ctcacacgcgatggagtttatcccaacccaaactttctacaataggagg
taccagcctcgaccttggactccgcgccctactatccaagtcatcagac
ccagaccgcgtccgcaaaggaaggccgggcaacttgcccagctgatctc
agcagttaataaactgacaatgcgcgtggtacctcaacagaagccgcgc
aagaatcggaagaataagaagcaaaagcaaaagcagcaggcgccacgaa
acaatacgaatcaaaagaagcagccccccaaaaagaaaccggttcaaaa
gaaaaagaagccgggccgcagagagagaatgtgcatgaaaatcgaaaat
gattgcatcttcgaagtcaagcatgaaggtaaggtaacaggttacgcgt
gcttggtaggggacaaagtaatgaagccagcacacgtaaaggggaccat
cgataacgcggacctggccaaattggccttcaagcggtcatctaagtac
gaccttgaatgcgcgcagatacccgtgcacatgaagtccgacgcttcga
agttcacccatgagaaaccggaggggtactacaactggcaccacggagc
agtacagtactcaggaggccgattcaccatccctacaggtgcgggcaaa
ccaggggatagtggtagaccgatcttcgacaacaaggggcgcgtggtgg
ccatagttttaggaggagctaatgaaggagcccgtacagccctctcggt
ggtgacctggaacaaagacatcgtcacgaaaatcacccctgagggggcc
gaagagtggagtctggccattccagttatgtgcctgctggcaaatacca
cgttcccctgctcccggcccccttgcacaccctgctgctacgaaaaaga
gccggagaaaaccttgcgcatgcttgaagacaatgtcatgagccccggg
tactatcagctgctacaagcatccttaacatgttctccccgacgccagc
ggcgcagtattaaggaccacttcaatgtctataaagccacaagaccgta
cctagctcactgtcccgactgtggagaagggcactcgtgccatagtccc
gtagcgctagaacgcatcagaaacgaagcgacagacgggacgttgaaaa
tccaggtttccttgcaaatcggaataaagacggatgatagccatgattg
gaccaagctgcgttatatggacaatcacatgccagcagacgcagagcgg
gccgggctatttgtaagaacgtcagcaccgtgcacgattactggaacaa
tgggacacttcattctggcccgatgtccgaaaggagaaactctgacggc
ggggttcactgacggtaggaagatcagtcactcatgtacgcacccattt
caccatgaccctcctgtgataggccgggaaaaattccattcccgaccgc
agcacggtagggaactaccttgcagcacgtacgcgcagagcaccgctgc
aactgccgaggagatagaggtacacatgcccccagacaccccagatcgc
acattaatgtcacaacagtccggcaatgtaaagatcacagtcaatagtc
agacggtgcggtacaagtgcaattgtggtgactcaagtgaaggattaac
cactacagataaagtgattaataactgcaaggtcgatcaatgccatgcc
gcggtcaccaatcacaaaaaatggcagtataattcccctctggtcccgc
gtaatgctgaattcggggaccggaaaggaaaagttcacattccatttcc
tctggcaaatgtgacatgcagggtgcctaaagcaagaaaccccaccgtg
acgtacggaaaaaaccaagtcatcatgttgctgtatcctgaccacccaa
cgctcctgtcctacaggaatatgggagaagaaccaaactatcaagaaga
gtgggtgacgcataagaaggagatcaggttaaccgtgccgactgagggg
ctcgaggtcacgtggggtaacaatgagccgtacaagtattggccgcagt
tatccacaaacggtacagcccacggccacccgcatgagataattctgta
ttattatgagctgtacccaactatgactgcggtagttttgtcagtggcc
tcgttcatactcctgtcgatggtgggtgtggcagtggggatgtgcatgt
gtgcacgacgcagatgcattacaccgtacgaactgacaccaggagctac
cgtccctttcctgcttagcctaatatgctgcattagaacagctaaagcg
gccacataccaggaggccgcggtatacctgtggaacgagcagcagcctt
tattttggatgcaagcccttattccgctggcagccctgattgtcctatg
taactgtctgagactcttaccatgctgttgtaaaatgttgactttttta
gccgtactgagcgtcggtgcccacactgtgagcgcgtacgaacacgtaa
cagtgatcccgaacacggtgggagtaccgtataagactctagtcaacag
accgggctacagccccatggtattggagatggagcttctgtctgtcacc
ttggaaccaacgctatcgcttgattacatcacgtgcgagtataaaaccg
ttatcccgtctccgtacgtgaaatgctgcggtacagcagagtgtaagga
caagagcctacctgattacagctgtaaggtcttcaccggcgtctaccca
ttcatgtggggcggcgcctactgcttctgcgacaccgaaaatacgcaat
tgagcgaagcacatgtggagaagtccgaatcatgcaaaacagaatttgc
atcagcatacagggctcataccgcatccgcatcagctaagctccgcgtc
ctttaccaaggaaataatatcactgtggctgcttatgcaaacggcgacc
atgccgtcacagttaaggacgctaaattcatagtggggccaatgtcttc
agcctggacacctttcgacaataaaatcgtggtgtacaaaggcgacgtc
tacaacatggactacccgcccttcggcgcaggaagaccaggacaatttg
gcgacatccaaagtcgcacgcctgagagcgaagacgtctatgctaatac
acaactggtactgcagagaccgtccgcgggtacggtgcacgtgccgtac
tctcaggcaccatctggcttcaagtattggctaaaagaacgaggggcgt
cgctgcagcacacagcaccatttggctgtcaaatagcaacaaacccggt
aagagcgatgaactgcgccgtagggaacatgcctatctccatcgacata
ccggacgcggcctttaccagggtcgtcgacgcgccatctttaacggaca
tgtcgtgtgaggtatcagcctgcacccattcctcagactttgggggcgt
agccatcattaaatatgcagccagtaagaaaggcaagtgtgcagtgcac
tcgatgactaacgccgtcactattcgggaagctgaaatagaagtagaag
ggaactctcagttgcaaatctctttttcgacggccctagccagcgccga
atttcgcgtacaagtctgttctacacaagtacactgtgcagccgagtgc
catcgcagaagatcacgggaggtgtgggactggttgtcgctgttgcagc
actgatcctaatcgtggtgctatgcgtgtcgtttagcaggcactaaccg
gttaagcggactatgacttagttgcgttacaccctttcttgacaaaacc
taacttgcgcagaaaacaagatgagattggcatggctttatttgttttt
tttgttttgttttggttttttttttttttttggcttgactcaggattta
aaaactggaacggtgaaggtgacagcagtcggttggagcgagcatcccc
caaagttcacaatgtggccgaggactttgattgcacattgttgtttttt
taatagtcattccaaatatgagatgcattgttacaggaagtcccttgcc
atcctaaaagccaccccacttctctctaaggagaatggcccagtcctct
cccaagtccacacaggggaggtgatagcattgctttcgtgtaaattatg
taatgcaaaatttttttaatcttcgccttaatacttttttattttgttt
tattttgaatgatgagccttcgtgcccccccttccccctttttgtcccc
caacttgagatgtatgaaggcttttggtctccctgggagtgggtggagg
cagccagggcttacctgtacactgacttgagaccagttgaataaaagtg
cacaccttaaaaaaaaaaaaaaaaaaaaaaaaaaaaaaaaaaaaaaaaa
aaaaaaaaaaaaaaaaaaaagggtcggcatggcatctccacctcctcgc
ggtccgacctgggcatccgaaggaggacgcacgtccactcggatggcta
agggagcctgcattcgcagaagccacccgcgctgctaacaaagcccgaa
aggaagctgagttggctgctgccaccgctgagcaataactagcataacc
ccttggggcctctaaacgggtcttgaggggttttttgctgaaaggagga
actatatgcgctcatagcggccgggtacctcgcgaatgcatctagatat
cggatcccgggcccgtcgactgcagaggcctgcatgcaagcttggcgta
atcatggtcatagctgtttcctgtgtgaaattgttatccgctcacaatt
ccacacaacatacgagccggaagcataaagtgtaaagcctggggtgcct
aatgagtgagctaactcacattaattgcgttgcgctcactgcccgcttt
ccagtcgggaaacctgtcgtgccagctgcattaatgaatcggccaacgc
gcggggagaggcggtttgcgtattgggcgctcttccgcttcctcgctca
ctgactcgctgcgctcggtcgttcggctgcggcgagcggtatcagctca
ctcaaaggcggtaatacggttatccacagaatcaggggataacgcagga
aagaacatgtgagcaaaaggccagcaaaaggccaggaaccgtaaaaagg
ccgcgttgctggcgtttttccataggctccgcccccctgacgagcatca
caaaaatcgacgctcaagtcagaggtggcgaaacccgacaggactataa
agataccaggcgtttccccctggaagctccctcgtgcgctctcctgttc
cgaccctgccgcttaccggatacctgtccgcctttctcccttcgggaag
cgtggcgctttctcatagctcacgctgtaggtatctcagttcggtgtag
gtcgttcgctccaagctgggctgtgtgcacgaaccccccgttcagcccg
accgctgcgccttatccggtaactatcgtcttgagtccaacccggtaag
acacgacttatcgccactggcagcagccactggtaacaggattagcaga
gcgaggtatgtaggcggtgctacagagttcttgaagtggtggcctaact
acggctacactagaagaacagtatttggtatctgcgctctgctgaagcc
agttaccttcggaaaaagagttggtagctcttgatccggcaaacaaacc
accgctggtagcggtggtttttttgtttgcaagcagcagattacgcgca
gaaaaaaaggatctcaagaagatcctttgatcttttctacggggtctga
cgctcagtggaacgaaaactcacgttaagggattttggtcatgagatta
tcaaaaaggatcttcacctagatccttttaaattaaaaatgaagtttta
aatcaatctaaagtatatatgagtaaacttggtctgacagttaccaatg
cttaatcagtgaggcacctatctcagcgatctgtctatttcgttcatcc
atagttgcctgactccccgtcgtgtagataactacgatacgggagggct
taccatctggccccagtgctgcaatgataccgcgagacccacgctcacc
ggctccagatttatcagcaataaaccagccagccggaagggccgagcgc
agaagtggtcctgcaactttatccgcctccatccagtctattaattgtt
gccgggaagctagagtaagtagttcgccagttaatagtttgcgcaacgt
tgttgccattgctacaggcatcgtggtgtcacgctcgtcgtttggtatg
gcttcattcagctccggttcccaacgatcaaggcgagttacatgatccc
ccatgttgtgcaaaaaagcggttagctccttcggtcctccgatcgttgt
cagaagtaagttggccgcagtgttatcactcatggttatggcagcactg
cataattctcttactgtcatgccatccgtaagatgcttttctgtgactg
gtgagtactcaaccaagtcattctgagaatagtgtatgcggcgaccgag
ttgctcttgcccggcgtcaatacgggataataccgcgccacatagcaga
actttaaaagtgctcatcattggaaaacgttcttcggggcgaaaactct
caaggatcttaccgctgttgagatccagttcgatgtaacccactcgtgc
acccaactgatcttcagcatcttttactttcaccagcgtttctgggtga
gcaaaaacaggaaggcaaaatgccgcaaaaaagggaataagggcgacac
ggaaatgttgaatactcatactcttcctttttcaatattattgaagcat
ttatcagggttattgtctcatgagcggatacatatttgaatgtatttag
aaaaataaacaaataggggttccgcgcacatttccccgaaaagtgccac
ctgacgtctaagaaaccattattatcatgacattaacctataaaaatag
gcgtatcacgaggccctttcgtc
CHIKV 181/25 plasmid
SEQ ID NO: 7
atggctgcgtgagacacacgtagcctaccagtttcttactgctctactc
tgcaaagcaagagattaataacccatcatggattctgtgtacgtggac
atagacgctgacagcgcctttttgaaggccctgcaacgtgcgtacccca
tgtttgaggtggaacctaggcaggtcacatcgaatgaccatgctaatgc
tagagcgttctcgcatctagccataaaactaatagagcaggaaattgat
cccgactcaaccatcctggatataggtagtgcgccagcaaggaggatga
tgtcggacaggaagtaccactgcgtttgcccgatgcgcagcgcagaaga
tcccgagagactcgctaattatgcgagaaagctcgcatctgccgcagga
aaagtcctggacagaaacatttctggaaagatcggggacttacaagcgg
tgatggccgtgccagacacggagacgccaacattttgcttacacacaga
tgtctcatgtagacagagagcagacgtcgcgatataccaagacgtctat
gctgtacacgcacccacgtcgctataccaccaggcgattaaaggagtcc
gagtggcgtactgggtagggttcgacacaaccccgttcatgtacaacgc
tatggcgggtgcctacccctcatactcgacaaattgggcggatgagcag
gtactgaaggctaagaacataggattatgttcaacagacctgacggaag
gtagacgaggcaaattgtctatcatgagagggaaaaagctaaaaccgtg
cgaccgtgtgctgttctcagtagggtcaacgctttacccggaaagccgc
acgctacttaagagctggcacctaccatcggtgttccatctaaagggca
agcttagcttcacatgccgctgtgacacagtggtttcgtgtgagggcta
cgtcgttaagagaataacgatgagcccaggcctttatggaaaaaccata
gggtatgcggtaacccaccacgcagacggattcttgatgtgcaagacta
ccgacacggttgacggcgaaagagtgtcattctcggtgtgcacgtacgt
gccggcgaccatttgtgatcaaatgaccggcatccttgctacagaagtc
acgccggaggatgcacagaagctgttggtggggctgaaccagaggatag
tggttaacggcagaacgcaacggaacacgaacaccatgaagaactacct
acttcccgtggtcgcccaggccttcagtaagtgggcaaaggagtgccgg
aaggacatggaagatgagaagcttctgggggtcagagaaagaacactaa
cctgctgctgtctatgggcatttaagaagcagaaaacacacacggtcta
caagaggcctgatacccagtcaatccagaaggttcaggccgaatttgac
agctttgtagtaccgggcctgtggtcgtccgggttgtcaatcccgttga
ggactagaatcaagtggttgttacgcaaggtgccgaaaacagacctgat
cccatacagcgggaatgcccaagaagcccaggatgcagaaaaagaagca
gaggaagaacgagaagcagaactgactcatgaggctctaccacccctac
aggcagcacaggaagatgtccaggtcgaaatcgacgtggaacagcttga
ggatagagctggtgctggaataatagagactccgagaggcgctatcaaa
gttactgcccaactaacagaccacgtcgtgggggagtacctggtacttt
ccccgcagaccgtactacgcagccagaagctcagcctgatccacgcttt
agcggagcaagtgaagacgtgtacgcacagcggacgagcagggaggtat
gcggtcgaagcgtacgatggccgagtcctagtgccctcaggctatgcaa
tttcgcctgaagacttccagagtctaagcgaaagcgcaacgatggtgta
caacgaaagagagttcgtaaacagaaagttacaccacattgcgatgcac
ggaccagccctgaacactgacgaagagtcgtatgagctggtgagggcag
agaggacagaacacgagtacgtctacgacgtggaccagagaagatgctg
taagaaggaagaagctgcaggactggtactggtgggcgacttgactaat
ccgccctaccacgaattcgcatacgaagggctaaaaattcgccccgcct
gcccatacaaaattgcagtcataggagtcttcggggtaccaggatctgg
caagtcagccattatcaagaacctagttaccaggcaagacctggtgact
agcggaaagaaagaaaactgccaagaaatcagcaccgacgtgatgagac
agagaggtctagagatatctgcacgtacggtagattcgctgctcttgaa
tggatgcaacagaccagtcgacgtgttgtacgtagacgaggcgtttgcg
tgccactctggaacgttacttgctttgatcgccttggtgagaccaagac
agaaagttgtactttgtggtgacccgaagcagtgcggcttcttcaatat
gatgcagatgaaagtcaactacaatcataacatctgcacccaagtgtac
cacaaaagtatctccaggcggtgtacactgcctgtgactgccattgtgt
catcgttgcattacgaaggcaaaatgcgcactacgaatgagtacaacat
gccgattgtagtggacactacaggctcaacgaaacctgaccctggagac
ctcgtgttaacgtgcttcagagggtgggttaaacaactgcaaattgact
atcgtggacacgaggtcatgacagcagccgcatcccaagggttaactag
aaaaggagtttacgcagttaggcaaaaagttaacgaaaacccactctat
gcatcaacatcagagcacgtcaacgtactcctaacgcgtacggaaggta
aactggtatggaagacactctctggtgacccgtggataaagacgctgca
gaacccaccgaaaggaaacttcaaagcaactattaaggagtgggaggtg
gagcacgcatcgataatggcgggcatctgcagtcaccaagtgacctttg
acacattccaaaacaaagccaacgtttgctgggctaagagcttggtccc
tatcctcgaaacagcggggataaaactaaatgataggcagtggtcccag
ataattcaagccttcaaagaagacaaagcatactcacccgaagtagccc
tgaatgaaatatgcacgcgcatgtatggggtggatctagacagtgggct
attctctaaaccgttggtatctgtgtattacgcggataaccattgggat
aataggccgggaggaaagatgttcggattcaaccctgaggcagcgtcca
ttctagaaagaaagtacccatttacaaaaggaaagtggaacatcaacaa
gcagatctgcgtgactaccaggaggatagaagacttcaaccctaccacc
aacattataccggtcaacaggagactaccacactcattagtggccgaac
accgcccagtaaaaggggaaagaatggaatggctggttaacaagataaa
cggacaccacgtactcctggttagcggctataaccttgcactgcctact
aagagagtcacctgggtagcgccactaggtgtccgcggagcggactata
catacaacctagagctgggtctaccagcaacgcttggtaggtatgacct
agtggtcataaacatccacacaccttttcgcatacaccattaccaacag
tgcgtagatcacgcaatgaaactgcaaatgctagggggtgactcactga
gactgctcaaaccgggtggctctctattgatcagagcatacggttacgc
agatagaaccagtgaacgagtcatctgcgtactgggacgcaagtttaga
tcgtctagagcattgaaaccaccatgtgtcaccagtaatactgagatgt
ttttcctatttagcaattttgacaatggcagaaggaattttacaacgca
tgtcatgaacaatcaactgaatgcagcctttgtaggacaggccacccga
gcaggatgtgcaccatcgtaccgggtaaaacgcatggacatcgcgaaga
acgatgaagagtgcgtggttaacgccgccaaccctcgcgggttaccagg
tgacggtgtttgcaaggcagtatataaaaagtggccggagtcctttaaa
aacagtgcaacaccagtaggaaccgcaaaaacagttatgtgcggtacgt
atccagtaatccacgccgtaggaccaaacttctcaaattattcggagtc
tgaaggggaccgggaattggcggctgcctatcgagaagtcgcaaaggaa
gtaactagactgggagtaaatagcgtagctatacctctcctctccacag
gtgtatactcaggagggaaagacaggctaacccagtcactgaaccacct
ctttacagccatggactcgacggatgcagacgtggtcatctactgccga
gacaaggaatgggagaagaaaatatctgaggccatacagatgcggaccc
aagtggagctgctggatgagcacatctccatagactgcgatgtcattcg
cgtgcaccctgacagtagcttggcaggcagaaaaggatacagcaccacg
gaaggcgcactgtattcatatctagaagggacacgttttcaccagacgg
cagtggatatggcagagatatacactatgtggccaaagcaaacagaggc
caatgagcaagtctgcctatatgccctgggggaaagtattgaatcaatc
aggcagaaatgcccggtggatgatgcagacgcatcatctcccccgaaaa
ctgtcccgtgtctttgccggtatgccatgactcctgaacgcgtcacccg
acttcgcatgaaccatgtcacaaatataattgtgtgttcttcatttccc
cttccaaagtacaagatagaaggagtgcaaaaagtcaaatgctccaagg
taatgttattcgatcacaatgtgccatcgcgcgtaagtccaagggaata
cagatcttcccaggagtctgtacaggaagtgagtacgacaacgtcattg
acgcatagccagtttgatctaagcgccgatggcgagacactgcctgtcc
cgtcagacctggatgctgacgccccagccctagaaccggccctagacga
cggggcggtacatacattaccaaccataatcggaaaccttgcggccgtg
tctgactgggtaatgagcaccgtacctgtcgcgccgcctagaagaagga
gagggagaaacctgactgtgacatgtgacgagagagaagggaatataac
acccatggctagcgtccgattctttagagcagagctgtgtccggccgta
caagaaacagcggagacgcgtgacacagctatttcccttcaggcaccgc
caagtaccaccatggaactgagccatccaccgatctccttcggagcacc
aagcgagacgttccccatcacatttggggacttcgacgaaggagaaatc
gaaagcttgtcttctgagctactaactttcggagacttcctacccggtg
aagtggatgatctgacagatagcgactggtccacgtgcccagacacgga
cgacgagttatgactagacagggcaggtgggtatatattctcgtcggac
actggtccaggccatttacaacagaagtcggtacgccagtcagtgctgc
cggtaaacaccctggaggaagtccacgaggagaagtgttacccacctaa
gctggatgaattaaaggagcaactactacttaagaaactccaggagagt
gcgtccatggccaatagaagcaggtatcagtcacgcaaagtggaaaata
tgaaagcaacaatcatccagagactaaagagaggctgtaaactgtattt
aatggcagagaccccgaaagtcccgacttatcggaccatatacccggcg
cctgtgtactcgcctccgatcaatgtccgattgtccaaccccgagtccg
cagtggcagcatgtaatgagttcttagctagaaactacccaactgtttc
atcataccaaatcaccgacgagtatgatgcatatctagacatggtggac
gggtcggagagttgcttggaccgagcgacattcaatccgtcaaaactta
ggagctacccgaaacaacatgcttatcacgcgccttctatcagaagcgc
tgtaccttccccattccagaacacactacagaatgtactggcagcagcc
acgaaaaggaactgcaacgtcacacagatgagggaattacccactttgg
actcagcagtattcaacgtggagtgttttaaaaaattcgcatgtaaccg
agaatactgggaagaatttgcagccagccctatcaggataacaactgag
aatctaacaacctatgtcactaaactaaaggggccaaaagcagcagcgc
tgtttgcaaaaacccataatctgctgccactgcaggatgtaccaatgga
taggttcacagtagatatgaaaagggatgtgaaggtaactcctggtaca
aagcatacagaggaaagacctaaggtgcaggttatacaggcggctgaac
ccttggcaacagcgtacctatgtggaattcacagagaactggttaggag
attgaacgccgtcctcctacccaatgtgcatacactatttgacatgtct
gccgaggacttcgatgccattatagccgcacacttcaagccaggagacg
ctgttttagaaacggacatagcctcctttgataagagccaagatgattc
acttgcgcttaccgccttaatgctgttagaagatttgggagtggatcac
tccctgttggacttgatagaggctgctttcggagagatttccagctgtc
atctgccgacaggtacgcgcttcaagttcggcgctatgatgaaatccgg
tatgttcctaactctgttcgtcaacacgttgttaaatatcaccatcgct
agccgggtgttggaagatcgtctgacaaaatccgcatgcgcggccttca
tcggcgacgacaacataatacatggtgtcgtctccgatgaattgatggc
agccagatgcgctacttggatgaacatggaagtgaagatcatagatgca
gttgtatcccagaaagctccttacttttgtggagggtttatactgcatg
atactgtgacaggaacagcttgcagagtggcggacccgctaaaaaggtt
atttaaattgggcaaaccgttagcggcaggtgacgaacaagatgaagac
agaagacgggcgctggctgatgaagtaatcagatggcaacgaacagggc
taatagatgagctggagaaagcggtgtactctaggtacgaagtgcaggg
tatatcagttgcggtaatgtccatggccacctttgcaagctccagatcc
aacttcgagaagctcagaggacccgtcataactttgtacggcggtccta
aataggtacgcactacagctacctattttgcagaagccgacagcaggta
cctaaataccaatcagccataatggagtttatcccaacccaaactttct
acaataggaggtaccagcctcgaccttggactccgcgccctactatcca
agttatcagacccagaccgcgtccgcaaaggaaagccgggcaacttgcc
cagctgatctcagcagttaataaactgacaatgcgcgcggtacctcaac
agaagccgcgcaagaatcggaagaataagaagcaaaagcaaaagcagca
ggcgccacgaaacaacatgaatcaaaagaagcagccccctaaaaagaaa
ccggctcaaaagaaaaagaagccgggccgtagagagagaatgtgcatga
aaatcgaaaatgattgcatcttcgaagtcaagcatgaaggtaaggtaac
aggttacgcgtgcttggtaggggacaaagtaatgaagccagcacacgta
aaggggaccatcgataatgcggacctggccaaattggccttcaagcggt
catctaagtacgaccttgaatgcgcgcagatacccgtgcacatgaagtc
cgacgcttcgaagttcacccatgagaaaccggaggggtactacaactgg
caccacggagcagtacagtactcaggaggccggttcaccatccctacag
gtgcgggcaaaccaggggacagcggtagaccgatcttcgacaacaaggg
gcgcgtggtggccatagttttaggaggagctaatgaaggagcccgtaca
gccctctcggtggtgacctggaacaaagacatcgtcacgaaaatcaccc
ctgagggggccgaagagtggagtcttgccattccagttatgtgcctgct
ggcaaataccacgttcccctgctcccagcccccttgcacaccctgctgc
tacgaaaaagagccggagaaaaccctgcgcatgctagaagacaacgtca
tgagccccgggtactatcagctgctacaagcatccttaacatgttctcc
ccgccgccagcgacgcagtattaaggacaacttcaatgtctataaagcc
ataagaccgtacctagctcactgtcccgactgtggagaagggcactcgt
gccatagtcccgtagcgctagaacgcatcagaaacgaagcgacagacgg
gacgctgaaaatccaggtttccttgcaaatcggaataaagacggatgat
agccatgattggaccaagctgcgttacatggacaatcatatgccagcag
acgcagagagggccaggctatttgtaagaacgtcagcaccgtgcacgat
tactggaacaatgggacacttcatcctggcccgatgtccgaaaggagaa
actctgacggtgggattcactgacggtaggaagatcagtcactcatgta
cgcacccatttcaccacgaccctcctgtgataggccgggaaaaatttca
ttcccgaccgcagcacggtagagaactaccttgcagcacgtacgcgcag
agcaccgctgcaactgccgaggagatagaggtacatatgcccccagaca
ccccagatcgcacattgatgtcacaacagtccggtaatgtaaagatcac
agtcaatagtcagacggtgcggtacaagtgtaattgcggtgactcaaat
gaaggactaaccactacagacaaagtgattaataactgcaaggttgatc
aatgccatgccgcggtcaccaatcacaaaaaatggcagtataattcccc
tctggtcccgcgtaatgctgaactcggggaccgaaaaggaaaagttcac
attccgtttcctctggcaaatgtgacatgcagggtgcctaaggcaagga
accccaccgtgacgtacggaaaaaaccaagtcatcatgctgctgtatcc
tgaccacccaacgctcctgtcctaccggaatatgggagaagaaccaaac
tatcaagaagagtgggtgacgcataagaaggagatcaggttaaccgtgc
cgactgaagggctcgaggtcacgtggggcaacaacgagccgtacaagta
ttggccgcagttatccacaaacggtacagcccacggccacccgcatgag
ataattttgtattattatgagctgtaccctactatgactgtggtagttg
tgtcagtggcctcgttcgtactcctgtcgatggtgggtgtggcagtggg
gatgtgcatgtgtgcacgacgcagatgcattacaccgtacgaactgaca
ccaggagctaccgtccctttcctgcttagcctaatatgctgcattagaa
cagctaaagcggccacataccaagaggctgcggtatacctgtggaacga
gcagcagcctttgttttggctgcaagcccttattccgctggcagccctg
attgtcctatgcaactgtctgagactcttaccatgcttttgtaaaacgt
tgacttttttagccgtaatgagcgtcggtgcccacactgtgagcgcgta
cgaacacgtaacagtgatcccgaacacggtgggagtaccgtataagact
ctagtcaacagaccgggctacagccccatggtactggagatggagcttc
tgtcagtcactttggagccaacgctatcgcttgattacatcacgtgcga
gtataaaaccgtcatcccgtctccgtacgtgaaatgctgcggtacagca
gagtgcaaggacaagagcctacctgattacagctgtaaggtcttcaccg
gcgtctacccattcatgtggggcggcgcctactgcttctgcgacactga
aaatacgcaattgagcgaagcacatgtggagaagtccgaatcatgcaaa
acagaatttgcatcagcatatagggctcataccgcatccgcatcagcta
agctccgcgtcctttaccaaggaaataatgttactgtatctgcttatgc
aaacggcgatcatgccgtcacagttaaggacgctaaattcattgtgggg
ccaatgtcttcagcctggacaccttttgacaataaaatcgtggtgtaca
aaggcgacgtctacaacatggactacccgcccttcggcgcaggaagacc
aggacaatttggcgacatccaaagtcgcacgcctgagagcgaagacgtc
tatgctaacacacaactggtactgcagagaccgtccgcgggtacggtgc
acgtgccgtactctcaggcaccatctggcttcaagtattggctaaaaga
acgaggggcgtcgctgcagcacacagcaccatttggctgtcaaatagca
acaaacccggtaagagcgatgaactgcgccgtagggaacatgcctatct
ccatcgacataccggacgcggccttcactagggtcgtcgacgcgccatc
tttaacggacatgtcgtgtgaggtaccagcctgcacccactcctcagac
tttgggggcgtagccatcattaaatatgcagccagcaagaaaggcaagt
gtgcggtgcattcgatgactaacgccgtcactattcgggaagctgaaat
agaagtagaagggaactctcagttgcaaatctctttttcgacggcccta
gccagcgccgaattccgcgtacaagtctgttctacacaagtacactgtg
cagccgagtgccatccaccgaaagaccataaggtgtgggactggttgtc
gctgttgcagcactgatcctaatcgtggtgctatgcgtgtcgtttagca
ggcactaacttgacaactaggtacgaaggtatatgtgtcccctaagaga
cacaccacatatagctaagaatcaatagataagtatagatcaaagggct
gaacaacccctgaatagtaacaaaatataaaaatcaacaaaaatcataa
aatagaaaaccagaaacagaagtaggtaagaaggtatatgtgtccccta
agagacacaccatatatagctaagaatcaatagataagtatagatcaaa
gggctgaataacccctgaataataacaaaatataaaaatcaataaaaat
cataaaatagaaaaccataaacagaagtagttcaaagggctataaaacc
cctgaatagtaacaaaacataaaactaataaaaatcaaatgaataccat
aattggcaatcggaagagatgtaggtacttaagcttcctaaaagcagcc
gaactcgctttgagatgtaggcgtagcacaccgaactcttccataattc
tccgaacccacagggacgtaggagatgttcaaagtggctataaaaccct
gaacagtaataaaacataaaattaataaggatcaaatgagtaccataat
tggcaaacggaagagatgtaggtacttaagcttcctaaaagcagccgaa
ctcactttgagatgtaggcatagcataccgaactcttccacaattctcc
gtacccatagggacgtaggagatgttattttgtttttaatatttcaaaa
aaaaaaaaaaaaaaaaaaaaaaaaaaaaaaaaaaaaagcggccgcttaa
ttaatcgaggggaattaattcttgaagacgaaagggccaggtggcactt
ttcggggaaatgtgcgcggaacccctatttgtttatttttctaaataca
ttcaaatatgtatccgctcatgagacaataaccctgataaatgcttcaa
taatattgaaaaaggaagagtatgagtattcaacatttccgtgtcgccc
ttattcccttttttgcggcattttgccttcctgtttttgctcacccaga
aacgctggtgaaagtaaaagatgctgaagatcagttgggtgcacgagtg
ggttacatcgaactggatctcaacagcggtaagatccttgagagttttc
gccccgaagaacgttttccaatgatgagcacttttaaagttctgctatg
tggcgcggtattatcccgtgttgacgccgggcaagagcaactcggtcgc
cgcatacactattctcagaatgacttggttgagtactcaccagtcacag
aaaagcatcttacggatggcatgacagtaagagaattatgcagtgctgc
cataaccatgagtgataacactgcggccaacttacttctgacaacgatc
ggaggaccgaaggagctaaccgcttttttgcacaacatgggggatcatg
taactcgccttgatcgttgggaaccggagctgaatgaagccataccaaa
cgacgagcgtgacaccacgatgcctgtagcaatggcaacaacgttgcgc
aaactattaactggcgaactacttactctagcttcccggcaacaattaa
tagactggatggaggcggataaagttgcaggaccacttctgcgctcggc
ccttccggctggctggtttattgctgataaatctggagccggtgagcgt
gggtctcgcggtatcattgcagcactggggccagatggtaagccctccc
gtatcgtagttatctacacgacggggagtcaggcaactatggatgaacg
aaatagacagatcgctgagataggtgcctcactgattaagcattggtaa
ctgtcagaccaagtttactcatatatactttagattgatttaaaacttc
atttttaatttaaaaggatctaggtgaagatcctttttgataatctcat
gaccaaaatcccttaacgtgagttttcgttccactgagcgtcagacccc
gtagaaaagatcaaaggatcttcttgagatcctttttttctgcgcgtaa
tctgctgcttgcaaacaaaaaaaccaccgctaccagcggtggtttgttt
gccggatcaagagctaccaactctttttccgaaggtaactggcttcagc
agagcgcagataccaaatactgtccttctagtgtagccgtagttaggcc
accacttcaagaactctgtagcaccgcctacatacctcgctctgctaat
cctgttaccagtggctgctgccagtggcgataagtcgtgtcttaccggg
ttggactcaagacgatagttaccggataaggcgcagcggtcgggctgaa
cggggggttcgtgcacacagcccagcttggagcgaacgacctacaccga
actgagatacctacagcgtgagctatgagaaagcgccacgcttcccgaa
gggagaaaggcggacaggtatccggtaagcggcagggtcggaacaggag
agcgcacgagggagcttccagggggaaacgcctggtatctttatagtcc
tgtcgggtttcgccacctctgacttgagcgtcgatttttgtgatgctcg
tcaggggggcggagcctatggaaaaacgccagcaacgcgagctcgtaat
acgactcactatagg
CHIKV 181/25-A5nsP3 plasmid
SEQ ID NO: 8
atggctgcgtgagacacacgtagcctaccagtttcttactgctctactc
tgcaaagcaagagattaataacccatcatggattctgtgtacgtggac
atagacgctgacagcgcctttttgaaggccctgcaacgtgcgtacccca
tgtttgaggtggaacctaggcaggtcacatcgaatgaccatgctaatgc
tagagcgttctcgcatctagccataaaactaatagagcaggaaattgat
cccgactcaaccatcctggatataggtagtgcgccagcaaggaggatga
tgtcggacaggaagtaccactgcgtttgcccgatgcgcagcgcagaaga
tcccgagagactcgctaattatgcgagaaagctcgcatctgccgcagga
aaagtcctggacagaaacatttctggaaagatcggggacttacaagcgg
tgatggccgtgccagacacggagacgccaacattttgcttacacacaga
tgtctcatgtagacagagagcagacgtcgcgatataccaagacgtctat
gctgtacacgcacccacgtcgctataccaccaggcgattaaaggagtcc
gagtggcgtactgggtagggttcgacacaaccccgttcatgtacaacgc
tatggcgggtgcctacccctcatactcgacaaattgggcggatgagcag
gtactgaaggctaagaacataggattatgttcaacagacctgacggaag
gtagacgaggcaaattgtctatcatgagagggaaaaagctaaaaccgtg
cgaccgtgtgctgttctcagtagggtcaacgctttacccggaaagccgc
acgctacttaagagctggcacctaccatcggtgttccatctaaagggca
agcttagcttcacatgccgctgtgacacagtggtttcgtgtgagggcta
cgtcgttaagagaataacgatgagcccaggcctttatggaaaaaccata
gggtatgcggtaacccaccacgcagacggattcttgatgtgcaagacta
ccgacacggttgacggcgaaagagtgtcattctcggtgtgcacgtacgt
gccggcgaccatttgtgatcaaatgaccggcatccttgctacagaagtc
acgccggaggatgcacagaagctgttggtggggctgaaccagaggatag
tggttaacggcagaacgcaacggaacacgaacaccatgaagaactacct
acttcccgtggtcgcccaggccttcagtaagtgggcaaaggagtgccgg
aaggacatggaagatgagaagcttctgggggtcagagaaagaacactaa
cctgctgctgtctatgggcatttaagaagcagaaaacacacacggtcta
caagaggcctgatacccagtcaatccagaaggttcaggccgaatttgac
agctttgtagtaccgggcctgtggtcgtccgggttgtcaatcccgttga
ggactagaatcaagtggttgttacgcaaggtgccgaaaacagacctgat
cccatacagcgggaatgcccaagaagcccaggatgcagaaaaagaagca
gaggaagaacgagaagcagaactgactcatgaggctctaccacccctac
aggcagcacaggaagatgtccaggtcgaaatcgacgtggaacagcttga
ggatagagctggtgctggaataatagagactccgagaggcgctatcaaa
gttactgcccaactaacagaccacgtcgtgggggagtacctggtacttt
ccccgcagaccgtactacgcagccagaagctcagcctgatccacgcttt
agcggagcaagtgaagacgtgtacgcacagcggacgagcagggaggtat
gcggtcgaagcgtacgatggccgagtcctagtgccctcaggctatgcaa
tttcgcctgaagacttccagagtctaagcgaaagcgcaacgatggtgta
caacgaaagagagttcgtaaacagaaagttacaccacattgcgatgcac
ggaccagccctgaacactgacgaagagtcgtatgagctggtgagggcag
agaggacagaacacgagtacgtctacgacgtggaccagagaagatgctg
taagaaggaagaagctgcaggactggtactggtgggcgacttgactaat
ccgccctaccacgaattcgcatacgaagggctaaaaattcgccccgcct
gcccatacaaaattgcagtcataggagtcttcggggtaccaggatctgg
caagtcagccattatcaagaacctagttaccaggcaagacctggtgact
agcggaaagaaagaaaactgccaagaaatcagcaccgacgtgatgagac
agagaggtctagagatatctgcacgtacggtagattcgctgctcttgaa
tggatgcaacagaccagtcgacgtgttgtacgtagacgaggcgtttgcg
tgccactctggaacgttacttgctttgatcgccttggtgagaccaagac
agaaagttgtactttgtggtgacccgaagcagtgcggcttcttcaatat
gatgcagatgaaagtcaactacaatcataacatctgcacccaagtgtac
cacaaaagtatctccaggcggtgtacactgcctgtgactgccattgtgt
catcgttgcattacgaaggcaaaatgcgcactacgaatgagtacaacat
gccgattgtagtggacactacaggctcaacgaaacctgaccctggagac
ctcgtgttaacgtgcttcagagggtgggttaaacaactgcaaattgact
atcgtggacacgaggtcatgacagcagccgcatcccaagggttaactag
aaaaggagtttacgcagttaggcaaaaagttaacgaaaacccactctat
gcatcaacatcagagcacgtcaacgtactcctaacgcgtacggaaggta
aactggtatggaagacactctctggtgacccgtggataaagacgctgca
gaacccaccgaaaggaaacttcaaagcaactattaaggagtgggaggtg
gagcacgcatcgataatggcgggcatctgcagtcaccaagtgacctttg
acacattccaaaacaaagccaacgtttgctgggctaagagcttggtccc
tatcctcgaaacagcggggataaaactaaatgataggcagtggtcccag
ataattcaagccttcaaagaagacaaagcatactcacccgaagtagccc
tgaatgaaatatgcacgcgcatgtatggggtggatctagacagtgggct
attctctaaaccgttggtatctgtgtattacgcggataaccattgggat
aataggccgggaggaaagatgttcggattcaaccctgaggcagcgtcca
ttctagaaagaaagtacccatttacaaaaggaaagtggaacatcaacaa
gcagatctgcgtgactaccaggaggatagaagacttcaaccctaccacc
aacattataccggtcaacaggagactaccacactcattagtggccgaac
accgcccagtaaaaggggaaagaatggaatggctggttaacaagataaa
cggacaccacgtactcctggttagcggctataaccttgcactgcctact
aagagagtcacctgggtagcgccactaggtgtccgcggagcggactata
catacaacctagagctgggtctaccagcaacgcttggtaggtatgacct
agtggtcataaacatccacacaccttttcgcatacaccattaccaacag
tgcgtagatcacgcaatgaaactgcaaatgctagggggtgactcactga
gactgctcaaaccgggtggctctctattgatcagagcatacggttacgc
agatagaaccagtgaacgagtcatctgcgtactgggacgcaagtttaga
tcgtctagagcattgaaaccaccatgtgtcaccagtaatactgagatgt
ttttcctatttagcaattttgacaatggcagaaggaattttacaacgca
tgtcatgaacaatcaactgaatgcagcctttgtaggacaggccacccga
gcaggatgtgcaccatcgtaccgggtaaaacgcatggacatcgcgaaga
acgatgaagagtgcgtggttaacgccgccaaccctcgcgggttaccagg
tgacggtgtttgcaaggcagtatataaaaagtggccggagtcctttaaa
aacagtgcaacaccagtaggaaccgcaaaaacagttatgtgcggtacgt
atccagtaatccacgccgtaggaccaaacttctcaaattattcggagtc
tgaaggggaccgggaattggcggctgcctatcgagaagtcgcaaaggaa
gtaactagactgggagtaaatagcgtagctatacctctcctctccacag
gtgtatactcaggagggaaagacaggctaacccagtcactgaaccacct
ctttacagccatggactcgacggatgcagacgtggtcatctactgccga
gacaaggaatgggagaagaaaatatctgaggccatacagatgcggaccc
aagtggagctgctggatgagcacatctccatagactgcgatgtcattcg
cgtgcaccctgacagtagcttggcaggcagaaaaggatacagcaccacg
gaaggcgcactgtattcatatctagaagggacacgttttcaccagacgg
cagtggatatggcagagatatacactatgtggccaaagcaaacagaggc
caatgagcaagtctgcctatatgccctgggggaaagtattgaatcaatc
aggcagaaatgcccggtggatgatgcagacgcatcatctcccccgaaaa
ctgtcccgtgtctttgccggtatgccatgactcctgaacgcgtcacccg
acttcgcatgaaccatgtcacaaatataattgtgtgttcttcatttccc
cttccaaagtacaagatagaaggagtgcaaaaagtcaaatgctccaagg
taatgttattcgatcacaatgtgccatcgcgcgtaagtccaagggcgta
tcgtgcagccgcaggcggaaaccttgcggccgtgtctgactgggtaatg
agcaccgtacctgtcgcgccgcctagaagaaggagagggagaaacctga
ctgtgacatgtgacgagagagaagggaatataacacccatggctagcgt
ccgattctttagagcagagctgtgtccggccgtacaagaaacagcggag
acgcgtgacacagctatttcccttcaggcaccgccaagtaccaccatgg
aactgagccatccaccgatctccttcggagcaccaagcgagacgttccc
catcacatttggggacttcgacgaaggagaaatcgaaagcttgtcttct
gagctactaactttcggagacttcctacccggtgaagtggatgatctga
cagatagcgactggtccacgtgcccagacacggacgacgagttatgact
agacagggcaggtgggtatatattctcgtcggacactggtccaggccat
ttacaacagaagtcggtacgccagtcagtgctgccggtaaacaccctgg
aggaagtccacgaggagaagtgttacccacctaagctggatgaattaaa
ggagcaactactacttaagaaactccaggagagtgcgtccatggccaat
agaagcaggtatcagtcacgcaaagtggaaaatatgaaagcaacaatca
tccagagactaaagagaggctgtaaactgtatttaatggcagagacccc
gaaagtcccgacttatcggaccatatacccggcgcctgtgtactcgcct
ccgatcaatgtccgattgtccaaccccgagtccgcagtggcagcatgta
atgagttcttagctagaaactacccaactgtttcatcataccaaatcac
cgacgagtatgatgcatatctagacatggtggacgggtcggagagttgc
ttggaccgagcgacattcaatccgtcaaaacttaggagctacccgaaac
aacatgcttatcacgcgccttctatcagaagcgctgtaccttccccatt
ccagaacacactacagaatgtactggcagcagccacgaaaaggaactgc
aacgtcacacagatgagggaattacccactttggactcagcagtattca
acgtggagtgttttaaaaaattcgcatgtaaccgagaatactgggaaga
atttgcagccagccctatcaggataacaactgagaatctaacaacctat
gtcactaaactaaaggggccaaaagcagcagcgctgtttgcaaaaaccc
ataatctgctgccactgcaggatgtaccaatggataggttcacagtaga
tatgaaaagggatgtgaaggtaactcctggtacaaagcatacagaggaa
agacctaaggtgcaggttatacaggcggctgaacccttggcaacagcgt
acctatgtggaattcacagagaactggttaggagattgaacgccgtcct
cctacccaatgtgcatacactatttgacatgtctgccgaggacttcgat
gccattatagccgcacacttcaagccaggagacgctgttttagaaacgg
acatagcctcctttgataagagccaagatgattcacttgcgcttaccgc
cttaatgctgttagaagatttgggagtggatcactccctgttggacttg
atagaggctgctttcggagagatttccagctgtcatctgccgacaggta
cgcgcttcaagttcggcgctatgatgaaatccggtatgttcctaactct
gttcgtcaacacgttgttaaatatcaccatcgctagccgggtgttggaa
gatcgtctgacaaaatccgcatgcgcggccttcatcggcgacgacaaca
taatacatggtgtcgtctccgatgaattgatggcagccagatgcgctac
ttggatgaacatggaagtgaagatcatagatgcagttgtatcccagaaa
gctccttacttttgtggagggtttatactgcatgatactgtgacaggaa
cagcttgcagagtggcggacccgctaaaaaggttatttaaattgggcaa
accgttagcggcaggtgacgaacaagatgaagacagaagacgggcgctg
gctgatgaagtaatcagatggcaacgaacagggctaatagatgagctgg
agaaagcggtgtactctaggtacgaagtgcagggtatatcagttgcggt
aatgtccatggccacctttgcaagctccagatccaacttcgagaagctc
agaggacccgtcataactttgtacggcggtcctaaataggtacgcacta
cagctacctattttgcagaagccgacagcaggtacctaaataccaatca
gccataatggagtttatcccaacccaaactttctacaataggaggtacc
agcctcgaccttggactccgcgccctactatccaagttatcagacccag
accgcgtccgcaaaggaaagccgggcaacttgcccagctgatctcagca
gttaataaactgacaatgcgcgcggtacctcaacagaagccgcgcaaga
atcggaagaataagaagcaaaagcaaaagcagcaggcgccacgaaacaa
catgaatcaaaagaagcagccccctaaaaagaaaccggctcaaaagaaa
aagaagccgggccgtagagagagaatgtgcatgaaaatcgaaaatgatt
gcatcttcgaagtcaagcatgaaggtaaggtaacaggttacgcgtgctt
ggtaggggacaaagtaatgaagccagcacacgtaaaggggaccatcgat
aatgcggacctggccaaattggccttcaagcggtcatctaagtacgacc
ttgaatgcgcgcagatacccgtgcacatgaagtccgacgcttcgaagtt
cacccatgagaaaccggaggggtactacaactggcaccacggagcagta
cagtactcaggaggccggttcaccatccctacaggtgcgggcaaaccag
gggacagcggtagaccgatcttcgacaacaaggggcgcgtggtggccat
agttttaggaggagctaatgaaggagcccgtacagccctctcggtggtg
acctggaacaaagacatcgtcacgaaaatcacccctgagggggccgaag
agtggagtcttgccattccagttatgtgcctgctggcaaataccacgtt
cccctgctcccagcccccttgcacaccctgctgctacgaaaaagagccg
gagaaaaccctgcgcatgctagaagacaacgtcatgagccccgggtact
atcagctgctacaagcatccttaacatgttctccccgccgccagcgacg
cagtattaaggacaacttcaatgtctataaagccataagaccgtaccta
gctcactgtcccgactgtggagaagggcactcgtgccatagtcccgtag
cgctagaacgcatcagaaacgaagcgacagacgggacgctgaaaatcca
ggtttccttgcaaatcggaataaagacggatgatagccatgattggacc
aagctgcgttacatggacaatcatatgccagcagacgcagagagggcca
ggctatttgtaagaacgtcagcaccgtgcacgattactggaacaatggg
acacttcatcctggcccgatgtccgaaaggagaaactctgacggtggga
ttcactgacggtaggaagatcagtcactcatgtacgcacccatttcacc
acgaccctcctgtgataggccgggaaaaatttcattcccgaccgcagca
cggtagagaactaccttgcagcacgtacgcgcagagcaccgctgcaact
gccgaggagatagaggtacatatgcccccagacaccccagatcgcacat
tgatgtcacaacagtccggtaatgtaaagatcacagtcaatagtcagac
ggtgcggtacaagtgtaattgcggtgactcaaatgaaggactaaccact
acagacaaagtgattaataactgcaaggttgatcaatgccatgccgcgg
tcaccaatcacaaaaaatggcagtataattcccctctggtcccgcgtaa
tgctgaactcggggaccgaaaaggaaaagttcacattccgtttcctctg
gcaaatgtgacatgcagggtgcctaaggcaaggaaccccaccgtgacgt
acggaaaaaaccaagtcatcatgctgctgtatcctgaccacccaacgct
cctgtcctaccggaatatgggagaagaaccaaactatcaagaagagtgg
gtgacgcataagaaggagatcaggttaaccgtgccgactgaagggctcg
aggtcacgtggggcaacaacgagccgtacaagtattggccgcagttatc
cacaaacggtacagcccacggccacccgcatgagataattttgtattat
tatgagctgtaccctactatgactgtggtagttgtgtcagtggcctcgt
tcgtactcctgtcgatggtgggtgtggcagtggggatgtgcatgtgtgc
acgacgcagatgcattacaccgtacgaactgacaccaggagctaccgtc
cctttcctgcttagcctaatatgctgcattagaacagctaaagcggcca
cataccaagaggctgcggtatacctgtggaacgagcagcagcctttgtt
ttggctgcaagcccttattccgctggcagccctgattgtcctatgcaac
tgtctgagactcttaccatgcttttgtaaaacgttgacttttttagccg
taatgagcgtcggtgcccacactgtgagcgcgtacgaacacgtaacagt
gatcccgaacacggtgggagtaccgtataagactctagtcaacagaccg
ggctacagccccatggtactggagatggagcttctgtcagtcactttgg
agccaacgctatcgcttgattacatcacgtgcgagtataaaaccgtcat
cccgtctccgtacgtgaaatgctgcggtacagcagagtgcaaggacaag
agcctacctgattacagctgtaaggtcttcaccggcgtctacccattca
tgtggggcggcgcctactgcttctgcgacactgaaaatacgcaattgag
cgaagcacatgtggagaagtccgaatcatgcaaaacagaatttgcatca
gcatatagggctcataccgcatccgcatcagctaagctccgcgtccttt
accaaggaaataatgttactgtatctgcttatgcaaacggcgatcatgc
cgtcacagttaaggacgctaaattcattgtggggccaatgtcttcagcc
tggacaccttttgacaataaaatcgtggtgtacaaaggcgacgtctaca
acatggactacccgcccttcggcgcaggaagaccaggacaatttggcga
catccaaagtcgcacgcctgagagcgaagacgtctatgctaacacacaa
ctggtactgcagagaccgtccgcgggtacggtgcacgtgccgtactctc
aggcaccatctggcttcaagtattggctaaaagaacgaggggcgtcgct
gcagcacacagcaccatttggctgtcaaatagcaacaaacccggtaaga
gcgatgaactgcgccgtagggaacatgcctatctccatcgacataccgg
acgcggccttcactagggtcgtcgacgcgccatctttaacggacatgtc
gtgtgaggtaccagcctgcacccactcctcagactttgggggcgtagcc
atcattaaatatgcagccagcaagaaaggcaagtgtgcggtgcattcga
tgactaacgccgtcactattcgggaagctgaaatagaagtagaagggaa
ctctcagttgcaaatctctttttcgacggccctagccagcgccgaattc
cgcgtacaagtctgttctacacaagtacactgtgcagccgagtgccatc
caccgaaagaccatatagtcaattacccggcgtcacacaccaccctcgg
ggtccaagacatttccgttacggcgatgtcatgggtgcagaagatcacg
ggaggtgtgggactggttgtcgctgttgcagcactgatcctaatcgtgg
tgctatgcgtgtcgtttagcaggcactaacttgacaactaggtacgaag
gtatatgtgtcccctaagagacacaccacatatagctaagaatcaatag
ataagtatagatcaaagggctgaacaacccctgaatagtaacaaaatat
aaaaatcaacaaaaatcataaaatagaaaaccagaaacagaagtaggta
agaaggtatatgtgtcccctaagagacacaccatatatagctaagaatc
aatagataagtatagatcaaagggctgaataacccctgaataataacaa
aatataaaaatcaataaaaatcataaaatagaaaaccataaacagaagt
agttcaaagggctataaaacccctgaatagtaacaaaacataaaactaa
taaaaatcaaatgaataccataattggcaatcggaagagatgtaggtac
ttaagcttcctaaaagcagccgaactcgctttgagatgtaggcgtagca
caccgaactcttccataattctccgaacccacagggacgtaggagatgt
tcaaagtggctataaaaccctgaacagtaataaaacataaaattaataa
ggatcaaatgagtaccataattggcaaacggaagagatgtaggtactta
agcttcctaaaagcagccgaactcactttgagatgtaggcatagcatac
cgaactcttccacaattctccgtacccatagggacgtaggagatgttat
tttgtttttaatatttcaaaaaaaaaaaaaaaaaaaaaaaaaaaaaaaa
aaaaaaaaagcggccgcttaattaatcgaggggaattaattcttgaaga
cgaaagggccaggtggcacttttcggggaaatgtgcgcggaacccctat
ttgtttatttttctaaatacattcaaatatgtatccgctcatgagacaa
taaccctgataaatgcttcaataatattgaaaaaggaagagtatgagta
ttcaacatttccgtgtcgcccttattcccttttttgcggcattttgcct
tcctgtttttgctcacccagaaacgctggtgaaagtaaaagatgctgaa
gatcagttgggtgcacgagtgggttacatcgaactggatctcaacagcg
gtaagatccttgagagttttcgccccgaagaacgttttccaatgatgag
cacttttaaagttctgctatgtggcgcggtattatcccgtgttgacgcc
gggcaagagcaactcggtcgccgcatacactattctcagaatgacttgg
ttgagtactcaccagtcacagaaaagcatcttacggatggcatgacagt
aagagaattatgcagtgctgccataaccatgagtgataacactgcggcc
aacttacttctgacaacgatcggaggaccgaaggagctaaccgcttttt
tgcacaacatgggggatcatgtaactcgccttgatcgttgggaaccgga
gctgaatgaagccataccaaacgacgagcgtgacaccacgatgcctgta
gcaatggcaacaacgttgcgcaaactattaactggcgaactacttactc
tagcttcccggcaacaattaatagactggatggaggcggataaagttgc
aggaccacttctgcgctcggcccttccggctggctggtttattgctgat
aaatctggagccggtgagcgtgggtctcgcggtatcattgcagcactgg
ggccagatggtaagccctcccgtatcgtagttatctacacgacggggag
tcaggcaactatggatgaacgaaatagacagatcgctgagataggtgcc
tcactgattaagcattggtaactgtcagaccaagtttactcatatatac
tttagattgatttaaaacttcatttttaatttaaaaggatctaggtgaa
gatcctttttgataatctcatgaccaaaatcccttaacgtgagttttcg
ttccactgagcgtcagaccccgtagaaaagatcaaaggatcttcttgag
atcctttttttctgcgcgtaatctgctgcttgcaaacaaaaaaaccacc
gctaccagcggtggtttgtttgccggatcaagagctaccaactcttttt
ccgaaggtaactggcttcagcagagcgcagataccaaatactgtccttc
tagtgtagccgtagttaggccaccacttcaagaactctgtagcaccgcc
tacatacctcgctctgctaatcctgttaccagtggctgctgccagtggc
gataagtcgtgtcttaccgggttggactcaagacgatagttaccggata
aggcgcagcggtcgggctgaacggggggttcgtgcacacagcccagctt
ggagcgaacgacctacaccgaactgagatacctacagcgtgagctatga
gaaagcgccacgcttcccgaagggagaaaggcggacaggtatccggtaa
gcggcagggtcggaacaggagagcgcacgagggagcttccagggggaaa
cgcctggtatctttatagtcctgtcgggtttcgccacctctgacttgag
cgtcgatttttgtgatgctcgtcaggggggcggagcctatggaaaaacg
ccagcaacgcgagctcgtaatacgactcactatagg
CHIKV A6K plasmid
SEQ ID NO: 9
atggctgcgtgagacacacgtagcctaccagtttcttactgctctactc
tgcaaagcaagagattaataacccatcatggattctgtgtacgtggac
atagacgctgacagcgcctttttgaaggccctgcaacgtgcgtacccca
tgtttgaggtggaacctaggcaggtcacatcgaatgaccatgctaatgc
tagagcgttctcgcatctagccataaaactaatagagcaggaaattgat
cccgactcaaccatcctggatataggtagtgcgccagcaaggaggatga
tgtcggacaggaagtaccactgcgtttgcccgatgcgcagcgcagaaga
tcccgagagactcgctaattatgcgagaaagctcgcatctgccgcagga
aaagtcctggacagaaacatttctggaagagagcagacgtcgcgatata
ccaagacgtctatgctgtacacgcacccacgtcgctataccaccaggcg
attaaaggagtccgagtggcgtactgggtagggttcgacacaaccccgt
tcatgtacaacgctatggcgggtgcctacccctcatactcgacaaattg
ggcggatgagcaggtactgaaggctaagaacataggattatgttcaaca
gacctgacggaaggtagacgaggcaaattgtctatcatgagagggaaaa
agctaaaaccgtgcgaccgtgtgctgttctcagtagggtcaacgcttta
cccggaaagccgcacgctacttaagagctggcacctaccatcggtgttc
catctaaagggcaagcttagcttcacatgccgctgtgacacagtggttt
cgtgtgagggctacgtcgttaagagaataacgatgagcccaggccttta
tggaaaaaccatagggtatgcggtaacccaccacgcagacggattcttg
atgtgcaagactaccgacacggttgacggcgaaagagtgtcattctcgg
tgtgcacgtacgtgccggcgaccatttgtgatcaaatgaccggcatcct
tgctacagaagtcacgccggaggatgcacagaagctgttggtggggctg
aaccagaggatagtggttaacggcagaacgcaacggaacacgaacacca
tgaagaactacctacttcccgtggtcgcccaggccttcagtaagtgggc
aaaggagtgccggaaggacatggaagatgagaagcttctgggggtcaga
gaaagaacactaacctgctgctgtctatgggcatttaagaagcagaaaa
cacacacggtctacaagaggcctgatacccagtcaatccagaaggttca
ggccgaatttgacagctttgtagtaccgggcctgtggtcgtccgggttg
tcaatcccgttgaggactagaatcaagtggttgttacgcaaggtgccga
aaacagacctgatcccatacagcgggaatgcccaagaagcccaggatgc
agaaaaagaagcagaggaagaacgagaagcagaactgactcatgaggct
ctaccacccctacaggcagcacaggaagatgtccaggtcgaaatcgacg
tggaacagcttgaggatagagctggtgctggaataatagagactccgag
aggcgctatcaaagttactgcccaactaacagaccacgtcgtgggggag
tacctggtactttccccgcagaccgtactacgcagccagaagctcagcc
tgatccacgctttagcggagcaagtgaagacgtgtacgcacagcggacg
agcagggaggtatgcggtcgaagcgtacgatggccgagtcctagtgccc
tcaggctatgcaatttcgcctgaagacttccagagtctaagcgaaagcg
caacgatggtgtacaacgaaagagagttcgtaaacagaaagttacacca
cattgcgatgcacggaccagccctgaacactgacgaagagtcgtatgag
ctggtgagggcagagaggacagaacacgagtacgtctacgacgtggacc
agagaagatgctgtaagaaggaagaagctgcaggactggtactggtggg
cgacttgactaatccgccctaccacgaattcgcatacgaagggctaaaa
attcgccccgcctgcccatacaaaattgcagtcataggagtcttcgggg
taccaggatctggcaagtcagccattatcaagaacctagttaccaggca
agacctggtgactagcggaaagaaagaaaactgccaagaaatcagcacc
gacgtgatgagacagagaggtctagagatatctgcacgtacggtagatt
cgctgctcttgaatggatgcaacagaccagtcgacgtgttgtacgtaga
cgaggcgtttgcgtgccactctggaacgttacttgctttgatcgccttg
gtgagaccaagacagaaagttgtactttgtggtgacccgaagcagtgcg
gcttcttcaatatgatgcagatgaaagtcaactacaatcataacatctg
cacccaagtgtaccacaaaagtatctccaggcggtgtacactgcctgtg
actgccattgtgtcatcgttgcattacgaaggcaaaatgcgcactacga
atgagtacaacatgccgattgtagtggacactacaggctcaacgaaacc
tgaccctggagacctcgtgttaacgtgcttcagagggtgggttaaacaa
ctgcaaattgactatcgtggacacgaggtcatgacagcagccgcatccc
aagggttaactagaaaaggagtttacgcagttaggcaaaaagttaacga
aaacccactctatgcatcaacatcagagcacgtcaacgtactcctaacg
cgtacggaaggtaaactggtatggaagacactctctggtgacccgtgga
taaagacgctgcagaacccaccgaaaggaaacttcaaagcaactattaa
ggagtgggaggtggagcacgcatcgataatggcgggcatctgcagtcac
caagtgacctttgacacattccaaaacaaagccaacgtttgctgggcta
agagcttggtccctatcctcgaaacagcggggataaaactaaatgatag
gcagtggtcccagataattcaagccttcaaagaagacaaagcatactca
cccgaagtagccctgaatgaaatatgcacgcgcatgtatggggtggatc
tagacagtgggctattctctaaaccgttggtatctgtgtattacgcgga
taaccattgggataataggccgggaggaaagatgttcggattcaaccct
gaggcagcgtccattctagaaagaaagtacccatttacaaaaggaaagt
ggaacatcaacaagcagatctgcgtgactaccaggaggatagaagactt
caaccctaccaccaacattataccggtcaacaggagactaccacactca
ttagtggccgaacaccgcccagtaaaaggggaaagaatggaatggctgg
ttaacaagataaacggacaccacgtactcctggttagcggctataacct
tgcactgcctactaagagagtcacctgggtagcgccactaggtgtccgc
ggagcggactatacatacaacctagagctgggtctaccagcaacgcttg
gtaggtatgacctagtggtcataaacatccacacaccttttcgcataca
ccattaccaacagtgcgtagatcacgcaatgaaactgcaaatgctaggg
ggtgactcactgagactgctcaaaccgggtggctctctattgatcagag
catacggttacgcagatagaaccagtgaacgagtcatctgcgtactggg
acgcaagtttagatcgtctagagcattgaaaccaccatgtgtcaccagt
aatactgagatgtttttcctatttagcaattttgacaatggcagaagga
attttacaacgcatgtcatgaacaatcaactgaatgcagcctttgtagg
acaggccacccgagcaggatgtgcaccatcgtaccgggtaaaacgcatg
gacatcgcgaagaacgatgaagagtgcgtggttaacgccgccaaccctc
gcgggttaccaggtgacggtgtttgcaaggcagtatataaaaagtggcc
ggagtcctttaaaaacagtgcaacaccagtaggaaccgcaaaaacagtt
atgtgcggtacgtatccagtaatccacgccgtaggaccaaacttctcaa
attattcggagtctgaaggggaccgggaattggcggctgcctatcgaga
agtcgcaaaggaagtaactagactgggagtaaatagcgtagctatacct
ctcctctccacaggtgtatactcaggagggaaagacaggctaacccagt
cactgaaccacctctttacagccatggactcgacggatgcagacgtggt
catctactgccgagacaaggaatgggagaagaaaatatctgaggccata
cagatgcggacccaagtggagctgctggatgagcacatctccatagact
gcgatgtcattcgcgtgcaccctgacagtagcttggcaggcagaaaagg
atacagcaccacggaaggcgcactgtattcatatctagaagggacacgt
tttcaccagacggcagtggatatggcagagatatacactatgtggccaa
agcaaacagaggccaatgagcaagtctgcctatatgccctgggggaaag
tattgaatcaatcaggcagaaatgcccggtggatgatgcagacgcatca
tctcccccgaaaactgtcccgtgtctttgccggtatgccatgactcctg
aacgcgtcacccgacttcgcatgaaccatgtcacaaatataattgtgtg
ttcttcatttccccttccaaagtacaagatagaaggagtgcaaaaagtc
aaatgctccaaggtaatgttattcgatcacaatgtgccatcgcgcgtaa
gtccaagggaatacagatcttcccaggagtctgtacaggaagtgagtac
gacaacgtcattgacgcatagccagtttgatctaagcgccgatggcgag
acactgcctgtcccgtcagacctggatgctgacgccccagccctagaac
cggccctagacgacggggggtacatacattaccaaccataatcggaaac
cttgcggccgtgtctgactgggtaatgagcaccgtacctgtcgcgccgc
ctagaagaaggagagggagaaacctgactgtgacatgtgacgagagaga
agggaatataacacccatggctagcgtccgattctttagagcagagctg
tgtccggccgtacaagaaacagcggagacgcgtgacacagctatttccc
ttcaggcaccgccaagtaccaccatggaactgagccatccaccgatctc
cttcggagcaccaagcgagacgttccccatcacatttggggacttcgac
gaaggagaaatcgaaagcttgtcttctgagctactaactttcggagact
tcctacccggtgaagtggatgatctgacagatagcgactggtccacgtg
cccagacacggacgacgagttatgactagacagggcaggtgggtatata
ttctcgtcggacactggtccaggccatttacaacagaagtcggtacgcc
agtcagtgctgccggtaaacaccctggaggaagtccacgaggagaagtg
ttacccacctaagctggatgaattaaaggagcaactactacttaagaaa
ctccaggagagtgcgtccatggccaatagaagcaggtatcagtcacgca
aagtggaaaatatgaaagcaacaatcatccagagactaaagagaggctg
taaactgtatttaatggcagagaccccgaaagtcccgacttatcggacc
atatacccggcgcctgtgtactcgcctccgatcaatgtccgattgtcca
accccgagtccgcagtggcagcatgtaatgagttcttagctagaaacta
cccaactgtttcatcataccaaatcaccgacgagtatgatgcatatcta
gacatggtggacgggtcggagagttgcttggaccgagcgacattcaatc
cgtcaaaacttaggagctacccgaaacaacatgcttatcacgcgccttc
tatcagaagcgctgtaccttccccattccagaacacactacagaatgta
ctggcagcagccacgaaaaggaactgcaacgtcacacagatgagggaat
tacccactttggactcagcagtattcaacgtggagtgttttaaaaaatt
cgcatgtaaccgagaatactgggaagaatttgcagccagccctatcagg
ataacaactgagaatctaacaacctatgtcactaaactaaaggggccaa
aagcagcagcgctgtttgcaaaaacccataatctgctgccactgcagga
tgtaccaatggataggttcacagtagatatgaaaagggatgtgaaggta
actcctggtacaaagcatacagaggaaagacctaaggtgcaggttatac
aggcggctgaacccttggcaacagcgtacctatgtggaattcacagaga
actggttaggagattgaacgccgtcctcctacccaatgtgcatacacta
tttgacatgtctgccgaggacttcgatgccattatagccgcacacttca
agccaggagacgctgttttagaaacggacatagcctcctttgataagag
ccaagatgattcacttgcgcttaccgccttaatgctgttagaagatttg
ggagtggatcactccctgttggacttgatagaggctgctttcggagaga
tttccagctgtcatctgccgacaggtacgcgcttcaagttcggcgctat
gatgaaatccggtatgttcctaactctgttcgtcaacacgttgttaaat
atcaccatcgctagccgggtgttggaagatcgtctgacaaaatccgcat
gcgcggccttcatcggcgacgacaacataatacatggtgtcgtctccga
tgaattgatggcagccagatgcgctacttggatgaacatggaagtgaag
atcatagatgcagttgtatcccagaaagctccttacttttgtggagggt
ttatactgcatgatactgtgacaggaacagcttgcagagtggcggaccc
gctaaaaaggttatttaaattgggcaaaccgttagcggcaggtgacgaa
caagatgaagacagaagacgggcgctggctgatgaagtaatcagatggc
aacgaacagggctaatagatgagctggagaaagcggtgtactctaggta
cgaagtgcagggtatatcagttgcggtaatgtccatggccacctttgca
agctccagatccaacttcgagaagctcagaggacccgtcataactttgt
acggcggtcctaaataggtacgcactacagctacctattttgcagaagc
cgacagcaggtacctaaataccaatcagccataatggagtttatcccaa
cccaaactttctacaataggaggtaccagcctcgaccttggactccgcg
ccctactatccaagttatcagacccagaccgcgtccgcaaaggaaagcc
gggcaacttgcccagctgatctcagcagttaataaactgacaatgcgcg
cggtacctcaacagaagccgcgcaagaatcggaagaataagaagcaaaa
gcaaaagcagcaggcgccacgaaacaacatgaatcaaaagaagcagccc
cctaaaaagaaaccggctcaaaagaaaaagaagccgggccgtagagaga
gaatgtgcatgaaaatcgaaaatgattgcatcttcgaagtcaagcatga
aggtaaggtaacaggttacgcgtgcttggtaggggacaaagtaatgaag
ccagcacacgtaaaggggaccatcgataatgcggacctggccaaattgg
ccttcaagcggtcatctaagtacgaccttgaatgcgcgcagatacccgt
gcacatgaagtccgacgcttcgaagttcacccatgagaaaccggagggg
tactacaactggcaccacggagcagtacagtactcaggaggccggttca
ccatccctacaggtgcgggcaaaccaggggacagcggtagaccgatctt
cgacaacaaggggcgcgtggtggccatagttttaggaggagctaatgaa
ggagcccgtacagccctctcggtggtgacctggaacaaagacatcgtca
cgaaaatcacccctgagggggccgaagagtggagtcttgccattccagt
tatgtgcctgctggcaaataccacgttcccctgctcccagcccccttgc
acaccctgctgctacgaaaaagagccggagaaaaccctgcgcatgctag
aagacaacgtcatgagccccgggtactatcagctgctacaagcatcctt
aacatgttctccccgccgccagcgacgcagtattaaggacaacttcaat
gtctataaagccataagaccgtacctagctcactgtcccgactgtggag
aagggcactcgtgccatagtcccgtagcgctagaacgcatcagaaacga
agcgacagacgggacgctgaaaatccaggtttccttgcaaatcggaata
aagacggatgatagccatgattggaccaagctgcgttacatggacaatc
atatgccagcagacgcagagagggccaggctatttgtaagaacgtcagc
accgtgcacgattactggaacaatgggacacttcatcctggcccgatgt
ccgaaaggagaaactctgacggtgggattcactgacggtaggaagatca
gtcactcatgtacgcacccatttcaccacgaccctcctgtgataggccg
ggaaaaatttcattcccgaccgcagcacggtagagaactaccttgcagc
acgtacgcgcagagcaccgctgcaactgccgaggagatagaggtacata
tgcccccagacaccccagatcgcacattgatgtcacaacagtccggtaa
tgtaaagatcacagtcaatagtcagacggtgcggtacaagtgtaattgc
ggtgactcaaatgaaggactaaccactacagacaaagtgattaataact
gcaaggttgatcaatgccatgccgcggtcaccaatcacaaaaaatggca
gtataattcccctctggtcccgcgtaatgctgaactcggggaccgaaaa
ggaaaagttcacattccgtttcctctggcaaatgtgacatgcagggtgc
ctaaggcaaggaaccccaccgtgacgtacggaaaaaaccaagtcatcat
gctgctgtatcctgaccacccaacgctcctgtcctaccggaatatggga
gaagaaccaaactatcaagaagagtgggtgacgcataagaaggagatca
ggttaaccgtgccgactgaagggctcgaggtcacgtggggcaacaacga
gccgtacaagtattggccgcagttatccacaaacggtacagcccacggc
cacccgcatgagataattttgtattattatgagctgtaccctactatga
ctgtggtagttgtgtcagtggcctcgttcgtactcctgtcgatggtggg
tgtggcagtggggatgtgcatgtgtgcacgacgcagatgcattacaccg
tacgaactgacaccaggagctaccgtccctttcctgcttagcctaatat
gctgcattagaacagctaaagcggcgtacgaacacgtaacagtgatccc
gaacacggtgggagtaccgtataagactctagtcaacagaccgggctac
agccccatggtactggagatggagcttctgtcagtcactttggagccaa
cgctatcgcttgattacatcacgtgcgagtataaaaccgtcatcccgtc
tccgtacgtgaaatgctgcggtacagcagagtgcaaggacaagagccta
cctgattacagctgtaaggtcttcaccggcgtctacccattcatgtggg
gcggcgcctactgcttctgcgacactgaaaatacgcaattgagcgaagc
acatgtggagaagtccgaatcatgcaaaacagaatttgcatcagcatat
agggctcataccgcatccgcatcagctaagctccgcgtcctttaccaag
gaaataatgttactgtatctgcttatgcaaacggcgatcatgccgtcac
agttaaggacgctaaattcattgtggggccaatgtcttcagcctggaca
ccttttgacaataaaatcgtggtgtacaaaggcgacgtctacaacatgg
actacccgcccttcggcgcaggaagaccaggacaatttggcgacatcca
aagtcgcacgcctgagagcgaagacgtctatgctaacacacaactggta
ctgcagagaccgtccgcgggtacggtgcacgtgccgtactctcaggcac
catctggcttcaagtattggctaaaagaacgaggggcgtcgctgcagca
cacagcaccatttggctgtcaaatagcaacaaacccggtaagagcgatg
aactgcgccgtagggaacatgcctatctccatcgacataccggacgcgg
ccttcactagggtcgtcgacgcgccatctttaacggacatgtcgtgtga
ggtaccagcctgcacccactcctcagactttgggggcgtagccatcatt
aaatatgcagccagcaagaaaggcaagtgtgcggtgcattcgatgacta
acgccgtcactattcgggaagctgaaatagaagtagaagggaactctca
gttgcaaatctctttttcgacggccctagccagcgccgaattccgcgta
caagtctgttctacacaagtacactgtgcagccgagtgccatccaccga
aagaccatatagtcagggactggttgtcgctgttgcagcactgatccta
atcgtggtgctatgcgtgtcgtttagcaggcactaacttgacaactagg
tacgaaggtatatgtgtcccctaagagacacaccacatatagctaagaa
tcaatagataagtatagatcaaagggctgaacaacccctgaatagtaac
aaaatataaaaatcaacaaaaatcataaaatagaaaaccagaaacagaa
gtaggtaagaaggtatatgtgtcccctaagagacacaccatatatagct
aagaatcaatagataagtatagatcaaagggctgaataacccctgaata
ataacaaaatataaaaatcaataaaaatcataaaatagaaaaccataaa
cagaagtagttcaaagggctataaaacccctgaatagtaacaaaacata
aaactaataaaaatcaaatgaataccataattggcaatcggaagagatg
taggtacttaagcttcctaaaagcagccgaactcgctttgagatgtagg
cgtagcacaccgaactcttccataattctccgaacccacagggacgtag
gagatgttcaaagtggctataaaaccctgaacagtaataaaacataaaa
ttaataaggatcaaatgagtaccataattggcaaacggaagagatgtag
gtacttaagcttcctaaaagcagccgaactcactttgagatgtaggcat
agcataccgaactcttccacaattctccgtacccatagggacgtaggag
atgttattttgtttttaatatttcaaaaaaaaaaaaaaaaaaaaaaaaa
aaaaaaaaaaaaaaaagcggccgcttaattaatcgaggggaattaattc
ttgaagacgaaagggccaggtggcacttttcggggaaatgtgcgcggaa
cccctatttgtttatttttctaaatacattcaaatatgtatccgctcat
gagacaataaccctgataaatgcttcaataatattgaaaaaggaagagt
atgagtattcaacatttccgtgtcgcccttattcccttttttgcggcat
tttgccttcctgtttttgctcacccagaaacgctggtgaaagtaaaaga
tgctgaagatcagttgggtgcacgagtgggttacatcgaactggatctc
aacagcggtaagatccttgagagttttcgccccgaagaacgttttccaa
tgatgagcacttttaaagttctgctatgtggcgcggtattatcccgtgt
tgacgccgggcaagagcaactcggtcgccgcatacactattctcagaat
gacttggttgagtactcaccagtcacagaaaagcatcttacggatggca
tgacagtaagagaattatgcagtgctgccataaccatgagtgataacac
tgcggccaacttacttctgacaacgatcggaggaccgaaggagctaacc
gcttttttgcacaacatgggggatcatgtaactcgccttgatcgttggg
aaccggagctgaatgaagccataccaaacgacgagcgtgacaccacgat
gcctgtagcaatggcaacaacgttgcgcaaactattaactggcgaacta
cttactctagcttcccggcaacaattaatagactggatggaggcggata
aagttgcaggaccacttctgcgctcggcccttccggctggctggtttat
tgctgataaatctggagccggtgagcgtgggtctcgcggtatcattgca
gcactggggccagatggtaagccctcccgtatcgtagttatctacacga
cggggagtcaggcaactatggatgaacgaaatagacagatcgctgagat
aggtgcctcactgattaagcattggtaactgtcagaccaagtttactca
tatatactttagattgatttaaaacttcatttttaatttaaaaggatct
aggtgaagatcctttttgataatctcatgaccaaaatcccttaacgtga
gttttcgttccactgagcgtcagaccccgtagaaaagatcaaaggatct
tcttgagatcctttttttctgcgcgtaatctgctgcttgcaaacaaaaa
aaccaccgctaccagcggtggtttgtttgccggatcaagagctaccaac
tctttttccgaaggtaactggcttcagcagagcgcagataccaaatact
gtccttctagtgtagccgtagttaggccaccacttcaagaactctgtag
caccgcctacatacctcgctctgctaatcctgttaccagtggctgctgc
cagtggcgataagtcgtgtcttaccgggttggactcaagacgatagtta
ccggataaggcgcagcggtcgggctgaacggggggttcgtgcacacagc
ccagcttggagcgaacgacctacaccgaactgagatacctacagcgtga
gctatgagaaagcgccacgcttcccgaagggagaaaggcggacaggtat
ccggtaagcggcagggtcggaacaggagagcgcacgagggagcttccag
ggggaaacgcctggtatctttatagtcctgtcgggtttcgccacctctg
acttgagcgtcgatttttgtgatgctcgtcaggggggcggagcctatgg
aaaaacgccagcaacgcgagctcgtaatacgactcactatagg
CHIKV 181/25-ECMV IRES plasmid
SEQ ID NO: 10
atggctgcgtgagacacacgtagcctaccagtttcttactgctctactc
tgcaaagcaagagattaataacccatcatggattctgtgtacgtggac
atagacgctgacagcgcctttttgaaggccctgcaacgtgcgtacccca
tgtttgaggtggaacctaggcaggtcacatcgaatgaccatgctaatgc
tagagcgttctcgcatctagccataaaactaatagagcaggaaattgat
cccgactcaaccatcctggatataggtagtgcgccagcaaggaggatga
tgtcggacaggaagtaccactgcgtttgcccgatgcgcagcgcagaaga
tcccgagagactcgctaattatgcgagaaagctcgcatctgccgcagga
aaagtcctggacagaaacatttctggaagagagcagacgtcgcgatata
ccaagacgtctatgctgtacacgcacccacgtcgctataccaccaggcg
attaaaggagtccgagtggcgtactgggtagggttcgacacaaccccgt
tcatgtacaacgctatggcgggtgcctacccctcatactcgacaaattg
ggcggatgagcaggtactgaaggctaagaacataggattatgttcaaca
gacctgacggaaggtagacgaggcaaattgtctatcatgagagggaaaa
agctaaaaccgtgcgaccgtgtgctgttctcagtagggtcaacgcttta
cccggaaagccgcacgctacttaagagctggcacctaccatcggtgttc
catctaaagggcaagcttagcttcacatgccgctgtgacacagtggttt
cgtgtgagggctacgtcgttaagagaataacgatgagcccaggccttta
tggaaaaaccatagggtatgcggtaacccaccacgcagacggattcttg
atgtgcaagactaccgacacggttgacggcgaaagagtgtcattctcgg
tgtgcacgtacgtgccggcgaccatttgtgatcaaatgaccggcatcct
tgctacagaagtcacgccggaggatgcacagaagctgttggtggggctg
aaccagaggatagtggttaacggcagaacgcaacggaacacgaacacca
tgaagaactacctacttcccgtggtcgcccaggccttcagtaagtgggc
aaaggagtgccggaaggacatggaagatgagaagcttctgggggtcaga
gaaagaacactaacctgctgctgtctatgggcatttaagaagcagaaaa
cacacacggtctacaagaggcctgatacccagtcaatccagaaggttca
ggccgaatttgacagctttgtagtaccgggcctgtggtcgtccgggttg
tcaatcccgttgaggactagaatcaagtggttgttacgcaaggtgccga
aaacagacctgatcccatacagcgggaatgcccaagaagcccaggatgc
agaaaaagaagcagaggaagaacgagaagcagaactgactcatgaggct
ctaccacccctacaggcagcacaggaagatgtccaggtcgaaatcgacg
tggaacagcttgaggatagagctggtgctggaataatagagactccgag
aggcgctatcaaagttactgcccaactaacagaccacgtcgtgggggag
tacctggtactttccccgcagaccgtactacgcagccagaagctcagcc
tgatccacgctttagcggagcaagtgaagacgtgtacgcacagcggacg
agcagggaggtatgcggtcgaagcgtacgatggccgagtcctagtgccc
tcaggctatgcaatttcgcctgaagacttccagagtctaagcgaaagcg
caacgatggtgtacaacgaaagagagttcgtaaacagaaagttacacca
cattgcgatgcacggaccagccctgaacactgacgaagagtcgtatgag
ctggtgagggcagagaggacagaacacgagtacgtctacgacgtggacc
agagaagatgctgtaagaaggaagaagctgcaggactggtactggtggg
cgacttgactaatccgccctaccacgaattcgcatacgaagggctaaaa
attcgccccgcctgcccatacaaaattgcagtcataggagtcttcgggg
taccaggatctggcaagtcagccattatcaagaacctagttaccaggca
agacctggtgactagcggaaagaaagaaaactgccaagaaatcagcacc
gacgtgatgagacagagaggtctagagatatctgcacgtacggtagatt
cgctgctcttgaatggatgcaacagaccagtcgacgtgttgtacgtaga
cgaggcgtttgcgtgccactctggaacgttacttgctttgatcgccttg
gtgagaccaagacagaaagttgtactttgtggtgacccgaagcagtgcg
gcttcttcaatatgatgcagatgaaagtcaactacaatcataacatctg
cacccaagtgtaccacaaaagtatctccaggcggtgtacactgcctgtg
actgccattgtgtcatcgttgcattacgaaggcaaaatgcgcactacga
atgagtacaacatgccgattgtagtggacactacaggctcaacgaaacc
tgaccctggagacctcgtgttaacgtgcttcagagggtgggttaaacaa
ctgcaaattgactatcgtggacacgaggtcatgacagcagccgcatccc
aagggttaactagaaaaggagtttacgcagttaggcaaaaagttaacga
aaacccactctatgcatcaacatcagagcacgtcaacgtactcctaacg
cgtacggaaggtaaactggtatggaagacactctctggtgacccgtgga
taaagacgctgcagaacccaccgaaaggaaacttcaaagcaactattaa
ggagtgggaggtggagcacgcatcgataatgggggcatctgcagtcacc
aagtgacctttgacacattccaaaacaaagccaacgtttgctgggctaa
gagcttggtccctatcctcgaaacagcggggataaaactaaatgatagg
cagtggtcccagataattcaagccttcaaagaagacaaagcatactcac
ccgaagtagccctgaatgaaatatgcacgcgcatgtatggggtggatct
agacagtgggctattctctaaaccgttggtatctgtgtattacgcggat
aaccattgggataataggccgggaggaaagatgttcggattcaaccctg
aggcagcgtccattctagaaagaaagtacccatttacaaaaggaaagtg
gaacatcaacaagcagatctgcgtgactaccaggaggatagaagacttc
aaccctaccaccaacattataccggtcaacaggagactaccacactcat
tagtggccgaacaccgcccagtaaaaggggaaagaatggaatggctggt
taacaagataaacggacaccacgtactcctggttagcggctataacctt
gcactgcctactaagagagtcacctgggtagcgccactaggtgtccgcg
gagcggactatacatacaacctagagctgggtctaccagcaacgcttgg
taggtatgacctagtggtcataaacatccacacaccttttcgcatacac
cattaccaacagtgcgtagatcacgcaatgaaactgcaaatgctagggg
gtgactcactgagactgctcaaaccgggtggctctctattgatcagagc
atacggttacgcagatagaaccagtgaacgagtcatctgcgtactggga
cgcaagtttagatcgtctagagcattgaaaccaccatgtgtcaccagta
atactgagatgtttttcctatttagcaattttgacaatggcagaaggaa
ttttacaacgcatgtcatgaacaatcaactgaatgcagcctttgtagga
caggccacccgagcaggatgtgcaccatcgtaccgggtaaaacgcatgg
acatcgcgaagaacgatgaagagtgcgtggttaacgccgccaaccctcg
cgggttaccaggtgacggtgtttgcaaggcagtatataaaaagtggccg
gagtcctttaaaaacagtgcaacaccagtaggaaccgcaaaaacagtta
tgtgcggtacgtatccagtaatccacgccgtaggaccaaacttctcaaa
ttattcggagtctgaaggggaccgggaattggcggctgcctatcgagaa
gtcgcaaaggaagtaactagactgggagtaaatagcgtagctatacctc
tcctctccacaggtgtatactcaggagggaaagacaggctaacccagtc
actgaaccacctctttacagccatggactcgacggatgcagacgtggtc
atctactgccgagacaaggaatgggagaagaaaatatctgaggccatac
agatgcggacccaagtggagctgctggatgagcacatctccatagactg
cgatgtcattcgcgtgcaccctgacagtagcttggcaggcagaaaagga
tacagcaccacggaaggcgcactgtattcatatctagaagggacacgtt
ttcaccagacggcagtggatatggcagagatatacactatgtggccaaa
gcaaacagaggccaatgagcaagtctgcctatatgccctgggggaaagt
attgaatcaatcaggcagaaatgcccggtggatgatgcagacgcatcat
ctcccccgaaaactgtcccgtgtctttgccggtatgccatgactcctga
acgcgtcacccgacttcgcatgaaccatgtcacaaatataattgtgtgt
tcttcatttccccttccaaagtacaagatagaaggagtgcaaaaagtca
aatgctccaaggtaatgttattcgatcacaatgtgccatcgcgcgtaag
tccaagggaatacagatcttcccaggagtctgtacaggaagtgagtacg
acaacgtcattgacgcatagccagtttgatctaagcgccgatggcgaga
cactgcctgtcccgtcagacctggatgctgacgccccagccctagaacc
ggccctagacgacggggcggtacatacattaccaaccataatcggaaac
cttgcggccgtgtctgactgggtaatgagcaccgtacctgtcgcgccgc
ctagaagaaggagagggagaaacctgactgtgacatgtgacgagagaga
agggaatataacacccatggctagcgtccgattctttagagcagagctg
tgtccggccgtacaagaaacagcggagacgcgtgacacagctatttccc
ttcaggcaccgccaagtaccaccatggaactgagccatccaccgatctc
cttcggagcaccaagcgagacgttccccatcacatttggggacttcgac
gaaggagaaatcgaaagcttgtcttctgagctactaactttcggagact
tcctacccggtgaagtggatgatctgacagatagcgactggtccacgtg
cccagacacggacgacgagttatgactagacagggcaggtgggtatata
ttctcgtcggacactggtccaggccatttacaacagaagtcggtacgcc
agtcagtgctgccggtaaacaccctggaggaagtccacgaggagaagtg
ttacccacctaagctggatgaattaaaggagcaactactacttaagaaa
ctccaggagagtgcgtccatggccaatagaagcaggtatcagtcacgca
aagtggaaaatatgaaagcaacaatcatccagagactaaagagaggctg
taaactgtatttaatggcagagaccccgaaagtcccgacttatcggacc
atatacccggcgcctgtgtactcgcctccgatcaatgtccgattgtcca
accccgagtccgcagtggcagcatgtaatgagttcttagctagaaacta
cccaactgtttcatcataccaaatcaccgacgagtatgatgcatatcta
gacatggtggacgggtcggagagttgcttggaccgagcgacattcaatc
cgtcaaaacttaggagctacccgaaacaacatgcttatcacgcgccttc
tatcagaagcgctgtaccttccccattccagaacacactacagaatgta
ctggcagcagccacgaaaaggaactgcaacgtcacacagatgagggaat
tacccactttggactcagcagtattcaacgtggagtgttttaaaaaatt
cgcatgtaaccgagaatactgggaagaatttgcagccagccctatcagg
ataacaactgagaatctaacaacctatgtcactaaactaaaggggccaa
aagcagcagcgctgtttgcaaaaacccataatctgctgccactgcagga
tgtaccaatggataggttcacagtagatatgaaaagggatgtgaaggta
actcctggtacaaagcatacagaggaaagacctaaggtgcaggttatac
aggcggctgaacccttggcaacagcgtacctatgtggaattcacagaga
actggttaggagattgaacgccgtcctcctacccaatgtgcatacacta
tttgacatgtctgccgaggacttcgatgccattatagccgcacacttca
agccaggagacgctgttttagaaacggacatagcctcctttgataagag
ccaagatgattcacttgcgcttaccgccttaatgctgttagaagatttg
ggagtggatcactccctgttggacttgatagaggctgctttcggagaga
tttccagctgtcatctgccgacaggtacgcgcttcaagttcggcgctat
gatgaaatccggtatgttcctaactctgttcgtcaacacgttgttaaat
atcaccatcgctagccgggtgttggaagatcgtctgacaaaatccgcat
gcgcggccttcatcggcgacgacaacataatacatggtgtcgtctccga
tgaattgatggcagccagatgcgctacttggatgaacatggaagtgaag
atcatagatgcagttgtatcccagaaagctccttacttttgtggagggt
ttatactgcatgatactgtgacaggaacagcttgcagagtggcggaccc
gctaaaaaggttatttaaattgggcaaaccgttagcggcaggtgacgaa
caagatgaagacagaagacgggcgctggctgatgaagtaatcagatggc
aacgaacagggctaatagatgagctggagaaagcggtgtactctaggta
cgaagtgcagggtatatcagttgcggtaatgtccatggccacctttgca
agctccagatccaacttcgagaagctcagaggacccgtcataacgcttt
atggaggaccgaagtaataacccctctccctcccccccccctaacgtta
ctggccgaagccgcttggaataaggccggtgtgcgtttgtctatatgtt
attttccaccatattgccgtcttttggcaatgtgagggcccggaaacct
ggccctgtcttcttgacgagcattcctaggggtctttcccctctcgcca
aaggaatgcaaggtctgttgaatgtcgtgaaggaagcagttcctctgga
agcttcttgaagacaaacaacgtctgtagcgaccctttgcaggcagcgg
aaccccccacctggcgacaggtgcctctgcggccaaaagccacgtgtat
aagatacacctgcaaaggcggcacaaccccagtgccacgttgtgagttg
gatagttgtggaaagagtcaaatggctctcctcaagcgtattcaacaag
gggctgaaggatgcccagaaggtaccccattgtatgggatctgatctgg
ggcctcggtacacatgctttacatgtgtttagtcgaggttaaaaaaacg
tctaggccccccgaaccacggggacgtggttttcctttgaaaaacacga
tgataatatggccacaaccatggagtttatcccaacccaaactttctac
aataggaggtaccagcctcgaccttggactccgcgccctactatccaag
ttatcagacccagaccgcgtccgcaaaggaaagccgggcaacttgccca
gctgatctcagcagttaataaactgacaatgcgcgcggtacctcaacag
aagccgcgcaagaatcggaagaataagaagcaaaagcaaaagcagcagg
cgccacgaaacaacatgaatcaaaagaagcagccccctaaaaagaaacc
ggctcaaaagaaaaagaagccgggccgtagagagagaatgtgcatgaaa
atcgaaaatgattgcatcttcgaagtcaagcatgaaggtaaggtaacag
gttacgcgtgcttggtaggggacaaagtaatgaagccagcacacgtaaa
ggggaccatcgataatgcggacctggccaaattggccttcaagcggtca
tctaagtacgaccttgaatgcgcgcagatacccgtgcacatgaagtccg
acgcttcgaagttcacccatgagaaaccggaggggtactacaactggca
ccacggagcagtacagtactcaggaggccggttcaccatccctacaggt
gcgggcaaaccaggggacagcggtagaccgatcttcgacaacaaggggc
gcgtggtggccatagttttaggaggagctaatgaaggagcccgtacagc
cctctcggtggtgacctggaacaaagacatcgtcacgaaaatcacccct
gagggggccgaagagtggagtcttgccattccagttatgtgcctgctgg
caaataccacgttcccctgctcccagcccccttgcacaccctgctgcta
cgaaaaagagccggagaaaaccctgcgcatgctagaagacaacgtcatg
agccccgggtactatcagctgctacaagcatccttaacatgttctcccc
gccgccagcgacgcagtattaaggacaacttcaatgtctataaagccat
aagaccgtacctagctcactgtcccgactgtggagaagggcactcgtgc
catagtcccgtagcgctagaacgcatcagaaacgaagcgacagacggga
cgctgaaaatccaggtttccttgcaaatcggaataaagacggatgatag
ccatgattggaccaagctgcgttacatggacaatcatatgccagcagac
gcagagagggccaggctatttgtaagaacgtcagcaccgtgcacgatta
ctggaacaatgggacacttcatcctggcccgatgtccgaaaggagaaac
tctgacggtgggattcactgacggtaggaagatcagtcactcatgtacg
cacccatttcaccacgaccctcctgtgataggccgggaaaaatttcatt
cccgaccgcagcacggtagagaactaccttgcagcacgtacgcgcagag
caccgctgcaactgccgaggagatagaggtacatatgcccccagacacc
ccagatcgcacattgatgtcacaacagtccggtaatgtaaagatcacag
tcaatagtcagacggtgcggtacaagtgtaattgcggtgactcaaatga
aggactaaccactacagacaaagtgattaataactgcaaggttgatcaa
tgccatgccgcggtcaccaatcacaaaaaatggcagtataattcccctc
tggtcccgcgtaatgctgaactcggggaccgaaaaggaaaagttcacat
tccgtttcctctggcaaatgtgacatgcagggtgcctaaggcaaggaac
cccaccgtgacgtacggaaaaaaccaagtcatcatgctgctgtatcctg
accacccaacgctcctgtcctaccggaatatgggagaagaaccaaacta
tcaagaagagtgggtgacgcataagaaggagatcaggttaaccgtgccg
actgaagggctcgaggtcacgtggggcaacaacgagccgtacaagtatt
ggccgcagttatccacaaacggtacagcccacggccacccgcatgagat
aattttgtattattatgagctgtaccctactatgactgtggtagttgtg
tcagtggcctcgttcgtactcctgtcgatggtgggtgtggcagtgggga
tgtgcatgtgtgcacgacgcagatgcattacaccgtacgaactgacacc
aggagctaccgtccctttcctgcttagcctaatatgctgcattagaaca
gctaaagcggccacataccaagaggctgcggtatacctgtggaacgagc
agcagcctttgttttggctgcaagcccttattccgctggcagccctgat
tgtcctatgcaactgtctgagactcttaccatgcttttgtaaaacgttg
acttttttagccgtaatgagcgtcggtgcccacactgtgagcgcgtacg
aacacgtaacagtgatcccgaacacggtgggagtaccgtataagactct
agtcaacagaccgggctacagccccatggtactggagatggagcttctg
tcagtcactttggagccaacgctatcgcttgattacatcacgtgcgagt
ataaaaccgtcatcccgtctccgtacgtgaaatgctgcggtacagcaga
gtgcaaggacaagagcctacctgattacagctgtaaggtcttcaccggc
gtctacccattcatgtggggcggcgcctactgcttctgcgacactgaaa
atacgcaattgagcgaagcacatgtggagaagtccgaatcatgcaaaac
agaatttgcatcagcatatagggctcataccgcatccgcatcagctaag
ctccgcgtcctttaccaaggaaataatgttactgtatctgcttatgcaa
acggcgatcatgccgtcacagttaaggacgctaaattcattgtggggcc
aatgtcttcagcctggacaccttttgacaataaaatcgtggtgtacaaa
ggcgacgtctacaacatggactacccgcccttcggcgcaggaagaccag
gacaatttggcgacatccaaagtcgcacgcctgagagcgaagacgtcta
tgctaacacacaactggtactgcagagaccgtccgcgggtacggtgcac
gtgccgtactctcaggcaccatctggcttcaagtattggctaaaagaac
gaggggcgtcgctgcagcacacagcaccatttggctgtcaaatagcaac
aaacccggtaagagcgatgaactgcgccgtagggaacatgcctatctcc
atcgacataccggacgcggccttcactagggtcgtcgacgcgccatctt
taacggacatgtcgtgtgaggtaccagcctgcacccactcctcagactt
tgggggcgtagccatcattaaatatgcagccagcaagaaaggcaagtgt
gcggtgcattcgatgactaacgccgtcactattcgggaagctgaaatag
aagtagaagggaactctcagttgcaaatctctttttcgacggccctagc
cagcgccgaattccgcgtacaagtctgttctacacaagtacactgtgca
gccgagtgccatccaccgaaagaccatatagtcaattacccggcgtcac
acaccaccctcggggtccaagacatttccgttacggcgatgtcatgggt
gcagaagatcacgggaggtgtgggactggttgtcgctgttgcagcactg
atcctaatcgtggtgctatgcgtgtcgtttagcaggcactaacttgaca
actaggtacgaaggtatatgtgtcccctaagagacacaccacatatagc
taagaatcaatagataagtatagatcaaagggctgaacaacccctgaat
agtaacaaaatataaaaatcaacaaaaatcataaaatagaaaaccagaa
acagaagtaggtaagaaggtatatgtgtcccctaagagacacaccatat
atagctaagaatcaatagataagtatagatcaaagggctgaataacccc
tgaataataacaaaatataaaaatcaataaaaatcataaaatagaaaac
cataaacagaagtagttcaaagggctataaaacccctgaatagtaacaa
aacataaaactaataaaaatcaaatgaataccataattggcaatcggaa
gagatgtaggtacttaagcttcctaaaagcagccgaactcgctttgaga
tgtaggcgtagcacaccgaactcttccataattctccgaacccacaggg
acgtaggagatgttcaaagtggctataaaaccctgaacagtaataaaac
ataaaattaataaggatcaaatgagtaccataattggcaaacggaagag
atgtaggtacttaagcttcctaaaagcagccgaactcactttgagatgt
aggcatagcataccgaactcttccacaattctccgtacccatagggacg
taggagatgttattttgtttttaatatttcaaaaaaaaaaaaaaaaaaa
aaaaaaaaaaaaaaaaaaaaaagcggccgcttaattaatcgaggggaat
taattcttgaagacgaaagggccaggtggcacttttcggggaaatgtgc
gcggaacccctatttgtttatttttctaaatacattcaaatatgtatcc
gctcatgagacaataaccctgataaatgcttcaataatattgaaaaagg
aagagtatgagtattcaacatttccgtgtcgcccttattcccttttttg
cggcattttgccttcctgtttttgctcacccagaaacgctggtgaaagt
aaaagatgctgaagatcagttgggtgcacgagtgggttacatcgaactg
gatctcaacagcggtaagatccttgagagttttcgccccgaagaacgtt
ttccaatgatgagcacttttaaagttctgctatgtggcgcggtattatc
ccgtgttgacgccgggcaagagcaactcggtcgccgcatacactattct
cagaatgacttggttgagtactcaccagtcacagaaaagcatcttacgg
atggcatgacagtaagagaattatgcagtgctgccataaccatgagtga
taacactgcggccaacttacttctgacaacgatcggaggaccgaaggag
ctaaccgcttttttgcacaacatgggggatcatgtaactcgccttgatc
gttgggaaccggagctgaatgaagccataccaaacgacgagcgtgacac
cacgatgcctgtagcaatggcaacaacgttgcgcaaactattaactggc
gaactacttactctagcttcccggcaacaattaatagactggatggagg
cggataaagttgcaggaccacttctgcgctcggcccttccggctggctg
gtttattgctgataaatctggagccggtgagcgtgggtctcgcggtatc
attgcagcactggggccagatggtaagccctcccgtatcgtagttatct
acacgacggggagtcaggcaactatggatgaacgaaatagacagatcgc
tgagataggtgcctcactgattaagcattggtaactgtcagaccaagtt
tactcatatatactttagattgatttaaaacttcatttttaatttaaaa
ggatctaggtgaagatcctttttgataatctcatgaccaaaatccctta
acgtgagttttcgttccactgagcgtcagaccccgtagaaaagatcaaa
ggatcttcttgagatcctttttttctgcgcgtaatctgctgcttgcaaa
caaaaaaaccaccgctaccagcggtggtttgtttgccggatcaagagct
accaactctttttccgaaggtaactggcttcagcagagcgcagatacca
aatactgtccttctagtgtagccgtagttaggccaccacttcaagaact
ctgtagcaccgcctacatacctcgctctgctaatcctgttaccagtggc
tgctgccagtggcgataagtcgtgtcttaccgggttggactcaagacga
tagttaccggataaggcgcagcggtcgggctgaacggggggttcgtgca
cacagcccagcttggagcgaacgacctacaccgaactgagatacctaca
gcgtgagctatgagaaagcgccacgcttcccgaagggagaaaggcggac
aggtatccggtaagcggcagggtcggaacaggagagcgcacgagggagc
ttccagggggaaacgcctggtatctttatagtcctgtcgggtttcgcca
cctctgacttgagcgtcgatttttgtgatgctcgtcaggggggcggagc
ctatggaaaaacgccagcaacgcgagctcgtaatacgactcactatagg
forward PCR primer to a region of the CHIKV NSP4
SEQ ID NO: 11
tcactccctgttggacttgataga
reverse PCR primer to a region of the CHIKV NSP4
SEQ ID NO: 12
ttgacgaacagagttaggaacatacc
full-length infectious live-attenuated yellow
fever 17D genome
SEQ ID NO: 13
aguuuuuuuuuucauccaaaggucugcuuauucuugagcaaacugugcu
cacagaccucuggaaaccucuagaccccgucuuucuaccaccacgugg
gagggugguagcggaggcuccguucuuuuuacucuggggugggaggucc
cagaaaccagguuuuucgcaaccuggaggucggcugucccagccugcac
ugccucacagcuuagcaguggucggauacgauggaugacuaaauggaug
ugggaggcccagguggcccuauuggucauuccaaucagugauccgcacc
cauguugugcguccuuguggaacccaugaggugggaacagcugaggaaa
cagccaaugacaguagccucaugucccuuuugugaaaauacaucaguga
ccacauguuggcauaggcuuugcugaggcaagcuguuuccuugaucauc
cagccguuuccuggagacacccuuccucucccaaugagcucguccuguu
cucggcaaggcaccacaauccuccugccauccuucagcuguaguucaug
gaaguggugggaacagaagggcacauucucccaaucauuccacccuuuu
gauggcugccauucagauauguccuuucuaaccuuggacauggcguuga
gaugggacagggccaggccgaaccugucaucgaugggccggaccacaca
gucgucuccacucaccgccauccucuucagucugucacauccgugcuca
gugagccaugccuccagcuuggacuuucaaguuggugaugguguucaga
gcauaagucacuaccugcccggauccucucuggucucgucgacuuugug
augcgcgugucccauccagcggugucauccgcguagaauccaccaccau
ccauugcagccaggucucugaucaaggaaucccagggccucaaacucaa
gauaccgcgcucccagccacauauaccauauggcacggcuucccuuugc
uuucucuuuucucuggccagguggcggaacagccaccuguugacaacuu
ucaugaucuuccuaguucccgcugguggaucgugucccaauugggagga
ugacgucagccuccagggucacuuuuccaguuggacgccucauucuccu
caucaggaggccggauguuugguucacaguaaaugugacauugcugcgg
gcuccagacacguaguacauuucaugaguggaauuccuggagagagggu
uccugaucacuguuccgccaaaccuccuuuggagcaauuccaguuucuc
gagaacaucuggcauguauggagcuaacaccuucacacagaaguuguca
accccacaagccagccauuuuucuacaguaucaagaacucucacggucc
uuucccccucugugaccgaugacgaugaugacucuccaaugucacacaa
aagggugucacauuucacugguu09cgcggccacaccccaggucaauca
cccuaccuuccagcuugacauagccacgcucauggaaccaccuuaacuu
ugcggucccccuggagaccgccaccccgguguccaccuucccuucggcc
aaaugccugcgugccguaucacgauccaccuccacaaugurcguccuuu
uauacaacucaaacugucgcuuguccaacagauucaguucccucuucca
gacuucacccaaaauucccccucaugacuccugucauggagacagccau
ggguccauuccaaagaaggcugguguuucccucuaugagcggcccuaag
gcagcugaugcuaggacaaugccuucagccaaugaaaagggcguucugc
acauggcaacagaagcuaggcugagagraagaaggagauauagagccag
uuucuucucauaaagggcaggcauuucaggagcuuccucaaugucaacu
guuggauucccaucaaccacaggguucucggcaacgccauggaacaccc
uucucugugcaagcuuugacugcugcgcuuugauuccagguaaaaugag
agaccaguggagcauggcgcacccuaugccacagagcagaggcaucacu
guuauuaaggacugaggcugacugggcuauuccagacagagacagguug
ccauauucgacuuugauccaguggugcaacauuggagagagcauuguaa
caaugccaacguacacuguccaggcagcuccuggcuucaggucaagauc
cggccaacuccaguugcccuggcucggggaucacaaccaccaucaggac
aaagaauaugagcaugacauaggagaugugaguggguuugacgccucca
aggaacaugagauauccacagccggccauugugcccaucgccauagaca
uucuacugaugccuuugggagacaugaaaaagaugaccauucccgaugu
caguaguccagccaguauaaacagcaugacuauugucauugccucaggc
aucauugauagugcauugcgguaagcccuagagccuuccucagagugga
ggaacacacugaugguauccauugcagcaaacuuaauaaauucagacag
cgcacucuggucagaugacacccuuucaucacaccaccuugggcgcaga
ggcuucuuugcuccuccaggagcccugcacuucacuguuucaccgcugu
cauucaagaucucauguuccucagggccuucaaaacaccacuuacgauc
auucgucuucaaaccagccuuggccacuugccacgaaagccaaacgggc
aggucacaauuccuucaacgccauagaguggggcgaccauuccaccccu
caccuccauguuguccaagagcauugaggccuccaaccagcccacaagc
acaggcuuaaaagccguccugcaauccagcacucgcuccacgcaaaggu
uggcucccauuucagcuaugucaccacacucuuuccagccuuacgcaaa
gaggcagccaugacauuugcagcucugauggauggaaggaaccaugccg
uauuucaccauuugaauguggaaauucaucacuagucccaggcggugug
gcugucaucaagauuguugcacuuucauuugcccuagcucugugcgcug
cccaaccucuagcggcuaugcuagcuggauccaaaaaaugggcuucauc
cauaaugaucacuucccaguuaacaacccuaguugguuccaacauccug
uaaguuaggguggcauggcacauggcaucaaugaucagaaagaacaacc
cuggugggggccaacacaagagugcgcaagcgucuccgugcgcacucgg
ccaagaucuguggcaaggaugccauugccguacagcccaaucaccucuc
cguuccuguuaacaauaggagauccugaagugccacucggaguucuuuc
cuggaacagccgcgaucaacuggaccucuuccucuccaucccaucugcc
uuccaacuuccaugagccaccuugugacaugccacauuguguggaacac
cccucccugugccacucccacuccucgcugggaggcccccaagaagguu
gacuggaauaugccauaaaucccauccuccagauguucacauuccucga
ugaucuuaggagugggaauaucccacaagacauccccacuucuccuagc
uccccugacaugaaacagccacccagcaaggaccagcagaagagcaaau
ggauggagggcagccccaaccaaggccagcgaggucaucacaaccuggu
cccauggcacuuucucuucagaaagcagcuugaacuccccuuguucacu
gagugccacaucauagcgggcggaacucccgcugaucuccgccuccucu
ucccaugaaacuucaccaagcuucuugagcucuagcccauccacccucc
cagccacgcuaaccagcaucaucaggaguccuccaacugcaaucggacc
aaggaaguucuccaucuccugaaaagccaguccugccagcacucccacu
agaccagcugcugcgagugccucauucacugggauacuccuucgcccaa
auaugcggguugccagaaaugcacacaggcccaaaaaagguugugucaa
gcccagguaagaugugagugugagggccaccagagguauagucuucugc
auggagguguccuugaaauucuggugaaggaccccuaugauaaccacgg
cacaaaagaacauugcggcaagucucaccucagccauagugacaggugu
caacagagccaugaggggcaagaugguauuugaugcuuuccuagaagca
acagcauuuauugucaggaugcagagagaaacugcauuuagauacuucc
acaggccgcccaucacgccacccaaggcaaucuccaccauggcugcucc
uagggucagcacaaggcguucccgagggcuccauaggguccugagccca
aagccgaugagcagcccuggucugauugaaaaggcagcaaucaacaagg
agaguuacuugcccgaccagcauugcucccaagagcacuacuccuccaa
ccaacauuugcuuuggucccugucauggaagcucacaggcggcauugug
caggagcggcaacaccauucaggaauaacuuucccgcuauccguggugg
auuagugguaccugcauccaagguccguucgucugaaccuuguauccag
ggauaugauugugagagcuaacugggccuagccaugggcacucuuuuuu
ccguucaccgcugcacccaagauagauccaucgcagucuaugguguauu
caaagacugcguccauguacacgcguguggugaacacucccgucccaaa
cuccucuaucuggaaagaauuccagacccgguuugauaccccaagucuu
ccaaccauacugcagaccaucccgaauucuggaaaauggaugaguuccu
cucugguaaacauucccacaucucaugcucaagggagucaacugaauuu
aggccacacuucccuucuucaaaagaggcuuucacuauugaugcuuucc
caaccgaagugaagaacccuccagcggagcugaaaucccaggcgguguc
ucccaugacggccaggcguucccuccucucccaacgauaauguagcugu
cuccaaaagguggguucaccucaaucagcacuucaucaucauugguuga
ggcagggggcuccuuuugacacuuucaccugcaucacaacagugccaug
gccagugucaguuggguucuugacaaaaaacauuuuucagugauauuuu
guaggauguccccuugagugucaaagcugacaauuucacucugcaagaa
acaugaaggagccuuccugguuucccagggccaguacucugauaguggc
ggcaugcggagguucaaauucgacaagaugaugcaucucucuccacacc
ccgccacuuccacucugccauggcagggucaaguccugggcccacuguc
uguccacuauccagcucucuguuuccaucucagcgauguaacuguuacc
aaaguccaccgcaguuugcaccuggcauuccaguguagccucauggauu
uggcacaagugaauuuggcgcaugccacaaugcucccuuucccaaauag
gccacagccauugccccaggcuacugucucuagugagauguccaaugaa
ggcuugucaggggccauaacagugacacacuugucuugcuccaggguag
cugaaacccaaguuccuccaugcacccccucaaugaaaucccugucagu
aauuccaaugcagugagcugaguaggccggaccaacagccaagaccagu
agggcaaucacgacucguugcgucauguugcuucccacaagguaggcaa
ugguccuagaccugccugcugagucacacuuaccauaugcgacucuaac
guuuuccaccccauagcaccagcaaucaaugucaucuguuuuuccgcac
caaggucacuccacccgucaucaacagcauucccaaaauuaggaauugc
acagucagaacaucaugggaacggcguuuccuugaggacaauccucuca
ucaaacuggccaccacucucuugacuuuccuuagaacagcagacauguu
cuggucaguucucugcuaaucgcucaacgaacgauuaaaauuaauccaa
auguguuuauugccuagcaacucgauuugcagaccaaugcaccucaauu
agcacacaggauuuacu
YF 17D plasmid
SEQ ID NO: 14
gttgacgccgggcaagagcaactcggtcgccgcatacactattctcaga
atgacttggttgagtactcaccagtcacagaaaagcatcttacggatg
gcatgacagtaagagaattatgcagtgctgccataaccatgagtgataa
cactgcggccaacttacttctgacaacgatcggaggaccgaaggagcta
accgcttttttgcacaacatgggggatcatgtaactcgccttgatcgtt
gggaaccggagctgaatgaagccataccaaacgacgagcgtgacaccac
gatgcctgcagcaatggcaacaacgttgcgcaaactattaactggcgaa
ctacttactctagcttcccggcaacaattaatagactggatggaggcgg
ataaagttgcaggaccacttctgcgctcggcccttccggctggctggtt
tattgctgataaatctggagccggtgagcgtgggtctcgcggtatcatt
gcagcactggggccagatggtaagccctcccgtatcgtagttatctaca
cgacggggagtcaggcaactatggatgaacgaaatagacagatcgctga
gataggtgcctcactgattaagcattggtaactgtcagaccaagtttac
tcatatatactttagattgatttaaaacttcatttttaatttaaaagga
tctaggtgaagatcctttttgataatctcatgaccaaaatcccttaacg
tgagttttcgttccactgagcgtcagaccccttaataagatgatcttct
tgagatcgttttggtctgcgcgtaatctcttgctctgaaaacgaaaaaa
ccgccttgcagggcggtttttcgaaggttctctgagctaccaactcttt
gaaccgaggtaactggcttggaggagcgcagtcaccaaaacttgtcctt
tcagtttagccttaaccggcgcatgacttcaagactaactcctctaaat
caattaccagtggctgctgccagtggtgcttttgcatgtctttccgggt
tggactcaagacgatagttaccggataaggcgcagcggtcggactgaac
ggggggttcgtgcatacagtccagcttggagcgaactgcctacccggaa
ctgagtgtcaggcgtggaatgagacaaacgcggccataacagcggaatg
acaccggtaaaccgaaaggcaggaacaggagagcgcacgagggagccgc
cagggggaaacgcctggtatctttatagtcctgtcgggtttcgccacca
ctgatttgagcgtcagatttcgtgatgcttgtcaggggggcggagccta
tggaaaaacggctttgccgcggccctctcacttccctgttaagtatctt
cctggcatcttccaggaaatctccgccccgttcgtaagccatttccgct
cgccgcagtcgaacgaccgagcgtagcgagtcagtgagcgaggaagcgg
aatatatcctgtatcacatattctgctgacgcaccggtgcagccttttt
tctcctgccacatgaagcacttcactgacaccctcatcagtgccaacat
agtaagccagtatacactccgctagcgctgaggtctgcctcgtgaagaa
ggtgttgctgactcataccaggcctgaatcgccccatcatccagccaga
aagtgagggagccacggttgatgagagctttgttgtaggtggaccagtt
ggtgattttgaacttttgctttgccacggaacggtctgcgttgtcggga
agatgcgtgatctgatccttcaactcagcaaaagttcgatttattcaac
aaagccacgttgtgtctcaaaatctctgatgttacattgcacaagataa
aaatatatcatcatgaacaataaaactgtctgcttacataaacagtaat
acaaggggtgttatgagccatattcaacgggaaacgtcttgcTCGAGag
tggttttgtgtttgtcatccaaaggtctgcttattcttgagcaaactgt
gctcacagacctctggaggaaaagcagagaaccactccggtctttccct
ggcgtcaatatggtcccactatttgttccctggagggtctcctctaacc
tctagaccccgtctttctaccaccacgtgggagggtggtagcggaggct
ccgttctttttactctggggtgggaggtcccagaaaccaggtttttcgc
aacctggaggtcggctgtcccagcctgcactgcctcacagcttagcagt
ggcaaaactcgtgtggggttctgggctgacaacttcttctgtctcaatg
tgtggagtccggttctccatccgtagtttttatcccggtttctgtttca
ttttaagtgcggagcccggttctccagccgtggtttatatcccggtttc
aggttgtggggagtccggttctccacccgtggtttgtatcccggttatt
cctgttagatggtgtttcagataagctcacccagttgcaggtcagcatc
cacagaatacctgtccatgactgttaggtagtcagtgtatttctcctgt
ccaatcagcgttcggatacgatggatgactaaatggatgtgggaggccc
aggtggccctattggtcattccaatcagtgatccgcacagcttgtcttg
tctcttggttagataagggacatctctccattttttcaccattgtcttg
tcctgcatgtgtgggttgttggttatccatactctgttccacacctcaa
gcatgtcttccgtggtcatccactcccctttcccatgaatcgaccatgt
tgtgcgtccttgtggaacccatgaggtgggaacagctgaggaaacagcc
aatgacagtagcctcatgtcccttttgtgaaaatacatcagtgaccaca
tgttggcataggctttgctgaggcaagctgtttccttgatcatccagcc
gtttcctggagacacccttcctcagggcacattctcccaatcattccac
ccttttgatggctgccattcagatatgtcctttctaaccttggacatgg
cgttgagatgggacagggccaggccgaacctgtcatcgatgggccggac
cacacagtcgtctccactcaccgccatcctcttcagtctgtcacatccg
tgctcagtgagccatgcctccagcctggtcagaactgattcatcacaat
cttgaacatgttggtgatgtatcaccatctctgcttctgccattctgat
caattggactttcaagttggtgatggtgttcagagcataagtcactacc
tgcccggatcctctctggtctcgtcgacttatgacatccatgtaggctt
tccctcctggggctggtctcaacactttcaccactttgttcttgtatgt
catttccatcactgcttgtgccagttttttgtgatgtgggctcatgtag
ttcaagatctcctgttcatcatcaaggtctgcctctgtgatgcgcgtgt
cccatccagcggtgtcatccgcgtagaatccaccaccatccattgcagc
caggtctctgatcacatatcctaggtattgtaagccaatgccttccact
cctcctcctgagttttccctggaagcccaatggtcctcattcaggaatc
ccagggcctcaaactcaagataccgcgctcccagccacatataccatat
ggcacggcttccctttgctttcccaaactctgacagcttcttctctctt
ttccccatcatgttgtacacacaagtccgacacctgccttgttggtgca
gcttcctttcttcatccaccagttcccagaactttgggtcttggacagc
ctcattggcagtcttccactgttcttgttcttccaggtaagctccaatg
gctgcatgacttcggacttttgcaataaattcttcctttgtgcacagtc
tggggttcttttctctggccaggtggcggaacagccacctgttgacaac
tttcatgatcttcctagttcccgctggtggatcctttgctctggtgtca
actttttctttaaacactctttgctgtccaaaaggggttgtgtcagtca
ttgccattcttgtgacctcctctatcctgtcccatggatatgtcagaat
tttaataacaccatttaccatgctcgccgcacttcctgaggtttttgtg
acataggagccacagtagtgccaggtcctgtaggggttgtcattgtcat
aaaaccaagaggtcatgtactcagattttatcctctcaaccctttcttc
tatggcctctttgtccaggggtcccttgtctgtctcaacactgcgtgtc
ccaattgggaggatgacgtcagcctccagggtcacttttccagttggac
gcctcattctcctcatcaggaggcgggatgtttggttcacagtaaatgt
gacattgctgcgggctccagacacgtagtacatttcatgagtggaattc
ctggagagagggttcctgatcactgttccgccaaacctcctttggagca
attccagtttctcgagaacatctggcatgtatggagctaacaccttcac
acagaagttgtcaaccccacaagccagccatttttctacagtatcaaga
actctcacggtcctttccccctctgtgaccgatgacgatgatgactctc
caatgtcacacaaaagggtgtcacatttcactggttctaggcggtggat
atcagttttgtccttgaaggtgatgatgttccatcccagactttgcaca
ttcatgggtttctcatggccgtctcttccaagagtaaatcctttgaccc
cactcacttccttttgcgcagcagcgtagtaacaccagcctccgcggcc
acaccccaggtcaatcaccctaccttccagcttgacatagccacgctca
tggaaccaccttaactttgcggtccccctggagaccgccaccccggtgt
ccaccttcccttcggccaaatgcctgcgtgccgtatcacgatccacctc
cacaatgtcggtccttttatacaactcaaactgtcgcttgtccaacaga
ttcagttccctcttccagacttcacccaaagtttttccattcgcgctcc
cccggcgtccagttttcatcttccatagattgtacatgactcccacaaa
agcatagtgattccccctcatgactcctgtcatggagacagccatgggt
ccattccaaagaaggctggtgtttccctctatgagcggccctaaggcag
ctgatgctaggacaatgccttcagccaatgaaaagggcgttctgcacat
ggcaacagaagctaggctgagagcaagaaggagatatagagccagtttc
ttctcataaagggcaggcatttcaggagcttcctcaatgtcaactgttg
gattcccatcaaccacagggttctcggcaacgccatggaacacccttct
ctgtgcaagctttgactgctgcgctttgattccaggtaaaatgagagac
cagtggagcatggcgcaccctatgccacagagcagaggcatcactgtta
ttgaattccagccactgaccagcagcattatgaccgagatattcatctt
catgaatggtatccccttgtccatgaaagaaaggactgaggctgactgg
gctattccagacagagacaggttgccatattcgactttgatccagtggt
gcaacattggagagagcattgtaacaatgccaacgtacactgtccaggc
agctcctggcttcaggtcaagatccggccaactccagggtgaagcacta
gatggaattaagttcttcttcccaaagaggtcctctttggttttctcca
gcatgcctagctcgttggctgccaccgctgaaaccagcgtcaggatgcc
aataatgaggtatgccacttggttgtcttggatggacctttgttgccct
ggctcggggatcacaaccaccatcaggacaaagaatatgagcatgacat
aggagatgtgagtgggtttgacgcctccaaggaacatgagatatccaca
gccggccattgtgcccatcgccatagacattctactgatgcctttggga
gacatgaaaaagatgaccattcccgatgtcagtagtccagccagtataa
acagcatgactattgtcattgcctcaggcatcattgatagtgcattgcg
gtaagccctagagccttcctcagagtggaggaacacactgatggtatcc
attgcctctccaccttttttagccaggaaatcagggagttcactcagca
caactagcacttcagcagctcccctcctaccttcagcaaacttaataaa
ttcagacagcgcactctggtcagatgacaccctttcatcacaccacctt
gggcgcagaggcttctttgctcctccaggagccctgcacttcactgttt
caccgctgtcattcaagatctcatgttcctcagggccttcaaaacacca
cttacgatcattcgtcttcaaaccagccttggccacttgccacgaaagc
caaacgggcaggtcacaattcctcactagttctctgaagactttcctct
ggtcatccctcagtctcatttcaccaggggaaactggtgttttagttcc
ttcaacgccatagagtggggcgaccattccacccctcacctccatgttg
tccaagagcattgaggcctccaaccagcagacgtggtgggcattatttt
cacttgtaggctcagaatagtagtatgagtctccatctctgttgggatt
tctcccaatgcgccccctcctttgagcagcagaggatgcggagatacga
agtggcccttttattgccaccttcctcccttcatccacaagcacaggct
taaaagccgtcctgcaatccagcactcgctccacgcaaaggttggctcc
catttcagctatgtcagtggccaatataaagtcaggtttcttctgcttt
atcgtggggtattctctctcaaaggttttcctgttcaggaccaccacac
tctttccagccttacgcaaagaggcagccatgacatttgcagctctgat
ggatggaaggaaccatgccgtgggccttttgtcagctaggatccagtca
tgccctgtgttccagggctcactgggtatgtccgtttgaacatcttcta
tttcaccatttgaatgtggaaattcatcactagtcccaggcggtgtggc
tgtcatcaagattgttgcactttcatttgccctagctctgtgcgctgcc
caacctctagcggctatgctagctggatccaaaaaatgggcttcatcca
taatgatcacttcccagttaacaaccctagttggttccaacatcctgta
agttagggtggcatggcacatggcatcaatgacttctctcccgctgccg
tgagcggaaaaagcctgtgtgtggaatttcacgtccaggccgtgaaaag
cctccttcatttcagaaagaacaaccctggtgggggccaacacaagagt
gcgcaagcgtctccgtgcgcactcggccaagatctgtgggaggaaacgt
cttgtcttcccagctccaggatgaaaatcaaggacagttgtcattcctt
tctttagcattgtcgggatctcttggagctcctcctttccttcttcctt
cacctcagtctgggatatggcggacacgaaggagttgtcaccgacaagg
atgccattgccgtacagcccaatcacctctccgttcctgttaacaatag
gagatcctgaagtgccactcggatagtcaagagcgacagccccgatttc
tcccccattcctcactttgaacaagctcggttttgtctggacgttgacc
acgttctttcctggaacagccgcgatcaactggacctcttcctctccat
cccatctgccttccaacttccatgagccaccataggcgacaaggtcttc
ctttactgaagcccaagatggaatcaacttcttgccattcctgacaagg
aaagctcctcttgtgacatgccacattgtgtggaacacccctccctgtg
ccactcccactcctcgctgggaggcccccaagaaggttgactggaatat
gccataaatcccatcctccagatgttcacattcctcgatgatcttagga
gtgggaatatcccacaagacatccccacttctcctagctcccctgacat
gaaacagccacccagcaaggaccagcagaagagcaaatggatggagggc
agccccaaccaaggccagcgaggtcatcacaacctggtcccatggcact
ttctcttcagaaagcagcttgaactccccttgttcactgagtgccacat
catagcgggcggaactcccgctgatctccgcctcctcttcccatgaaac
ttcaccaagcttcttgagctctagcccatccaccctcccagccacgcta
accagcatcatcaggagtcctccaactgcaatcggaccaaggaagttct
ccatctcctgaaaagccagtcctgccagcactcccactagaccagctgc
tgcgagtgcctcattcactgggatactccttcgcccaaatatgcgggtt
gccagaaatgcacacaggcccaaaaaaggttgtgtcaagcccaggtaag
atgtgagtgtgagggccaccagaggtatagtcttctgcatggaggtgtc
cttgaaattctggtgaaggacccctatgataaccacggcacaaaagaac
attgcggcaagtctcacctcagccatagtgacaggtgtcaacagagcca
tgaggggcaagatggtatttgatgctttcctagaagcaacagcatttat
tgtcaggatgcagagagaaactgcatttagatacttccacaggccgccc
atcacgccacccaaggcaatctccaccatggctgctcctagggtcagca
caaggcgttcccgagggctccatagggtcctgagcccaaagccgatgag
cagccctggtctgattgaaaaggcagcaatcaacgccatatacatggcg
tctcctccattgttcatctcatggaaatgcaatcccacagccactgtga
gtttcagcaaatcaaggagagttacttgcccgaccagcattgctcccaa
gagcactactcctccaaccaacatttgctttggtccctgtcttttcctt
aggaccacttccattgctatcatcatgctcaccaaaccaaaagggacag
catgtatttctccagctgtaacccaggagcgcaccagatggctttcatg
cgttttccttggcctaatttccatgggataccaacacccatcactacca
tggaagctcacaggcggcattgtgcaggagcggcaacaccattcaggaa
taactttcccgctatccgtggtggatctggttgattttccccgtccatc
acagttgccatcaatgatcacgctagtccctgggcaagcttctctcttc
acttctagtggtacctgcatccaaggtccgttcgtctgaaccttgtatc
cagggatatgattgtgagagctaactgggcctccgattgatctcggcat
gaacatttcactctcttcaactgatgttccaatcgtatgtgtcagtggc
cactcacactccttgtaatctaatgcctccaaggtgtggatcatccatg
tcccatttacttcatgacttcccatccaaaatgttggagagccatgggc
actcttttttccgttcaccgctgcacccaagatagatccatcgcagtct
atggtgtattcaaagactgcgtccatgtacacgcgtgtggtgaacactc
ccgtcccaaactcctctatctggaaagaattccagacccggtttgaaaa
cgggcattctttcctggactttccatctatgatgaagcttccattcttc
ctccctggggagaacacaaggttcttaccccaagtcttccaaccatact
gcagaccatcccgaattctggaaaatggatgagttcctctctggtaaac
attctttggatcctgcacgacaacagaaatgtccacctcgttttcctca
aaaatggcattgatctcatctgccctcacaggatcttctggatagtatg
agtacttgttcagccagtcatcagagtctctaaatatgaagataccatc
tccgcacttgagctctctcttgccaaagttgatggcgcatccttgatcc
gccccaactcctagagacaaaaacatcatgatcactcctaccaagatca
tgctcatggacattgtcatgtttcttgtgttgatgccaacccatataag
taccgcccccatgatgacctttgttatccagttcaagccgccaaatagc
ccctgaaaggcagagccaaacaccgtatgaattcctttcccaaccgaag
tgaagaaccctccagcggagctgaaatcccaggcggtgtctcccatgac
ggccaggcgttccacgcctttcatggtctgagtgaacaactttcctatt
gagcttccctctttgtgccactggtaagtgagacgtgaatctcctctcc
caacgataatgtagctgtctccaaaaggtgggttcacctcaatcagcac
ttcatcatcattggttgaggcgatggggttaactgtaaccaaaatgcct
ttattgattgccgctgtaagatcatcagctactatcactggaatcctgc
agggggctccttttgacactttcacctgcatcacaacagtgccatggcc
agtgtcagttgggttcttgacaaaaaacattttgtcagtgcatattttg
taggatgtccccttgagtgtcaaagctgacaatttcactctgcaagaaa
catgtccaccatgtagtttgtaaaggttgttgtcatttgtgtcctttgt
aaccctcattgcgccagtaagagctgttttcaaggagccttcctggttt
cccagggccagtactctgatagtggcggcatgcggaggttcaaattcga
caagatgatgcatctctctccacaccccgccacttccactctgccatgg
cagggtcaagtcctgggcccactgtctgtccactatccagctctctgtt
tccatctcagcgatgtaactgttaccaaagtccaccgcagtttgcacct
ggcattccagtgtagcttttccatacccaatgaactcgacttcctggga
gcctgacagggcatcaaacttgagagtcttaatgtcggtattccaattt
tcctgcttggcccctacatgcaattgtgctctgatgacatactgaattt
tggtctgatcaacctcaaacaaactcatggatttggcacaagtgaattt
ggcgcatgccacaatgctccctttcccaaataggccacagccattgccc
cagcctctatcagaataagtgcgcttgcacgcattgtccccttcgttct
cttcagctaggtgggcctctccagtgctggggcacttgtcattaatctt
cacatgagtgagaactgcattgtaacacactttcctcacctcagcaggt
ctatcaatggctactgtctctagtgagatgtccaatgaaggcttgtcag
gggccataacagtgacacacttgtcttgctccagggtagctgaaaccca
agttcctccatgcaccccctcaatgaaatccctgtcagtaattccaatg
cagtgagctgagtaggccggaccaacagccaagaccagtagggcaatca
cgactcgttgcgtcatgttgcttcccacaaggtaggcaatggtcagagc
cgtcactgcaaaaaaggggttcctcacgaaccatctctcaatcttttgg
agttgcctttcacccattcttccagtcatccatttttcttgccgggtct
tcaaaccatggttttcatgcgtaggcaagtcaatggcccttcttgacct
cctagacctgcctgctgagtcacacttaccatatgcgactctaacgttt
tccaccccatagcaccagcaatcaatgtcatctggctcctctcttggac
tgagattgggacagttgtattccattgagtctgggcaccagtacttggc
ttccaaaatgtttgttgtgcagttgcctgtgcccacagagaatgttttc
ccgaggtcctcagatgtcacatttaggagcaaccatctgtttttccgca
ccaaggtcactccacccgtcatcaacagcattcccaaaattaggaattg
cacagtcagaacatcatgggaacggcgtttccttgaggacaatcctctc
atcaaactggccaccactctcttgactttccttagaacagccaagcctt
gtcttgggtccagcattttccacaacctctttaggtgggctgtgatctt
ttttccagtcaaaatgttgaacaaaaagaaaaagataaatccttgaaca
cctcttgaaggtccaggtctgtttccaatttgttttgttttttgtttta
ttttgtttgacaaggagcgaactcctcgtcgtaccatattgacgcccag
ggtttttccctgagctttacgaccagacatgttctggtcagttctctgc
taatcgctcaacgaacgattaaaattaatccaaatgtgtttattgccta
gcaactcgatttgcagaccaatgcacctcaattagcacacaggatttac
tcctatagtgagtcgtattagcggccgccaggtggcacttttcggggaa
atgtgcgcggaacccctatttgtttatttttctaaatacattcaaatat
gtatccgctcatgagacaataaccctgataaatgcttcaataatattga
aaaaggaagagtatgagtattcaacatttccgtgtcgcccttattccct
tttttgcggcattttgccttcctgtttttgctcacccagaaacgctggt
gaaagtaaaagatgctgaagatcagttgggtgcacgagtgggttacatc
gaactggatctcaacagcggtaagatccttgagagttttcgccccgaag
aacgttttccaatgatgagcacttttaaagttctgctatgtggcgcggt
attatcccgt

### CONCLUSION

Although the subject matter has been described in language specific to features 25 and/or methodological acts, it is to be understood that the subject matter defined in the appended claims is not necessarily limited to the specific features or acts described above. Rather, the specific features and acts are disclosed as example forms of implementing the claims.

Certain implementations are described herein, including the best mode known to 30 the inventors for carrying out the invention. Of course, variations on these described implementations will become apparent to those of ordinary skill in the art upon reading the foregoing description. Skilled artisans will know how to employ such variations as appropriate, and the implementations disclosed herein may be practiced otherwise than specifically described. Accordingly, all modifications and equivalents of the subject matter recited in the claims appended hereto are included within the scope of this disclosure. Moreover, any combination of the above-described elements in all possible variations thereof is encompassed by the invention unless otherwise indicated herein or otherwise clearly contradicted by context.

All references listed herein, including patent applications and patent publications are herein incorporated by reference in their entirety, as if each individual reference is specifically and individually indicated to be incorporated by reference.

1. A composition for causing viral infection in a subject, the composition comprising:
a. a ribonucleic acid (RNA) polynucleotide encoding a replication-competent viral genome; and
b. an artificial RNA delivery system,
wherein the RNA is present in an amount sufficient to cause to viral replication in the subject.

2. The composition of claim 1, wherein the RNA is transcribed from a DNA plasmid.

3. The composition of any of claims 1-2, wherein the viral genome is a genome of an attenuated virus.

4. The composition of claim 3, wherein the viral genome is a full-length genome.

5. The composition of any of claims 1-4, wherein the RNA is single-stranded.

6. The composition of any of claims 1-5, wherein the RNA is present in an amount sufficient to induce neutralizing antibodies in the subject.

7. The composition of claim 6, wherein a titer of neutralizing antibodies is the same as induced by live viral vaccination.

8. The composition of claim 6, wherein a titer of the neutralizing antibodies exceeds a titer that is a correlate of protection.

9. The composition of any of claims 1-8, wherein the composition does not include an additional adjuvant.

10. The composition of any of claims 1-9, wherein the viral genome is a genome of a positive strand virus.

11. The composition of claim 10, wherein the positive strand virus is an Alphavirus.

12. The composition of claim 11, wherein the alphavirus is Chikungunya (CHIKV).

13. The composition of claim 12, wherein the CHIKV is CHIKV 181/25.

14. The composition of claim 12, wherein the CHIKV is CHIKV-Δ5nsp3.

15. The composition of claim 12, wherein the CHIKV is CHIKV-Δ6K.

16. The composition of claim 10, wherein the positive strand virus is a flavivirus.

17. The composition of claim 16, wherein the flavivirus is yellow fever virus, Zika virus, Japanese encephalitis virus, West Nile virus, hepatitis C virus, tick-borne encephalitis, Powassan virus, or dengue virus.

18. The composition of claim 17, wherein the positive strand virus is yellow fever.

19. The composition of claim 18, wherein the yellow fever is YF17D.

20. The composition of claim 10, wherein the positive strand virus is a coronavirus.

21. The composition of claim 20, wherein the coronavirus is MERS, SARS, or SARS-CoV-2.

22. A Chikungunya virus (CHIKV) vaccine, comprising:
a. a ribonucleic acid (RNA) polynucleotide encoding an attenuated, replication-competent CHIKV genome; and
b. an artificial RNA delivery system,
wherein the RNA is present in an amount sufficient to cause to viral replication in the subject.

23. The vaccine of claim 22, wherein the CHIKV genome is CHIKV 181/25.

24. The vaccine of claim 22, wherein the CHIKV genome is CHIKV-Δ5nsp3.

25. The vaccine of claim 22, wherein the CHIKV genome is CHIKV-Δ6K.

26. A yellow fever virus vaccine, comprising:
a. a ribonucleic acid (RNA) polynucleotide encoding an attenuated, replication-competent yellow fever genome; and
b. an artificial RNA delivery system,
wherein the RNA is present in an amount sufficient to cause to viral replication in the subject.

27. The vaccine of claim 26, wherein the yellow fever genome is YF17D.

28. The vaccine of any of claims 22-27, wherein the RNA is transcribed from a DNA plasmid.

29. The vaccine of any of claims 22-29, wherein the viral genome is a full-length genome.

30. The vaccine of any of claims 22-29, wherein the RNA is single-stranded.

31. The vaccine of any of claims 22-30, wherein the RNA is present in an amount sufficient to induce neutralizing antibodies in a subject.

32. The vaccine of claim 31, wherein a titer of neutralizing antibodies is the same as induced by live viral vaccination.

33. The vaccine of claim 31, wherein a titer of neutralizing antibodies exceeds a titer that is a correlate of protection.

34. The vaccine of any of claims 22-33, wherein the composition does not include an additional adjuvant.

35. The composition or vaccine of any of claims 1-34, wherein the artificial RNA delivery system comprises a lipid particle.

36. The composition or vaccine of claim 35, wherein the lipid particle is a lipid nanoparticle (LNP).

37. The composition or vaccine of claim 35, wherein the lipid particle is a nanostructured lipid carrier (NLC).

38. The composition or vaccine of claim 37, wherein the NLC comprises a liquid oil, a solid lipid, a hydrophobic sorbitan ester, a hydrophilic ethoxylated sorbitan ester, and a cationic lipid.

39. The composition or vaccine of claim 38, wherein liquid oil is squalene or synthetic squalene, solid lipid is Glyceryl trimyristate, the hydrophobic sorbitan ester is sorbitan monostearate, the hydrophilic ethoxylated sorbitan ester is polysorbate 80, and the cationic lipid is DOTAP (N-[1-[2,3-Dioleoyloxy)propyl]-N,N,N-trimethylammonium chloride).

40. The composition or vaccine of claim 35, wherein the lipid particle is a cationic nanoemulsion (CNE).

41. The composition or vaccine of any of claims 1-35, wherein the artificial RNA delivery system comprises amphiphilic diblock oligomers containing a sequence of lipid monomers and a sequence of cationic monomers.

42. A pharmaceutical composition comprising the composition or vaccine of any of claims 1-41, and at least one pharmaceutically acceptable carrier, excipient, and/or adjuvant.

43. A method of inducing an immune response in a subject comprising, administering to the subject ribonucleic acid (RNA) polynucleotide encoding a replication-competent viral genome in an amount sufficient to cause to viral replication in the subject.

44. A method of causing a viral infection in a cell, comprising contacting the cell with ribonucleic acid (RNA) polynucleotide encoding a replication-competent viral genome complexed with or contained within an artificial RNA delivery system.

45. The method of any of claims 43-44, wherein the RNA is transcribed from a DNA plasmid.

46. The composition of any of claims 43-45, wherein the viral genome is a genome of an attenuated virus.

47. The method of claim 46, wherein the viral genome is a full-length genome.

48. The method of any of claims 43-47, wherein the RNA is single-stranded.

49. The method of any of claims 43-44, wherein the viral genome is a genome of a positive strand virus.

50. The method of claim 45, wherein the positive strand virus is an Alphavirus.

51. The method of claim 50, wherein the alphavirus is Chikungunya (CHIKV).

52. The method of claim 51, wherein the CHIKV is CHIKV 181/25.

53. The method of claim 51, wherein the CHIKV is CHIKV-Δ5nsp3.

54. The method of claim 51, wherein the CHIKV is CHIKV-Δ6K.

55. The method of claim 45, wherein the positive strand virus is a flavivirus.

56. The method of claim 55, wherein the flavivirus is yellow fever virus, ZIKA virus, Japanese encephalitis virus, West Nile virus, hepatitis C virus, tick-borne encephalitis, or dengue virus.

57. The method of claim 45, wherein the positive strand virus is yellow fever.

58. The method of claim 57, wherein the yellow fever is YF17D.

59. The method of claim 45, wherein the positive strand virus is a coronavirus.

60. The method of claim 59, wherein the coronavirus is MERS, SARS, or SARS-CoV-2.

61. A method of inducing protective immunity in a subject against Chikungunya virus (CHIKV) comprising, administering to the subject a ribonucleic acid (RNA) polynucleotide encoding an attenuated, replication-competent CHIKV genome in an amount sufficient to cause to viral replication in the subject.

62. The method of claim 61, wherein the CHIKV genome is CHIKV 181/25.

63. The method of claim 61, wherein the CHIKV genome is CHIKV-Δ5nsp3.

64. The method of claim 61, wherein the CHIKV genome is CHIKV-Δ6K.

65. A method of inducing protective immunity in a subject against yellow fever comprising, administering to the subject a ribonucleic acid (RNA) polynucleotide encoding an attenuated, replication-competent yellow fever genome in an amount sufficient to cause to viral replication in the subject.

66. The method of claim 65, wherein the yellow fever genome is YF17D.

67. The method of any of claims 43-66, wherein the RNA administered to the subject is complexed with or contained within an artificial RNA delivery system.

68. The method of claim 67, wherein the artificial RNA delivery system comprises a lipid particle.

69. The method of claim 68, wherein the lipid particle is a lipid nanoparticle (LNP).

70. The method of claim 68, wherein the lipid particle is a nanostructure lipid carrier (NLC).

71. The method of claim 70, wherein the NLC comprises a liquid oil, a solid lipid, a hydrophobic sorbitan ester, a hydrophilic ethoxylated sorbitan ester, and a cationic lipid.

72. The method of claim 71, wherein liquid oil is squalene or synthetic squalene, solid lipid is Glyceryl trimyristate, the hydrophobic sorbitan ester is sorbitan monostearate, the hydrophilic ethoxylated sorbitan ester is polysorbate 80, and the cationic lipid is DOTAP (N-[1-[2,3-Dioleoyloxy)propyl]-N,N,N-trimethylammonium chloride).

73. The method of claim 68, wherein the lipid particle is a cationic nanoemulsion (CNE).

74. The method of claim 67, wherein the artificial RNA delivery system comprises amphiphilic diblock oligomers containing a sequence of lipid monomers and a sequence of cationic monomers.

75. The method of any one of claims 43-74, wherein the immune response is induced after a single dose.

76. The method of any one of claims 43-75, wherein the administering does not include electroporation.

77. The method of any one of claims 43-76, wherein the administering does not include a biolistic particle delivery system.

78. The method of any one of claims 43-77, wherein the immune response comprises neutralizing antibodies.

79. The method of claim 78, wherein a titer of the neutralizing antibodies is the same as induced by live viral vaccination.

80. The method of claim 78, wherein a titer of the neutralizing antibodies exceeds a titer that is a correlate of protection.

81. The method of any one of claims 43-80, wherein the administering is intramuscular.

82. The method of any one of claims 43-80, wherein the administering is subcutaneous.

83. The method of any one of claims 43-80, wherein the administering is intranasal.

84. The method of any one of claims 43-83, wherein the amount is 1 μg.

85. The method of any one of claims 43-83, wherein the amount is 10 μg.

86. The method of any one of claims 43-83, wherein the amount is 100 μg.


SEQUENCE LISTING (14 sequences)

SEQ ID NO: 1 - RNA, 12036 nt, Alphavirus Chikungunya virus
auggcugcgugagacacacguagccuaccaguuucuuacugcucuacucugcaaagcaagagauuaauaacccaucauggauucuguguacguggacauagacgcugacagcgccuuuuugaaggcccugcaacgugcguaccccauguuugagguggaaccuaggcaggucacaucgaaugaccaugcuaaugcuagagcguucucgcaucuagccauaaaacuaauagagcaggaaauugaucccgacucaaccauccuggauauagguagugcgccagcaaggaggaugaugucggacaggaaguaccacugcguuugcccgaugcgcagcgcagaagaucccgagagacucgcuaauuaugcgagaaagcucgcaucugccgcaggaaaaguccuggacagaaacauuucuggaaagaucggggacuuacaagcggugauggccgugccagacacggagacgccaacauuuugcuuacacacagaugucucauguagacagagagcagacgucgcgauauaccaagacgucuaugcuguacacgcacccacgucgcuauaccaccaggcgauuaaaggaguccgaguggcguacuggguaggguucgacacaaccccguucauguacaacgcuauggcgggugccuaccccucauacucgacaaauugggcggaugagcagguacugaaggcuaagaacauaggauuauguucaacagaccugacggaagguagacgaggcaaauugucuaucaugagagggaaaaagcuaaaaccgugcgaccgugugcuguucucaguagggucaacgcuuuacccggaaagccgcacgcuacuuaagagcuggcaccuaccaucgguguuccaucuaaagggcaagcuuagcuucacaugccgcugugacacagugguuucgugugagggcuacgucguuaagagaauaacgaugagcccaggccuuuauggaaaaaccauaggguaugcgguaacccaccacgcagacggauucuugaugugcaagacuaccgacacgguugacggcgaaagagugucauucucggugugcacguacgugccggcgaccauuugugaucaaaugaccggcauccuugcuacagaagucacgccggaggaugcacagaagcuguugguggggcugaaccagaggauagugguuaacggcagaacgcaacggaacacgaacaccaugaagaacuaccuacuucccguggucgcccaggccuucaguaagugggcaaaggagugccggaaggacauggaagaugagaagcuucugggggucagagaaagaacacuaaccugcugcugucuaugggcauuuaagaagcagaaaacacacacggucuacaagaggccugauacccagucaauccagaagguucaggccgaauuugacagcuuuguaguaccgggccuguggucguccggguugucaaucccguugaggacuagaaucaagugguuguuacgcaaggugccgaaaacagaccugaucccauacagcgggaaugcccaagaagcccaggaugcagaaaaagaagcagaggaagaacgagaagcagaacugacucaugaggcucuaccaccccuacaggcagcacaggaagauguccaggucgaaaucgacguggaacagcuugaggauagagcuggugcuggaauaauagagacuccgagaggcgcuaucaaaguuacugcccaacuaacagaccacgucgugggggaguaccugguacuuuccccgcagaccguacuacgcagccagaagcucagccugauccacgcuuuagcggagcaagugaagacguguacgcacagcggacgagcagggagguaugcggucgaagcguacgauggccgaguccuagugcccucaggcuaugcaauuucgccugaagacuuccagagucuaagcgaaagcgcaacgaugguguacaacgaaagagaguucguaaacagaaaguuacaccacauugcgaugcacggaccagcccugaacacugacgaagagucguaugagcuggugagggcagagaggacagaacacgaguacgucuacgacguggaccagagaagaugcuguaagaaggaagaagcugcaggacugguacuggugggcgacuugacuaauccgcccuaccacgaauucgcauacgaagggcuaaaaauucgccccgccugcccauacaaaauugcagucauaggagucuucgggguaccaggaucuggcaagucagccauuaucaagaaccuaguuaccaggcaagaccuggugacuagcggaaagaaagaaaacugccaagaaaucagcaccgacgugaugagacagagaggucuagagauaucugcacguacgguagauucgcugcucuugaauggaugcaacagaccagucgacguguuguacguagacgaggcguuugcgugccacucuggaacguuacuugcuuugaucgccuuggugagaccaagacagaaaguuguacuuuguggugacccgaagcagugcggcuucuucaauaugaugcagaugaaagucaacuacaaucauaacaucugcacccaaguguaccacaaaaguaucuccaggcgguguacacugccugugacugccauugugucaucguugcauuacgaaggcaaaaugcgcacuacgaaugaguacaacaugccgauuguaguggacacuacaggcucaacgaaaccugacccuggagaccucguguuaacgugcuucagaggguggguuaaacaacugcaaauugacuaucguggacacgaggucaugacagcagccgcaucccaaggguuaacuagaaaaggaguuuacgcaguuaggcaaaaaguuaacgaaaacccacucuaugcaucaacaucagagcacgucaacguacuccuaacgcguacggaagguaaacugguauggaagacacucucuggugacccguggauaaagacgcugcagaacccaccgaaaggaaacuucaaagcaacuauuaaggagugggagguggagcacgcaucgauaauggcgggcaucugcagucaccaagugaccuuugacacauuccaaaacaaagccaacguuugcugggcuaagagcuuggucccuauccucgaaacagcggggauaaaacuaaaugauaggcaguggucccagauaauucaagccuucaaagaagacaaagcauacucacccgaaguagcccugaaugaaauaugcacgcgcauguaugggguggaucuagacagugggcuauucucuaaaccguugguaucuguguauuacgcggauaaccauugggauaauaggccgggaggaaagauguucggauucaacccugaggcagcguccauucuagaaagaaaguacccauuuacaaaaggaaaguggaacaucaacaagcagaucugcgugacuaccaggaggauagaagacuucaacccuaccaccaacauuauaccggucaacaggagacuaccacacucauuaguggccgaacaccgcccaguaaaaggggaaagaauggaauggcugguuaacaagauaaacggacaccacguacuccugguuagcggcuauaaccuugcacugccuacuaagagagucaccuggguagcgccacuagguguccgcggagcggacuauacauacaaccuagagcugggucuaccagcaacgcuugguagguaugaccuaguggucauaaacauccacacaccuuuucgcauacaccauuaccaacagugcguagaucacgcaaugaaacugcaaaugcuagggggugacucacugagacugcucaaaccggguggcucucuauugaucagagcauacgguuacgcagauagaaccagugaacgagucaucugcguacugggacgcaaguuuagaucgucuagagcauugaaaccaccaugugucaccaguaauacugagauguuuuuccuauuuagcaauuuugacaauggcagaaggaauuuuacaacgcaugucaugaacaaucaacugaaugcagccuuuguaggacaggccacccgagcaggaugugcaccaucguaccggguaaaacgcauggacaucgcgaagaacgaugaagagugcgugguuaacgccgccaacccucgcggguuaccaggugacgguguuugcaaggcaguauauaaaaaguggccggaguccuuuaaaaacagugcaacaccaguaggaaccgcaaaaacaguuaugugcgguacguauccaguaauccacgccguaggaccaaacuucucaaauuauucggagucugaaggggaccgggaauuggcggcugccuaucgagaagucgcaaaggaaguaacuagacugggaguaaauagcguagcuauaccucuccucuccacagguguauacucaggagggaaagacaggcuaacccagucacugaaccaccucuuuacagccauggacucgacggaugcagacguggucaucuacugccgagacaaggaaugggagaagaaaauaucugaggccauacagaugcggacccaaguggagcugcuggaugagcacaucuccauagacugcgaugucauucgcgugcacccugacaguagcuuggcaggcagaaaaggauacagcaccacggaaggcgcacuguauucauaucuagaagggacacguuuucaccagacggcaguggauauggcagagauauacacuauguggccaaagcaaacagaggccaaugagcaagucugccuauaugcccugggggaaaguauugaaucaaucaggcagaaaugcccgguggaugaugcagacgcaucaucucccccgaaaacugucccgugucuuugccgguaugccaugacuccugaacgcgucacccgacuucgcaugaaccaugucacaaauauaauuguguguucuucauuuccccuuccaaaguacaagauagaaggagugcaaaaagucaaaugcuccaagguaauguuauucgaucacaaugugccaucgcgcguaaguccaagggaauacagaucuucccaggagucuguacaggaagugaguacgacaacgucauugacgcauagccaguuugaucuaagcgccgauggcgagacacugccugucccgucagaccuggaugcugacgccccagcccuagaaccggcccuagacgacggggcgguacauacauuaccaaccauaaucggaaaccuugcggccgugucugacuggguaaugagcaccguaccugucgcgccgccuagaagaaggagagggagaaaccugacugugacaugugacgagagagaagggaauauaacacccauggcuagcguccgauucuuuagagcagagcuguguccggccguacaagaaacagcggagacgcgugacacagcuauuucccuucaggcaccgccaaguaccaccauggaacugagccauccaccgaucuccuucggagcaccaagcgagacguuccccaucacauuuggggacuucgacgaaggagaaaucgaaagcuugucuucugagcuacuaacuuucggagacuuccuacccggugaaguggaugaucugacagauagcgacugguccacgugcccagacacggacgacgaguuaugacuagacagggcagguggguauauauucucgucggacacugguccaggccauuuacaacagaagucgguacgccagucagugcugccgguaaacacccuggaggaaguccacgaggagaaguguuacccaccuaagcuggaugaauuaaaggagcaacuacuacuuaagaaacuccaggagagugcguccauggccaauagaagcagguaucagucacgcaaaguggaaaauaugaaagcaacaaucauccagagacuaaagagaggcuguaaacuguauuuaauggcagagaccccgaaagucccgacuuaucggaccauauacccggcgccuguguacucgccuccgaucaauguccgauuguccaaccccgaguccgcaguggcagcauguaaugaguucuuagcuagaaacuacccaacuguuucaucauaccaaaucaccgacgaguaugaugcauaucuagacaugguggacgggucggagaguugcuuggaccgagcgacauucaauccgucaaaacuuaggagcuacccgaaacaacaugcuuaucacgcgccuucuaucagaagcgcuguaccuuccccauuccagaacacacuacagaauguacuggcagcagccacgaaaaggaacugcaacgucacacagaugagggaauuacccacuuuggacucagcaguauucaacguggaguguuuuaaaaaauucgcauguaaccgagaauacugggaagaauuugcagccagcccuaucaggauaacaacugagaaucuaacaaccuaugucacuaaacuaaaggggccaaaagcagcagcgcuguuugcaaaaacccauaaucugcugccacugcaggauguaccaauggauagguucacaguagauaugaaaagggaugugaagguaacuccugguacaaagcauacagaggaaagaccuaaggugcagguuauacaggcggcugaacccuuggcaacagcguaccuauguggaauucacagagaacugguuaggagauugaacgccguccuccuacccaaugugcauacacuauuugacaugucugccgaggacuucgaugccauuauagccgcacacuucaagccaggagacgcuguuuuagaaacggacauagccuccuuugauaagagccaagaugauucacuugcgcuuaccgccuuaaugcuguuagaagauuugggaguggaucacucccuguuggacuugauagaggcugcuuucggagagauuuccagcugucaucugccgacagguacgcgcuucaaguucggcgcuaugaugaaauccgguauguuccuaacucuguucgucaacacguuguuaaauaucaccaucgcuagccggguguuggaagaucgucugacaaaauccgcaugcgcggccuucaucggcgacgacaacauaauacauggugucgucuccgaugaauugauggcagccagaugcgcuacuuggaugaacauggaagugaagaucauagaugcaguuguaucccagaaagcuccuuacuuuuguggaggguuuauacugcaugauacugugacaggaacagcuugcagaguggcggacccgcuaaaaagguuauuuaaauugggcaaaccguuagcggcaggugacgaacaagaugaagacagaagacgggcgcuggcugaugaaguaaucagauggcaacgaacagggcuaauagaugagcuggagaaagcgguguacucuagguacgaagugcaggguauaucaguugcgguaauguccauggccaccuuugcaagcuccagauccaacuucgagaagcucagaggacccgucauaacuuuguacggcgguccuaaauagguacgcacuacagcuaccuauuuugcagaagccgacagcagguaccuaaauaccaaucagccauaauggaguuuaucccaacccaaacuuucuacaauaggagguaccagccucgaccuuggacuccgcgcccuacuauccaaguuaucagacccagaccgcguccgcaaaggaaagccgggcaacuugcccagcugaucucagcaguuaauaaacugacaaugcgcgcgguaccucaacagaagccgcgcaagaaucggaagaauaagaagcaaaagcaaaagcagcaggcgccacgaaacaacaugaaucaaaagaagcagcccccuaaaaagaaaccggcucaaaagaaaaagaagccgggccguagagagagaaugugcaugaaaaucgaaaaugauugcaucuucgaagucaagcaugaagguaagguaacagguuacgcgugcuugguaggggacaaaguaaugaagccagcacacguaaaggggaccaucgauaaugcggaccuggccaaauuggccuucaagcggucaucuaaguacgaccuugaaugcgcgcagauacccgugcacaugaaguccgacgcuucgaaguucacccaugagaaaccggagggguacuacaacuggcaccacggagcaguacaguacucaggaggccgguucaccaucccuacaggugcgggcaaaccaggggacagcgguagaccgaucuucgacaacaaggggcgcgugguggccauaguuuuaggaggagcuaaugaaggagcccguacagcccucucgguggugaccuggaacaaagacaucgucacgaaaaucaccccugagggggccgaagaguggagucuugccauuccaguuaugugccugcuggcaaauaccacguuccccugcucccagcccccuugcacacccugcugcuacgaaaaagagccggagaaaacccugcgcaugcuagaagacaacgucaugagccccggguacuaucagcugcuacaagcauccuuaacauguucuccccgccgccagcgacgcaguauuaaggacaacuucaaugucuauaaagccauaagaccguaccuagcucacugucccgacuguggagaagggcacucgugccauagucccguagcgcuagaacgcaucagaaacgaagcgacagacgggacgcugaaaauccagguuuccuugcaaaucggaauaaagacggaugauagccaugauuggaccaagcugcguuacauggacaaucauaugccagcagacgcagagagggccaggcuauuuguaagaacgucagcaccgugcacgauuacuggaacaaugggacacuucauccuggcccgauguccgaaaggagaaacucugacggugggauucacugacgguaggaagaucagucacucauguacgcacccauuucaccacgacccuccugugauaggccgggaaaaauuucauucccgaccgcagcacgguagagaacuaccuugcagcacguacgcgcagagcaccgcugcaacugccgaggagauagagguacauaugcccccagacaccccagaucgcacauugaugucacaacaguccgguaauguaaagaucacagucaauagucagacggugcgguacaaguguaauugcggugacucaaaugaaggacuaaccacuacagacaaagugauuaauaacugcaagguugaucaaugccaugccgcggucaccaaucacaaaaaauggcaguauaauuccccucuggucccgcguaaugcugaacucggggaccgaaaaggaaaaguucacauuccguuuccucuggcaaaugugacaugcagggugccuaaggcaaggaaccccaccgugacguacggaaaaaaccaagucaucaugcugcuguauccugaccacccaacgcuccuguccuaccggaauaugggagaagaaccaaacuaucaagaagagugggugacgcauaagaaggagaucagguuaaccgugccgacugaagggcucgaggucacguggggcaacaacgagccguacaaguauuggccgcaguuauccacaaacgguacagcccacggccacccgcaugagauaauuuuguauuauuaugagcuguacccuacuaugacugugguaguugugucaguggccucguucguacuccugucgaugguggguguggcaguggggaugugcaugugugcacgacgcagaugcauuacaccguacgaacugacaccaggagcuaccgucccuuuccugcuuagccuaauaugcugcauuagaacagcuaaagcggccacauaccaagaggcugcgguauaccuguggaacgagcagcagccuuuguuuuggcugcaagcccuuauuccgcuggcagcccugauuguccuaugcaacugucugagacucuuaccaugcuuuuguaaaacguugacuuuuuuagccguaaugagcgucggugcccacacugugagcgcguacgaacacguaacagugaucccgaacacggugggaguaccguauaagacucuagucaacagaccgggcuacagccccaugguacuggagauggagcuucugucagucacuuuggagccaacgcuaucgcuugauuacaucacgugcgaguauaaaaccgucaucccgucuccguacgugaaaugcugcgguacagcagagugcaaggacaagagccuaccugauuacagcuguaaggucuucaccggcgucuacccauucauguggggcggcgccuacugcuucugcgacacugaaaauacgcaauugagcgaagcacauguggagaaguccgaaucaugcaaaacagaauuugcaucagcauauagggcucauaccgcauccgcaucagcuaagcuccgcguccuuuaccaaggaaauaauguuacuguaucugcuuaugcaaacggcgaucaugccgucacaguuaaggacgcuaaauucauuguggggccaaugucuucagccuggacaccuuuugacaauaaaaucgugguguacaaaggcgacgucuacaacauggacuacccgcccuucggcgcaggaagaccaggacaauuuggcgacauccaaagucgcacgccugagagcgaagacgucuaugcuaacacacaacugguacugcagagaccguccgcggguacggugcacgugccguacucucaggcaccaucuggcuucaaguauuggcuaaaagaacgaggggcgucgcugcagcacacagcaccauuuggcugucaaauagcaacaaacccgguaagagcgaugaacugcgccguagggaacaugccuaucuccaucgacauaccggacgcggccuucacuagggucgucgacgcgccaucuuuaacggacaugucgugugagguaccagccugcacccacuccucagacuuugggggcguagccaucauuaaauaugcagccagcaagaaaggcaagugugcggugcauucgaugacuaacgccgucacuauucgggaagcugaaauagaaguagaagggaacucucaguugcaaaucucuuuuucgacggcccuagccagcgccgaauuccgcguacaagucuguucuacacaaguacacugugcagccgagugccauccaccgaaagaccauauagucaauuacccggcgucacacaccacccucgggguccaagacauuuccguuacggcgaugucaugggugcagaagaucacgggaggugugggacugguugucgcuguugcagcacugauccuaaucguggugcuaugcgugucguuuagcaggcacuaacuugacaacuagguacgaagguauauguguccccuaagagacacaccacauauagcuaagaaucaauagauaaguauagaucaaagggcugaacaaccccugaauaguaacaaaauauaaaaaucaacaaaaaucauaaaauagaaaaccagaaacagaaguagguaagaagguauauguguccccuaagagacacaccauauauagcuaagaaucaauagauaaguauagaucaaagggcugaauaaccccugaauaauaacaaaauauaaaaaucaauaaaaaucauaaaauagaaaaccauaaacagaaguaguucaaagggcuauaaaaccccugaauaguaacaaaacauaaaacuaauaaaaaucaaaugaauaccauaauuggcaaucggaagagauguagguacuuaagcuuccuaaaagcagccgaacucgcuuugagauguaggcguagcacaccgaacucuuccauaauucuccgaacccacagggacguaggagauguucaaaguggcuauaaaacccugaacaguaauaaaacauaaaauuaauaaggaucaaaugaguaccauaauuggcaaacggaagagauguagguacuuaagcuuccuaaaagcagccgaacucacuuugagauguaggcauagcauaccgaacucuuccacaauucuccguacccauagggacguaggagauguuauuuuguuuuuaauauuuc

SEQ ID NO: 2 - RNA, 11874 nt, Alphavirus Chikungunya virus
auggcugcgugagacacacguagccuaccaguuucuuacugcucuacucugcaaagcaagagauuaauaacccaucauggauucuguguacguggacauagacgcugacagcgccuuuuugaaggcccugcaacgugcguaccccauguuugagguggaaccuaggcaggucacaucgaaugaccaugcuaaugcuagagcguucucgcaucuagccauaaaacuaauagagcaggaaauugaucccgacucaaccauccuggauauagguagugcgccagcaaggaggaugaugucggacaggaaguaccacugcguuugcccgaugcgcagcgcagaagaucccgagagacucgcuaauuaugcgagaaagcucgcaucugccgcaggaaaaguccuggacagaaacauuucuggaaagaucggggacuuacaagcggugauggccgugccagacacggagacgccaacauuuugcuuacacacagaugucucauguagacagagagcagacgucgcgauauaccaagacgucuaugcuguacacgcacccacgucgcuauaccaccaggcgauuaaaggaguccgaguggcguacuggguaggguucgacacaaccccguucauguacaacgcuauggcgggugccuaccccucauacucgacaaauugggcggaugagcagguacugaaggcuaagaacauaggauuauguucaacagaccugacggaagguagacgaggcaaauugucuaucaugagagggaaaaagcuaaaaccgugcgaccgugugcuguucucaguagggucaacgcuuuacccggaaagccgcacgcuacuuaagagcuggcaccuaccaucgguguuccaucuaaagggcaagcuuagcuucacaugccgcugugacacagugguuucgugugagggcuacgucguuaagagaauaacgaugagcccaggccuuuauggaaaaaccauaggguaugcgguaacccaccacgcagacggauucuugaugugcaagacuaccgacacgguugacggcgaaagagugucauucucggugugcacguacgugccggcgaccauuugugaucaaaugaccggcauccuugcuacagaagucacgccggaggaugcacagaagcuguugguggggcugaaccagaggauagugguuaacggcagaacgcaacggaacacgaacaccaugaagaacuaccuacuucccguggucgcccaggccuucaguaagugggcaaaggagugccggaaggacauggaagaugagaagcuucugggggucagagaaagaacacuaaccugcugcugucuaugggcauuuaagaagcagaaaacacacacggucuacaagaggccugauacccagucaauccagaagguucaggccgaauuugacagcuuuguaguaccgggccuguggucguccggguugucaaucccguugaggacuagaaucaagugguuguuacgcaaggugccgaaaacagaccugaucccauacagcgggaaugcccaagaagcccaggaugcagaaaaagaagcagaggaagaacgagaagcagaacugacucaugaggcucuaccaccccuacaggcagcacaggaagauguccaggucgaaaucgacguggaacagcuugaggauagagcuggugcuggaauaauagagacuccgagaggcgcuaucaaaguuacugcccaacuaacagaccacgucgugggggaguaccugguacuuuccccgcagaccguacuacgcagccagaagcucagccugauccacgcuuuagcggagcaagugaagacguguacgcacagcggacgagcagggagguaugcggucgaagcguacgauggccgaguccuagugcccucaggcuaugcaauuucgccugaagacuuccagagucuaagcgaaagcgcaacgaugguguacaacgaaagagaguucguaaacagaaaguuacaccacauugcgaugcacggaccagcccugaacacugacgaagagucguaugagcuggugagggcagagaggacagaacacgaguacgucuacgacguggaccagagaagaugcuguaagaaggaagaagcugcaggacugguacuggugggcgacuugacuaauccgcccuaccacgaauucgcauacgaagggcuaaaaauucgccccgccugcccauacaaaauugcagucauaggagucuucgggguaccaggaucuggcaagucagccauuaucaagaaccuaguuaccaggcaagaccuggugacuagcggaaagaaagaaaacugccaagaaaucagcaccgacgugaugagacagagaggucuagagauaucugcacguacgguagauucgcugcucuugaauggaugcaacagaccagucgacguguuguacguagacgaggcguuugcgugccacucuggaacguuacuugcuuugaucgccuuggugagaccaagacagaaaguuguacuuuguggugacccgaagcagugcggcuucuucaauaugaugcagaugaaagucaacuacaaucauaacaucugcacccaaguguaccacaaaaguaucuccaggcgguguacacugccugugacugccauugugucaucguugcauuacgaaggcaaaaugcgcacuacgaaugaguacaacaugccgauuguaguggacacuacaggcucaacgaaaccugacccuggagaccucguguuaacgugcuucagaggguggguuaaacaacugcaaauugacuaucguggacacgaggucaugacagcagccgcaucccaaggguuaacuagaaaaggaguuuacgcaguuaggcaaaaaguuaacgaaaacccacucuaugcaucaacaucagagcacgucaacguacuccuaacgcguacggaagguaaacugguauggaagacacucucuggugacccguggauaaagacgcugcagaacccaccgaaaggaaacuucaaagcaacuauuaaggagugggagguggagcacgcaucgauaauggcgggcaucugcagucaccaagugaccuuugacacauuccaaaacaaagccaacguuugcugggcuaagagcuuggucccuauccucgaaacagcggggauaaaacuaaaugauaggcaguggucccagauaauucaagccuucaaagaagacaaagcauacucacccgaaguagcccugaaugaaauaugcacgcgcauguaugggguggaucuagacagugggcuauucucuaaaccguugguaucuguguauuacgcggauaaccauugggauaauaggccgggaggaaagauguucggauucaacccugaggcagcguccauucuagaaagaaaguacccauuuacaaaaggaaaguggaacaucaacaagcagaucugcgugacuaccaggaggauagaagacuucaacccuaccaccaacauuauaccggucaacaggagacuaccacacucauuaguggccgaacaccgcccaguaaaaggggaaagaauggaauggcugguuaacaagauaaacggacaccacguacuccugguuagcggcuauaaccuugcacugccuacuaagagagucaccuggguagcgccacuagguguccgcggagcggacuauacauacaaccuagagcugggucuaccagcaacgcuugguagguaugaccuaguggucauaaacauccacacaccuuuucgcauacaccauuaccaacagugcguagaucacgcaaugaaacugcaaaugcuagggggugacucacugagacugcucaaaccggguggcucucuauugaucagagcauacgguuacgcagauagaaccagugaacgagucaucugcguacugggacgcaaguuuagaucgucuagagcauugaaaccaccaugugucaccaguaauacugagauguuuuuccuauuuagcaauuuugacaauggcagaaggaauuuuacaacgcaugucaugaacaaucaacugaaugcagccuuuguaggacaggccacccgagcaggaugugcaccaucguaccggguaaaacgcauggacaucgcgaagaacgaugaagagugcgugguuaacgccgccaacccucgcggguuaccaggugacgguguuugcaaggcaguauauaaaaaguggccggaguccuuuaaaaacagugcaacaccaguaggaaccgcaaaaacaguuaugugcgguacguauccaguaauccacgccguaggaccaaacuucucaaauuauucggagucugaaggggaccgggaauuggcggcugccuaucgagaagucgcaaaggaaguaacuagacugggaguaaauagcguagcuauaccucuccucuccacagguguauacucaggagggaaagacaggcuaacccagucacugaaccaccucuuuacagccauggacucgacggaugcagacguggucaucuacugccgagacaaggaaugggagaagaaaauaucugaggccauacagaugcggacccaaguggagcugcuggaugagcacaucuccauagacugcgaugucauucgcgugcacccugacaguagcuuggcaggcagaaaaggauacagcaccacggaaggcgcacuguauucauaucuagaagggacacguuuucaccagacggcaguggauauggcagagauauacacuauguggccaaagcaaacagaggccaaugagcaagucugccuauaugcccugggggaaaguauugaaucaaucaggcagaaaugcccgguggaugaugcagacgcaucaucucccccgaaaacugucccgugucuuugccgguaugccaugacuccugaacgcgucacccgacuucgcaugaaccaugucacaaauauaauuguguguucuucauuuccccuuccaaaguacaagauagaaggagugcaaaaagucaaaugcuccaagguaauguuauucgaucacaaugugccaucgcgcguaaguccaagggcguaucgugcagccgcaggcggaaaccuugcggccgugucugacuggguaaugagcaccguaccugucgcgccgccuagaagaaggagagggagaaaccugacugugacaugugacgagagagaagggaauauaacacccauggcuagcguccgauucuuuagagcagagcuguguccggccguacaagaaacagcggagacgcgugacacagcuauuucccuucaggcaccgccaaguaccaccauggaacugagccauccaccgaucuccuucggagcaccaagcgagacguuccccaucacauuuggggacuucgacgaaggagaaaucgaaagcuugucuucugagcuacuaacuuucggagacuuccuacccggugaaguggaugaucugacagauagcgacugguccacgugcccagacacggacgacgaguuaugacuagacagggcagguggguauauauucucgucggacacugguccaggccauuuacaacagaagucgguacgccagucagugcugccgguaaacacccuggaggaaguccacgaggagaaguguuacccaccuaagcuggaugaauuaaaggagcaacuacuacuuaagaaacuccaggagagugcguccauggccaauagaagcagguaucagucacgcaaaguggaaaauaugaaagcaacaaucauccagagacuaaagagaggcuguaaacuguauuuaauggcagagaccccgaaagucccgacuuaucggaccauauacccggcgccuguguacucgccuccgaucaauguccgauuguccaaccccgaguccgcaguggcagcauguaaugaguucuuagcuagaaacuacccaacuguuucaucauaccaaaucaccgacgaguaugaugcauaucuagacaugguggacgggucggagaguugcuuggaccgagcgacauucaauccgucaaaacuuaggagcuacccgaaacaacaugcuuaucacgcgccuucuaucagaagcgcuguaccuuccccauuccagaacacacuacagaauguacuggcagcagccacgaaaaggaacugcaacgucacacagaugagggaauuacccacuuuggacucagcaguauucaacguggaguguuuuaaaaaauucgcauguaaccgagaauacugggaagaauuugcagccagcccuaucaggauaacaacugagaaucuaacaaccuaugucacuaaacuaaaggggccaaaagcagcagcgcuguuugcaaaaacccauaaucugcugccacugcaggauguaccaauggauagguucacaguagauaugaaaagggaugugaagguaacuccugguacaaagcauacagaggaaagaccuaaggugcagguuauacaggcggcugaacccuuggcaacagcguaccuauguggaauucacagagaacugguuaggagauugaacgccguccuccuacccaaugugcauacacuauuugacaugucugccgaggacuucgaugccauuauagccgcacacuucaagccaggagacgcuguuuuagaaacggacauagccuccuuugauaagagccaagaugauucacuugcgcuuaccgccuuaaugcuguuagaagauuugggaguggaucacucccuguuggacuugauagaggcugcuuucggagagauuuccagcugucaucugccgacagguacgcgcuucaaguucggcgcuaugaugaaauccgguauguuccuaacucuguucgucaacacguuguuaaauaucaccaucgcuagccggguguuggaagaucgucugacaaaauccgcaugcgcggccuucaucggcgacgacaacauaauacauggugucgucuccgaugaauugauggcagccagaugcgcuacuuggaugaacauggaagugaagaucauagaugcaguuguaucccagaaagcuccuuacuuuuguggaggguuuauacugcaugauacugugacaggaacagcuugcagaguggcggacccgcuaaaaagguuauuuaaauugggcaaaccguuagcggcaggugacgaacaagaugaagacagaagacgggcgcuggcugaugaaguaaucagauggcaacgaacagggcuaauagaugagcuggagaaagcgguguacucuagguacgaagugcaggguauaucaguugcgguaauguccauggccaccuuugcaagcuccagauccaacuucgagaagcucagaggacccgucauaacuuuguacggcgguccuaaauagguacgcacuacagcuaccuauuuugcagaagccgacagcagguaccuaaauaccaaucagccauaauggaguuuaucccaacccaaacuuucuacaauaggagguaccagccucgaccuuggacuccgcgcccuacuauccaaguuaucagacccagaccgcguccgcaaaggaaagccgggcaacuugcccagcugaucucagcaguuaauaaacugacaaugcgcgcgguaccucaacagaagccgcgcaagaaucggaagaauaagaagcaaaagcaaaagcagcaggcgccacgaaacaacaugaaucaaaagaagcagcccccuaaaaagaaaccggcucaaaagaaaaagaagccgggccguagagagagaaugugcaugaaaaucgaaaaugauugcaucuucgaagucaagcaugaagguaagguaacagguuacgcgugcuugguaggggacaaaguaaugaagccagcacacguaaaggggaccaucgauaaugcggaccuggccaaauuggccuucaagcggucaucuaaguacgaccuugaaugcgcgcagauacccgugcacaugaaguccgacgcuucgaaguucacccaugagaaaccggagggguacuacaacuggcaccacggagcaguacaguacucaggaggccgguucaccaucccuacaggugcgggcaaaccaggggacagcgguagaccgaucuucgacaacaaggggcgcgugguggccauaguuuuaggaggagcuaaugaaggagcccguacagcccucucgguggugaccuggaacaaagacaucgucacgaaaaucaccccugagggggccgaagaguggagucuugccauuccaguuaugugccugcuggcaaauaccacguuccccugcucccagcccccuugcacacccugcugcuacgaaaaagagccggagaaaacccugcgcaugcuagaagacaacgucaugagccccggguacuaucagcugcuacaagcauccuuaacauguucuccccgccgccagcgacgcaguauuaaggacaacuucaaugucuauaaagccauaagaccguaccuagcucacugucccgacuguggagaagggcacucgugccauagucccguagcgcuagaacgcaucagaaacgaagcgacagacgggacgcugaaaauccagguuuccuugcaaaucggaauaaagacggaugauagccaugauuggaccaagcugcguuacauggacaaucauaugccagcagacgcagagagggccaggcuauuuguaagaacgucagcaccgugcacgauuacuggaacaaugggacacuucauccuggcccgauguccgaaaggagaaacucugacggugggauucacugacgguaggaagaucagucacucauguacgcacccauuucaccacgacccuccugugauaggccgggaaaaauuucauucccgaccgcagcacgguagagaacuaccuugcagcacguacgcgcagagcaccgcugcaacugccgaggagauagagguacauaugcccccagacaccccagaucgcacauugaugucacaacaguccgguaauguaaagaucacagucaauagucagacggugcgguacaaguguaauugcggugacucaaaugaaggacuaaccacuacagacaaagugauuaauaacugcaagguugaucaaugccaugccgcggucaccaaucacaaaaaauggcaguauaauuccccucuggucccgcguaaugcugaacucggggaccgaaaaggaaaaguucacauuccguuuccucuggcaaaugugacaugcagggugccuaaggcaaggaaccccaccgugacguacggaaaaaaccaagucaucaugcugcuguauccugaccacccaacgcuccuguccuaccggaauaugggagaagaaccaaacuaucaagaagagugggugacgcauaagaaggagaucagguuaaccgugccgacugaagggcucgaggucacguggggcaacaacgagccguacaaguauuggccgcaguuauccacaaacgguacagcccacggccacccgcaugagauaauuuuguauuauuaugagcuguacccuacuaugacugugguaguugugucaguggccucguucguacuccugucgaugguggguguggcaguggggaugugcaugugugcacgacgcagaugcauuacaccguacgaacugacaccaggagcuaccgucccuuuccugcuuagccuaauaugcugcauuagaacagcuaaagcggccacauaccaagaggcugcgguauaccuguggaacgagcagcagccuuuguuuuggcugcaagcccuuauuccgcuggcagcccugauuguccuaugcaacugucugagacucuuaccaugcuuuuguaaaacguugacuuuuuuagccguaaugagcgucggugcccacacugugagcgcguacgaacacguaacagugaucccgaacacggugggaguaccguauaagacucuagucaacagaccgggcuacagccccaugguacuggagauggagcuucugucagucacuuuggagccaacgcuaucgcuugauuacaucacgugcgaguauaaaaccgucaucccgucuccguacgugaaaugcugcgguacagcagagugcaaggacaagagccuaccugauuacagcuguaaggucuucaccggcgucuacccauucauguggggcggcgccuacugcuucugcgacacugaaaauacgcaauugagcgaagcacauguggagaaguccgaaucaugcaaaacagaauuugcaucagcauauagggcucauaccgcauccgcaucagcuaagcuccgcguccuuuaccaaggaaauaauguuacuguaucugcuuaugcaaacggcgaucaugccgucacaguuaaggacgcuaaauucauuguggggccaaugucuucagccuggacaccuuuugacaauaaaaucgugguguacaaaggcgacgucuacaacauggacuacccgcccuucggcgcaggaagaccaggacaauuuggcgacauccaaagucgcacgccugagagcgaagacgucuaugcuaacacacaacugguacugcagagaccguccgcggguacggugcacgugccguacucucaggcaccaucuggcuucaaguauuggcuaaaagaacgaggggcgucgcugcagcacacagcaccauuuggcugucaaauagcaacaaacccgguaagagcgaugaacugcgccguagggaacaugccuaucuccaucgacauaccggacgcggccuucacuagggucgucgacgcgccaucuuuaacggacaugucgugugagguaccagccugcacccacuccucagacuuugggggcguagccaucauuaaauaugcagccagcaagaaaggcaagugugcggugcauucgaugacuaacgccgucacuauucgggaagcugaaauagaaguagaagggaacucucaguugcaaaucucuuuuucgacggcccuagccagcgccgaauuccgcguacaagucuguucuacacaaguacacugugcagccgagugccauccaccgaaagaccauauagucaauuacccggcgucacacaccacccucgggguccaagacauuuccguuacggcgaugucaugggugcagaagaucacgggaggugugggacugguugucgcuguugcagcacugauccuaaucguggugcuaugcgugucguuuagcaggcacuaacuugacaacuagguacgaagguauauguguccccuaagagacacaccacauauagcuaagaaucaauagauaaguauagaucaaagggcugaacaaccccugaauaguaacaaaauauaaaaaucaacaaaaaucauaaaauagaaaaccagaaacagaaguagguaagaagguauauguguccccuaagagacacaccauauauagcuaagaaucaauagauaaguauagaucaaagggcugaauaaccccugaauaauaacaaaauauaaaaaucaauaaaaaucauaaaauagaaaaccauaaacagaaguaguucaaagggcuauaaaaccccugaauaguaacaaaacauaaaacuaauaaaaaucaaaugaauaccauaauuggcaaucggaagagauguagguacuuaagcuuccuaaaagcagccgaacucgcuuugagauguaggcguagcacaccgaacucuuccauaauucuccgaacccacagggacguaggagauguucaaaguggcuauaaaacccugaacaguaauaaaacauaaaauuaauaaggaucaaaugaguaccauaauuggcaaacggaagagauguagguacuuaagcuuccuaaaagcagccgaacucacuuugagauguaggcauagcauaccgaacucuuccacaauucuccguacccauagggacguaggagauguuauuuuguuuuuaauauuuc

SEQ ID NO: 3 - RNA, 11856 nt, Alphavirus Chikungunya virus
auggcugcgugagacacacguagccuaccaguuucuuacugcucuacucugcaaagcaagagauuaauaacccaucauggauucuguguacguggacauagacgcugacagcgccuuuuugaaggcccugcaacgugcguaccccauguuugagguggaaccuaggcaggucacaucgaaugaccaugcuaaugcuagagcguucucgcaucuagccauaaaacuaauagagcaggaaauugaucccgacucaaccauccuggauauagguagugcgccagcaaggaggaugaugucggacaggaaguaccacugcguuugcccgaugcgcagcgcagaagaucccgagagacucgcuaauuaugcgagaaagcucgcaucugccgcaggaaaaguccuggacagaaacauuucuggaaagaucggggacuuacaagcggugauggccgugccagacacggagacgccaacauuuugcuuacacacagaugucucauguagacagagagcagacgucgcgauauaccaagacgucuaugcuguacacgcacccacgucgcuauaccaccaggcgauuaaaggaguccgaguggcguacuggguaggguucgacacaaccccguucauguacaacgcuauggcgggugccuaccccucauacucgacaaauugggcggaugagcagguacugaaggcuaagaacauaggauuauguucaacagaccugacggaagguagacgaggcaaauugucuaucaugagagggaaaaagcuaaaaccgugcgaccgugugcuguucucaguagggucaacgcuuuacccggaaagccgcacgcuacuuaagagcuggcaccuaccaucgguguuccaucuaaagggcaagcuuagcuucacaugccgcugugacacagugguuucgugugagggcuacgucguuaagagaauaacgaugagcccaggccuuuauggaaaaaccauaggguaugcgguaacccaccacgcagacggauucuugaugugcaagacuaccgacacgguugacggcgaaagagugucauucucggugugcacguacgugccggcgaccauuugugaucaaaugaccggcauccuugcuacagaagucacgccggaggaugcacagaagcuguugguggggcugaaccagaggauagugguuaacggcagaacgcaacggaacacgaacaccaugaagaacuaccuacuucccguggucgcccaggccuucaguaagugggcaaaggagugccggaaggacauggaagaugagaagcuucugggggucagagaaagaacacuaaccugcugcugucuaugggcauuuaagaagcagaaaacacacacggucuacaagaggccugauacccagucaauccagaagguucaggccgaauuugacagcuuuguaguaccgggccuguggucguccggguugucaaucccguugaggacuagaaucaagugguuguuacgcaaggugccgaaaacagaccugaucccauacagcgggaaugcccaagaagcccaggaugcagaaaaagaagcagaggaagaacgagaagcagaacugacucaugaggcucuaccaccccuacaggcagcacaggaagauguccaggucgaaaucgacguggaacagcuugaggauagagcuggugcuggaauaauagagacuccgagaggcgcuaucaaaguuacugcccaacuaacagaccacgucgugggggaguaccugguacuuuccccgcagaccguacuacgcagccagaagcucagccugauccacgcuuuagcggagcaagugaagacguguacgcacagcggacgagcagggagguaugcggucgaagcguacgauggccgaguccuagugcccucaggcuaugcaauuucgccugaagacuuccagagucuaagcgaaagcgcaacgaugguguacaacgaaagagaguucguaaacagaaaguuacaccacauugcgaugcacggaccagcccugaacacugacgaagagucguaugagcuggugagggcagagaggacagaacacgaguacgucuacgacguggaccagagaagaugcuguaagaaggaagaagcugcaggacugguacuggugggcgacuugacuaauccgcccuaccacgaauucgcauacgaagggcuaaaaauucgccccgccugcccauacaaaauugcagucauaggagucuucgggguaccaggaucuggcaagucagccauuaucaagaaccuaguuaccaggcaagaccuggugacuagcggaaagaaagaaaacugccaagaaaucagcaccgacgugaugagacagagaggucuagagauaucugcacguacgguagauucgcugcucuugaauggaugcaacagaccagucgacguguuguacguagacgaggcguuugcgugccacucuggaacguuacuugcuuugaucgccuuggugagaccaagacagaaaguuguacuuuguggugacccgaagcagugcggcuucuucaauaugaugcagaugaaagucaacuacaaucauaacaucugcacccaaguguaccacaaaaguaucuccaggcgguguacacugccugugacugccauugugucaucguugcauuacgaaggcaaaaugcgcacuacgaaugaguacaacaugccgauuguaguggacacuacaggcucaacgaaaccugacccuggagaccucguguuaacgugcuucagaggguggguuaaacaacugcaaauugacuaucguggacacgaggucaugacagcagccgcaucccaaggguuaacuagaaaaggaguuuacgcaguuaggcaaaaaguuaacgaaaacccacucuaugcaucaacaucagagcacgucaacguacuccuaacgcguacggaagguaaacugguauggaagacacucucuggugacccguggauaaagacgcugcagaacccaccgaaaggaaacuucaaagcaacuauuaaggagugggagguggagcacgcaucgauaauggcgggcaucugcagucaccaagugaccuuugacacauuccaaaacaaagccaacguuugcugggcuaagagcuuggucccuauccucgaaacagcggggauaaaacuaaaugauaggcaguggucccagauaauucaagccuucaaagaagacaaagcauacucacccgaaguagcccugaaugaaauaugcacgcgcauguaugggguggaucuagacagugggcuauucucuaaaccguugguaucuguguauuacgcggauaaccauugggauaauaggccgggaggaaagauguucggauucaacccugaggcagcguccauucuagaaagaaaguacccauuuacaaaaggaaaguggaacaucaacaagcagaucugcgugacuaccaggaggauagaagacuucaacccuaccaccaacauuauaccggucaacaggagacuaccacacucauuaguggccgaacaccgcccaguaaaaggggaaagaauggaauggcugguuaacaagauaaacggacaccacguacuccugguuagcggcuauaaccuugcacugccuacuaagagagucaccuggguagcgccacuagguguccgcggagcggacuauacauacaaccuagagcugggucuaccagcaacgcuugguagguaugaccuaguggucauaaacauccacacaccuuuucgcauacaccauuaccaacagugcguagaucacgcaaugaaacugcaaaugcuagggggugacucacugagacugcucaaaccggguggcucucuauugaucagagcauacgguuacgcagauagaaccagugaacgagucaucugcguacugggacgcaaguuuagaucgucuagagcauugaaaccaccaugugucaccaguaauacugagauguuuuuccuauuuagcaauuuugacaauggcagaaggaauuuuacaacgcaugucaugaacaaucaacugaaugcagccuuuguaggacaggccacccgagcaggaugugcaccaucguaccggguaaaacgcauggacaucgcgaagaacgaugaagagugcgugguuaacgccgccaacccucgcggguuaccaggugacgguguuugcaaggcaguauauaaaaaguggccggaguccuuuaaaaacagugcaacaccaguaggaaccgcaaaaacaguuaugugcgguacguauccaguaauccacgccguaggaccaaacuucucaaauuauucggagucugaaggggaccgggaauuggcggcugccuaucgagaagucgcaaaggaaguaacuagacugggaguaaauagcguagcuauaccucuccucuccacagguguauacucaggagggaaagacaggcuaacccagucacugaaccaccucuuuacagccauggacucgacggaugcagacguggucaucuacugccgagacaaggaaugggagaagaaaauaucugaggccauacagaugcggacccaaguggagcugcuggaugagcacaucuccauagacugcgaugucauucgcgugcacccugacaguagcuuggcaggcagaaaaggauacagcaccacggaaggcgcacuguauucauaucuagaagggacacguuuucaccagacggcaguggauauggcagagauauacacuauguggccaaagcaaacagaggccaaugagcaagucugccuauaugcccugggggaaaguauugaaucaaucaggcagaaaugcccgguggaugaugcagacgcaucaucucccccgaaaacugucccgugucuuugccgguaugccaugacuccugaacgcgucacccgacuucgcaugaaccaugucacaaauauaauuguguguucuucauuuccccuuccaaaguacaagauagaaggagugcaaaaagucaaaugcuccaagguaauguuauucgaucacaaugugccaucgcgcguaaguccaagggaauacagaucuucccaggagucuguacaggaagugaguacgacaacgucauugacgcauagccaguuugaucuaagcgccgauggcgagacacugccugucccgucagaccuggaugcugacgccccagcccuagaaccggcccuagacgacggggcgguacauacauuaccaaccauaaucggaaaccuugcggccgugucugacuggguaaugagcaccguaccugucgcgccgccuagaagaaggagagggagaaaccugacugugacaugugacgagagagaagggaauauaacacccauggcuagcguccgauucuuuagagcagagcuguguccggccguacaagaaacagcggagacgcgugacacagcuauuucccuucaggcaccgccaaguaccaccauggaacugagccauccaccgaucuccuucggagcaccaagcgagacguuccccaucacauuuggggacuucgacgaaggagaaaucgaaagcuugucuucugagcuacuaacuuucggagacuuccuacccggugaaguggaugaucugacagauagcgacugguccacgugcccagacacggacgacgaguuaugacuagacagggcagguggguauauauucucgucggacacugguccaggccauuuacaacagaagucgguacgccagucagugcugccgguaaacacccuggaggaaguccacgaggagaaguguuacccaccuaagcuggaugaauuaaaggagcaacuacuacuuaagaaacuccaggagagugcguccauggccaauagaagcagguaucagucacgcaaaguggaaaauaugaaagcaacaaucauccagagacuaaagagaggcuguaaacuguauuuaauggcagagaccccgaaagucccgacuuaucggaccauauacccggcgccuguguacucgccuccgaucaauguccgauuguccaaccccgaguccgcaguggcagcauguaaugaguucuuagcuagaaacuacccaacuguuucaucauaccaaaucaccgacgaguaugaugcauaucuagacaugguggacgggucggagaguugcuuggaccgagcgacauucaauccgucaaaacuuaggagcuacccgaaacaacaugcuuaucacgcgccuucuaucagaagcgcuguaccuuccccauuccagaacacacuacagaauguacuggcagcagccacgaaaaggaacugcaacgucacacagaugagggaauuacccacuuuggacucagcaguauucaacguggaguguuuuaaaaaauucgcauguaaccgagaauacugggaagaauuugcagccagcccuaucaggauaacaacugagaaucuaacaaccuaugucacuaaacuaaaggggccaaaagcagcagcgcuguuugcaaaaacccauaaucugcugccacugcaggauguaccaauggauagguucacaguagauaugaaaagggaugugaagguaacuccugguacaaagcauacagaggaaagaccuaaggugcagguuauacaggcggcugaacccuuggcaacagcguaccuauguggaauucacagagaacugguuaggagauugaacgccguccuccuacccaaugugcauacacuauuugacaugucugccgaggacuucgaugccauuauagccgcacacuucaagccaggagacgcuguuuuagaaacggacauagccuccuuugauaagagccaagaugauucacuugcgcuuaccgccuuaaugcuguuagaagauuugggaguggaucacucccuguuggacuugauagaggcugcuuucggagagauuuccagcugucaucugccgacagguacgcgcuucaaguucggcgcuaugaugaaauccgguauguuccuaacucuguucgucaacacguuguuaaauaucaccaucgcuagccggguguuggaagaucgucugacaaaauccgcaugcgcggccuucaucggcgacgacaacauaauacauggugucgucuccgaugaauugauggcagccagaugcgcuacuuggaugaacauggaagugaagaucauagaugcaguuguaucccagaaagcuccuuacuuuuguggaggguuuauacugcaugauacugugacaggaacagcuugcagaguggcggacccgcuaaaaagguuauuuaaauugggcaaaccguuagcggcaggugacgaacaagaugaagacagaagacgggcgcuggcugaugaaguaaucagauggcaacgaacagggcuaauagaugagcuggagaaagcgguguacucuagguacgaagugcaggguauaucaguugcgguaauguccauggccaccuuugcaagcuccagauccaacuucgagaagcucagaggacccgucauaacuuuguacggcgguccuaaauagguacgcacuacagcuaccuauuuugcagaagccgacagcagguaccuaaauaccaaucagccauaauggaguuuaucccaacccaaacuuucuacaauaggagguaccagccucgaccuuggacuccgcgcccuacuauccaaguuaucagacccagaccgcguccgcaaaggaaagccgggcaacuugcccagcugaucucagcaguuaauaaacugacaaugcgcgcgguaccucaacagaagccgcgcaagaaucggaagaauaagaagcaaaagcaaaagcagcaggcgccacgaaacaacaugaaucaaaagaagcagcccccuaaaaagaaaccggcucaaaagaaaaagaagccgggccguagagagagaaugugcaugaaaaucgaaaaugauugcaucuucgaagucaagcaugaagguaagguaacagguuacgcgugcuugguaggggacaaaguaaugaagccagcacacguaaaggggaccaucgauaaugcggaccuggccaaauuggccuucaagcggucaucuaaguacgaccuugaaugcgcgcagauacccgugcacaugaaguccgacgcuucgaaguucacccaugagaaaccggagggguacuacaacuggcaccacggagcaguacaguacucaggaggccgguucaccaucccuacaggugcgggcaaaccaggggacagcgguagaccgaucuucgacaacaaggggcgcgugguggccauaguuuuaggaggagcuaaugaaggagcccguacagcccucucgguggugaccuggaacaaagacaucgucacgaaaaucaccccugagggggccgaagaguggagucuugccauuccaguuaugugccugcuggcaaauaccacguuccccugcucccagcccccuugcacacccugcugcuacgaaaaagagccggagaaaacccugcgcaugcuagaagacaacgucaugagccccggguacuaucagcugcuacaagcauccuuaacauguucuccccgccgccagcgacgcaguauuaaggacaacuucaaugucuauaaagccauaagaccguaccuagcucacugucccgacuguggagaagggcacucgugccauagucccguagcgcuagaacgcaucagaaacgaagcgacagacgggacgcugaaaauccagguuuccuugcaaaucggaauaaagacggaugauagccaugauuggaccaagcugcguuacauggacaaucauaugccagcagacgcagagagggccaggcuauuuguaagaacgucagcaccgugcacgauuacuggaacaaugggacacuucauccuggcccgauguccgaaaggagaaacucugacggugggauucacugacgguaggaagaucagucacucauguacgcacccauuucaccacgacccuccugugauaggccgggaaaaauuucauucccgaccgcagcacgguagagaacuaccuugcagcacguacgcgcagagcaccgcugcaacugccgaggagauagagguacauaugcccccagacaccccagaucgcacauugaugucacaacaguccgguaauguaaagaucacagucaauagucagacggugcgguacaaguguaauugcggugacucaaaugaaggacuaaccacuacagacaaagugauuaauaacugcaagguugaucaaugccaugccgcggucaccaaucacaaaaaauggcaguauaauuccccucuggucccgcguaaugcugaacucggggaccgaaaaggaaaaguucacauuccguuuccucuggcaaaugugacaugcagggugccuaaggcaaggaaccccaccgugacguacggaaaaaaccaagucaucaugcugcuguauccugaccacccaacgcuccuguccuaccggaauaugggagaagaaccaaacuaucaagaagagugggugacgcauaagaaggagaucagguuaaccgugccgacugaagggcucgaggucacguggggcaacaacgagccguacaaguauuggccgcaguuauccacaaacgguacagcccacggccacccgcaugagauaauuuuguauuauuaugagcuguacccuacuaugacugugguaguugugucaguggccucguucguacuccugucgaugguggguguggcaguggggaugugcaugugugcacgacgcagaugcauuacaccguacgaacugacaccaggagcuaccgucccuuuccugcuuagccuaauaugcugcauuagaacagcuaaagcggcguacgaacacguaacagugaucccgaacacggugggaguaccguauaagacucuagucaacagaccgggcuacagccccaugguacuggagauggagcuucugucagucacuuuggagccaacgcuaucgcuugauuacaucacgugcgaguauaaaaccgucaucccgucuccguacgugaaaugcugcgguacagcagagugcaaggacaagagccuaccugauuacagcuguaaggucuucaccggcgucuacccauucauguggggcggcgccuacugcuucugcgacacugaaaauacgcaauugagcgaagcacauguggagaaguccgaaucaugcaaaacagaauuugcaucagcauauagggcucauaccgcauccgcaucagcuaagcuccgcguccuuuaccaaggaaauaauguuacuguaucugcuuaugcaaacggcgaucaugccgucacaguuaaggacgcuaaauucauuguggggccaaugucuucagccuggacaccuuuugacaauaaaaucgugguguacaaaggcgacgucuacaacauggacuacccgcccuucggcgcaggaagaccaggacaauuuggcgacauccaaagucgcacgccugagagcgaagacgucuaugcuaacacacaacugguacugcagagaccguccgcggguacggugcacgugccguacucucaggcaccaucuggcuucaaguauuggcuaaaagaacgaggggcgucgcugcagcacacagcaccauuuggcugucaaauagcaacaaacccgguaagagcgaugaacugcgccguagggaacaugccuaucuccaucgacauaccggacgcggccuucacuagggucgucgacgcgccaucuuuaacggacaugucgugugagguaccagccugcacccacuccucagacuuugggggcguagccaucauuaaauaugcagccagcaagaaaggcaagugugcggugcauucgaugacuaacgccgucacuauucgggaagcugaaauagaaguagaagggaacucucaguugcaaaucucuuuuucgacggcccuagccagcgccgaauuccgcguacaagucuguucuacacaaguacacugugcagccgagugccauccaccgaaagaccauauagucaauuacccggcgucacacaccacccucgggguccaagacauuuccguuacggcgaugucaugggugcagaagaucacgggaggugugggacugguugucgcuguugcagcacugauccuaaucguggugcuaugcgugucguuuagcaggcacuaacuugacaacuagguacgaagguauauguguccccuaagagacacaccacauauagcuaagaaucaauagauaaguauagaucaaagggcugaacaaccccugaauaguaacaaaauauaaaaaucaacaaaaaucauaaaauagaaaaccagaaacagaaguagguaagaagguauauguguccccuaagagacacaccauauauagcuaagaaucaauagauaaguauagaucaaagggcugaauaaccccugaauaauaacaaaauauaaaaaucaauaaaaaucauaaaauagaaaaccauaaacagaaguaguucaaagggcuauaaaaccccugaauaguaacaaaacauaaaacuaauaaaaaucaaaugaauaccauaauuggcaaucggaagagauguagguacuuaagcuuccuaaaagcagccgaacucgcuuugagauguaggcguagcacaccgaacucuuccauaauucuccgaacccacagggacguaggagauguucaaaguggcuauaaaacccugaacaguaauaaaacauaaaauuaauaaggaucaaaugaguaccauaauuggcaaacggaagagauguagguacuuaagcuuccuaaaagcagccgaacucacuuugagauguaggcauagcauaccgaacucuuccacaauucuccguacccauagggacguaggagauguuauuuuguuuuuaauauuuc

SEQ ID NO: 4 - RNA, 12561 nt, Alphavirus Chikungunya virus
auggcugcgugagacacacguagccuaccaguuucuuacugcucuacucugcaaagcaagagauuaauaacccaucauggauucuguguacguggacauagacgcugacagcgccuuuuugaaggcccugcaacgugcguaccccauguuugagguggaaccuaggcaggucacaucgaaugaccaugcuaaugcuagagcguucucgcaucuagccauaaaacuaauagagcaggaaauugaucccgacucaaccauccuggauauagguagugcgccagcaaggaggaugaugucggacaggaaguaccacugcguuugcccgaugcgcagcgcagaagaucccgagagacucgcuaauuaugcgagaaagcucgcaucugccgcaggaaaaguccuggacagaaacauuucuggaaagaucggggacuuacaagcggugauggccgugccagacacggagacgccaacauuuugcuuacacacagaugucucauguagacagagagcagacgucgcgauauaccaagacgucuaugcuguacacgcacccacgucgcuauaccaccaggcgauuaaaggaguccgaguggcguacuggguaggguucgacacaaccccguucauguacaacgcuauggcgggugccuaccccucauacucgacaaauugggcggaugagcagguacugaaggcuaagaacauaggauuauguucaacagaccugacggaagguagacgaggcaaauugucuaucaugagagggaaaaagcuaaaaccgugcgaccgugugcuguucucaguagggucaacgcuuuacccggaaagccgcacgcuacuuaagagcuggcaccuaccaucgguguuccaucuaaagggcaagcuuagcuucacaugccgcugugacacagugguuucgugugagggcuacgucguuaagagaauaacgaugagcccaggccuuuauggaaaaaccauaggguaugcgguaacccaccacgcagacggauucuugaugugcaagacuaccgacacgguugacggcgaaagagugucauucucggugugcacguacgugccggcgaccauuugugaucaaaugaccggcauccuugcuacagaagucacgccggaggaugcacagaagcuguugguggggcugaaccagaggauagugguuaacggcagaacgcaacggaacacgaacaccaugaagaacuaccuacuucccguggucgcccaggccuucaguaagugggcaaaggagugccggaaggacauggaagaugagaagcuucugggggucagagaaagaacacuaaccugcugcugucuaugggcauuuaagaagcagaaaacacacacggucuacaagaggccugauacccagucaauccagaagguucaggccgaauuugacagcuuuguaguaccgggccuguggucguccggguugucaaucccguugaggacuagaaucaagugguuguuacgcaaggugccgaaaacagaccugaucccauacagcgggaaugcccaagaagcccaggaugcagaaaaagaagcagaggaagaacgagaagcagaacugacucaugaggcucuaccaccccuacaggcagcacaggaagauguccaggucgaaaucgacguggaacagcuugaggauagagcuggugcuggaauaauagagacuccgagaggcgcuaucaaaguuacugcccaacuaacagaccacgucgugggggaguaccugguacuuuccccgcagaccguacuacgcagccagaagcucagccugauccacgcuuuagcggagcaagugaagacguguacgcacagcggacgagcagggagguaugcggucgaagcguacgauggccgaguccuagugcccucaggcuaugcaauuucgccugaagacuuccagagucuaagcgaaagcgcaacgaugguguacaacgaaagagaguucguaaacagaaaguuacaccacauugcgaugcacggaccagcccugaacacugacgaagagucguaugagcuggugagggcagagaggacagaacacgaguacgucuacgacguggaccagagaagaugcuguaagaaggaagaagcugcaggacugguacuggugggcgacuugacuaauccgcccuaccacgaauucgcauacgaagggcuaaaaauucgccccgccugcccauacaaaauugcagucauaggagucuucgggguaccaggaucuggcaagucagccauuaucaagaaccuaguuaccaggcaagaccuggugacuagcggaaagaaagaaaacugccaagaaaucagcaccgacgugaugagacagagaggucuagagauaucugcacguacgguagauucgcugcucuugaauggaugcaacagaccagucgacguguuguacguagacgaggcguuugcgugccacucuggaacguuacuugcuuugaucgccuuggugagaccaagacagaaaguuguacuuuguggugacccgaagcagugcggcuucuucaauaugaugcagaugaaagucaacuacaaucauaacaucugcacccaaguguaccacaaaaguaucuccaggcgguguacacugccugugacugccauugugucaucguugcauuacgaaggcaaaaugcgcacuacgaaugaguacaacaugccgauuguaguggacacuacaggcucaacgaaaccugacccuggagaccucguguuaacgugcuucagaggguggguuaaacaacugcaaauugacuaucguggacacgaggucaugacagcagccgcaucccaaggguuaacuagaaaaggaguuuacgcaguuaggcaaaaaguuaacgaaaacccacucuaugcaucaacaucagagcacgucaacguacuccuaacgcguacggaagguaaacugguauggaagacacucucuggugacccguggauaaagacgcugcagaacccaccgaaaggaaacuucaaagcaacuauuaaggagugggagguggagcacgcaucgauaauggcgggcaucugcagucaccaagugaccuuugacacauuccaaaacaaagccaacguuugcugggcuaagagcuuggucccuauccucgaaacagcggggauaaaacuaaaugauaggcaguggucccagauaauucaagccuucaaagaagacaaagcauacucacccgaaguagcccugaaugaaauaugcacgcgcauguaugggguggaucuagacagugggcuauucucuaaaccguugguaucuguguauuacgcggauaaccauugggauaauaggccgggaggaaagauguucggauucaacccugaggcagcguccauucuagaaagaaaguacccauuuacaaaaggaaaguggaacaucaacaagcagaucugcgugacuaccaggaggauagaagacuucaacccuaccaccaacauuauaccggucaacaggagacuaccacacucauuaguggccgaacaccgcccaguaaaaggggaaagaauggaauggcugguuaacaagauaaacggacaccacguacuccugguuagcggcuauaaccuugcacugccuacuaagagagucaccuggguagcgccacuagguguccgcggagcggacuauacauacaaccuagagcugggucuaccagcaacgcuugguagguaugaccuaguggucauaaacauccacacaccuuuucgcauacaccauuaccaacagugcguagaucacgcaaugaaacugcaaaugcuagggggugacucacugagacugcucaaaccggguggcucucuauugaucagagcauacgguuacgcagauagaaccagugaacgagucaucugcguacugggacgcaaguuuagaucgucuagagcauugaaaccaccaugugucaccaguaauacugagauguuuuuccuauuuagcaauuuugacaauggcagaaggaauuuuacaacgcaugucaugaacaaucaacugaaugcagccuuuguaggacaggccacccgagcaggaugugcaccaucguaccggguaaaacgcauggacaucgcgaagaacgaugaagagugcgugguuaacgccgccaacccucgcggguuaccaggugacgguguuugcaaggcaguauauaaaaaguggccggaguccuuuaaaaacagugcaacaccaguaggaaccgcaaaaacaguuaugugcgguacguauccaguaauccacgccguaggaccaaacuucucaaauuauucggagucugaaggggaccgggaauuggcggcugccuaucgagaagucgcaaaggaaguaacuagacugggaguaaauagcguagcuauaccucuccucuccacagguguauacucaggagggaaagacaggcuaacccagucacugaaccaccucuuuacagccauggacucgacggaugcagacguggucaucuacugccgagacaaggaaugggagaagaaaauaucugaggccauacagaugcggacccaaguggagcugcuggaugagcacaucuccauagacugcgaugucauucgcgugcacccugacaguagcuuggcaggcagaaaaggauacagcaccacggaaggcgcacuguauucauaucuagaagggacacguuuucaccagacggcaguggauauggcagagauauacacuauguggccaaagcaaacagaggccaaugagcaagucugccuauaugcccugggggaaaguauugaaucaaucaggcagaaaugcccgguggaugaugcagacgcaucaucucccccgaaaacugucccgugucuuugccgguaugccaugacuccugaacgcgucacccgacuucgcaugaaccaugucacaaauauaauuguguguucuucauuuccccuuccaaaguacaagauagaaggagugcaaaaagucaaaugcuccaagguaauguuauucgaucacaaugugccaucgcgcguaaguccaagggaauacagaucuucccaggagucuguacaggaagugaguacgacaacgucauugacgcauagccaguuugaucuaagcgccgauggcgagacacugccugucccgucagaccuggaugcugacgccccagcccuagaaccggcccuagacgacggggcgguacauacauuaccaaccauaaucggaaaccuugcggccgugucugacuggguaaugagcaccguaccugucgcgccgccuagaagaaggagagggagaaaccugacugugacaugugacgagagagaagggaauauaacacccauggcuagcguccgauucuuuagagcagagcuguguccggccguacaagaaacagcggagacgcgugacacagcuauuucccuucaggcaccgccaaguaccaccauggaacugagccauccaccgaucuccuucggagcaccaagcgagacguuccccaucacauuuggggacuucgacgaaggagaaaucgaaagcuugucuucugagcuacuaacuuucggagacuuccuacccggugaaguggaugaucugacagauagcgacugguccacgugcccagacacggacgacgaguuaugacuagacagggcagguggguauauauucucgucggacacugguccaggccauuuacaacagaagucgguacgccagucagugcugccgguaaacacccuggaggaaguccacgaggagaaguguuacccaccuaagcuggaugaauuaaaggagcaacuacuacuuaagaaacuccaggagagugcguccauggccaauagaagcagguaucagucacgcaaaguggaaaauaugaaagcaacaaucauccagagacuaaagagaggcuguaaacuguauuuaauggcagagaccccgaaagucccgacuuaucggaccauauacccggcgccuguguacucgccuccgaucaauguccgauuguccaaccccgaguccgcaguggcagcauguaaugaguucuuagcuagaaacuacccaacuguuucaucauaccaaaucaccgacgaguaugaugcauaucuagacaugguggacgggucggagaguugcuuggaccgagcgacauucaauccgucaaaacuuaggagcuacccgaaacaacaugcuuaucacgcgccuucuaucagaagcgcuguaccuuccccauuccagaacacacuacagaauguacuggcagcagccacgaaaaggaacugcaacgucacacagaugagggaauuacccacuuuggacucagcaguauucaacguggaguguuuuaaaaaauucgcauguaaccgagaauacugggaagaauuugcagccagcccuaucaggauaacaacugagaaucuaacaaccuaugucacuaaacuaaaggggccaaaagcagcagcgcuguuugcaaaaacccauaaucugcugccacugcaggauguaccaauggauagguucacaguagauaugaaaagggaugugaagguaacuccugguacaaagcauacagaggaaagaccuaaggugcagguuauacaggcggcugaacccuuggcaacagcguaccuauguggaauucacagagaacugguuaggagauugaacgccguccuccuacccaaugugcauacacuauuugacaugucugccgaggacuucgaugccauuauagccgcacacuucaagccaggagacgcuguuuuagaaacggacauagccuccuuugauaagagccaagaugauucacuugcgcuuaccgccuuaaugcuguuagaagauuugggaguggaucacucccuguuggacuugauagaggcugcuuucggagagauuuccagcugucaucugccgacagguacgcgcuucaaguucggcgcuaugaugaaauccgguauguuccuaacucuguucgucaacacguuguuaaauaucaccaucgcuagccggguguuggaagaucgucugacaaaauccgcaugcgcggccuucaucggcgacgacaacauaauacauggugucgucuccgaugaauugauggcagccagaugcgcuacuuggaugaacauggaagugaagaucauagaugcaguuguaucccagaaagcuccuuacuuuuguggaggguuuauacugcaugauacugugacaggaacagcuugcagaguggcggacccgcuaaaaagguuauuuaaauugggcaaaccguuagcggcaggugacgaacaagaugaagacagaagacgggcgcuggcugaugaaguaaucagauggcaacgaacagggcuaauagaugagcuggagaaagcgguguacucuagguacgaagugcaggguauaucaguugcgguaauguccauggccaccuuugcaagcuccagauccaacuucgagaagcucagaggacccgucauaacgcuuuauggaggaccgaaguaauaaccccucucccuccccccccccuaacguuacuggccgaagccgcuuggaauaaggccggugugcguuugucuauauguuauuuuccaccauauugccgucuuuuggcaaugugagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucgguacacaugcuuuacauguguuuagucgagguuaaaaaaacgucuaggccccccgaaccacggggacgugguuuuccuuugaaaaacacgaugauaauauggccacaaccauggaguuuaucccaacccaaacuuucuacaauaggagguaccagccucgaccuuggacuccgcgcccuacuauccaaguuaucagacccagaccgcguccgcaaaggaaagccgggcaacuugcccagcugaucucagcaguuaauaaacugacaaugcgcgcgguaccucaacagaagccgcgcaagaaucggaagaauaagaagcaaaagcaaaagcagcaggcgccacgaaacaacaugaaucaaaagaagcagcccccuaaaaagaaaccggcucaaaagaaaaagaagccgggccguagagagagaaugugcaugaaaaucgaaaaugauugcaucuucgaagucaagcaugaagguaagguaacagguuacgcgugcuugguaggggacaaaguaaugaagccagcacacguaaaggggaccaucgauaaugcggaccuggccaaauuggccuucaagcggucaucuaaguacgaccuugaaugcgcgcagauacccgugcacaugaaguccgacgcuucgaaguucacccaugagaaaccggagggguacuacaacuggcaccacggagcaguacaguacucaggaggccgguucaccaucccuacaggugcgggcaaaccaggggacagcgguagaccgaucuucgacaacaaggggcgcgugguggccauaguuuuaggaggagcuaaugaaggagcccguacagcccucucgguggugaccuggaacaaagacaucgucacgaaaaucaccccugagggggccgaagaguggagucuugccauuccaguuaugugccugcuggcaaauaccacguuccccugcucccagcccccuugcacacccugcugcuacgaaaaagagccggagaaaacccugcgcaugcuagaagacaacgucaugagccccggguacuaucagcugcuacaagcauccuuaacauguucuccccgccgccagcgacgcaguauuaaggacaacuucaaugucuauaaagccauaagaccguaccuagcucacugucccgacuguggagaagggcacucgugccauagucccguagcgcuagaacgcaucagaaacgaagcgacagacgggacgcugaaaauccagguuuccuugcaaaucggaauaaagacggaugauagccaugauuggaccaagcugcguuacauggacaaucauaugccagcagacgcagagagggccaggcuauuuguaagaacgucagcaccgugcacgauuacuggaacaaugggacacuucauccuggcccgauguccgaaaggagaaacucugacggugggauucacugacgguaggaagaucagucacucauguacgcacccauuucaccacgacccuccugugauaggccgggaaaaauuucauucccgaccgcagcacgguagagaacuaccuugcagcacguacgcgcagagcaccgcugcaacugccgaggagauagagguacauaugcccccagacaccccagaucgcacauugaugucacaacaguccgguaauguaaagaucacagucaauagucagacggugcgguacaaguguaauugcggugacucaaaugaaggacuaaccacuacagacaaagugauuaauaacugcaagguugaucaaugccaugccgcggucaccaaucacaaaaaauggcaguauaauuccccucuggucccgcguaaugcugaacucggggaccgaaaaggaaaaguucacauuccguuuccucuggcaaaugugacaugcagggugccuaaggcaaggaaccccaccgugacguacggaaaaaaccaagucaucaugcugcuguauccugaccacccaacgcuccuguccuaccggaauaugggagaagaaccaaacuaucaagaagagugggugacgcauaagaaggagaucagguuaaccgugccgacugaagggcucgaggucacguggggcaacaacgagccguacaaguauuggccgcaguuauccacaaacgguacagcccacggccacccgcaugagauaauuuuguauuauuaugagcuguacccuacuaugacugugguaguugugucaguggccucguucguacuccugucgaugguggguguggcaguggggaugugcaugugugcacgacgcagaugcauuacaccguacgaacugacaccaggagcuaccgucccuuuccugcuuagccuaauaugcugcauuagaacagcuaaagcggccacauaccaagaggcugcgguauaccuguggaacgagcagcagccuuuguuuuggcugcaagcccuuauuccgcuggcagcccugauuguccuaugcaacugucugagacucuuaccaugcuuuuguaaaacguugacuuuuuuagccguaaugagcgucggugcccacacugugagcgcguacgaacacguaacagugaucccgaacacggugggaguaccguauaagacucuagucaacagaccgggcuacagccccaugguacuggagauggagcuucugucagucacuuuggagccaacgcuaucgcuugauuacaucacgugcgaguauaaaaccgucaucccgucuccguacgugaaaugcugcgguacagcagagugcaaggacaagagccuaccugauuacagcuguaaggucuucaccggcgucuacccauucauguggggcggcgccuacugcuucugcgacacugaaaauacgcaauugagcgaagcacauguggagaaguccgaaucaugcaaaacagaauuugcaucagcauauagggcucauaccgcauccgcaucagcuaagcuccgcguccuuuaccaaggaaauaauguuacuguaucugcuuaugcaaacggcgaucaugccgucacaguuaaggacgcuaaauucauuguggggccaaugucuucagccuggacaccuuuugacaauaaaaucgugguguacaaaggcgacgucuacaacauggacuacccgcccuucggcgcaggaagaccaggacaauuuggcgacauccaaagucgcacgccugagagcgaagacgucuaugcuaacacacaacugguacugcagagaccguccgcggguacggugcacgugccguacucucaggcaccaucuggcuucaaguauuggcuaaaagaacgaggggcgucgcugcagcacacagcaccauuuggcugucaaauagcaacaaacccgguaagagcgaugaacugcgccguagggaacaugccuaucuccaucgacauaccggacgcggccuucacuagggucgucgacgcgccaucuuuaacggacaugucgugugagguaccagccugcacccacuccucagacuuugggggcguagccaucauuaaauaugcagccagcaagaaaggcaagugugcggugcauucgaugacuaacgccgucacuauucgggaagcugaaauagaaguagaagggaacucucaguugcaaaucucuuuuucgacggcccuagccagcgccgaauuccgcguacaagucuguucuacacaaguacacugugcagccgagugccauccaccgaaagaccauauagucaauuacccggcgucacacaccacccucgggguccaagacauuuccguuacggcgaugucaugggugcagaagaucacgggaggugugggacugguugucgcuguugcagcacugauccuaaucguggugcuaugcgugucguuuagcaggcacuaacuugacaacuagguacgaagguauauguguccccuaagagacacaccacauauagcuaagaaucaauagauaaguauagaucaaagggcugaacaaccccugaauaguaacaaaauauaaaaaucaacaaaaaucauaaaauagaaaaccagaaacagaaguagguaagaagguauauguguccccuaagagacacaccauauauagcuaagaaucaauagauaaguauagaucaaagggcugaauaaccccugaauaauaacaaaauauaaaaaucaauaaaaaucauaaaauagaaaaccauaaacagaaguaguucaaagggcuauaaaaccccugaauaguaacaaaacauaaaacuaauaaaaaucaaaugaauaccauaauuggcaaucggaagagauguagguacuuaagcuuccuaaaagcagccgaacucgcuuugagauguaggcguagcacaccgaacucuuccauaauucuccgaacccacagggacguaggagauguucaaaguggcuauaaaacccugaacaguaauaaaacauaaaauuaauaaggaucaaaugaguaccauaauuggcaaacggaagagauguagguacuuaagcuuccuaaaagcagccgaacucacuuugagauguaggcauagcauaccgaacucuuccacaauucuccguacccauagggacguaggagauguuauuuuguuuuuaauauuuc

SEQ ID NO: 5 - RNA, 3747 nt, Alphavirus Chikungunya virus
auggaguuuaucccaacccaaacuuucuacaauaggagguaccagccucgaccuuggacuccgcgcccuacuauccaagucaucagacccagaccgcguccgcaaaggaaggccgggcaacuugcccagcugaucucagcaguuaauaaacugacaaugcgcgugguaccucaacagaagccgcgcaagaaucggaagaauaagaagcaaaagcaaaagcagcaggcgccacgaaacaauacgaaucaaaagaagcagccccccaaaaagaaaccgguucaaaagaaaaagaagccgggccgcagagagagaaugugcaugaaaaucgaaaaugauugcaucuucgaagucaagcaugaagguaagguaacagguuacgcgugcuugguaggggacaaaguaaugaagccagcacacguaaaggggaccaucgauaacgcggaccuggccaaauuggccuucaagcggucaucuaaguacgaccuugaaugcgcgcagauacccgugcacaugaaguccgacgcuucgaaguucacccaugagaaaccggagggguacuacaacuggcaccacggagcaguacaguacucaggaggccgauucaccaucccuacaggugcgggcaaaccaggggauagugguagaccgaucuucgacaacaaggggcgcgugguggccauaguuuuaggaggagcuaaugaaggagcccguacagcccucucgguggugaccuggaacaaagacaucgucacgaaaaucaccccugagggggccgaagaguggagucuggccauuccaguuaugugccugcuggcaaauaccacguuccccugcucccggcccccuugcacacccugcugcuacgaaaaagagccggagaaaaccuugcgcaugcuugaagacaaugucaugagccccggguacuaucagcugcuacaagcauccuuaacauguucuccccgacgccagcggcgcaguauuaaggaccacuucaaugucuauaaagccacaagaccguaccuagcucacugucccgacuguggagaagggcacucgugccauagucccguagcgcuagaacgcaucagaaacgaagcgacagacgggacguugaaaauccagguuuccuugcaaaucggaauaaagacggaugauagccaugauuggaccaagcugcguuauauggacaaucacaugccagcagacgcagagcgggccgggcuauuuguaagaacgucagcaccgugcacgauuacuggaacaaugggacacuucauucuggcccgauguccgaaaggagaaacucugacggcgggguucacugacgguaggaagaucagucacucauguacgcacccauuucaccaugacccuccugugauaggccgggaaaaauuccauucccgaccgcagcacgguagggaacuaccuugcagcacguacgcgcagagcaccgcugcaacugccgaggagauagagguacacaugcccccagacaccccagaucgcacauuaaugucacaacaguccggcaauguaaagaucacagucaauagucagacggugcgguacaagugcaauuguggugacucaagugaaggauuaaccacuacagauaaagugauuaauaacugcaaggucgaucaaugccaugccgcggucaccaaucacaaaaaauggcaguauaauuccccucuggucccgcguaaugcugaauucggggaccggaaaggaaaaguucacauuccauuuccucuggcaaaugugacaugcagggugccuaaagcaagaaaccccaccgugacguacggaaaaaaccaagucaucauguugcuguauccugaccacccaacgcuccuguccuacaggaauaugggagaagaaccaaacuaucaagaagagugggugacgcauaagaaggagaucagguuaaccgugccgacugaggggcucgaggucacgugggguaacaaugagccguacaaguauuggccgcaguuauccacaaacgguacagcccacggccacccgcaugagauaauucuguauuauuaugagcuguacccaacuaugacugcgguaguuuugucaguggccucguucauacuccugucgaugguggguguggcaguggggaugugcaugugugcacgacgcagaugcauuacaccguacgaacugacaccaggagcuaccgucccuuuccugcuuagccuaauaugcugcauuagaacagcuaaagcggccacauaccaggaggccgcgguauaccuguggaacgagcagcagccuuuauuuuggaugcaagcccuuauuccgcuggcagcccugauuguccuauguaacugucugagacucuuaccaugcuguuguaaaauguugacuuuuuuagccguacugagcgucggugcccacacugugagcgcguacgaacacguaacagugaucccgaacacggugggaguaccguauaagacucuagucaacagaccgggcuacagccccaugguauuggagauggagcuucugucugucaccuuggaaccaacgcuaucgcuugauuacaucacgugcgaguauaaaaccguuaucccgucuccguacgugaaaugcugcgguacagcagaguguaaggacaagagccuaccugauuacagcuguaaggucuucaccggcgucuacccauucauguggggcggcgccuacugcuucugcgacaccgaaaauacgcaauugagcgaagcacauguggagaaguccgaaucaugcaaaacagaauuugcaucagcauacagggcucauaccgcauccgcaucagcuaagcuccgcguccuuuaccaaggaaauaauaucacuguggcugcuuaugcaaacggcgaccaugccgucacaguuaaggacgcuaaauucauaguggggccaaugucuucagccuggacaccuuucgacaauaaaaucgugguguacaaaggcgacgucuacaacauggacuacccgcccuucggcgcaggaagaccaggacaauuuggcgacauccaaagucgcacgccugagagcgaagacgucuaugcuaauacacaacugguacugcagagaccguccgcggguacggugcacgugccguacucucaggcaccaucuggcuucaaguauuggcuaaaagaacgaggggcgucgcugcagcacacagcaccauuuggcugucaaauagcaacaaacccgguaagagcgaugaacugcgccguagggaacaugccuaucuccaucgacauaccggacgcggccuuuaccagggucgucgacgcgccaucuuuaacggacaugucgugugagguaucagccugcacccauuccucagacuuugggggcguagccaucauuaaauaugcagccaguaagaaaggcaagugugcagugcacucgaugacuaacgccgucacuauucgggaagcugaaauagaaguagaagggaacucucaguugcaaaucucuuuuucgacggcccuagccagcgccgaauuucgcguacaagucuguucuacacaaguacacugugcagccgagugccauccaccgaaagaccauauagucaauuacccggcgucacacaccacccucgggguccaagacauuuccgcuacggcgaugucaugggugcagaagaucacgggaggugugggacugguugucgcuguugcagcacugauccuaaucguggugcuaugcgugucguuuagcaggcacuaa

SEQ ID NO: 6 - DNA, 7457 bp, Artificial Sequence
  plasmid construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcggtaatacgactcactataggcgcgtccgccccgcgagcacagagcctcgcctttgccgatccgccgcccgtccacacccgccgccagctcacacgcgatggagtttatcccaacccaaactttctacaataggaggtaccagcctcgaccttggactccgcgccctactatccaagtcatcagacccagaccgcgtccgcaaaggaaggccgggcaacttgcccagctgatctcagcagttaataaactgacaatgcgcgtggtacctcaacagaagccgcgcaagaatcggaagaataagaagcaaaagcaaaagcagcaggcgccacgaaacaatacgaatcaaaagaagcagccccccaaaaagaaaccggttcaaaagaaaaagaagccgggccgcagagagagaatgtgcatgaaaatcgaaaatgattgcatcttcgaagtcaagcatgaaggtaaggtaacaggttacgcgtgcttggtaggggacaaagtaatgaagccagcacacgtaaaggggaccatcgataacgcggacctggccaaattggccttcaagcggtcatctaagtacgaccttgaatgcgcgcagatacccgtgcacatgaagtccgacgcttcgaagttcacccatgagaaaccggaggggtactacaactggcaccacggagcagtacagtactcaggaggccgattcaccatccctacaggtgcgggcaaaccaggggatagtggtagaccgatcttcgacaacaaggggcgcgtggtggccatagttttaggaggagctaatgaaggagcccgtacagccctctcggtggtgacctggaacaaagacatcgtcacgaaaatcacccctgagggggccgaagagtggagtctggccattccagttatgtgcctgctggcaaataccacgttcccctgctcccggcccccttgcacaccctgctgctacgaaaaagagccggagaaaaccttgcgcatgcttgaagacaatgtcatgagccccgggtactatcagctgctacaagcatccttaacatgttctccccgacgccagcggcgcagtattaaggaccacttcaatgtctataaagccacaagaccgtacctagctcactgtcccgactgtggagaagggcactcgtgccatagtcccgtagcgctagaacgcatcagaaacgaagcgacagacgggacgttgaaaatccaggtttccttgcaaatcggaataaagacggatgatagccatgattggaccaagctgcgttatatggacaatcacatgccagcagacgcagagcgggccgggctatttgtaagaacgtcagcaccgtgcacgattactggaacaatgggacacttcattctggcccgatgtccgaaaggagaaactctgacggcggggttcactgacggtaggaagatcagtcactcatgtacgcacccatttcaccatgaccctcctgtgataggccgggaaaaattccattcccgaccgcagcacggtagggaactaccttgcagcacgtacgcgcagagcaccgctgcaactgccgaggagatagaggtacacatgcccccagacaccccagatcgcacattaatgtcacaacagtccggcaatgtaaagatcacagtcaatagtcagacggtgcggtacaagtgcaattgtggtgactcaagtgaaggattaaccactacagataaagtgattaataactgcaaggtcgatcaatgccatgccgcggtcaccaatcacaaaaaatggcagtataattcccctctggtcccgcgtaatgctgaattcggggaccggaaaggaaaagttcacattccatttcctctggcaaatgtgacatgcagggtgcctaaagcaagaaaccccaccgtgacgtacggaaaaaaccaagtcatcatgttgctgtatcctgaccacccaacgctcctgtcctacaggaatatgggagaagaaccaaactatcaagaagagtgggtgacgcataagaaggagatcaggttaaccgtgccgactgaggggctcgaggtcacgtggggtaacaatgagccgtacaagtattggccgcagttatccacaaacggtacagcccacggccacccgcatgagataattctgtattattatgagctgtacccaactatgactgcggtagttttgtcagtggcctcgttcatactcctgtcgatggtgggtgtggcagtggggatgtgcatgtgtgcacgacgcagatgcattacaccgtacgaactgacaccaggagctaccgtccctttcctgcttagcctaatatgctgcattagaacagctaaagcggccacataccaggaggccgcggtatacctgtggaacgagcagcagcctttattttggatgcaagcccttattccgctggcagccctgattgtcctatgtaactgtctgagactcttaccatgctgttgtaaaatgttgacttttttagccgtactgagcgtcggtgcccacactgtgagcgcgtacgaacacgtaacagtgatcccgaacacggtgggagtaccgtataagactctagtcaacagaccgggctacagccccatggtattggagatggagcttctgtctgtcaccttggaaccaacgctatcgcttgattacatcacgtgcgagtataaaaccgttatcccgtctccgtacgtgaaatgctgcggtacagcagagtgtaaggacaagagcctacctgattacagctgtaaggtcttcaccggcgtctacccattcatgtggggcggcgcctactgcttctgcgacaccgaaaatacgcaattgagcgaagcacatgtggagaagtccgaatcatgcaaaacagaatttgcatcagcatacagggctcataccgcatccgcatcagctaagctccgcgtcctttaccaaggaaataatatcactgtggctgcttatgcaaacggcgaccatgccgtcacagttaaggacgctaaattcatagtggggccaatgtcttcagcctggacacctttcgacaataaaatcgtggtgtacaaaggcgacgtctacaacatggactacccgcccttcggcgcaggaagaccaggacaatttggcgacatccaaagtcgcacgcctgagagcgaagacgtctatgctaatacacaactggtactgcagagaccgtccgcgggtacggtgcacgtgccgtactctcaggcaccatctggcttcaagtattggctaaaagaacgaggggcgtcgctgcagcacacagcaccatttggctgtcaaatagcaacaaacccggtaagagcgatgaactgcgccgtagggaacatgcctatctccatcgacataccggacgcggcctttaccagggtcgtcgacgcgccatctttaacggacatgtcgtgtgaggtatcagcctgcacccattcctcagactttgggggcgtagccatcattaaatatgcagccagtaagaaaggcaagtgtgcagtgcactcgatgactaacgccgtcactattcgggaagctgaaatagaagtagaagggaactctcagttgcaaatctctttttcgacggccctagccagcgccgaatttcgcgtacaagtctgttctacacaagtacactgtgcagccgagtgccatccaccgaaagaccatatagtcaattacccggcgtcacacaccaccctcggggtccaagacatttccgctacggcgatgtcatgggtgcagaagatcacgggaggtgtgggactggttgtcgctgttgcagcactgatcctaatcgtggtgctatgcgtgtcgtttagcaggcactaaccggttaagcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcattgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttccccctttttgtcccccaacttgagatgtatgaaggcttttggtctccctgggagtgggtggaggcagccagggcttacctgtacactgacttgagaccagttgaataaaagtgcacaccttaaaaaaaaaaaaaaaaaaaaaaaaaaaaaaaaaaaaaaaaaaaaaaaaaaaaaaaaaaaaagggtcggcatggcatctccacctcctcgcggtccgacctgggcatccgaaggaggacgcacgtccactcggatggctaagggagcctgcattcgcagaagccacccgcgctgctaacaaagcccgaaaggaagctgagttggctgctgccaccgctgagcaataactagcataaccccttggggcctctaaacgggtcttgaggggttttttgctgaaaggaggaactatatgcgctcatagcggccgggtacctcgcgaatgcatctagatatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 7 - DNA, 13917 bp, Artificial Sequence
  plasmid construct
atggctgcgtgagacacacgtagcctaccagtttcttactgctctactctgcaaagcaagagattaataacccatcatggattctgtgtacgtggacatagacgctgacagcgcctttttgaaggccctgcaacgtgcgtaccccatgtttgaggtggaacctaggcaggtcacatcgaatgaccatgctaatgctagagcgttctcgcatctagccataaaactaatagagcaggaaattgatcccgactcaaccatcctggatataggtagtgcgccagcaaggaggatgatgtcggacaggaagtaccactgcgtttgcccgatgcgcagcgcagaagatcccgagagactcgctaattatgcgagaaagctcgcatctgccgcaggaaaagtcctggacagaaacatttctggaaagatcggggacttacaagcggtgatggccgtgccagacacggagacgccaacattttgcttacacacagatgtctcatgtagacagagagcagacgtcgcgatataccaagacgtctatgctgtacacgcacccacgtcgctataccaccaggcgattaaaggagtccgagtggcgtactgggtagggttcgacacaaccccgttcatgtacaacgctatggcgggtgcctacccctcatactcgacaaattgggcggatgagcaggtactgaaggctaagaacataggattatgttcaacagacctgacggaaggtagacgaggcaaattgtctatcatgagagggaaaaagctaaaaccgtgcgaccgtgtgctgttctcagtagggtcaacgctttacccggaaagccgcacgctacttaagagctggcacctaccatcggtgttccatctaaagggcaagcttagcttcacatgccgctgtgacacagtggtttcgtgtgagggctacgtcgttaagagaataacgatgagcccaggcctttatggaaaaaccatagggtatgcggtaacccaccacgcagacggattcttgatgtgcaagactaccgacacggttgacggcgaaagagtgtcattctcggtgtgcacgtacgtgccggcgaccatttgtgatcaaatgaccggcatccttgctacagaagtcacgccggaggatgcacagaagctgttggtggggctgaaccagaggatagtggttaacggcagaacgcaacggaacacgaacaccatgaagaactacctacttcccgtggtcgcccaggccttcagtaagtgggcaaaggagtgccggaaggacatggaagatgagaagcttctgggggtcagagaaagaacactaacctgctgctgtctatgggcatttaagaagcagaaaacacacacggtctacaagaggcctgatacccagtcaatccagaaggttcaggccgaatttgacagctttgtagtaccgggcctgtggtcgtccgggttgtcaatcccgttgaggactagaatcaagtggttgttacgcaaggtgccgaaaacagacctgatcccatacagcgggaatgcccaagaagcccaggatgcagaaaaagaagcagaggaagaacgagaagcagaactgactcatgaggctctaccacccctacaggcagcacaggaagatgtccaggtcgaaatcgacgtggaacagcttgaggatagagctggtgctggaataatagagactccgagaggcgctatcaaagttactgcccaactaacagaccacgtcgtgggggagtacctggtactttccccgcagaccgtactacgcagccagaagctcagcctgatccacgctttagcggagcaagtgaagacgtgtacgcacagcggacgagcagggaggtatgcggtcgaagcgtacgatggccgagtcctagtgccctcaggctatgcaatttcgcctgaagacttccagagtctaagcgaaagcgcaacgatggtgtacaacgaaagagagttcgtaaacagaaagttacaccacattgcgatgcacggaccagccctgaacactgacgaagagtcgtatgagctggtgagggcagagaggacagaacacgagtacgtctacgacgtggaccagagaagatgctgtaagaaggaagaagctgcaggactggtactggtgggcgacttgactaatccgccctaccacgaattcgcatacgaagggctaaaaattcgccccgcctgcccatacaaaattgcagtcataggagtcttcggggtaccaggatctggcaagtcagccattatcaagaacctagttaccaggcaagacctggtgactagcggaaagaaagaaaactgccaagaaatcagcaccgacgtgatgagacagagaggtctagagatatctgcacgtacggtagattcgctgctcttgaatggatgcaacagaccagtcgacgtgttgtacgtagacgaggcgtttgcgtgccactctggaacgttacttgctttgatcgccttggtgagaccaagacagaaagttgtactttgtggtgacccgaagcagtgcggcttcttcaatatgatgcagatgaaagtcaactacaatcataacatctgcacccaagtgtaccacaaaagtatctccaggcggtgtacactgcctgtgactgccattgtgtcatcgttgcattacgaaggcaaaatgcgcactacgaatgagtacaacatgccgattgtagtggacactacaggctcaacgaaacctgaccctggagacctcgtgttaacgtgcttcagagggtgggttaaacaactgcaaattgactatcgtggacacgaggtcatgacagcagccgcatcccaagggttaactagaaaaggagtttacgcagttaggcaaaaagttaacgaaaacccactctatgcatcaacatcagagcacgtcaacgtactcctaacgcgtacggaaggtaaactggtatggaagacactctctggtgacccgtggataaagacgctgcagaacccaccgaaaggaaacttcaaagcaactattaaggagtgggaggtggagcacgcatcgataatggcgggcatctgcagtcaccaagtgacctttgacacattccaaaacaaagccaacgtttgctgggctaagagcttggtccctatcctcgaaacagcggggataaaactaaatgataggcagtggtcccagataattcaagccttcaaagaagacaaagcatactcacccgaagtagccctgaatgaaatatgcacgcgcatgtatggggtggatctagacagtgggctattctctaaaccgttggtatctgtgtattacgcggataaccattgggataataggccgggaggaaagatgttcggattcaaccctgaggcagcgtccattctagaaagaaagtacccatttacaaaaggaaagtggaacatcaacaagcagatctgcgtgactaccaggaggatagaagacttcaaccctaccaccaacattataccggtcaacaggagactaccacactcattagtggccgaacaccgcccagtaaaaggggaaagaatggaatggctggttaacaagataaacggacaccacgtactcctggttagcggctataaccttgcactgcctactaagagagtcacctgggtagcgccactaggtgtccgcggagcggactatacatacaacctagagctgggtctaccagcaacgcttggtaggtatgacctagtggtcataaacatccacacaccttttcgcatacaccattaccaacagtgcgtagatcacgcaatgaaactgcaaatgctagggggtgactcactgagactgctcaaaccgggtggctctctattgatcagagcatacggttacgcagatagaaccagtgaacgagtcatctgcgtactgggacgcaagtttagatcgtctagagcattgaaaccaccatgtgtcaccagtaatactgagatgtttttcctatttagcaattttgacaatggcagaaggaattttacaacgcatgtcatgaacaatcaactgaatgcagcctttgtaggacaggccacccgagcaggatgtgcaccatcgtaccgggtaaaacgcatggacatcgcgaagaacgatgaagagtgcgtggttaacgccgccaaccctcgcgggttaccaggtgacggtgtttgcaaggcagtatataaaaagtggccggagtcctttaaaaacagtgcaacaccagtaggaaccgcaaaaacagttatgtgcggtacgtatccagtaatccacgccgtaggaccaaacttctcaaattattcggagtctgaaggggaccgggaattggcggctgcctatcgagaagtcgcaaaggaagtaactagactgggagtaaatagcgtagctatacctctcctctccacaggtgtatactcaggagggaaagacaggctaacccagtcactgaaccacctctttacagccatggactcgacggatgcagacgtggtcatctactgccgagacaaggaatgggagaagaaaatatctgaggccatacagatgcggacccaagtggagctgctggatgagcacatctccatagactgcgatgtcattcgcgtgcaccctgacagtagcttggcaggcagaaaaggatacagcaccacggaaggcgcactgtattcatatctagaagggacacgttttcaccagacggcagtggatatggcagagatatacactatgtggccaaagcaaacagaggccaatgagcaagtctgcctatatgccctgggggaaagtattgaatcaatcaggcagaaatgcccggtggatgatgcagacgcatcatctcccccgaaaactgtcccgtgtctttgccggtatgccatgactcctgaacgcgtcacccgacttcgcatgaaccatgtcacaaatataattgtgtgttcttcatttccccttccaaagtacaagatagaaggagtgcaaaaagtcaaatgctccaaggtaatgttattcgatcacaatgtgccatcgcgcgtaagtccaagggaatacagatcttcccaggagtctgtacaggaagtgagtacgacaacgtcattgacgcatagccagtttgatctaagcgccgatggcgagacactgcctgtcccgtcagacctggatgctgacgccccagccctagaaccggccctagacgacggggcggtacatacattaccaaccataatcggaaaccttgcggccgtgtctgactgggtaatgagcaccgtacctgtcgcgccgcctagaagaaggagagggagaaacctgactgtgacatgtgacgagagagaagggaatataacacccatggctagcgtccgattctttagagcagagctgtgtccggccgtacaagaaacagcggagacgcgtgacacagctatttcccttcaggcaccgccaagtaccaccatggaactgagccatccaccgatctccttcggagcaccaagcgagacgttccccatcacatttggggacttcgacgaaggagaaatcgaaagcttgtcttctgagctactaactttcggagacttcctacccggtgaagtggatgatctgacagatagcgactggtccacgtgcccagacacggacgacgagttatgactagacagggcaggtgggtatatattctcgtcggacactggtccaggccatttacaacagaagtcggtacgccagtcagtgctgccggtaaacaccctggaggaagtccacgaggagaagtgttacccacctaagctggatgaattaaaggagcaactactacttaagaaactccaggagagtgcgtccatggccaatagaagcaggtatcagtcacgcaaagtggaaaatatgaaagcaacaatcatccagagactaaagagaggctgtaaactgtatttaatggcagagaccccgaaagtcccgacttatcggaccatatacccggcgcctgtgtactcgcctccgatcaatgtccgattgtccaaccccgagtccgcagtggcagcatgtaatgagttcttagctagaaactacccaactgtttcatcataccaaatcaccgacgagtatgatgcatatctagacatggtggacgggtcggagagttgcttggaccgagcgacattcaatccgtcaaaacttaggagctacccgaaacaacatgcttatcacgcgccttctatcagaagcgctgtaccttccccattccagaacacactacagaatgtactggcagcagccacgaaaaggaactgcaacgtcacacagatgagggaattacccactttggactcagcagtattcaacgtggagtgttttaaaaaattcgcatgtaaccgagaatactgggaagaatttgcagccagccctatcaggataacaactgagaatctaacaacctatgtcactaaactaaaggggccaaaagcagcagcgctgtttgcaaaaacccataatctgctgccactgcaggatgtaccaatggataggttcacagtagatatgaaaagggatgtgaaggtaactcctggtacaaagcatacagaggaaagacctaaggtgcaggttatacaggcggctgaacccttggcaacagcgtacctatgtggaattcacagagaactggttaggagattgaacgccgtcctcctacccaatgtgcatacactatttgacatgtctgccgaggacttcgatgccattatagccgcacacttcaagccaggagacgctgttttagaaacggacatagcctcctttgataagagccaagatgattcacttgcgcttaccgccttaatgctgttagaagatttgggagtggatcactccctgttggacttgatagaggctgctttcggagagatttccagctgtcatctgccgacaggtacgcgcttcaagttcggcgctatgatgaaatccggtatgttcctaactctgttcgtcaacacgttgttaaatatcaccatcgctagccgggtgttggaagatcgtctgacaaaatccgcatgcgcggccttcatcggcgacgacaacataatacatggtgtcgtctccgatgaattgatggcagccagatgcgctacttggatgaacatggaagtgaagatcatagatgcagttgtatcccagaaagctccttacttttgtggagggtttatactgcatgatactgtgacaggaacagcttgcagagtggcggacccgctaaaaaggttatttaaattgggcaaaccgttagcggcaggtgacgaacaagatgaagacagaagacgggcgctggctgatgaagtaatcagatggcaacgaacagggctaatagatgagctggagaaagcggtgtactctaggtacgaagtgcagggtatatcagttgcggtaatgtccatggccacctttgcaagctccagatccaacttcgagaagctcagaggacccgtcataactttgtacggcggtcctaaataggtacgcactacagctacctattttgcagaagccgacagcaggtacctaaataccaatcagccataatggagtttatcccaacccaaactttctacaataggaggtaccagcctcgaccttggactccgcgccctactatccaagttatcagacccagaccgcgtccgcaaaggaaagccgggcaacttgcccagctgatctcagcagttaataaactgacaatgcgcgcggtacctcaacagaagccgcgcaagaatcggaagaataagaagcaaaagcaaaagcagcaggcgccacgaaacaacatgaatcaaaagaagcagccccctaaaaagaaaccggctcaaaagaaaaagaagccgggccgtagagagagaatgtgcatgaaaatcgaaaatgattgcatcttcgaagtcaagcatgaaggtaaggtaacaggttacgcgtgcttggtaggggacaaagtaatgaagccagcacacgtaaaggggaccatcgataatgcggacctggccaaattggccttcaagcggtcatctaagtacgaccttgaatgcgcgcagatacccgtgcacatgaagtccgacgcttcgaagttcacccatgagaaaccggaggggtactacaactggcaccacggagcagtacagtactcaggaggccggttcaccatccctacaggtgcgggcaaaccaggggacagcggtagaccgatcttcgacaacaaggggcgcgtggtggccatagttttaggaggagctaatgaaggagcccgtacagccctctcggtggtgacctggaacaaagacatcgtcacgaaaatcacccctgagggggccgaagagtggagtcttgccattccagttatgtgcctgctggcaaataccacgttcccctgctcccagcccccttgcacaccctgctgctacgaaaaagagccggagaaaaccctgcgcatgctagaagacaacgtcatgagccccgggtactatcagctgctacaagcatccttaacatgttctccccgccgccagcgacgcagtattaaggacaacttcaatgtctataaagccataagaccgtacctagctcactgtcccgactgtggagaagggcactcgtgccatagtcccgtagcgctagaacgcatcagaaacgaagcgacagacgggacgctgaaaatccaggtttccttgcaaatcggaataaagacggatgatagccatgattggaccaagctgcgttacatggacaatcatatgccagcagacgcagagagggccaggctatttgtaagaacgtcagcaccgtgcacgattactggaacaatgggacacttcatcctggcccgatgtccgaaaggagaaactctgacggtgggattcactgacggtaggaagatcagtcactcatgtacgcacccatttcaccacgaccctcctgtgataggccgggaaaaatttcattcccgaccgcagcacggtagagaactaccttgcagcacgtacgcgcagagcaccgctgcaactgccgaggagatagaggtacatatgcccccagacaccccagatcgcacattgatgtcacaacagtccggtaatgtaaagatcacagtcaatagtcagacggtgcggtacaagtgtaattgcggtgactcaaatgaaggactaaccactacagacaaagtgattaataactgcaaggttgatcaatgccatgccgcggtcaccaatcacaaaaaatggcagtataattcccctctggtcccgcgtaatgctgaactcggggaccgaaaaggaaaagttcacattccgtttcctctggcaaatgtgacatgcagggtgcctaaggcaaggaaccccaccgtgacgtacggaaaaaaccaagtcatcatgctgctgtatcctgaccacccaacgctcctgtcctaccggaatatgggagaagaaccaaactatcaagaagagtgggtgacgcataagaaggagatcaggttaaccgtgccgactgaagggctcgaggtcacgtggggcaacaacgagccgtacaagtattggccgcagttatccacaaacggtacagcccacggccacccgcatgagataattttgtattattatgagctgtaccctactatgactgtggtagttgtgtcagtggcctcgttcgtactcctgtcgatggtgggtgtggcagtggggatgtgcatgtgtgcacgacgcagatgcattacaccgtacgaactgacaccaggagctaccgtccctttcctgcttagcctaatatgctgcattagaacagctaaagcggccacataccaagaggctgcggtatacctgtggaacgagcagcagcctttgttttggctgcaagcccttattccgctggcagccctgattgtcctatgcaactgtctgagactcttaccatgcttttgtaaaacgttgacttttttagccgtaatgagcgtcggtgcccacactgtgagcgcgtacgaacacgtaacagtgatcccgaacacggtgggagtaccgtataagactctagtcaacagaccgggctacagccccatggtactggagatggagcttctgtcagtcactttggagccaacgctatcgcttgattacatcacgtgcgagtataaaaccgtcatcccgtctccgtacgtgaaatgctgcggtacagcagagtgcaaggacaagagcctacctgattacagctgtaaggtcttcaccggcgtctacccattcatgtggggcggcgcctactgcttctgcgacactgaaaatacgcaattgagcgaagcacatgtggagaagtccgaatcatgcaaaacagaatttgcatcagcatatagggctcataccgcatccgcatcagctaagctccgcgtcctttaccaaggaaataatgttactgtatctgcttatgcaaacggcgatcatgccgtcacagttaaggacgctaaattcattgtggggccaatgtcttcagcctggacaccttttgacaataaaatcgtggtgtacaaaggcgacgtctacaacatggactacccgcccttcggcgcaggaagaccaggacaatttggcgacatccaaagtcgcacgcctgagagcgaagacgtctatgctaacacacaactggtactgcagagaccgtccgcgggtacggtgcacgtgccgtactctcaggcaccatctggcttcaagtattggctaaaagaacgaggggcgtcgctgcagcacacagcaccatttggctgtcaaatagcaacaaacccggtaagagcgatgaactgcgccgtagggaacatgcctatctccatcgacataccggacgcggccttcactagggtcgtcgacgcgccatctttaacggacatgtcgtgtgaggtaccagcctgcacccactcctcagactttgggggcgtagccatcattaaatatgcagccagcaagaaaggcaagtgtgcggtgcattcgatgactaacgccgtcactattcgggaagctgaaatagaagtagaagggaactctcagttgcaaatctctttttcgacggccctagccagcgccgaattccgcgtacaagtctgttctacacaagtacactgtgcagccgagtgccatccaccgaaagaccatatagtcaattacccggcgtcacacaccaccctcggggtccaagacatttccgttacggcgatgtcatgggtgcagaagatcacgggaggtgtgggactggttgtcgctgttgcagcactgatcctaatcgtggtgctatgcgtgtcgtttagcaggcactaacttgacaactaggtacgaaggtatatgtgtcccctaagagacacaccacatatagctaagaatcaatagataagtatagatcaaagggctgaacaacccctgaatagtaacaaaatataaaaatcaacaaaaatcataaaatagaaaaccagaaacagaagtaggtaagaaggtatatgtgtcccctaagagacacaccatatatagctaagaatcaatagataagtatagatcaaagggctgaataacccctgaataataacaaaatataaaaatcaataaaaatcataaaatagaaaaccataaacagaagtagttcaaagggctataaaacccctgaatagtaacaaaacataaaactaataaaaatcaaatgaataccataattggcaatcggaagagatgtaggtacttaagcttcctaaaagcagccgaactcgctttgagatgtaggcgtagcacaccgaactcttccataattctccgaacccacagggacgtaggagatgttcaaagtggctataaaaccctgaacagtaataaaacataaaattaataaggatcaaatgagtaccataattggcaaacggaagagatgtaggtacttaagcttcctaaaagcagccgaactcactttgagatgtaggcatagcataccgaactcttccacaattctccgtacccatagggacgtaggagatgttattttgtttttaatatttcaaaaaaaaaaaaaaaaaaaaaaaaaaaaaaaaaaaaaaaaagcggccgcttaattaatcgaggggaattaattcttgaagacgaaagggccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgagctcgtaatacgactcactatagg

SEQ ID NO: 8 - DNA, 13755 bp, Artificial Sequence
  plasmid construct
atggctgcgtgagacacacgtagcctaccagtttcttactgctctactctgcaaagcaagagattaataacccatcatggattctgtgtacgtggacatagacgctgacagcgcctttttgaaggccctgcaacgtgcgtaccccatgtttgaggtggaacctaggcaggtcacatcgaatgaccatgctaatgctagagcgttctcgcatctagccataaaactaatagagcaggaaattgatcccgactcaaccatcctggatataggtagtgcgccagcaaggaggatgatgtcggacaggaagtaccactgcgtttgcccgatgcgcagcgcagaagatcccgagagactcgctaattatgcgagaaagctcgcatctgccgcaggaaaagtcctggacagaaacatttctggaaagatcggggacttacaagcggtgatggccgtgccagacacggagacgccaacattttgcttacacacagatgtctcatgtagacagagagcagacgtcgcgatataccaagacgtctatgctgtacacgcacccacgtcgctataccaccaggcgattaaaggagtccgagtggcgtactgggtagggttcgacacaaccccgttcatgtacaacgctatggcgggtgcctacccctcatactcgacaaattgggcggatgagcaggtactgaaggctaagaacataggattatgttcaacagacctgacggaaggtagacgaggcaaattgtctatcatgagagggaaaaagctaaaaccgtgcgaccgtgtgctgttctcagtagggtcaacgctttacccggaaagccgcacgctacttaagagctggcacctaccatcggtgttccatctaaagggcaagcttagcttcacatgccgctgtgacacagtggtttcgtgtgagggctacgtcgttaagagaataacgatgagcccaggcctttatggaaaaaccatagggtatgcggtaacccaccacgcagacggattcttgatgtgcaagactaccgacacggttgacggcgaaagagtgtcattctcggtgtgcacgtacgtgccggcgaccatttgtgatcaaatgaccggcatccttgctacagaagtcacgccggaggatgcacagaagctgttggtggggctgaaccagaggatagtggttaacggcagaacgcaacggaacacgaacaccatgaagaactacctacttcccgtggtcgcccaggccttcagtaagtgggcaaaggagtgccggaaggacatggaagatgagaagcttctgggggtcagagaaagaacactaacctgctgctgtctatgggcatttaagaagcagaaaacacacacggtctacaagaggcctgatacccagtcaatccagaaggttcaggccgaatttgacagctttgtagtaccgggcctgtggtcgtccgggttgtcaatcccgttgaggactagaatcaagtggttgttacgcaaggtgccgaaaacagacctgatcccatacagcgggaatgcccaagaagcccaggatgcagaaaaagaagcagaggaagaacgagaagcagaactgactcatgaggctctaccacccctacaggcagcacaggaagatgtccaggtcgaaatcgacgtggaacagcttgaggatagagctggtgctggaataatagagactccgagaggcgctatcaaagttactgcccaactaacagaccacgtcgtgggggagtacctggtactttccccgcagaccgtactacgcagccagaagctcagcctgatccacgctttagcggagcaagtgaagacgtgtacgcacagcggacgagcagggaggtatgcggtcgaagcgtacgatggccgagtcctagtgccctcaggctatgcaatttcgcctgaagacttccagagtctaagcgaaagcgcaacgatggtgtacaacgaaagagagttcgtaaacagaaagttacaccacattgcgatgcacggaccagccctgaacactgacgaagagtcgtatgagctggtgagggcagagaggacagaacacgagtacgtctacgacgtggaccagagaagatgctgtaagaaggaagaagctgcaggactggtactggtgggcgacttgactaatccgccctaccacgaattcgcatacgaagggctaaaaattcgccccgcctgcccatacaaaattgcagtcataggagtcttcggggtaccaggatctggcaagtcagccattatcaagaacctagttaccaggcaagacctggtgactagcggaaagaaagaaaactgccaagaaatcagcaccgacgtgatgagacagagaggtctagagatatctgcacgtacggtagattcgctgctcttgaatggatgcaacagaccagtcgacgtgttgtacgtagacgaggcgtttgcgtgccactctggaacgttacttgctttgatcgccttggtgagaccaagacagaaagttgtactttgtggtgacccgaagcagtgcggcttcttcaatatgatgcagatgaaagtcaactacaatcataacatctgcacccaagtgtaccacaaaagtatctccaggcggtgtacactgcctgtgactgccattgtgtcatcgttgcattacgaaggcaaaatgcgcactacgaatgagtacaacatgccgattgtagtggacactacaggctcaacgaaacctgaccctggagacctcgtgttaacgtgcttcagagggtgggttaaacaactgcaaattgactatcgtggacacgaggtcatgacagcagccgcatcccaagggttaactagaaaaggagtttacgcagttaggcaaaaagttaacgaaaacccactctatgcatcaacatcagagcacgtcaacgtactcctaacgcgtacggaaggtaaactggtatggaagacactctctggtgacccgtggataaagacgctgcagaacccaccgaaaggaaacttcaaagcaactattaaggagtgggaggtggagcacgcatcgataatggcgggcatctgcagtcaccaagtgacctttgacacattccaaaacaaagccaacgtttgctgggctaagagcttggtccctatcctcgaaacagcggggataaaactaaatgataggcagtggtcccagataattcaagccttcaaagaagacaaagcatactcacccgaagtagccctgaatgaaatatgcacgcgcatgtatggggtggatctagacagtgggctattctctaaaccgttggtatctgtgtattacgcggataaccattgggataataggccgggaggaaagatgttcggattcaaccctgaggcagcgtccattctagaaagaaagtacccatttacaaaaggaaagtggaacatcaacaagcagatctgcgtgactaccaggaggatagaagacttcaaccctaccaccaacattataccggtcaacaggagactaccacactcattagtggccgaacaccgcccagtaaaaggggaaagaatggaatggctggttaacaagataaacggacaccacgtactcctggttagcggctataaccttgcactgcctactaagagagtcacctgggtagcgccactaggtgtccgcggagcggactatacatacaacctagagctgggtctaccagcaacgcttggtaggtatgacctagtggtcataaacatccacacaccttttcgcatacaccattaccaacagtgcgtagatcacgcaatgaaactgcaaatgctagggggtgactcactgagactgctcaaaccgggtggctctctattgatcagagcatacggttacgcagatagaaccagtgaacgagtcatctgcgtactgggacgcaagtttagatcgtctagagcattgaaaccaccatgtgtcaccagtaatactgagatgtttttcctatttagcaattttgacaatggcagaaggaattttacaacgcatgtcatgaacaatcaactgaatgcagcctttgtaggacaggccacccgagcaggatgtgcaccatcgtaccgggtaaaacgcatggacatcgcgaagaacgatgaagagtgcgtggttaacgccgccaaccctcgcgggttaccaggtgacggtgtttgcaaggcagtatataaaaagtggccggagtcctttaaaaacagtgcaacaccagtaggaaccgcaaaaacagttatgtgcggtacgtatccagtaatccacgccgtaggaccaaacttctcaaattattcggagtctgaaggggaccgggaattggcggctgcctatcgagaagtcgcaaaggaagtaactagactgggagtaaatagcgtagctatacctctcctctccacaggtgtatactcaggagggaaagacaggctaacccagtcactgaaccacctctttacagccatggactcgacggatgcagacgtggtcatctactgccgagacaaggaatgggagaagaaaatatctgaggccatacagatgcggacccaagtggagctgctggatgagcacatctccatagactgcgatgtcattcgcgtgcaccctgacagtagcttggcaggcagaaaaggatacagcaccacggaaggcgcactgtattcatatctagaagggacacgttttcaccagacggcagtggatatggcagagatatacactatgtggccaaagcaaacagaggccaatgagcaagtctgcctatatgccctgggggaaagtattgaatcaatcaggcagaaatgcccggtggatgatgcagacgcatcatctcccccgaaaactgtcccgtgtctttgccggtatgccatgactcctgaacgcgtcacccgacttcgcatgaaccatgtcacaaatataattgtgtgttcttcatttccccttccaaagtacaagatagaaggagtgcaaaaagtcaaatgctccaaggtaatgttattcgatcacaatgtgccatcgcgcgtaagtccaagggcgtatcgtgcagccgcaggcggaaaccttgcggccgtgtctgactgggtaatgagcaccgtacctgtcgcgccgcctagaagaaggagagggagaaacctgactgtgacatgtgacgagagagaagggaatataacacccatggctagcgtccgattctttagagcagagctgtgtccggccgtacaagaaacagcggagacgcgtgacacagctatttcccttcaggcaccgccaagtaccaccatggaactgagccatccaccgatctccttcggagcaccaagcgagacgttccccatcacatttggggacttcgacgaaggagaaatcgaaagcttgtcttctgagctactaactttcggagacttcctacccggtgaagtggatgatctgacagatagcgactggtccacgtgcccagacacggacgacgagttatgactagacagggcaggtgggtatatattctcgtcggacactggtccaggccatttacaacagaagtcggtacgccagtcagtgctgccggtaaacaccctggaggaagtccacgaggagaagtgttacccacctaagctggatgaattaaaggagcaactactacttaagaaactccaggagagtgcgtccatggccaatagaagcaggtatcagtcacgcaaagtggaaaatatgaaagcaacaatcatccagagactaaagagaggctgtaaactgtatttaatggcagagaccccgaaagtcccgacttatcggaccatatacccggcgcctgtgtactcgcctccgatcaatgtccgattgtccaaccccgagtccgcagtggcagcatgtaatgagttcttagctagaaactacccaactgtttcatcataccaaatcaccgacgagtatgatgcatatctagacatggtggacgggtcggagagttgcttggaccgagcgacattcaatccgtcaaaacttaggagctacccgaaacaacatgcttatcacgcgccttctatcagaagcgctgtaccttccccattccagaacacactacagaatgtactggcagcagccacgaaaaggaactgcaacgtcacacagatgagggaattacccactttggactcagcagtattcaacgtggagtgttttaaaaaattcgcatgtaaccgagaatactgggaagaatttgcagccagccctatcaggataacaactgagaatctaacaacctatgtcactaaactaaaggggccaaaagcagcagcgctgtttgcaaaaacccataatctgctgccactgcaggatgtaccaatggataggttcacagtagatatgaaaagggatgtgaaggtaactcctggtacaaagcatacagaggaaagacctaaggtgcaggttatacaggcggctgaacccttggcaacagcgtacctatgtggaattcacagagaactggttaggagattgaacgccgtcctcctacccaatgtgcatacactatttgacatgtctgccgaggacttcgatgccattatagccgcacacttcaagccaggagacgctgttttagaaacggacatagcctcctttgataagagccaagatgattcacttgcgcttaccgccttaatgctgttagaagatttgggagtggatcactccctgttggacttgatagaggctgctttcggagagatttccagctgtcatctgccgacaggtacgcgcttcaagttcggcgctatgatgaaatccggtatgttcctaactctgttcgtcaacacgttgttaaatatcaccatcgctagccgggtgttggaagatcgtctgacaaaatccgcatgcgcggccttcatcggcgacgacaacataatacatggtgtcgtctccgatgaattgatggcagccagatgcgctacttggatgaacatggaagtgaagatcatagatgcagttgtatcccagaaagctccttacttttgtggagggtttatactgcatgatactgtgacaggaacagcttgcagagtggcggacccgctaaaaaggttatttaaattgggcaaaccgttagcggcaggtgacgaacaagatgaagacagaagacgggcgctggctgatgaagtaatcagatggcaacgaacagggctaatagatgagctggagaaagcggtgtactctaggtacgaagtgcagggtatatcagttgcggtaatgtccatggccacctttgcaagctccagatccaacttcgagaagctcagaggacccgtcataactttgtacggcggtcctaaataggtacgcactacagctacctattttgcagaagccgacagcaggtacctaaataccaatcagccataatggagtttatcccaacccaaactttctacaataggaggtaccagcctcgaccttggactccgcgccctactatccaagttatcagacccagaccgcgtccgcaaaggaaagccgggcaacttgcccagctgatctcagcagttaataaactgacaatgcgcgcggtacctcaacagaagccgcgcaagaatcggaagaataagaagcaaaagcaaaagcagcaggcgccacgaaacaacatgaatcaaaagaagcagccccctaaaaagaaaccggctcaaaagaaaaagaagccgggccgtagagagagaatgtgcatgaaaatcgaaaatgattgcatcttcgaagtcaagcatgaaggtaaggtaacaggttacgcgtgcttggtaggggacaaagtaatgaagccagcacacgtaaaggggaccatcgataatgcggacctggccaaattggccttcaagcggtcatctaagtacgaccttgaatgcgcgcagatacccgtgcacatgaagtccgacgcttcgaagttcacccatgagaaaccggaggggtactacaactggcaccacggagcagtacagtactcaggaggccggttcaccatccctacaggtgcgggcaaaccaggggacagcggtagaccgatcttcgacaacaaggggcgcgtggtggccatagttttaggaggagctaatgaaggagcccgtacagccctctcggtggtgacctggaacaaagacatcgtcacgaaaatcacccctgagggggccgaagagtggagtcttgccattccagttatgtgcctgctggcaaataccacgttcccctgctcccagcccccttgcacaccctgctgctacgaaaaagagccggagaaaaccctgcgcatgctagaagacaacgtcatgagccccgggtactatcagctgctacaagcatccttaacatgttctccccgccgccagcgacgcagtattaaggacaacttcaatgtctataaagccataagaccgtacctagctcactgtcccgactgtggagaagggcactcgtgccatagtcccgtagcgctagaacgcatcagaaacgaagcgacagacgggacgctgaaaatccaggtttccttgcaaatcggaataaagacggatgatagccatgattggaccaagctgcgttacatggacaatcatatgccagcagacgcagagagggccaggctatttgtaagaacgtcagcaccgtgcacgattactggaacaatgggacacttcatcctggcccgatgtccgaaaggagaaactctgacggtgggattcactgacggtaggaagatcagtcactcatgtacgcacccatttcaccacgaccctcctgtgataggccgggaaaaatttcattcccgaccgcagcacggtagagaactaccttgcagcacgtacgcgcagagcaccgctgcaactgccgaggagatagaggtacatatgcccccagacaccccagatcgcacattgatgtcacaacagtccggtaatgtaaagatcacagtcaatagtcagacggtgcggtacaagtgtaattgcggtgactcaaatgaaggactaaccactacagacaaagtgattaataactgcaaggttgatcaatgccatgccgcggtcaccaatcacaaaaaatggcagtataattcccctctggtcccgcgtaatgctgaactcggggaccgaaaaggaaaagttcacattccgtttcctctggcaaatgtgacatgcagggtgcctaaggcaaggaaccccaccgtgacgtacggaaaaaaccaagtcatcatgctgctgtatcctgaccacccaacgctcctgtcctaccggaatatgggagaagaaccaaactatcaagaagagtgggtgacgcataagaaggagatcaggttaaccgtgccgactgaagggctcgaggtcacgtggggcaacaacgagccgtacaagtattggccgcagttatccacaaacggtacagcccacggccacccgcatgagataattttgtattattatgagctgtaccctactatgactgtggtagttgtgtcagtggcctcgttcgtactcctgtcgatggtgggtgtggcagtggggatgtgcatgtgtgcacgacgcagatgcattacaccgtacgaactgacaccaggagctaccgtccctttcctgcttagcctaatatgctgcattagaacagctaaagcggccacataccaagaggctgcggtatacctgtggaacgagcagcagcctttgttttggctgcaagcccttattccgctggcagccctgattgtcctatgcaactgtctgagactcttaccatgcttttgtaaaacgttgacttttttagccgtaatgagcgtcggtgcccacactgtgagcgcgtacgaacacgtaacagtgatcccgaacacggtgggagtaccgtataagactctagtcaacagaccgggctacagccccatggtactggagatggagcttctgtcagtcactttggagccaacgctatcgcttgattacatcacgtgcgagtataaaaccgtcatcccgtctccgtacgtgaaatgctgcggtacagcagagtgcaaggacaagagcctacctgattacagctgtaaggtcttcaccggcgtctacccattcatgtggggcggcgcctactgcttctgcgacactgaaaatacgcaattgagcgaagcacatgtggagaagtccgaatcatgcaaaacagaatttgcatcagcatatagggctcataccgcatccgcatcagctaagctccgcgtcctttaccaaggaaataatgttactgtatctgcttatgcaaacggcgatcatgccgtcacagttaaggacgctaaattcattgtggggccaatgtcttcagcctggacaccttttgacaataaaatcgtggtgtacaaaggcgacgtctacaacatggactacccgcccttcggcgcaggaagaccaggacaatttggcgacatccaaagtcgcacgcctgagagcgaagacgtctatgctaacacacaactggtactgcagagaccgtccgcgggtacggtgcacgtgccgtactctcaggcaccatctggcttcaagtattggctaaaagaacgaggggcgtcgctgcagcacacagcaccatttggctgtcaaatagcaacaaacccggtaagagcgatgaactgcgccgtagggaacatgcctatctccatcgacataccggacgcggccttcactagggtcgtcgacgcgccatctttaacggacatgtcgtgtgaggtaccagcctgcacccactcctcagactttgggggcgtagccatcattaaatatgcagccagcaagaaaggcaagtgtgcggtgcattcgatgactaacgccgtcactattcgggaagctgaaatagaagtagaagggaactctcagttgcaaatctctttttcgacggccctagccagcgccgaattccgcgtacaagtctgttctacacaagtacactgtgcagccgagtgccatccaccgaaagaccatatagtcaattacccggcgtcacacaccaccctcggggtccaagacatttccgttacggcgatgtcatgggtgcagaagatcacgggaggtgtgggactggttgtcgctgttgcagcactgatcctaatcgtggtgctatgcgtgtcgtttagcaggcactaacttgacaactaggtacgaaggtatatgtgtcccctaagagacacaccacatatagctaagaatcaatagataagtatagatcaaagggctgaacaacccctgaatagtaacaaaatataaaaatcaacaaaaatcataaaatagaaaaccagaaacagaagtaggtaagaaggtatatgtgtcccctaagagacacaccatatatagctaagaatcaatagataagtatagatcaaagggctgaataacccctgaataataacaaaatataaaaatcaataaaaatcataaaatagaaaaccataaacagaagtagttcaaagggctataaaacccctgaatagtaacaaaacataaaactaataaaaatcaaatgaataccataattggcaatcggaagagatgtaggtacttaagcttcctaaaagcagccgaactcgctttgagatgtaggcgtagcacaccgaactcttccataattctccgaacccacagggacgtaggagatgttcaaagtggctataaaaccctgaacagtaataaaacataaaattaataaggatcaaatgagtaccataattggcaaacggaagagatgtaggtacttaagcttcctaaaagcagccgaactcactttgagatgtaggcatagcataccgaactcttccacaattctccgtacccatagggacgtaggagatgttattttgtttttaatatttcaaaaaaaaaaaaaaaaaaaaaaaaaaaaaaaaaaaaaaaaagcggccgcttaattaatcgaggggaattaattcttgaagacgaaagggccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgagctcgtaatacgactcactatagg

SEQ ID NO: 9 - DNA, 13737 bp, Artificial Sequence
  plasmid construct
atggctgcgtgagacacacgtagcctaccagtttcttactgctctactctgcaaagcaagagattaataacccatcatggattctgtgtacgtggacatagacgctgacagcgcctttttgaaggccctgcaacgtgcgtaccccatgtttgaggtggaacctaggcaggtcacatcgaatgaccatgctaatgctagagcgttctcgcatctagccataaaactaatagagcaggaaattgatcccgactcaaccatcctggatataggtagtgcgccagcaaggaggatgatgtcggacaggaagtaccactgcgtttgcccgatgcgcagcgcagaagatcccgagagactcgctaattatgcgagaaagctcgcatctgccgcaggaaaagtcctggacagaaacatttctggaaagatcggggacttacaagcggtgatggccgtgccagacacggagacgccaacattttgcttacacacagatgtctcatgtagacagagagcagacgtcgcgatataccaagacgtctatgctgtacacgcacccacgtcgctataccaccaggcgattaaaggagtccgagtggcgtactgggtagggttcgacacaaccccgttcatgtacaacgctatggcgggtgcctacccctcatactcgacaaattgggcggatgagcaggtactgaaggctaagaacataggattatgttcaacagacctgacggaaggtagacgaggcaaattgtctatcatgagagggaaaaagctaaaaccgtgcgaccgtgtgctgttctcagtagggtcaacgctttacccggaaagccgcacgctacttaagagctggcacctaccatcggtgttccatctaaagggcaagcttagcttcacatgccgctgtgacacagtggtttcgtgtgagggctacgtcgttaagagaataacgatgagcccaggcctttatggaaaaaccatagggtatgcggtaacccaccacgcagacggattcttgatgtgcaagactaccgacacggttgacggcgaaagagtgtcattctcggtgtgcacgtacgtgccggcgaccatttgtgatcaaatgaccggcatccttgctacagaagtcacgccggaggatgcacagaagctgttggtggggctgaaccagaggatagtggttaacggcagaacgcaacggaacacgaacaccatgaagaactacctacttcccgtggtcgcccaggccttcagtaagtgggcaaaggagtgccggaaggacatggaagatgagaagcttctgggggtcagagaaagaacactaacctgctgctgtctatgggcatttaagaagcagaaaacacacacggtctacaagaggcctgatacccagtcaatccagaaggttcaggccgaatttgacagctttgtagtaccgggcctgtggtcgtccgggttgtcaatcccgttgaggactagaatcaagtggttgttacgcaaggtgccgaaaacagacctgatcccatacagcgggaatgcccaagaagcccaggatgcagaaaaagaagcagaggaagaacgagaagcagaactgactcatgaggctctaccacccctacaggcagcacaggaagatgtccaggtcgaaatcgacgtggaacagcttgaggatagagctggtgctggaataatagagactccgagaggcgctatcaaagttactgcccaactaacagaccacgtcgtgggggagtacctggtactttccccgcagaccgtactacgcagccagaagctcagcctgatccacgctttagcggagcaagtgaagacgtgtacgcacagcggacgagcagggaggtatgcggtcgaagcgtacgatggccgagtcctagtgccctcaggctatgcaatttcgcctgaagacttccagagtctaagcgaaagcgcaacgatggtgtacaacgaaagagagttcgtaaacagaaagttacaccacattgcgatgcacggaccagccctgaacactgacgaagagtcgtatgagctggtgagggcagagaggacagaacacgagtacgtctacgacgtggaccagagaagatgctgtaagaaggaagaagctgcaggactggtactggtgggcgacttgactaatccgccctaccacgaattcgcatacgaagggctaaaaattcgccccgcctgcccatacaaaattgcagtcataggagtcttcggggtaccaggatctggcaagtcagccattatcaagaacctagttaccaggcaagacctggtgactagcggaaagaaagaaaactgccaagaaatcagcaccgacgtgatgagacagagaggtctagagatatctgcacgtacggtagattcgctgctcttgaatggatgcaacagaccagtcgacgtgttgtacgtagacgaggcgtttgcgtgccactctggaacgttacttgctttgatcgccttggtgagaccaagacagaaagttgtactttgtggtgacccgaagcagtgcggcttcttcaatatgatgcagatgaaagtcaactacaatcataacatctgcacccaagtgtaccacaaaagtatctccaggcggtgtacactgcctgtgactgccattgtgtcatcgttgcattacgaaggcaaaatgcgcactacgaatgagtacaacatgccgattgtagtggacactacaggctcaacgaaacctgaccctggagacctcgtgttaacgtgcttcagagggtgggttaaacaactgcaaattgactatcgtggacacgaggtcatgacagcagccgcatcccaagggttaactagaaaaggagtttacgcagttaggcaaaaagttaacgaaaacccactctatgcatcaacatcagagcacgtcaacgtactcctaacgcgtacggaaggtaaactggtatggaagacactctctggtgacccgtggataaagacgctgcagaacccaccgaaaggaaacttcaaagcaactattaaggagtgggaggtggagcacgcatcgataatggcgggcatctgcagtcaccaagtgacctttgacacattccaaaacaaagccaacgtttgctgggctaagagcttggtccctatcctcgaaacagcggggataaaactaaatgataggcagtggtcccagataattcaagccttcaaagaagacaaagcatactcacccgaagtagccctgaatgaaatatgcacgcgcatgtatggggtggatctagacagtgggctattctctaaaccgttggtatctgtgtattacgcggataaccattgggataataggccgggaggaaagatgttcggattcaaccctgaggcagcgtccattctagaaagaaagtacccatttacaaaaggaaagtggaacatcaacaagcagatctgcgtgactaccaggaggatagaagacttcaaccctaccaccaacattataccggtcaacaggagactaccacactcattagtggccgaacaccgcccagtaaaaggggaaagaatggaatggctggttaacaagataaacggacaccacgtactcctggttagcggctataaccttgcactgcctactaagagagtcacctgggtagcgccactaggtgtccgcggagcggactatacatacaacctagagctgggtctaccagcaacgcttggtaggtatgacctagtggtcataaacatccacacaccttttcgcatacaccattaccaacagtgcgtagatcacgcaatgaaactgcaaatgctagggggtgactcactgagactgctcaaaccgggtggctctctattgatcagagcatacggttacgcagatagaaccagtgaacgagtcatctgcgtactgggacgcaagtttagatcgtctagagcattgaaaccaccatgtgtcaccagtaatactgagatgtttttcctatttagcaattttgacaatggcagaaggaattttacaacgcatgtcatgaacaatcaactgaatgcagcctttgtaggacaggccacccgagcaggatgtgcaccatcgtaccgggtaaaacgcatggacatcgcgaagaacgatgaagagtgcgtggttaacgccgccaaccctcgcgggttaccaggtgacggtgtttgcaaggcagtatataaaaagtggccggagtcctttaaaaacagtgcaacaccagtaggaaccgcaaaaacagttatgtgcggtacgtatccagtaatccacgccgtaggaccaaacttctcaaattattcggagtctgaaggggaccgggaattggcggctgcctatcgagaagtcgcaaaggaagtaactagactgggagtaaatagcgtagctatacctctcctctccacaggtgtatactcaggagggaaagacaggctaacccagtcactgaaccacctctttacagccatggactcgacggatgcagacgtggtcatctactgccgagacaaggaatgggagaagaaaatatctgaggccatacagatgcggacccaagtggagctgctggatgagcacatctccatagactgcgatgtcattcgcgtgcaccctgacagtagcttggcaggcagaaaaggatacagcaccacggaaggcgcactgtattcatatctagaagggacacgttttcaccagacggcagtggatatggcagagatatacactatgtggccaaagcaaacagaggccaatgagcaagtctgcctatatgccctgggggaaagtattgaatcaatcaggcagaaatgcccggtggatgatgcagacgcatcatctcccccgaaaactgtcccgtgtctttgccggtatgccatgactcctgaacgcgtcacccgacttcgcatgaaccatgtcacaaatataattgtgtgttcttcatttccccttccaaagtacaagatagaaggagtgcaaaaagtcaaatgctccaaggtaatgttattcgatcacaatgtgccatcgcgcgtaagtccaagggaatacagatcttcccaggagtctgtacaggaagtgagtacgacaacgtcattgacgcatagccagtttgatctaagcgccgatggcgagacactgcctgtcccgtcagacctggatgctgacgccccagccctagaaccggccctagacgacggggcggtacatacattaccaaccataatcggaaaccttgcggccgtgtctgactgggtaatgagcaccgtacctgtcgcgccgcctagaagaaggagagggagaaacctgactgtgacatgtgacgagagagaagggaatataacacccatggctagcgtccgattctttagagcagagctgtgtccggccgtacaagaaacagcggagacgcgtgacacagctatttcccttcaggcaccgccaagtaccaccatggaactgagccatccaccgatctccttcggagcaccaagcgagacgttccccatcacatttggggacttcgacgaaggagaaatcgaaagcttgtcttctgagctactaactttcggagacttcctacccggtgaagtggatgatctgacagatagcgactggtccacgtgcccagacacggacgacgagttatgactagacagggcaggtgggtatatattctcgtcggacactggtccaggccatttacaacagaagtcggtacgccagtcagtgctgccggtaaacaccctggaggaagtccacgaggagaagtgttacccacctaagctggatgaattaaaggagcaactactacttaagaaactccaggagagtgcgtccatggccaatagaagcaggtatcagtcacgcaaagtggaaaatatgaaagcaacaatcatccagagactaaagagaggctgtaaactgtatttaatggcagagaccccgaaagtcccgacttatcggaccatatacccggcgcctgtgtactcgcctccgatcaatgtccgattgtccaaccccgagtccgcagtggcagcatgtaatgagttcttagctagaaactacccaactgtttcatcataccaaatcaccgacgagtatgatgcatatctagacatggtggacgggtcggagagttgcttggaccgagcgacattcaatccgtcaaaacttaggagctacccgaaacaacatgcttatcacgcgccttctatcagaagcgctgtaccttccccattccagaacacactacagaatgtactggcagcagccacgaaaaggaactgcaacgtcacacagatgagggaattacccactttggactcagcagtattcaacgtggagtgttttaaaaaattcgcatgtaaccgagaatactgggaagaatttgcagccagccctatcaggataacaactgagaatctaacaacctatgtcactaaactaaaggggccaaaagcagcagcgctgtttgcaaaaacccataatctgctgccactgcaggatgtaccaatggataggttcacagtagatatgaaaagggatgtgaaggtaactcctggtacaaagcatacagaggaaagacctaaggtgcaggttatacaggcggctgaacccttggcaacagcgtacctatgtggaattcacagagaactggttaggagattgaacgccgtcctcctacccaatgtgcatacactatttgacatgtctgccgaggacttcgatgccattatagccgcacacttcaagccaggagacgctgttttagaaacggacatagcctcctttgataagagccaagatgattcacttgcgcttaccgccttaatgctgttagaagatttgggagtggatcactccctgttggacttgatagaggctgctttcggagagatttccagctgtcatctgccgacaggtacgcgcttcaagttcggcgctatgatgaaatccggtatgttcctaactctgttcgtcaacacgttgttaaatatcaccatcgctagccgggtgttggaagatcgtctgacaaaatccgcatgcgcggccttcatcggcgacgacaacataatacatggtgtcgtctccgatgaattgatggcagccagatgcgctacttggatgaacatggaagtgaagatcatagatgcagttgtatcccagaaagctccttacttttgtggagggtttatactgcatgatactgtgacaggaacagcttgcagagtggcggacccgctaaaaaggttatttaaattgggcaaaccgttagcggcaggtgacgaacaagatgaagacagaagacgggcgctggctgatgaagtaatcagatggcaacgaacagggctaatagatgagctggagaaagcggtgtactctaggtacgaagtgcagggtatatcagttgcggtaatgtccatggccacctttgcaagctccagatccaacttcgagaagctcagaggacccgtcataactttgtacggcggtcctaaataggtacgcactacagctacctattttgcagaagccgacagcaggtacctaaataccaatcagccataatggagtttatcccaacccaaactttctacaataggaggtaccagcctcgaccttggactccgcgccctactatccaagttatcagacccagaccgcgtccgcaaaggaaagccgggcaacttgcccagctgatctcagcagttaataaactgacaatgcgcgcggtacctcaacagaagccgcgcaagaatcggaagaataagaagcaaaagcaaaagcagcaggcgccacgaaacaacatgaatcaaaagaagcagccccctaaaaagaaaccggctcaaaagaaaaagaagccgggccgtagagagagaatgtgcatgaaaatcgaaaatgattgcatcttcgaagtcaagcatgaaggtaaggtaacaggttacgcgtgcttggtaggggacaaagtaatgaagccagcacacgtaaaggggaccatcgataatgcggacctggccaaattggccttcaagcggtcatctaagtacgaccttgaatgcgcgcagatacccgtgcacatgaagtccgacgcttcgaagttcacccatgagaaaccggaggggtactacaactggcaccacggagcagtacagtactcaggaggccggttcaccatccctacaggtgcgggcaaaccaggggacagcggtagaccgatcttcgacaacaaggggcgcgtggtggccatagttttaggaggagctaatgaaggagcccgtacagccctctcggtggtgacctggaacaaagacatcgtcacgaaaatcacccctgagggggccgaagagtggagtcttgccattccagttatgtgcctgctggcaaataccacgttcccctgctcccagcccccttgcacaccctgctgctacgaaaaagagccggagaaaaccctgcgcatgctagaagacaacgtcatgagccccgggtactatcagctgctacaagcatccttaacatgttctccccgccgccagcgacgcagtattaaggacaacttcaatgtctataaagccataagaccgtacctagctcactgtcccgactgtggagaagggcactcgtgccatagtcccgtagcgctagaacgcatcagaaacgaagcgacagacgggacgctgaaaatccaggtttccttgcaaatcggaataaagacggatgatagccatgattggaccaagctgcgttacatggacaatcatatgccagcagacgcagagagggccaggctatttgtaagaacgtcagcaccgtgcacgattactggaacaatgggacacttcatcctggcccgatgtccgaaaggagaaactctgacggtgggattcactgacggtaggaagatcagtcactcatgtacgcacccatttcaccacgaccctcctgtgataggccgggaaaaatttcattcccgaccgcagcacggtagagaactaccttgcagcacgtacgcgcagagcaccgctgcaactgccgaggagatagaggtacatatgcccccagacaccccagatcgcacattgatgtcacaacagtccggtaatgtaaagatcacagtcaatagtcagacggtgcggtacaagtgtaattgcggtgactcaaatgaaggactaaccactacagacaaagtgattaataactgcaaggttgatcaatgccatgccgcggtcaccaatcacaaaaaatggcagtataattcccctctggtcccgcgtaatgctgaactcggggaccgaaaaggaaaagttcacattccgtttcctctggcaaatgtgacatgcagggtgcctaaggcaaggaaccccaccgtgacgtacggaaaaaaccaagtcatcatgctgctgtatcctgaccacccaacgctcctgtcctaccggaatatgggagaagaaccaaactatcaagaagagtgggtgacgcataagaaggagatcaggttaaccgtgccgactgaagggctcgaggtcacgtggggcaacaacgagccgtacaagtattggccgcagttatccacaaacggtacagcccacggccacccgcatgagataattttgtattattatgagctgtaccctactatgactgtggtagttgtgtcagtggcctcgttcgtactcctgtcgatggtgggtgtggcagtggggatgtgcatgtgtgcacgacgcagatgcattacaccgtacgaactgacaccaggagctaccgtccctttcctgcttagcctaatatgctgcattagaacagctaaagcggcgtacgaacacgtaacagtgatcccgaacacggtgggagtaccgtataagactctagtcaacagaccgggctacagccccatggtactggagatggagcttctgtcagtcactttggagccaacgctatcgcttgattacatcacgtgcgagtataaaaccgtcatcccgtctccgtacgtgaaatgctgcggtacagcagagtgcaaggacaagagcctacctgattacagctgtaaggtcttcaccggcgtctacccattcatgtggggcggcgcctactgcttctgcgacactgaaaatacgcaattgagcgaagcacatgtggagaagtccgaatcatgcaaaacagaatttgcatcagcatatagggctcataccgcatccgcatcagctaagctccgcgtcctttaccaaggaaataatgttactgtatctgcttatgcaaacggcgatcatgccgtcacagttaaggacgctaaattcattgtggggccaatgtcttcagcctggacaccttttgacaataaaatcgtggtgtacaaaggcgacgtctacaacatggactacccgcccttcggcgcaggaagaccaggacaatttggcgacatccaaagtcgcacgcctgagagcgaagacgtctatgctaacacacaactggtactgcagagaccgtccgcgggtacggtgcacgtgccgtactctcaggcaccatctggcttcaagtattggctaaaagaacgaggggcgtcgctgcagcacacagcaccatttggctgtcaaatagcaacaaacccggtaagagcgatgaactgcgccgtagggaacatgcctatctccatcgacataccggacgcggccttcactagggtcgtcgacgcgccatctttaacggacatgtcgtgtgaggtaccagcctgcacccactcctcagactttgggggcgtagccatcattaaatatgcagccagcaagaaaggcaagtgtgcggtgcattcgatgactaacgccgtcactattcgggaagctgaaatagaagtagaagggaactctcagttgcaaatctctttttcgacggccctagccagcgccgaattccgcgtacaagtctgttctacacaagtacactgtgcagccgagtgccatccaccgaaagaccatatagtcaattacccggcgtcacacaccaccctcggggtccaagacatttccgttacggcgatgtcatgggtgcagaagatcacgggaggtgtgggactggttgtcgctgttgcagcactgatcctaatcgtggtgctatgcgtgtcgtttagcaggcactaacttgacaactaggtacgaaggtatatgtgtcccctaagagacacaccacatatagctaagaatcaatagataagtatagatcaaagggctgaacaacccctgaatagtaacaaaatataaaaatcaacaaaaatcataaaatagaaaaccagaaacagaagtaggtaagaaggtatatgtgtcccctaagagacacaccatatatagctaagaatcaatagataagtatagatcaaagggctgaataacccctgaataataacaaaatataaaaatcaataaaaatcataaaatagaaaaccataaacagaagtagttcaaagggctataaaacccctgaatagtaacaaaacataaaactaataaaaatcaaatgaataccataattggcaatcggaagagatgtaggtacttaagcttcctaaaagcagccgaactcgctttgagatgtaggcgtagcacaccgaactcttccataattctccgaacccacagggacgtaggagatgttcaaagtggctataaaaccctgaacagtaataaaacataaaattaataaggatcaaatgagtaccataattggcaaacggaagagatgtaggtacttaagcttcctaaaagcagccgaactcactttgagatgtaggcatagcataccgaactcttccacaattctccgtacccatagggacgtaggagatgttattttgtttttaatatttcaaaaaaaaaaaaaaaaaaaaaaaaaaaaaaaaaaaaaaaaagcggccgcttaattaatcgaggggaattaattcttgaagacgaaagggccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgagctcgtaatacgactcactatagg

SEQ ID NO: 10 - DNA, 14442 bp, Artificial Sequence
  plasmid construct
atggctgcgtgagacacacgtagcctaccagtttcttactgctctactctgcaaagcaagagattaataacccatcatggattctgtgtacgtggacatagacgctgacagcgcctttttgaaggccctgcaacgtgcgtaccccatgtttgaggtggaacctaggcaggtcacatcgaatgaccatgctaatgctagagcgttctcgcatctagccataaaactaatagagcaggaaattgatcccgactcaaccatcctggatataggtagtgcgccagcaaggaggatgatgtcggacaggaagtaccactgcgtttgcccgatgcgcagcgcagaagatcccgagagactcgctaattatgcgagaaagctcgcatctgccgcaggaaaagtcctggacagaaacatttctggaaagatcggggacttacaagcggtgatggccgtgccagacacggagacgccaacattttgcttacacacagatgtctcatgtagacagagagcagacgtcgcgatataccaagacgtctatgctgtacacgcacccacgtcgctataccaccaggcgattaaaggagtccgagtggcgtactgggtagggttcgacacaaccccgttcatgtacaacgctatggcgggtgcctacccctcatactcgacaaattgggcggatgagcaggtactgaaggctaagaacataggattatgttcaacagacctgacggaaggtagacgaggcaaattgtctatcatgagagggaaaaagctaaaaccgtgcgaccgtgtgctgttctcagtagggtcaacgctttacccggaaagccgcacgctacttaagagctggcacctaccatcggtgttccatctaaagggcaagcttagcttcacatgccgctgtgacacagtggtttcgtgtgagggctacgtcgttaagagaataacgatgagcccaggcctttatggaaaaaccatagggtatgcggtaacccaccacgcagacggattcttgatgtgcaagactaccgacacggttgacggcgaaagagtgtcattctcggtgtgcacgtacgtgccggcgaccatttgtgatcaaatgaccggcatccttgctacagaagtcacgccggaggatgcacagaagctgttggtggggctgaaccagaggatagtggttaacggcagaacgcaacggaacacgaacaccatgaagaactacctacttcccgtggtcgcccaggccttcagtaagtgggcaaaggagtgccggaaggacatggaagatgagaagcttctgggggtcagagaaagaacactaacctgctgctgtctatgggcatttaagaagcagaaaacacacacggtctacaagaggcctgatacccagtcaatccagaaggttcaggccgaatttgacagctttgtagtaccgggcctgtggtcgtccgggttgtcaatcccgttgaggactagaatcaagtggttgttacgcaaggtgccgaaaacagacctgatcccatacagcgggaatgcccaagaagcccaggatgcagaaaaagaagcagaggaagaacgagaagcagaactgactcatgaggctctaccacccctacaggcagcacaggaagatgtccaggtcgaaatcgacgtggaacagcttgaggatagagctggtgctggaataatagagactccgagaggcgctatcaaagttactgcccaactaacagaccacgtcgtgggggagtacctggtactttccccgcagaccgtactacgcagccagaagctcagcctgatccacgctttagcggagcaagtgaagacgtgtacgcacagcggacgagcagggaggtatgcggtcgaagcgtacgatggccgagtcctagtgccctcaggctatgcaatttcgcctgaagacttccagagtctaagcgaaagcgcaacgatggtgtacaacgaaagagagttcgtaaacagaaagttacaccacattgcgatgcacggaccagccctgaacactgacgaagagtcgtatgagctggtgagggcagagaggacagaacacgagtacgtctacgacgtggaccagagaagatgctgtaagaaggaagaagctgcaggactggtactggtgggcgacttgactaatccgccctaccacgaattcgcatacgaagggctaaaaattcgccccgcctgcccatacaaaattgcagtcataggagtcttcggggtaccaggatctggcaagtcagccattatcaagaacctagttaccaggcaagacctggtgactagcggaaagaaagaaaactgccaagaaatcagcaccgacgtgatgagacagagaggtctagagatatctgcacgtacggtagattcgctgctcttgaatggatgcaacagaccagtcgacgtgttgtacgtagacgaggcgtttgcgtgccactctggaacgttacttgctttgatcgccttggtgagaccaagacagaaagttgtactttgtggtgacccgaagcagtgcggcttcttcaatatgatgcagatgaaagtcaactacaatcataacatctgcacccaagtgtaccacaaaagtatctccaggcggtgtacactgcctgtgactgccattgtgtcatcgttgcattacgaaggcaaaatgcgcactacgaatgagtacaacatgccgattgtagtggacactacaggctcaacgaaacctgaccctggagacctcgtgttaacgtgcttcagagggtgggttaaacaactgcaaattgactatcgtggacacgaggtcatgacagcagccgcatcccaagggttaactagaaaaggagtttacgcagttaggcaaaaagttaacgaaaacccactctatgcatcaacatcagagcacgtcaacgtactcctaacgcgtacggaaggtaaactggtatggaagacactctctggtgacccgtggataaagacgctgcagaacccaccgaaaggaaacttcaaagcaactattaaggagtgggaggtggagcacgcatcgataatggcgggcatctgcagtcaccaagtgacctttgacacattccaaaacaaagccaacgtttgctgggctaagagcttggtccctatcctcgaaacagcggggataaaactaaatgataggcagtggtcccagataattcaagccttcaaagaagacaaagcatactcacccgaagtagccctgaatgaaatatgcacgcgcatgtatggggtggatctagacagtgggctattctctaaaccgttggtatctgtgtattacgcggataaccattgggataataggccgggaggaaagatgttcggattcaaccctgaggcagcgtccattctagaaagaaagtacccatttacaaaaggaaagtggaacatcaacaagcagatctgcgtgactaccaggaggatagaagacttcaaccctaccaccaacattataccggtcaacaggagactaccacactcattagtggccgaacaccgcccagtaaaaggggaaagaatggaatggctggttaacaagataaacggacaccacgtactcctggttagcggctataaccttgcactgcctactaagagagtcacctgggtagcgccactaggtgtccgcggagcggactatacatacaacctagagctgggtctaccagcaacgcttggtaggtatgacctagtggtcataaacatccacacaccttttcgcatacaccattaccaacagtgcgtagatcacgcaatgaaactgcaaatgctagggggtgactcactgagactgctcaaaccgggtggctctctattgatcagagcatacggttacgcagatagaaccagtgaacgagtcatctgcgtactgggacgcaagtttagatcgtctagagcattgaaaccaccatgtgtcaccagtaatactgagatgtttttcctatttagcaattttgacaatggcagaaggaattttacaacgcatgtcatgaacaatcaactgaatgcagcctttgtaggacaggccacccgagcaggatgtgcaccatcgtaccgggtaaaacgcatggacatcgcgaagaacgatgaagagtgcgtggttaacgccgccaaccctcgcgggttaccaggtgacggtgtttgcaaggcagtatataaaaagtggccggagtcctttaaaaacagtgcaacaccagtaggaaccgcaaaaacagttatgtgcggtacgtatccagtaatccacgccgtaggaccaaacttctcaaattattcggagtctgaaggggaccgggaattggcggctgcctatcgagaagtcgcaaaggaagtaactagactgggagtaaatagcgtagctatacctctcctctccacaggtgtatactcaggagggaaagacaggctaacccagtcactgaaccacctctttacagccatggactcgacggatgcagacgtggtcatctactgccgagacaaggaatgggagaagaaaatatctgaggccatacagatgcggacccaagtggagctgctggatgagcacatctccatagactgcgatgtcattcgcgtgcaccctgacagtagcttggcaggcagaaaaggatacagcaccacggaaggcgcactgtattcatatctagaagggacacgttttcaccagacggcagtggatatggcagagatatacactatgtggccaaagcaaacagaggccaatgagcaagtctgcctatatgccctgggggaaagtattgaatcaatcaggcagaaatgcccggtggatgatgcagacgcatcatctcccccgaaaactgtcccgtgtctttgccggtatgccatgactcctgaacgcgtcacccgacttcgcatgaaccatgtcacaaatataattgtgtgttcttcatttccccttccaaagtacaagatagaaggagtgcaaaaagtcaaatgctccaaggtaatgttattcgatcacaatgtgccatcgcgcgtaagtccaagggaatacagatcttcccaggagtctgtacaggaagtgagtacgacaacgtcattgacgcatagccagtttgatctaagcgccgatggcgagacactgcctgtcccgtcagacctggatgctgacgccccagccctagaaccggccctagacgacggggcggtacatacattaccaaccataatcggaaaccttgcggccgtgtctgactgggtaatgagcaccgtacctgtcgcgccgcctagaagaaggagagggagaaacctgactgtgacatgtgacgagagagaagggaatataacacccatggctagcgtccgattctttagagcagagctgtgtccggccgtacaagaaacagcggagacgcgtgacacagctatttcccttcaggcaccgccaagtaccaccatggaactgagccatccaccgatctccttcggagcaccaagcgagacgttccccatcacatttggggacttcgacgaaggagaaatcgaaagcttgtcttctgagctactaactttcggagacttcctacccggtgaagtggatgatctgacagatagcgactggtccacgtgcccagacacggacgacgagttatgactagacagggcaggtgggtatatattctcgtcggacactggtccaggccatttacaacagaagtcggtacgccagtcagtgctgccggtaaacaccctggaggaagtccacgaggagaagtgttacccacctaagctggatgaattaaaggagcaactactacttaagaaactccaggagagtgcgtccatggccaatagaagcaggtatcagtcacgcaaagtggaaaatatgaaagcaacaatcatccagagactaaagagaggctgtaaactgtatttaatggcagagaccccgaaagtcccgacttatcggaccatatacccggcgcctgtgtactcgcctccgatcaatgtccgattgtccaaccccgagtccgcagtggcagcatgtaatgagttcttagctagaaactacccaactgtttcatcataccaaatcaccgacgagtatgatgcatatctagacatggtggacgggtcggagagttgcttggaccgagcgacattcaatccgtcaaaacttaggagctacccgaaacaacatgcttatcacgcgccttctatcagaagcgctgtaccttccccattccagaacacactacagaatgtactggcagcagccacgaaaaggaactgcaacgtcacacagatgagggaattacccactttggactcagcagtattcaacgtggagtgttttaaaaaattcgcatgtaaccgagaatactgggaagaatttgcagccagccctatcaggataacaactgagaatctaacaacctatgtcactaaactaaaggggccaaaagcagcagcgctgtttgcaaaaacccataatctgctgccactgcaggatgtaccaatggataggttcacagtagatatgaaaagggatgtgaaggtaactcctggtacaaagcatacagaggaaagacctaaggtgcaggttatacaggcggctgaacccttggcaacagcgtacctatgtggaattcacagagaactggttaggagattgaacgccgtcctcctacccaatgtgcatacactatttgacatgtctgccgaggacttcgatgccattatagccgcacacttcaagccaggagacgctgttttagaaacggacatagcctcctttgataagagccaagatgattcacttgcgcttaccgccttaatgctgttagaagatttgggagtggatcactccctgttggacttgatagaggctgctttcggagagatttccagctgtcatctgccgacaggtacgcgcttcaagttcggcgctatgatgaaatccggtatgttcctaactctgttcgtcaacacgttgttaaatatcaccatcgctagccgggtgttggaagatcgtctgacaaaatccgcatgcgcggccttcatcggcgacgacaacataatacatggtgtcgtctccgatgaattgatggcagccagatgcgctacttggatgaacatggaagtgaagatcatagatgcagttgtatcccagaaagctccttacttttgtggagggtttatactgcatgatactgtgacaggaacagcttgcagagtggcggacccgctaaaaaggttatttaaattgggcaaaccgttagcggcaggtgacgaacaagatgaagacagaagacgggcgctggctgatgaagtaatcagatggcaacgaacagggctaatagatgagctggagaaagcggtgtactctaggtacgaagtgcagggtatatcagttgcggtaatgtccatggccacctttgcaagctccagatccaacttcgagaagctcagaggacccgtcataacgctttatggaggaccgaagtaataacccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatggagtttatcccaacccaaactttctacaataggaggtaccagcctcgaccttggactccgcgccctactatccaagttatcagacccagaccgcgtccgcaaaggaaagccgggcaacttgcccagctgatctcagcagttaataaactgacaatgcgcgcggtacctcaacagaagccgcgcaagaatcggaagaataagaagcaaaagcaaaagcagcaggcgccacgaaacaacatgaatcaaaagaagcagccccctaaaaagaaaccggctcaaaagaaaaagaagccgggccgtagagagagaatgtgcatgaaaatcgaaaatgattgcatcttcgaagtcaagcatgaaggtaaggtaacaggttacgcgtgcttggtaggggacaaagtaatgaagccagcacacgtaaaggggaccatcgataatgcggacctggccaaattggccttcaagcggtcatctaagtacgaccttgaatgcgcgcagatacccgtgcacatgaagtccgacgcttcgaagttcacccatgagaaaccggaggggtactacaactggcaccacggagcagtacagtactcaggaggccggttcaccatccctacaggtgcgggcaaaccaggggacagcggtagaccgatcttcgacaacaaggggcgcgtggtggccatagttttaggaggagctaatgaaggagcccgtacagccctctcggtggtgacctggaacaaagacatcgtcacgaaaatcacccctgagggggccgaagagtggagtcttgccattccagttatgtgcctgctggcaaataccacgttcccctgctcccagcccccttgcacaccctgctgctacgaaaaagagccggagaaaaccctgcgcatgctagaagacaacgtcatgagccccgggtactatcagctgctacaagcatccttaacatgttctccccgccgccagcgacgcagtattaaggacaacttcaatgtctataaagccataagaccgtacctagctcactgtcccgactgtggagaagggcactcgtgccatagtcccgtagcgctagaacgcatcagaaacgaagcgacagacgggacgctgaaaatccaggtttccttgcaaatcggaataaagacggatgatagccatgattggaccaagctgcgttacatggacaatcatatgccagcagacgcagagagggccaggctatttgtaagaacgtcagcaccgtgcacgattactggaacaatgggacacttcatcctggcccgatgtccgaaaggagaaactctgacggtgggattcactgacggtaggaagatcagtcactcatgtacgcacccatttcaccacgaccctcctgtgataggccgggaaaaatttcattcccgaccgcagcacggtagagaactaccttgcagcacgtacgcgcagagcaccgctgcaactgccgaggagatagaggtacatatgcccccagacaccccagatcgcacattgatgtcacaacagtccggtaatgtaaagatcacagtcaatagtcagacggtgcggtacaagtgtaattgcggtgactcaaatgaaggactaaccactacagacaaagtgattaataactgcaaggttgatcaatgccatgccgcggtcaccaatcacaaaaaatggcagtataattcccctctggtcccgcgtaatgctgaactcggggaccgaaaaggaaaagttcacattccgtttcctctggcaaatgtgacatgcagggtgcctaaggcaaggaaccccaccgtgacgtacggaaaaaaccaagtcatcatgctgctgtatcctgaccacccaacgctcctgtcctaccggaatatgggagaagaaccaaactatcaagaagagtgggtgacgcataagaaggagatcaggttaaccgtgccgactgaagggctcgaggtcacgtggggcaacaacgagccgtacaagtattggccgcagttatccacaaacggtacagcccacggccacccgcatgagataattttgtattattatgagctgtaccctactatgactgtggtagttgtgtcagtggcctcgttcgtactcctgtcgatggtgggtgtggcagtggggatgtgcatgtgtgcacgacgcagatgcattacaccgtacgaactgacaccaggagctaccgtccctttcctgcttagcctaatatgctgcattagaacagctaaagcggccacataccaagaggctgcggtatacctgtggaacgagcagcagcctttgttttggctgcaagcccttattccgctggcagccctgattgtcctatgcaactgtctgagactcttaccatgcttttgtaaaacgttgacttttttagccgtaatgagcgtcggtgcccacactgtgagcgcgtacgaacacgtaacagtgatcccgaacacggtgggagtaccgtataagactctagtcaacagaccgggctacagccccatggtactggagatggagcttctgtcagtcactttggagccaacgctatcgcttgattacatcacgtgcgagtataaaaccgtcatcccgtctccgtacgtgaaatgctgcggtacagcagagtgcaaggacaagagcctacctgattacagctgtaaggtcttcaccggcgtctacccattcatgtggggcggcgcctactgcttctgcgacactgaaaatacgcaattgagcgaagcacatgtggagaagtccgaatcatgcaaaacagaatttgcatcagcatatagggctcataccgcatccgcatcagctaagctccgcgtcctttaccaaggaaataatgttactgtatctgcttatgcaaacggcgatcatgccgtcacagttaaggacgctaaattcattgtggggccaatgtcttcagcctggacaccttttgacaataaaatcgtggtgtacaaaggcgacgtctacaacatggactacccgcccttcggcgcaggaagaccaggacaatttggcgacatccaaagtcgcacgcctgagagcgaagacgtctatgctaacacacaactggtactgcagagaccgtccgcgggtacggtgcacgtgccgtactctcaggcaccatctggcttcaagtattggctaaaagaacgaggggcgtcgctgcagcacacagcaccatttggctgtcaaatagcaacaaacccggtaagagcgatgaactgcgccgtagggaacatgcctatctccatcgacataccggacgcggccttcactagggtcgtcgacgcgccatctttaacggacatgtcgtgtgaggtaccagcctgcacccactcctcagactttgggggcgtagccatcattaaatatgcagccagcaagaaaggcaagtgtgcggtgcattcgatgactaacgccgtcactattcgggaagctgaaatagaagtagaagggaactctcagttgcaaatctctttttcgacggccctagccagcgccgaattccgcgtacaagtctgttctacacaagtacactgtgcagccgagtgccatccaccgaaagaccatatagtcaattacccggcgtcacacaccaccctcggggtccaagacatttccgttacggcgatgtcatgggtgcagaagatcacgggaggtgtgggactggttgtcgctgttgcagcactgatcctaatcgtggtgctatgcgtgtcgtttagcaggcactaacttgacaactaggtacgaaggtatatgtgtcccctaagagacacaccacatatagctaagaatcaatagataagtatagatcaaagggctgaacaacccctgaatagtaacaaaatataaaaatcaacaaaaatcataaaatagaaaaccagaaacagaagtaggtaagaaggtatatgtgtcccctaagagacacaccatatatagctaagaatcaatagataagtatagatcaaagggctgaataacccctgaataataacaaaatataaaaatcaataaaaatcataaaatagaaaaccataaacagaagtagttcaaagggctataaaacccctgaatagtaacaaaacataaaactaataaaaatcaaatgaataccataattggcaatcggaagagatgtaggtacttaagcttcctaaaagcagccgaactcgctttgagatgtaggcgtagcacaccgaactcttccataattctccgaacccacagggacgtaggagatgttcaaagtggctataaaaccctgaacagtaataaaacataaaattaataaggatcaaatgagtaccataattggcaaacggaagagatgtaggtacttaagcttcctaaaagcagccgaactcactttgagatgtaggcatagcataccgaactcttccacaattctccgtacccatagggacgtaggagatgttattttgtttttaatatttcaaaaaaaaaaaaaaaaaaaaaaaaaaaaaaaaaaaaaaaaagcggccgcttaattaatcgaggggaattaattcttgaagacgaaagggccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgagctcgtaatacgactcactatagg

SEQ ID NO: 11 - DNA, 24 bp, Artificial Sequence
  PCR primer
tcactccctgttggacttgataga

SEQ ID NO: 12 - DNA, 26 bp, Artificial Sequence
  PCR primer
ttgacgaacagagttaggaacatacc

SEQ ID NO: 13 - RNA, 10862 nt, Yellow fever virus
agugguuuuguguuugucauccaaaggucugcuuauucuugagcaaacugugcucacagaccucuggaggaaaagcagagaaccacuccggucuuucccuggcgucaauauggucccacuauuuguucccuggagggucuccucuaaccucuagaccccgucuuucuaccaccacgugggagggugguagcggaggcuccguucuuuuuacucuggggugggaggucccagaaaccagguuuuucgcaaccuggaggucggcugucccagccugcacugccucacagcuuagcaguggcaaaacucgugugggguucugggcugacaacuucuucugucucaauguguggaguccgguucuccauccguaguuuuuaucccgguuucuguuucauuuuaagugcggagcccgguucuccagccgugguuuauaucccgguuucagguuguggggaguccgguucuccacccgugguuuguaucccgguuauuccuguuagaugguguuucagauaagcucacccaguugcaggucagcauccacagaauaccuguccaugacuguuagguagucaguguauuucuccuguccaaucagcguucggauacgauggaugacuaaauggaugugggaggcccagguggcccuauuggucauuccaaucagugauccgcacagcuugucuugucucuugguuagauaagggacaucucuccauuuuuucaccauugucuuguccugcauguguggguuguugguuauccauacucuguuccacaccucaagcaugucuuccguggucauccacuccccuuucccaugaaucgaccauguugugcguccuuguggaacccaugaggugggaacagcugaggaaacagccaaugacaguagccucaugucccuuuugugaaaauacaucagugaccacauguuggcauaggcuuugcugaggcaagcuguuuccuugaucauccagccguuuccuggagacacccuuccucucccaaugagcucguccuguucucggcaaggcaccacaauccuccugccauccuucagcuguaguucauggaaguggugggaacagaagggcacauucucccaaucauuccacccuuuugauggcugccauucagauauguccuuucuaaccuuggacauggcguugagaugggacagggccaggccgaaccugucaucgaugggccggaccacacagucgucuccacucaccgccauccucuucagucugucacauccgugcucagugagccaugccuccagccuggucagaacugauucaucacaaucuugaacauguuggugauguaucaccaucucugcuucugccauucugaucaauuggacuuucaaguuggugaugguguucagagcauaagucacuaccugcccggauccucucuggucucgucgacuuaugacauccauguaggcuuucccuccuggggcuggucucaacacuuucaccacuuuguucuuguaugucauuuccaucacugcuugugccaguuuuuugugaugugggcucauguaguucaagaucuccuguucaucaucaaggucugccucugugaugcgcgugucccauccagcggugucauccgcguagaauccaccaccauccauugcagccaggucucugaucacauauccuagguauuguaagccaaugccuuccacuccuccuccugaguuuucccuggaagcccaaugguccucauucaggaaucccagggccucaaacucaagauaccgcgcucccagccacauauaccauauggcacggcuucccuuugcuuucccaaacucugacagcuucuucucucuuuuccccaucauguuguacacacaaguccgacaccugccuuguuggugcagcuuccuuucuucauccaccaguucccagaacuuugggucuuggacagccucauuggcagucuuccacuguucuuguucuuccagguaagcuccaauggcugcaugacuucggacuuuugcaauaaauucuuccuuugugcacagucugggguucuuuucucuggccagguggcggaacagccaccuguugacaacuuucaugaucuuccuaguucccgcugguggauccuuugcucuggugucaacuuuuucuuuaaacacucuuugcuguccaaaagggguugugucagucauugccauucuugugaccuccucuauccugucccauggauaugucagaauuuuaauaacaccauuuaccaugcucgccgcacuuccugagguuuuugugacauaggagccacaguagugccagguccuguagggguugucauugucauaaaaccaagaggucauguacucagauuuuauccucucaacccuuucuucuauggccucuuuguccaggggucccuugucugucucaacacugcgugucccaauugggaggaugacgucagccuccagggucacuuuuccaguuggacgccucauucuccucaucaggaggcgggauguuugguucacaguaaaugugacauugcugcgggcuccagacacguaguacauuucaugaguggaauuccuggagagaggguuccugaucacuguuccgccaaaccuccuuuggagcaauuccaguuucucgagaacaucuggcauguauggagcuaacaccuucacacagaaguugucaaccccacaagccagccauuuuucuacaguaucaagaacucucacgguccuuucccccucugugaccgaugacgaugaugacucuccaaugucacacaaaagggugucacauuucacugguucuaggcgguggauaucaguuuuguccuugaaggugaugauguuccaucccagacuuugcacauucauggguuucucauggccgucucuuccaagaguaaauccuuugaccccacucacuuccuuuugcgcagcagcguaguaacaccagccuccgcggccacaccccaggucaaucacccuaccuuccagcuugacauagccacgcucauggaaccaccuuaacuuugcggucccccuggagaccgccaccccgguguccaccuucccuucggccaaaugccugcgugccguaucacgauccaccuccacaaugucgguccuuuuauacaacucaaacugucgcuuguccaacagauucaguucccucuuccagacuucacccaaaguuuuuccauucgcgcucccccggcguccaguuuucaucuuccauagauuguacaugacucccacaaaagcauagugauucccccucaugacuccugucauggagacagccauggguccauuccaaagaaggcugguguuucccucuaugagcggcccuaaggcagcugaugcuaggacaaugccuucagccaaugaaaagggcguucugcacauggcaacagaagcuaggcugagagcaagaaggagauauagagccaguuucuucucauaaagggcaggcauuucaggagcuuccucaaugucaacuguuggauucccaucaaccacaggguucucggcaacgccauggaacacccuucucugugcaagcuuugacugcugcgcuuugauuccagguaaaaugagagaccaguggagcauggcgcacccuaugccacagagcagaggcaucacuguuauugaauuccagccacugaccagcagcauuaugaccgagauauucaucuucaugaaugguauccccuuguccaugaaagaaaggacugaggcugacugggcuauuccagacagagacagguugccauauucgacuuugauccaguggugcaacauuggagagagcauuguaacaaugccaacguacacuguccaggcagcuccuggcuucaggucaagauccggccaacuccagggugaagcacuagauggaauuaaguucuucuucccaaagagguccucuuugguuuucuccagcaugccuagcucguuggcugccaccgcugaaaccagcgucaggaugccaauaaugagguaugccacuugguugucuuggauggaccuuuguugcccuggcucggggaucacaaccaccaucaggacaaagaauaugagcaugacauaggagaugugaguggguuugacgccuccaaggaacaugagauauccacagccggccauugugcccaucgccauagacauucuacugaugccuuugggagacaugaaaaagaugaccauucccgaugucaguaguccagccaguauaaacagcaugacuauugucauugccucaggcaucauugauagugcauugcgguaagcccuagagccuuccucagaguggaggaacacacugaugguauccauugccucuccaccuuuuuuagccaggaaaucagggaguucacucagcacaacuagcacuucagcagcuccccuccuaccuucagcaaacuuaauaaauucagacagcgcacucuggucagaugacacccuuucaucacaccaccuugggcgcagaggcuucuuugcuccuccaggagcccugcacuucacuguuucaccgcugucauucaagaucucauguuccucagggccuucaaaacaccacuuacgaucauucgucuucaaaccagccuuggccacuugccacgaaagccaaacgggcaggucacaauuccucacuaguucucugaagacuuuccucuggucaucccucagucucauuucaccaggggaaacugguguuuuaguuccuucaacgccauagaguggggcgaccauuccaccccucaccuccauguuguccaagagcauugaggccuccaaccagcagacguggugggcauuauuuucacuuguaggcucagaauaguaguaugagucuccaucucuguugggauuucucccaaugcgcccccuccuuugagcagcagaggaugcggagauacgaaguggcccuuuuauugccaccuuccucccuucauccacaagcacaggcuuaaaagccguccugcaauccagcacucgcuccacgcaaagguuggcucccauuucagcuaugucaguggccaauauaaagucagguuucuucugcuuuaucgugggguauucucucucaaagguuuuccuguucaggaccaccacacucuuuccagccuuacgcaaagaggcagccaugacauuugcagcucugauggauggaaggaaccaugccgugggccuuuugucagcuaggauccagucaugcccuguguuccagggcucacuggguauguccguuugaacaucuucuauuucaccauuugaauguggaaauucaucacuagucccaggcgguguggcugucaucaagauuguugcacuuucauuugcccuagcucugugcgcugcccaaccucuagcggcuaugcuagcuggauccaaaaaaugggcuucauccauaaugaucacuucccaguuaacaacccuaguugguuccaacauccuguaaguuaggguggcauggcacauggcaucaaugacuucucucccgcugccgugagcggaaaaagccuguguguggaauuucacguccaggccgugaaaagccuccuucauuucagaaagaacaacccuggugggggccaacacaagagugcgcaagcgucuccgugcgcacucggccaagaucugugggaggaaacgucuugucuucccagcuccaggaugaaaaucaaggacaguugucauuccuuucuuuagcauugucgggaucucuuggagcuccuccuuuccuucuuccuucaccucagucugggauauggcggacacgaaggaguugucaccgacaaggaugccauugccguacagcccaaucaccucuccguuccuguuaacaauaggagauccugaagugccacucggauagucaagagcgacagccccgauuucucccccauuccucacuuugaacaagcucgguuuugucuggacguugaccacguucuuuccuggaacagccgcgaucaacuggaccucuuccucuccaucccaucugccuuccaacuuccaugagccaccauaggcgacaaggucuuccuuuacugaagcccaagauggaaucaacuucuugccauuccugacaaggaaagcuccucuugugacaugccacauuguguggaacaccccucccugugccacucccacuccucgcugggaggcccccaagaagguugacuggaauaugccauaaaucccauccuccagauguucacauuccucgaugaucuuaggagugggaauaucccacaagacauccccacuucuccuagcuccccugacaugaaacagccacccagcaaggaccagcagaagagcaaauggauggagggcagccccaaccaaggccagcgaggucaucacaaccuggucccauggcacuuucucuucagaaagcagcuugaacuccccuuguucacugagugccacaucauagcgggcggaacucccgcugaucuccgccuccucuucccaugaaacuucaccaagcuucuugagcucuagcccauccacccucccagccacgcuaaccagcaucaucaggaguccuccaacugcaaucggaccaaggaaguucuccaucuccugaaaagccaguccugccagcacucccacuagaccagcugcugcgagugccucauucacugggauacuccuucgcccaaauaugcggguugccagaaaugcacacaggcccaaaaaagguugugucaagcccagguaagaugugagugugagggccaccagagguauagucuucugcauggagguguccuugaaauucuggugaaggaccccuaugauaaccacggcacaaaagaacauugcggcaagucucaccucagccauagugacaggugucaacagagccaugaggggcaagaugguauuugaugcuuuccuagaagcaacagcauuuauugucaggaugcagagagaaacugcauuuagauacuuccacaggccgcccaucacgccacccaaggcaaucuccaccauggcugcuccuagggucagcacaaggcguucccgagggcuccauaggguccugagcccaaagccgaugagcagcccuggucugauugaaaaggcagcaaucaacgccauauacauggcgucuccuccauuguucaucucauggaaaugcaaucccacagccacugugaguuucagcaaaucaaggagaguuacuugcccgaccagcauugcucccaagagcacuacuccuccaaccaacauuugcuuuggucccugucuuuuccuuaggaccacuuccauugcuaucaucaugcucaccaaaccaaaagggacagcauguauuucuccagcuguaacccaggagcgcaccagauggcuuucaugcguuuuccuuggccuaauuuccaugggauaccaacacccaucacuaccauggaagcucacaggcggcauugugcaggagcggcaacaccauucaggaauaacuuucccgcuauccgugguggaucugguugauuuuccccguccaucacaguugccaucaaugaucacgcuagucccugggcaagcuucucucuucacuucuagugguaccugcauccaagguccguucgucugaaccuuguauccagggauaugauugugagagcuaacugggccuccgauugaucucggcaugaacauuucacucucuucaacugauguuccaaucguaugugucaguggccacucacacuccuuguaaucuaaugccuccaagguguggaucauccaugucccauuuacuucaugacuucccauccaaaauguuggagagccaugggcacucuuuuuuccguucaccgcugcacccaagauagauccaucgcagucuaugguguauucaaagacugcguccauguacacgcguguggugaacacucccgucccaaacuccucuaucuggaaagaauuccagacccgguuugaaaacgggcauucuuuccuggacuuuccaucuaugaugaagcuuccauucuuccucccuggggagaacacaagguucuuaccccaagucuuccaaccauacugcagaccaucccgaauucuggaaaauggaugaguuccucucugguaaacauucuuuggauccugcacgacaacagaaauguccaccucguuuuccucaaaaauggcauugaucucaucugcccugcuucuccacaucucaugcucaagggagucaacugaauuuaggccacacuucccuucuucaaaagaggcuuucacuauugaugcaagcuucacaggaucuucuggauaguaugaguacuuguucagccagucaucagagucucuaaauaugaagauaccaucuccgcacuugagcucucucuugccaaaguugauggcgcauccuugauccgccccaacuccuagagacaaaaacaucaugaucacuccuaccaagaucaugcucauggacauugucauguuucuuguguugaugccaacccauauaaguaccgcccccaugaugaccuuuguuauccaguucaagccgccaaauagccccugaaaggcagagccaaacaccguaugaauuccuuucccaaccgaagugaagaacccuccagcggagcugaaaucccaggcggugucucccaugacggccaggcguuccacgccuuucauggucugagugaacaacuuuccuauugagcuucccucuuugugccacugguaagugagacgugaaucuccucucccaacgauaauguagcugucuccaaaagguggguucaccucaaucagcacuucaucaucauugguugaggcgaugggguuaacuguaaccaaaaugccuuuauugauugccgcuguaagaucaucagcuacuaucacuggaauccugcagggggcuccuuuugacacuuucaccugcaucacaacagugccauggccagugucaguuggguucuugacaaaaaacauuuugucagugcauauuuuguaggauguccccuugagugucaaagcugacaauuucacucugcaagaaacauguccaccauguaguuuguaaagguuguugucauuuguguccuuuguaacccucauugcgccaguaagagcuguuuucaaggagccuuccugguuucccagggccaguacucugauaguggcggcaugcggagguucaaauucgacaagaugaugcaucucucuccacaccccgccacuuccacucugccauggcagggucaaguccugggcccacugucuguccacuauccagcucucuguuuccaucucagcgauguaacuguuaccaaaguccaccgcaguuugcaccuggcauuccaguguagcuuuuccauacccaaugaacucgacuuccugggagccugacagggcaucaaacuugagagucuuaaugucgguauuccaauuuuccugcuuggccccuacaugcaauugugcucugaugacauacugaauuuuggucugaucaaccucaaacaaacucauggauuuggcacaagugaauuuggcgcaugccacaaugcucccuuucccaaauaggccacagccauugccccagccucuaucagaauaagugcgcuugcacgcauuguccccuucguucucuucagcuaggugggccucuccagugcuggggcacuugucauuaaucuucacaugagugagaacugcauuguaacacacuuuccucaccucagcaggucuaucaauggcuacugucucuagugagauguccaaugaaggcuugucaggggccauaacagugacacacuugucuugcuccaggguagcugaaacccaaguuccuccaugcacccccucaaugaaaucccugucaguaauuccaaugcagugagcugaguaggccggaccaacagccaagaccaguagggcaaucacgacucguugcgucauguugcuucccacaagguaggcaauggucagagccgucacugcaaaaaagggguuccucacgaaccaucucucaaucuuuuggaguugccuuucacccauucuuccagucauccauuuuucuugccgggucuucaaaccaugguuuucaugcguaggcaagucaauggcccuucuugaccuccuagaccugccugcugagucacacuuaccauaugcgacucuaacguuuuccaccccauagcaccagcaaucaaugucaucuggcuccucucuuggacugagauugggacaguuguauuccauugagucugggcaccaguacuuggcuuccaaaauguuuguugugcaguugccugugcccacagagaauguuuucccgagguccucagaugucacauuuaggagcaaccaucuguuuuuccgcaccaaggucacuccacccgucaucaacagcauucccaaaauuaggaauugcacagucagaacaucaugggaacggcguuuccuugaggacaauccucucaucaaacuggccaccacucucuugacuuuccuuagaacagccaagccuugucuuggguccagcauuuuccacaaccucuuuaggugggcugugaucuuuuuuccagucaaaauguugaacaaaaagaaaaagauaaauccuugaacaccucuugaagguccaggucuguuuccaauuuguuuuguuuuuuguuuuauuuuguuugacaaggagcgaacuccucgucguaccauauugacgcccaggguuuuucccugagcuuuacgaccagacauguucuggucaguucucugcuaaucgcucaacgaacgauuaaaauuaauccaaauguguuuauugccuagcaacucgauuugcagaccaaugcaccucaauuagcacacaggauuuacu

SEQ ID NO: 14 - DNA, 13220 bp, Artificial Sequence
  plasmid construct
gttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgagagtggttttgtgtttgtcatccaaaggtctgcttattcttgagcaaactgtgctcacagacctctggaggaaaagcagagaaccactccggtctttccctggcgtcaatatggtcccactatttgttccctggagggtctcctctaacctctagaccccgtctttctaccaccacgtgggagggtggtagcggaggctccgttctttttactctggggtgggaggtcccagaaaccaggtttttcgcaacctggaggtcggctgtcccagcctgcactgcctcacagcttagcagtggcaaaactcgtgtggggttctgggctgacaacttcttctgtctcaatgtgtggagtccggttctccatccgtagtttttatcccggtttctgtttcattttaagtgcggagcccggttctccagccgtggtttatatcccggtttcaggttgtggggagtccggttctccacccgtggtttgtatcccggttattcctgttagatggtgtttcagataagctcacccagttgcaggtcagcatccacagaatacctgtccatgactgttaggtagtcagtgtatttctcctgtccaatcagcgttcggatacgatggatgactaaatggatgtgggaggcccaggtggccctattggtcattccaatcagtgatccgcacagcttgtcttgtctcttggttagataagggacatctctccattttttcaccattgtcttgtcctgcatgtgtgggttgttggttatccatactctgttccacacctcaagcatgtcttccgtggtcatccactcccctttcccatgaatcgaccatgttgtgcgtccttgtggaacccatgaggtgggaacagctgaggaaacagccaatgacagtagcctcatgtcccttttgtgaaaatacatcagtgaccacatgttggcataggctttgctgaggcaagctgtttccttgatcatccagccgtttcctggagacacccttcctctcccaatgagctcgtcctgttctcggcaaggcaccacaatcctcctgccatccttcagctgtagttcatggaagtggtgggaacagaagggcacattctcccaatcattccacccttttgatggctgccattcagatatgtcctttctaaccttggacatggcgttgagatgggacagggccaggccgaacctgtcatcgatgggccggaccacacagtcgtctccactcaccgccatcctcttcagtctgtcacatccgtgctcagtgagccatgcctccagcctggtcagaactgattcatcacaatcttgaacatgttggtgatgtatcaccatctctgcttctgccattctgatcaattggactttcaagttggtgatggtgttcagagcataagtcactacctgcccggatcctctctggtctcgtcgacttatgacatccatgtaggctttccctcctggggctggtctcaacactttcaccactttgttcttgtatgtcatttccatcactgcttgtgccagttttttgtgatgtgggctcatgtagttcaagatctcctgttcatcatcaaggtctgcctctgtgatgcgcgtgtcccatccagcggtgtcatccgcgtagaatccaccaccatccattgcagccaggtctctgatcacatatcctaggtattgtaagccaatgccttccactcctcctcctgagttttccctggaagcccaatggtcctcattcaggaatcccagggcctcaaactcaagataccgcgctcccagccacatataccatatggcacggcttccctttgctttcccaaactctgacagcttcttctctcttttccccatcatgttgtacacacaagtccgacacctgccttgttggtgcagcttcctttcttcatccaccagttcccagaactttgggtcttggacagcctcattggcagtcttccactgttcttgttcttccaggtaagctccaatggctgcatgacttcggacttttgcaataaattcttcctttgtgcacagtctggggttcttttctctggccaggtggcggaacagccacctgttgacaactttcatgatcttcctagttcccgctggtggatcctttgctctggtgtcaactttttctttaaacactctttgctgtccaaaaggggttgtgtcagtcattgccattcttgtgacctcctctatcctgtcccatggatatgtcagaattttaataacaccatttaccatgctcgccgcacttcctgaggtttttgtgacataggagccacagtagtgccaggtcctgtaggggttgtcattgtcataaaaccaagaggtcatgtactcagattttatcctctcaaccctttcttctatggcctctttgtccaggggtcccttgtctgtctcaacactgcgtgtcccaattgggaggatgacgtcagcctccagggtcacttttccagttggacgcctcattctcctcatcaggaggcgggatgtttggttcacagtaaatgtgacattgctgcgggctccagacacgtagtacatttcatgagtggaattcctggagagagggttcctgatcactgttccgccaaacctcctttggagcaattccagtttctcgagaacatctggcatgtatggagctaacaccttcacacagaagttgtcaaccccacaagccagccatttttctacagtatcaagaactctcacggtcctttccccctctgtgaccgatgacgatgatgactctccaatgtcacacaaaagggtgtcacatttcactggttctaggcggtggatatcagttttgtccttgaaggtgatgatgttccatcccagactttgcacattcatgggtttctcatggccgtctcttccaagagtaaatcctttgaccccactcacttccttttgcgcagcagcgtagtaacaccagcctccgcggccacaccccaggtcaatcaccctaccttccagcttgacatagccacgctcatggaaccaccttaactttgcggtccccctggagaccgccaccccggtgtccaccttcccttcggccaaatgcctgcgtgccgtatcacgatccacctccacaatgtcggtccttttatacaactcaaactgtcgcttgtccaacagattcagttccctcttccagacttcacccaaagtttttccattcgcgctcccccggcgtccagttttcatcttccatagattgtacatgactcccacaaaagcatagtgattccccctcatgactcctgtcatggagacagccatgggtccattccaaagaaggctggtgtttccctctatgagcggccctaaggcagctgatgctaggacaatgccttcagccaatgaaaagggcgttctgcacatggcaacagaagctaggctgagagcaagaaggagatatagagccagtttcttctcataaagggcaggcatttcaggagcttcctcaatgtcaactgttggattcccatcaaccacagggttctcggcaacgccatggaacacccttctctgtgcaagctttgactgctgcgctttgattccaggtaaaatgagagaccagtggagcatggcgcaccctatgccacagagcagaggcatcactgttattgaattccagccactgaccagcagcattatgaccgagatattcatcttcatgaatggtatccccttgtccatgaaagaaaggactgaggctgactgggctattccagacagagacaggttgccatattcgactttgatccagtggtgcaacattggagagagcattgtaacaatgccaacgtacactgtccaggcagctcctggcttcaggtcaagatccggccaactccagggtgaagcactagatggaattaagttcttcttcccaaagaggtcctctttggttttctccagcatgcctagctcgttggctgccaccgctgaaaccagcgtcaggatgccaataatgaggtatgccacttggttgtcttggatggacctttgttgccctggctcggggatcacaaccaccatcaggacaaagaatatgagcatgacataggagatgtgagtgggtttgacgcctccaaggaacatgagatatccacagccggccattgtgcccatcgccatagacattctactgatgcctttgggagacatgaaaaagatgaccattcccgatgtcagtagtccagccagtataaacagcatgactattgtcattgcctcaggcatcattgatagtgcattgcggtaagccctagagccttcctcagagtggaggaacacactgatggtatccattgcctctccaccttttttagccaggaaatcagggagttcactcagcacaactagcacttcagcagctcccctcctaccttcagcaaacttaataaattcagacagcgcactctggtcagatgacaccctttcatcacaccaccttgggcgcagaggcttctttgctcctccaggagccctgcacttcactgtttcaccgctgtcattcaagatctcatgttcctcagggccttcaaaacaccacttacgatcattcgtcttcaaaccagccttggccacttgccacgaaagccaaacgggcaggtcacaattcctcactagttctctgaagactttcctctggtcatccctcagtctcatttcaccaggggaaactggtgttttagttccttcaacgccatagagtggggcgaccattccacccctcacctccatgttgtccaagagcattgaggcctccaaccagcagacgtggtgggcattattttcacttgtaggctcagaatagtagtatgagtctccatctctgttgggatttctcccaatgcgccccctcctttgagcagcagaggatgcggagatacgaagtggcccttttattgccaccttcctcccttcatccacaagcacaggcttaaaagccgtcctgcaatccagcactcgctccacgcaaaggttggctcccatttcagctatgtcagtggccaatataaagtcaggtttcttctgctttatcgtggggtattctctctcaaaggttttcctgttcaggaccaccacactctttccagccttacgcaaagaggcagccatgacatttgcagctctgatggatggaaggaaccatgccgtgggccttttgtcagctaggatccagtcatgccctgtgttccagggctcactgggtatgtccgtttgaacatcttctatttcaccatttgaatgtggaaattcatcactagtcccaggcggtgtggctgtcatcaagattgttgcactttcatttgccctagctctgtgcgctgcccaacctctagcggctatgctagctggatccaaaaaatgggcttcatccataatgatcacttcccagttaacaaccctagttggttccaacatcctgtaagttagggtggcatggcacatggcatcaatgacttctctcccgctgccgtgagcggaaaaagcctgtgtgtggaatttcacgtccaggccgtgaaaagcctccttcatttcagaaagaacaaccctggtgggggccaacacaagagtgcgcaagcgtctccgtgcgcactcggccaagatctgtgggaggaaacgtcttgtcttcccagctccaggatgaaaatcaaggacagttgtcattcctttctttagcattgtcgggatctcttggagctcctcctttccttcttccttcacctcagtctgggatatggcggacacgaaggagttgtcaccgacaaggatgccattgccgtacagcccaatcacctctccgttcctgttaacaataggagatcctgaagtgccactcggatagtcaagagcgacagccccgatttctcccccattcctcactttgaacaagctcggttttgtctggacgttgaccacgttctttcctggaacagccgcgatcaactggacctcttcctctccatcccatctgccttccaacttccatgagccaccataggcgacaaggtcttcctttactgaagcccaagatggaatcaacttcttgccattcctgacaaggaaagctcctcttgtgacatgccacattgtgtggaacacccctccctgtgccactcccactcctcgctgggaggcccccaagaaggttgactggaatatgccataaatcccatcctccagatgttcacattcctcgatgatcttaggagtgggaatatcccacaagacatccccacttctcctagctcccctgacatgaaacagccacccagcaaggaccagcagaagagcaaatggatggagggcagccccaaccaaggccagcgaggtcatcacaacctggtcccatggcactttctcttcagaaagcagcttgaactccccttgttcactgagtgccacatcatagcgggcggaactcccgctgatctccgcctcctcttcccatgaaacttcaccaagcttcttgagctctagcccatccaccctcccagccacgctaaccagcatcatcaggagtcctccaactgcaatcggaccaaggaagttctccatctcctgaaaagccagtcctgccagcactcccactagaccagctgctgcgagtgcctcattcactgggatactccttcgcccaaatatgcgggttgccagaaatgcacacaggcccaaaaaaggttgtgtcaagcccaggtaagatgtgagtgtgagggccaccagaggtatagtcttctgcatggaggtgtccttgaaattctggtgaaggacccctatgataaccacggcacaaaagaacattgcggcaagtctcacctcagccatagtgacaggtgtcaacagagccatgaggggcaagatggtatttgatgctttcctagaagcaacagcatttattgtcaggatgcagagagaaactgcatttagatacttccacaggccgcccatcacgccacccaaggcaatctccaccatggctgctcctagggtcagcacaaggcgttcccgagggctccatagggtcctgagcccaaagccgatgagcagccctggtctgattgaaaaggcagcaatcaacgccatatacatggcgtctcctccattgttcatctcatggaaatgcaatcccacagccactgtgagtttcagcaaatcaaggagagttacttgcccgaccagcattgctcccaagagcactactcctccaaccaacatttgctttggtccctgtcttttccttaggaccacttccattgctatcatcatgctcaccaaaccaaaagggacagcatgtatttctccagctgtaacccaggagcgcaccagatggctttcatgcgttttccttggcctaatttccatgggataccaacacccatcactaccatggaagctcacaggcggcattgtgcaggagcggcaacaccattcaggaataactttcccgctatccgtggtggatctggttgattttccccgtccatcacagttgccatcaatgatcacgctagtccctgggcaagcttctctcttcacttctagtggtacctgcatccaaggtccgttcgtctgaaccttgtatccagggatatgattgtgagagctaactgggcctccgattgatctcggcatgaacatttcactctcttcaactgatgttccaatcgtatgtgtcagtggccactcacactccttgtaatctaatgcctccaaggtgtggatcatccatgtcccatttacttcatgacttcccatccaaaatgttggagagccatgggcactcttttttccgttcaccgctgcacccaagatagatccatcgcagtctatggtgtattcaaagactgcgtccatgtacacgcgtgtggtgaacactcccgtcccaaactcctctatctggaaagaattccagacccggtttgaaaacgggcattctttcctggactttccatctatgatgaagcttccattcttcctccctggggagaacacaaggttcttaccccaagtcttccaaccatactgcagaccatcccgaattctggaaaatggatgagttcctctctggtaaacattctttggatcctgcacgacaacagaaatgtccacctcgttttcctcaaaaatggcattgatctcatctgccctgcttctccacatctcatgctcaagggagtcaactgaatttaggccacacttcccttcttcaaaagaggctttcactattgatgcaagcttcacaggatcttctggatagtatgagtacttgttcagccagtcatcagagtctctaaatatgaagataccatctccgcacttgagctctctcttgccaaagttgatggcgcatccttgatccgccccaactcctagagacaaaaacatcatgatcactcctaccaagatcatgctcatggacattgtcatgtttcttgtgttgatgccaacccatataagtaccgcccccatgatgacctttgttatccagttcaagccgccaaatagcccctgaaaggcagagccaaacaccgtatgaattcctttcccaaccgaagtgaagaaccctccagcggagctgaaatcccaggcggtgtctcccatgacggccaggcgttccacgcctttcatggtctgagtgaacaactttcctattgagcttccctctttgtgccactggtaagtgagacgtgaatctcctctcccaacgataatgtagctgtctccaaaaggtgggttcacctcaatcagcacttcatcatcattggttgaggcgatggggttaactgtaaccaaaatgcctttattgattgccgctgtaagatcatcagctactatcactggaatcctgcagggggctccttttgacactttcacctgcatcacaacagtgccatggccagtgtcagttgggttcttgacaaaaaacattttgtcagtgcatattttgtaggatgtccccttgagtgtcaaagctgacaatttcactctgcaagaaacatgtccaccatgtagtttgtaaaggttgttgtcatttgtgtcctttgtaaccctcattgcgccagtaagagctgttttcaaggagccttcctggtttcccagggccagtactctgatagtggcggcatgcggaggttcaaattcgacaagatgatgcatctctctccacaccccgccacttccactctgccatggcagggtcaagtcctgggcccactgtctgtccactatccagctctctgtttccatctcagcgatgtaactgttaccaaagtccaccgcagtttgcacctggcattccagtgtagcttttccatacccaatgaactcgacttcctgggagcctgacagggcatcaaacttgagagtcttaatgtcggtattccaattttcctgcttggcccctacatgcaattgtgctctgatgacatactgaattttggtctgatcaacctcaaacaaactcatggatttggcacaagtgaatttggcgcatgccacaatgctccctttcccaaataggccacagccattgccccagcctctatcagaataagtgcgcttgcacgcattgtccccttcgttctcttcagctaggtgggcctctccagtgctggggcacttgtcattaatcttcacatgagtgagaactgcattgtaacacactttcctcacctcagcaggtctatcaatggctactgtctctagtgagatgtccaatgaaggcttgtcaggggccataacagtgacacacttgtcttgctccagggtagctgaaacccaagttcctccatgcaccccctcaatgaaatccctgtcagtaattccaatgcagtgagctgagtaggccggaccaacagccaagaccagtagggcaatcacgactcgttgcgtcatgttgcttcccacaaggtaggcaatggtcagagccgtcactgcaaaaaaggggttcctcacgaaccatctctcaatcttttggagttgcctttcacccattcttccagtcatccatttttcttgccgggtcttcaaaccatggttttcatgcgtaggcaagtcaatggcccttcttgacctcctagacctgcctgctgagtcacacttaccatatgcgactctaacgttttccaccccatagcaccagcaatcaatgtcatctggctcctctcttggactgagattgggacagttgtattccattgagtctgggcaccagtacttggcttccaaaatgtttgttgtgcagttgcctgtgcccacagagaatgttttcccgaggtcctcagatgtcacatttaggagcaaccatctgtttttccgcaccaaggtcactccacccgtcatcaacagcattcccaaaattaggaattgcacagtcagaacatcatgggaacggcgtttccttgaggacaatcctctcatcaaactggccaccactctcttgactttccttagaacagccaagccttgtcttgggtccagcattttccacaacctctttaggtgggctgtgatcttttttccagtcaaaatgttgaacaaaaagaaaaagataaatccttgaacacctcttgaaggtccaggtctgtttccaatttgttttgttttttgttttattttgtttgacaaggagcgaactcctcgtcgtaccatattgacgcccagggtttttccctgagctttacgaccagacatgttctggtcagttctctgctaatcgctcaacgaacgattaaaattaatccaaatgtgtttattgcctagcaactcgatttgcagaccaatgcacctcaattagcacacaggatttactcctatagtgagtcgtattagcggccgccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgt